(19) United States
(12) Patent Application Publication
(10) Pub. No.: US 2025/0066386 A1
(43) Pub. Date: Feb. 27, 2025

(54) INHIBITORS OF KRAS

(71) Applicant: BIOMEA FUSION, INC., Redwood City, CA (US)

(72) Inventors: Thorsten A. Kirschberg, San Carlos, CA (US); Solomon B. Ungashe, Sunnyvale, CA (US); Thu Phan, Fremont, CA (US); Yongli Su, Foster City, CA (US); Xiaodong Wang, Millbrae, CA (US); James T. Palmer, Warrandyte (AU); Thomas Butler, Redwood City, CA (US); Ravindra B. Upasani, San Jose, CA (US); Nan-Horng Lin, St. Vernon Hill, IL (US); David Sperandio, San Carlos, CA (US); Neil Howard Squires, San Francisco, CA (US); Amna Trinity-Turjuman Adam, Mountain View, CA (US)

(21) Appl. No.: 18/690,517

(22) PCT Filed: Nov. 8, 2022
(86) PCT No.: PCT/US2022/049314

§ 371 Date: Mar. 8, 2024

(51) Int. Cl. C07D 498/18; A61K 31/4439; A61K 31/504; A61K 31/5377; C07D 487/22; C07D 513/22

(52) U.S. Cl. CPC C07D 498/18; A61K 31/4439; A61K 31/504; A61K 31/5377; C07D 487/22; C07D 513/22

(57) ABSTRACT

Disclosed herein are macrocyclic compounds that inhibit the binding of KRas. Also disclosed are pharmaceutical compositions that include the compounds. Methods of using the KRas inhibitors are disclosed, alone or in combination with other therapeutic agents, for the treatment of autoimmune diseases or conditions, heteroimmune diseases or conditions, cancer, including lymphoma, leukemia, lung cancer, colorectal cancer, pancreatic cancer, and other diseases or conditions dependent on KRas interaction.

### CROSS REFERENCE

The present application claims the benefit of US provisional application nos. 63/277,581, filed Nov. 9, 2021, and 63/376,234, filed Sep. 19, 2022, the entire contents of which are hereby incorporated by reference in their entireties.

### FIELD

Described herein are compounds, methods of making such compounds, pharmaceutical compositions, and medicaments containing such compounds, and methods of using such compounds and compositions to inhibit the activity of KRas.

### BACKGROUND

Kirsten Rat Sarcoma 2 Viral Oncogene Homolog (“KRas”) was identified in 1982 as an oncogene encoding the p21 transforming protein of Kirsten murine sarcoma virus. Tsuchida et al., 1982, Science 217:937-939. A single amino acid substitution activates the oncogene, which is implicated in a number of cancers including lung adenocarcinoma, mucinous adenoma, ductal carcinoma of the pancreas, and colorectal cancer. Chiosea et al., 2011, Modern Pathology. 24: 1571-7; Hartman et al., 2012, International Journal of Cancer. 131:1810-7; and Krasinskas et al., 2013, Modern Pathology. 26: 1346-54. KRas mutations underlie up to 20% of human cancers. Cox et al., 2014, Nature Reviews. Drug Discovery. 13: 828-51

AMG-510 (Sotorasib) was recently approved for treatment of adult patients with KRAS G12C-mutated locally advanced or metastatic non-small cell lung cancer (NSCLC).

Several additional clinical trials are underway targeting KRas or mutant KRas. MRTX849 (Adagrasib) is being developed for the potential treatment of patients with non-small cell lung cancer (NSCLC). Since KRas is implicated in a large number of human cancers, additional candidate therapeutics are needed.

### SUMMARY

In one aspect, described herein are inhibitors of KRas G12C. Also described herein are specific heterocyclic inhibitors of KRas G12C. In some embodiments, the inhibitors of KRas G12C are irreversible inhibitors. In some embodiments, the inhibitors of KRas G12C are reversible inhibitors.

In another aspect, described herein are methods for synthesizing such inhibitors, methods for using such inhibitors in the treatment of diseases (including diseases wherein inhibition of KRas G12C provides therapeutic benefit to a patient having the disease). Further described are pharmaceutical compositions that include an inhibitor of KRas G12C. Specifically, described herein are compounds and methods of use thereof to inhibit KRas G12C.

In another aspect, described herein are inhibitors of KRas G12D. Also described herein are specific heterocyclic inhibitors of KRas G12D. In some embodiments, the inhibitors of KRas G12D are irreversible inhibitors. In some embodiments, the inhibitors of KRas G12D are reversible inhibitors.

In another aspect, described herein are inhibitors of KRas G12V, G12R, G12A, G13C, G13D, Q61H or Q61R. Also described herein are specific heterocyclic inhibitors of KRas G12V, G12R, G12A, G13C, G13D, Q61H or Q61R. In some embodiments, the inhibitors of KRas G12V, G12R, G12A, G13C, G13D, Q61H or Q61R D are irreversible inhibitors. In some embodiments, the inhibitors of KRas G12V, G12R, G12A, G13C, G13D, Q61H or Q61R are reversible inhibitors.

In another aspect, described herein are methods for synthesizing such inhibitors, methods for using such inhibitors in the treatment of diseases (including diseases wherein inhibition of KRas G12D provides therapeutic benefit to a patient having the disease). Further described are pharmaceutical compositions that include an inhibitor of KRas G12D. Specifically, described herein are compounds and methods of use thereof to inhibit KRas G12D.

In a particular aspect described herein are compounds and methods of use thereof to inhibit KRas G12C.

Thus, in some embodiments, provided herein are compounds according to Formula (B-I) having the structure:

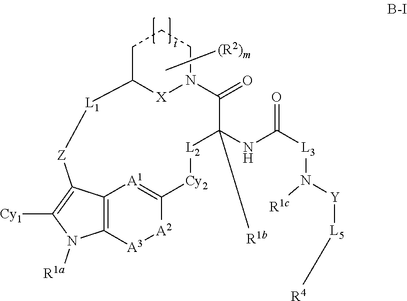

or a stereoisomer or a pharmaceutically acceptable salt thereof,
wherein:
each A¹, A², and A³ is independently —CR³=, or —N=; each R is independently H, halo, cyano, alkyl, haloalkyl, hydroxy, or alkoxy;
X is —CR^{1e}R^{1f}—, or —NR^{1g}—;
Z is absent or substituted or unsubstituted C₁-C₄ alkylene;
each Cy¹ and Cy² is independently substituted or unsubstituted aryl or heteroaryl;
Cy³ is substituted or unsubstituted cycloalkylene, or substituted or unsubstituted heterocycloalkyl;
L¹ is —C(O)—O—, —C(O)—N(R^{1d})—, or -Cy⁴-; Cy⁴ is substituted or unsubstituted heteroaryl; and —O— of —C(O)—O—, or —N(R^{1d})— of —C(O)—N(R^{1d})— is attached to Z; or —O— of —C(O)—O—, or —N(R^{1d})— of —C(O)—N(R^{1d})— is attached to the pyrrolo ring when Z is absent;
each L₂, and L₃ is independently substituted or unsubstituted C₁-C₄ alkylene;
Y is —C(O)—, —C(H)(CH₂F)—, —C(H)(CHF₂)—, or —C(H)(CF₃)—;
L₅ is substituted or unsubstituted C₁-C₆ alkylene, or Cy₃;
each R^{1a}, R^{1b}, R^{1c}, R^{1d}, R^{1e}, R^{1f}, and R^{1g} is independently H, or substituted or unsubstituted C₁-C₄ alkyl; each R² is independently halo, cyano, alkyl, haloalkyl, or alkoxy; m is 0, 1, or 2;
R⁴ is any one of groups selected from i), and ii):
i) —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl;
ii) -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e},
L₄ is C(O), substituted or unsubstituted C₁-C₄ alkylene, or substituted or unsubstituted cycloalkyl;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, unsubstituted or substituted with one or more groups selected from substituted or unsubstituted amino, and substituted or unsubstituted heterocycloalkyl having 1-2 heteroatoms independently selected from nitrogen, oxygen, and sulfur;
R^{6d} is substituted or unsubstituted amino, substituted or unsubstituted heterocycloalkyl having 1-2 heteroatoms independently selected from nitrogen, oxygen, and sulfur, substituted or unsubstituted aryl, substituted or unsubstituted heteroaryl, or

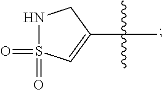 [SMILES: *C1=CS(=O)(=O)NC1]

R^{6e} is substituted or unsubstituted alkyl, substituted or unsubstituted aryl, substituted or unsubstituted heteroaryl, or

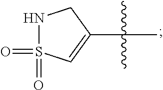 [SMILES: *C1=CS(=O)(=O)NC1]

each dotted bond is a single or a double bond; and
the subscript t is 0, 1, or 2;
provided that when
i) X is —NR^{1g}—, R^{1g} is H, each dotted bond is a single bond, and t is 1; then R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

In some particular embodiments, provided herein are compounds according to Formula (I) having the structure:

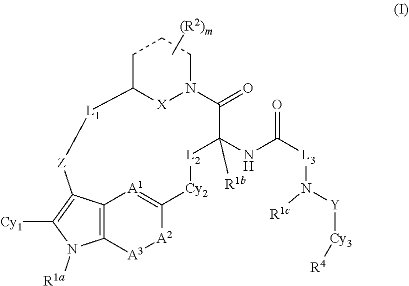

or a stereoisomer or a pharmaceutically acceptable salt thereof,
wherein:
each A¹, A², and A³ is independently —CR³═, or —N═; each R is independently H, halo, cyano, alkyl, haloalkyl, hydroxy, or alkoxy;
X is —CR^{1e}R^{1f}—, or —NR^{1g}—;
Z is absent or substituted or unsubstituted C₁-C₄ alkylene;
each Cy¹ and Cy² is independently substituted or unsubstituted aryl or heteroaryl;
Cy³ is substituted or unsubstituted heterocycloalkyl;
L¹ is —C(O)—O—, —C(O)—N(R^{1d})—, or -Cy⁴-; Cy⁴ is substituted or unsubstituted heteroaryl; and —O— of —C(O)—O—, or —N(R^{1d})— of —C(O)—N(R^{1d})— is attached to Z; or —O— of —C(O)—O—, or —N(R^{1d})— of —C(O)—N(R^{1d})— is attached to the pyrrolo ring when Z is absent;
each L², L³, and L⁴ is independently substituted or unsubstituted C₁-C₄ alkylene;
Y is —C(O)—, or —C(H)CH₂F, or —C(H)CF₃;
each R^{1a}, R^{1b}, R^{1c}, R^{1d}, R^{1e}, R^{1f}, and R^{1g} is independently H, or substituted or unsubstituted C₁-C₄ alkyl;
each R² is independently halo, cyano, alkyl, haloalkyl, or alkoxy; m is 0, 1, or 2;
R⁴ is any one of groups selected from i), and ii):
i) —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl;
ii) -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e},
L₄ is C(O), substituted or unsubstituted C₁-C₄ alkylene, or substituted or unsubstituted cycloalkyl;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, unsubstituted or substituted with one or more groups selected from substituted or unsubstituted amino, and substituted or unsubstituted heterocycloalkyl having 1-2 heteroatoms independently selected from nitrogen, oxygen, and sulfur;
R^{6a} is substituted or unsubstituted amino, substituted or unsubstituted heterocycloalkyl having 1-2 heteroatoms independently selected from nitrogen, oxygen, and sulfur, substituted or unsubstituted aryl, substituted or unsubstituted heteroaryl, or

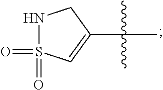 [SMILES: *C1=CS(=O)(=O)NC1]

R^{6e} is substituted or unsubstituted alkyl, substituted or unsubstituted aryl, substituted or unsubstituted heteroaryl, or

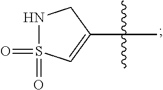 [SMILES: *C1=CS(=O)(=O)NC1]

and each dotted bond is a single or a double bond;
provided that when
i) X is —NR^{1g}—, R^{1g} is H, and each dotted bond is a single bond; then R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

In some embodiments, provided herein is a compound according to Formula (VIIa) or (VIIb) having the structure:

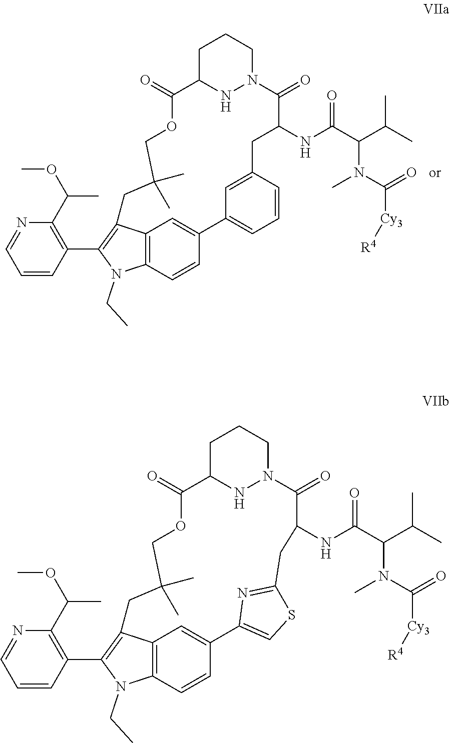

or a stereoisomer or a pharmaceutically acceptable salt thereof, and wherein R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

In some embodiments, provided herein is a compound according to Formula (VIIIa) or (VIIIb) having the structure:

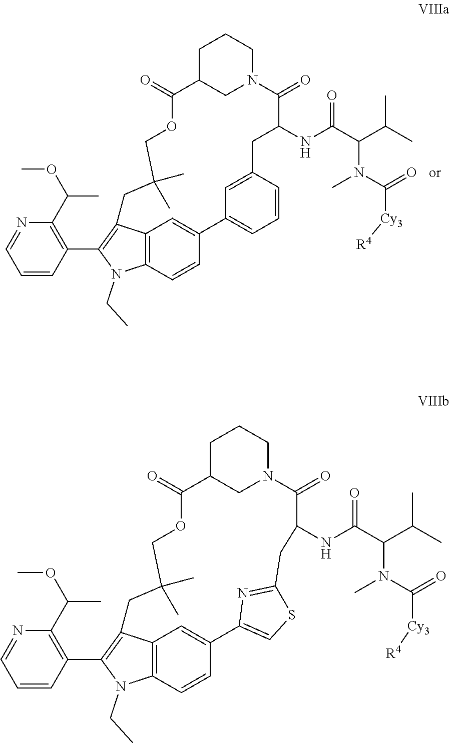

or a stereoisomer or a pharmaceutically acceptable salt thereof, and wherein R⁴ is —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl; -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

In some embodiments, provided herein is a compound according to Formula (XIa), (XIb), or (XIc) having the structure:

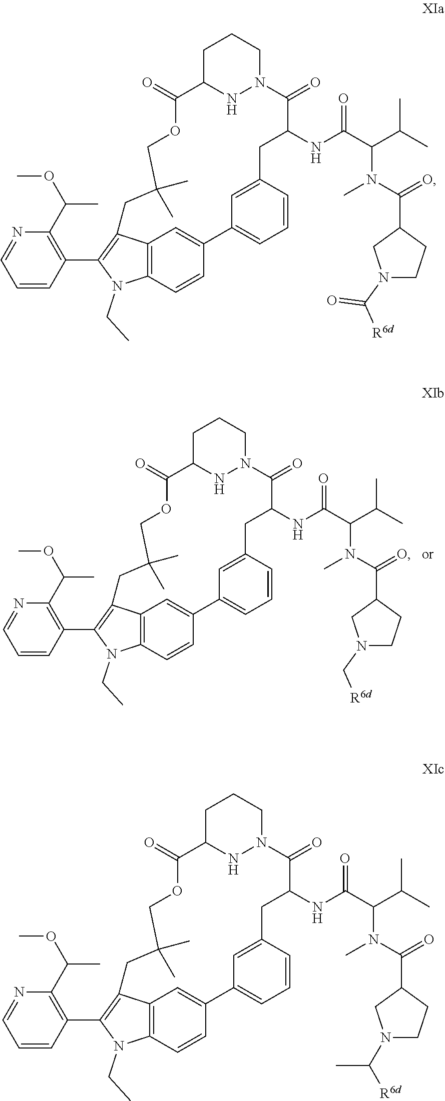

or a stereoisomer thereof; or a pharmaceutically acceptable salt thereof.

In some embodiments, provided herein is a compound according to Formula (XIIa) or (XIIb) having the structure:

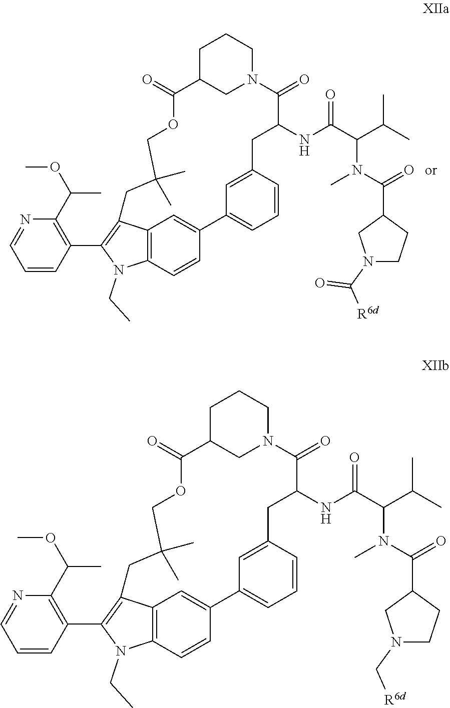

or a stereoisomer or a pharmaceutically acceptable salt thereof.

In some embodiments, the compound is according to formula (XIIIa) or (XIIIb):

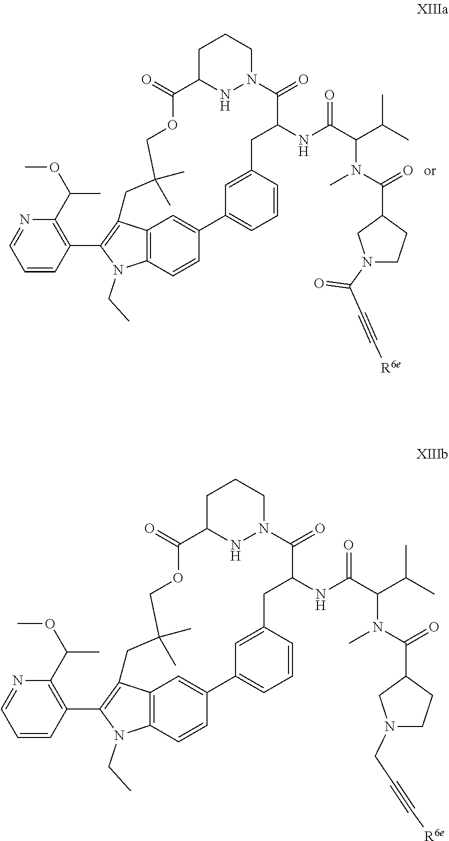

or a stereoisomer or a pharmaceutically acceptable salt thereof.

In some embodiments, the compound is according to formula (XIVa) or (XIVb):

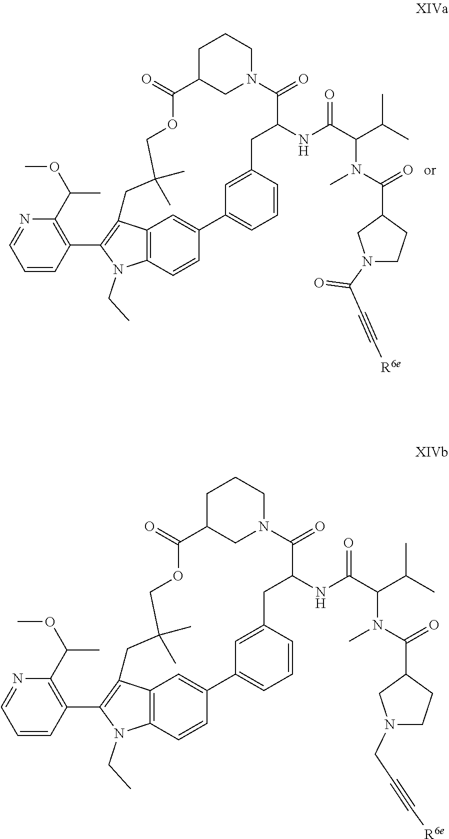

or a stereoisomer or a pharmaceutically acceptable salt thereof.

In some embodiments, the compound is according to formula (XVa), (XVb), (XVc), or (XVd):

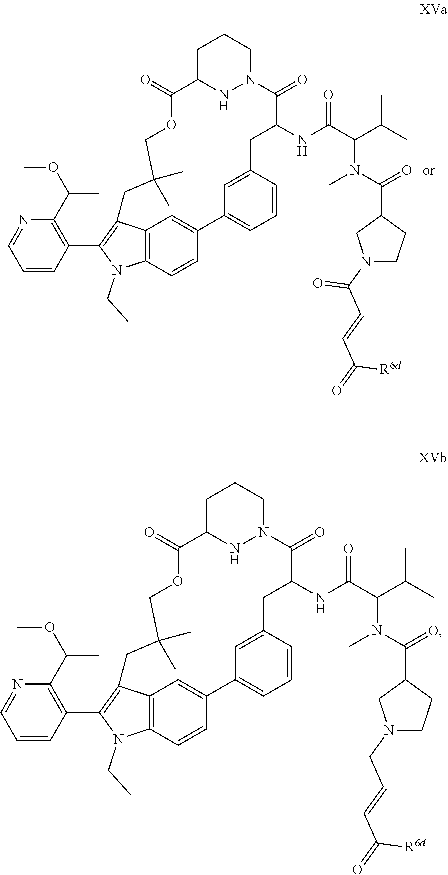

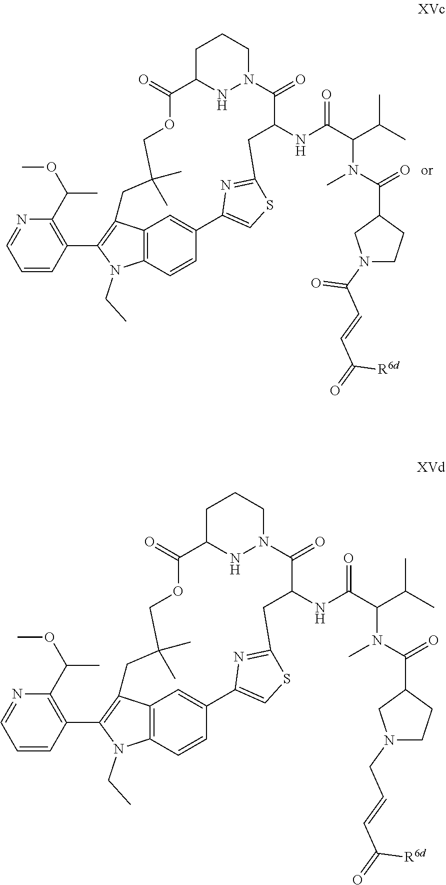

or a stereoisomer or a pharmaceutically acceptable salt thereof.

In some embodiments, the compound is according to formula (XVIa) or (XVIb):

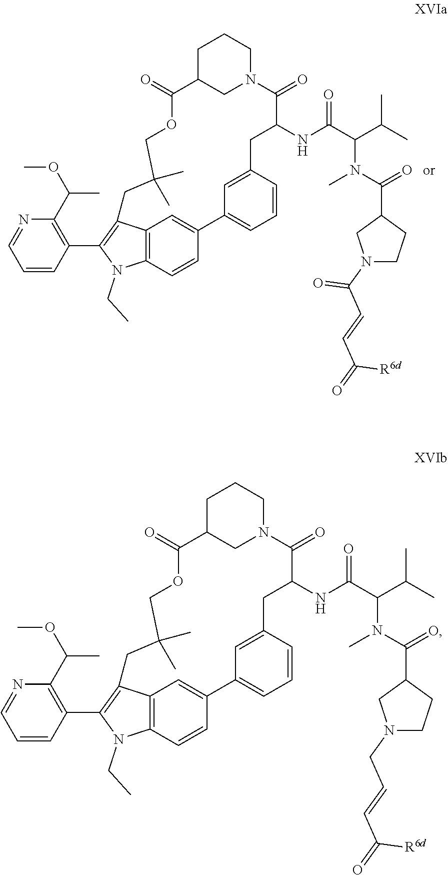

or a stereoisomer or a pharmaceutically acceptable salt thereof.

In some embodiments, the compound is according to formula (XVIIa), (XVIIb), (XVIIc), or (XVIId):

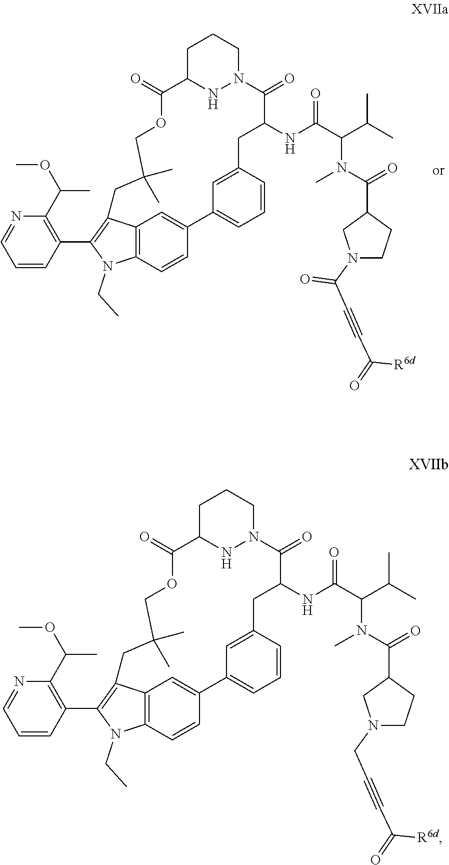

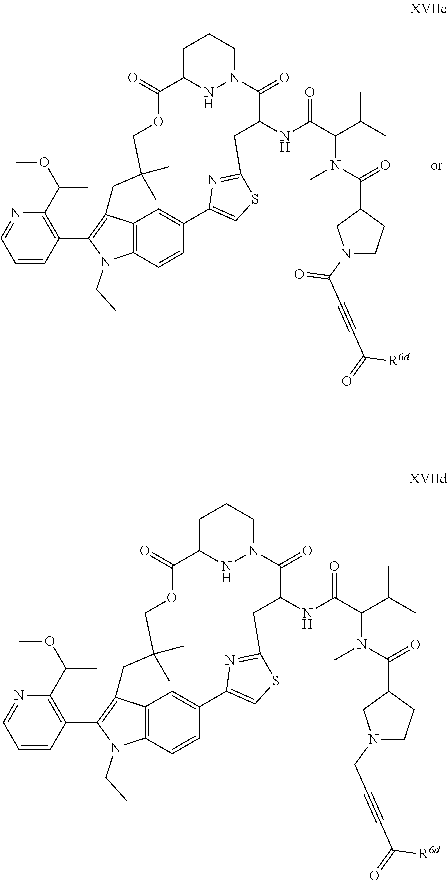

or a stereoisomer or a pharmaceutically acceptable salt thereof.

In some embodiments, the compound is according to formula (XVIIIa) or (XVIIIb):

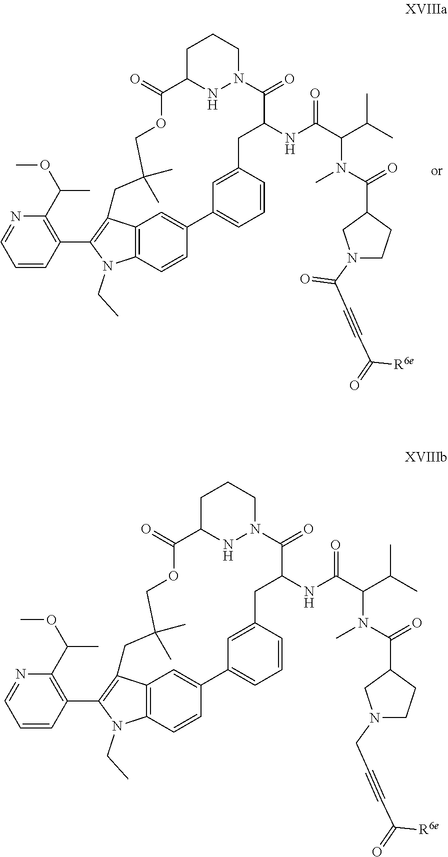

or a stereoisomer or a pharmaceutically acceptable salt thereof.

In some embodiments, the compound is according to formula (XXIa) or (XXIb):

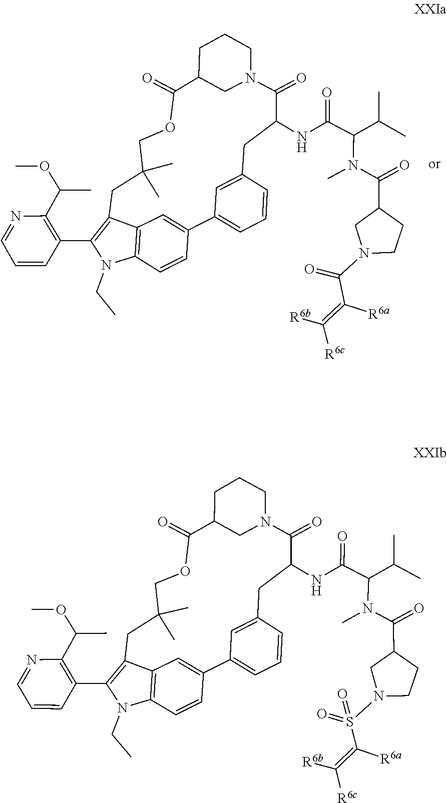

or a stereoisomer or a pharmaceutically acceptable salt thereof.

In some embodiments, the compound is according to formula (XXIIa) or (XXIIb):

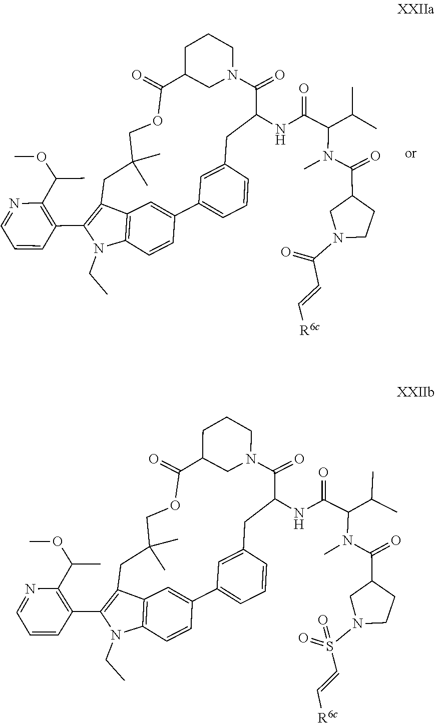

or a stereoisomer or a pharmaceutically acceptable salt thereof.

In some embodiments, the compound is according to formula (XXIIIa) or (XXIIIb):

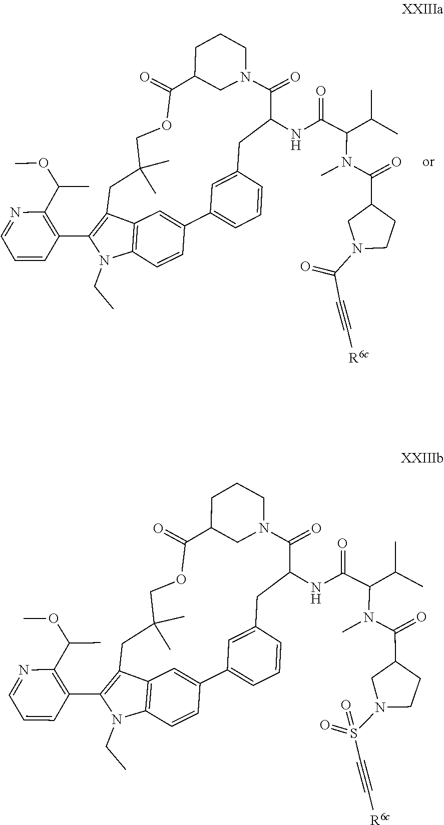

or a stereoisomer or a pharmaceutically acceptable salt thereof.

In some embodiments, the compound is according to formula (XXVIa), (XXVIb), (XXVIc), or (XXVId):

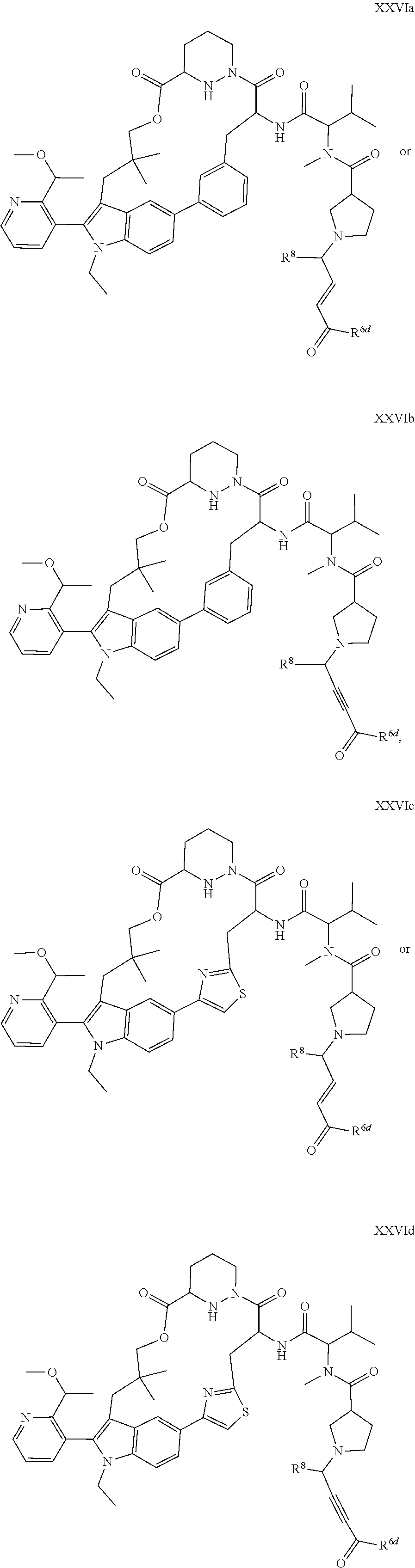

or a stereoisomer or a pharmaceutically acceptable salt thereof; wherein R⁸ is H, or C₁-C₄ alkyl; and R^{6d} is alkylamino or dialkylamino, or a substituted or unsubstituted heterocycle.

In some embodiments, provided herein are methods for preventing, treating or ameliorating in a mammal a disease or condition that is causally related to the aberrant activity of a KRas G12C in vivo, which comprises administering to the mammal an effective disease-treating or condition-treating amount of a compound according to Formula (I).

In some embodiments, provided herein are methods for preventing, treating or ameliorating in a mammal a Ras protein related disease or condition, which comprises administering to the mammal an effective disease-treating or condition-treating amount of a compound according to Formula (I).

In some embodiments, provided herein are methods for inhibiting a Ras protein in a cell, which comprises administering to the mammal an effective disease-treating or condition-treating amount of a compound according to Formula (I).

In some embodiments, the disease or condition is cancer.

In some embodiments, the compounds provided herein may also serve as anti-tumor agents through off-target activity by impacting other protein-protein interactions as well as kinases.

In some embodiments, provided herein are pharmaceutical compositions comprising a therapeutically effective amount of a compound of Formula (I) and a pharmaceutically acceptable excipient. In some embodiments, the pharmaceutical composition comprising the compound of Formula (I) is formulated for a route of administration selected from oral administration, parenteral administration, buccal administration, nasal administration, topical administration, or rectal administration. In some embodiments, provided herein are methods for treating an autoimmune disease or condition comprising administering to a patient in need a therapeutically effective amount of a compound of Formula (I). In some embodiments provided herein is a method for treating a cancer comprising administering to a patient in need a therapeutically effective amount of a compound of Formula (I). In some embodiments, the cancer is a myeloid line of blood cells. In some embodiments, the cancer is a lymphoid line of blood cell.

In some embodiments, the cancer is a B-cell proliferative disorder. In some embodiments, the cancer is a lymphoid line of blood cells. In some embodiments, the cancer is lung cancer. In some embodiments, the cancer is colorectal cancer. In some embodiments, the cancer is pancreatic cancer.

In some embodiments the myeloid line of blood cells is acute myeloid leukemia. In some embodiments the lymphoid line of blood cells is acute lymphoblastic leukemia. In some embodiments the B-cell proliferative disorder is diffuse large B cell lymphoma, follicular lymphoma or chronic lymphocytic leukemia. In some embodiments the cancer (soft tissue) is glioblastoma and pancreatic cancer. In some embodiments the cancer is renal cell carcinoma.

In some embodiments, provided herein is a method for treating mastocytosis comprising administering to a patient in need a therapeutically effective amount of a compound of Formula (I).

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some embodiments, provided herein are pharmaceutical compositions, which include a therapeutically effective amount of at least one of any of the compounds herein, or a pharmaceutically acceptable salt, pharmaceutically active metabolite, pharmaceutically acceptable prodrug, or pharmaceutically acceptable solvate. In certain embodiments, compositions provided herein further include a pharmaceutically acceptable diluent, excipient and/or binder.

Pharmaceutical compositions formulated for administration by an appropriate route and means containing effective concentrations of one or more of the compounds provided herein, or pharmaceutically effective derivatives thereof, that deliver amounts effective for the treatment, prevention, or amelioration of one or more symptoms of diseases, disorders or conditions that are modulated or otherwise affected by KRas G12C activity, or in which KRas G12C activity is implicated, are provided. The effective amounts and concentrations are effective for ameliorating any of the symptoms of any of the diseases, disorders or conditions disclosed herein.

In certain embodiments, provided herein is a pharmaceutical composition containing: i) a physiologically acceptable carrier, diluent, and/or excipient; and ii) one or more compounds provided herein.

In some embodiments, provided herein are methods for treating a patient by administering a compound provided herein. In some embodiments, provided herein is a method of inhibiting the activity of KRas G12C, or of treating a disease, disorder, or condition, which would benefit from inhibition of KRas G12C activity, in a patient, which includes administering to the patient a therapeutically effective amount of at least one of any of the compounds herein, or pharmaceutically acceptable salt, pharmaceutically active metabolite, pharmaceutically acceptable prodrug, or pharmaceutically acceptable solvate.

In some embodiments, provided herein is the use of a compound disclosed herein for inhibiting KRas G12C activity or for the treatment of a disease, disorder, or condition, which would benefit from inhibition of KRas G12C activity.

In some embodiments, compounds provided herein are administered to a human.

In some embodiments, compounds provided herein are orally administered.

In some embodiments, compounds provided herein are used for the formulation of a medicament for the inhibition of KRas G12C activity. In some embodiments, compounds provided herein are used for the formulation of a medicament for the inhibition of KRas G12C activity.

Articles of manufacture including packaging material, a compound or composition or pharmaceutically acceptable derivative thereof provided herein, which is effective for inhibiting the activity of KRas G12C, within the packaging material, and a label that indicates that the compound or composition, or pharmaceutically acceptable salt, pharmaceutically active metabolite, pharmaceutically acceptable prodrug, or pharmaceutically acceptable solvate thereof, is used for inhibiting the activity of KRas G12C, are provided.

In some embodiments, provided herein is a method for inhibiting KRas G12C activity in a subject in need thereof by administering to the subject thereof a composition containing a therapeutically effective amount of at least one compound having the structure of Formula (I).

Pharmaceutical compositions formulated for administration by an appropriate route and means containing effective concentrations of one or more of the compounds provided herein, or pharmaceutically effective derivatives thereof, that deliver amounts effective for the treatment, prevention, or amelioration of one or more symptoms of diseases, disorders or conditions that are modulated or otherwise affected by KRas G12D activity, or in which KRas G12D activity is implicated, are provided. The effective amounts and concentrations are effective for ameliorating any of the symptoms of any of the diseases, disorders or conditions disclosed herein.

In certain embodiments, provided herein is a pharmaceutical composition containing: i) a physiologically acceptable carrier, diluent, and/or excipient; and ii) one or more compounds provided herein.

In some embodiments, provided herein are methods for treating a patient by administering a compound provided herein. In some embodiments, provided herein is a method of inhibiting the activity of KRas G12D, or of treating a disease, disorder, or condition, which would benefit from inhibition of KRas G12D activity, in a patient, which includes administering to the patient a therapeutically effective amount of at least one of any of the compounds herein, or pharmaceutically acceptable salt, pharmaceutically active metabolite, pharmaceutically acceptable prodrug, or pharmaceutically acceptable solvate.

In some embodiments, provided herein is the use of a compound disclosed herein for inhibiting KRas G12D activity or for the treatment of a disease, disorder, or condition, which would benefit from inhibition of KRas G12D activity.

In some embodiments, compounds provided herein are administered to a human.

In some embodiments, compounds provided herein are orally administered.

In some embodiments, compounds provided herein are used for the formulation of a medicament for the inhibition of KRas G12D activity. In some embodiments, compounds provided herein are used for the formulation of a medicament for the inhibition of KRas G12D activity.

Articles of manufacture including packaging material, a compound or composition or pharmaceutically acceptable derivative thereof provided herein, which is effective for inhibiting the activity of KRas G12D, within the packaging material, and a label that indicates that the compound or composition, or pharmaceutically acceptable salt, pharmaceutically active metabolite, pharmaceutically acceptable prodrug, or pharmaceutically acceptable solvate thereof, is used for inhibiting the activity of KRas G12D, are provided.

In some embodiments, the subject in need is suffering from a cancer. In some embodiments, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds.

In some embodiments, provided herein is a method for treating an autoimmune disease by administering to a subject in need thereof a composition containing a therapeutically effective amount of at least one compound having the structure of Formula (B-I), (I)—(XXVIIId).

In some embodiments, provided herein is a method for treating a cancer by administering to a subject in need thereof a composition containing a therapeutically effective amount of at least one compound having the structure of Formula (B-I), (I)—(XXVIIId). In some embodiments, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds.

In some embodiments, the cancer is acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, or bladder cancer.

In some embodiments, the cancer is bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, or cardiac tumors, craniopharyngioma, cutaneous T-cell lymphoma, extrahepatic ductal carcinoma in situ (DCIS), embryonal tumors, Ewing sarcoma, extracranial germ cell tumor, extragonadal germ cell tumor, gastrointestinal carcinoid tumor, gastrointestinal stromal tumors (GIST), germ cell tumor, gestational trophoblastic tumor, Hodgkin's lymphoma, islet cell tumors, pancreatic neuroendocrine tumors, midline tract carcinoma, non-Hodgkin's lymphoma.

In some embodiments, the cancer is multiple endocrine neoplasia syndromes, multiple myeloma/plasma cell neoplasm, mycosis fungoides, myelodysplasia syndromes, myelodysplastic/myeloproliferative neoplasms, multiple myeloma.

In some embodiments, the cancer is merkel cell carcinoma, malignant mesothelioma, malignant fibrous histiocytoma of bone and osteosarcoma, nasal cavity and paranasal sinus cancer, nasopharyngeal cancer, neuroblastoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, or primary central nervous system (CNS).

In some embodiments, the cancer is hairy cell leukemia, head and neck cancer, heart cancer, liver cancer, hypopharyngeal cancer, intraocular melanoma, kidney cancer, laryngeal cancer, lip and oral cavity cancer, liver cancer, lobular carcinoma in situ (LCIS), lung cancer, lymphoma, metastatic squamous neck cancer with occult primary, or mouth cancer.

In some embodiments, the cancer is multiple endocrine neoplasia syndromes, multiple myeloma/plasma cell neoplasm, mycosis fungoides, myelodysplasia syndromes, myelodysplastic/myeloproliferative neoplasms, multiple myeloma, lymphoma, prostate cancer, rectal cancer, or transitional cell cancer,

In some embodiments, the cancer is retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some embodiments, the cancer is chronic lymphocytic leukemia (CLL), chronic myelogenous leukemia (CML), chronic myleoproliferative disorders, colon cancer, colorectal cancer.

In some embodiments, the cancer is CNS cancer, endometrial cancer, ependymoma, esophageal cancer, esthesioneuroblastoma, eye cancer, fibrous histiocytoma of bone, gall bladder cancer, gastric cancer

In some embodiments are methods for treating inflammation comprising administering to the mammal at least once an effective amount of at least one compound having the structure of Formula (B-I), (I)—(XXVIIId).

In some embodiments, provided herein are methods for the treatment of cancer comprising administering to the mammal at least once an effective amount of at least one compound having the structure of Formula (B-I), (I)—(XXVIIId). The type of cancer may include, but is not limited to, pancreatic cancer and other solid or hematological tumors.

In certain embodiments, provided herein are methods for treating the following diseases or conditions comprising administering to the mammal a compound provided herein. In some embodiments, the disease or condition is ALL (Acute Lymphoblastic Lymphoma), DLBCL (Diffuse Large B-Cell Lymphoma), FL (Follicular Lymphoma), RCC (Renal Cell Carcinoma), Childhood Medulloblastoma, Glioblastoma, Pancreatic tumor or cancer, Liver cancer (Hepatocellular Carcinoma), Prostate Cancer (Myc), Triple Negative Breast (Myc), AML (Acute Myeloid Leukemia), or MDS (Myelo Dyslplastic Syndrome). In some embodiments, the disease or condition is Early-onset Dystonia. In yet some embodiments, the disease or condition is Kabuki Syndrome.

In some embodiments, the disease or condition is p53 driven tumor.

In some embodiments, the disease or condition is MYC driven tumor. MYC is documented to be involved broadly in many cancers, in which its expression is estimated to be elevated or deregulated in up to 70% of human cancers. High levels of MYC expression have been linked to aggressive human prostate cancer and triple negative breast cancer (Gurel et al., Mod Pathol. 2008 September; 21(9):1156-67; Palaskas et al., Cancer Res. 2011 Aug. 1; 71(15):5164-74). Experimental models of Myc-mediated tumorigenesis suggest that established tumors are addicted to Myc and that deregulated expression of Myc result in an addiction not only to Myc but also to nutrients. These Myc-induced changes provide a unique opportunity for new therapeutic strategies. Notwithstanding the fact that normal proliferating cells (stem cell compartments and immune cells) also use MYC for renewal, many studies have focused on targeting Myc for cancer therapeutics. Strategies have emerged to inhibit MYC expression, to interrupt Myc-Max dimerization, to inhibit Myc-Max DNA binding, and to interfere with key Myc target genes (Dang et al. Cell. 2012, 149(1): 22-35).

In any of the aforementioned embodiments are some embodiments in which administration is enteral, parenteral, or both, and wherein (a) an effective amount of a provided compound is systemically administered to the mammal; (b) an effective amount of a provided compound is administered orally to the mammal; (c) an effective amount of a provided compound is intravenously administered to the mammal; (d) an effective amount of a provided compound is administered by inhalation; (e) an effective amount of a provided compound is administered by nasal administration; or (f) an effective amount of a provided compound is administered by injection to the mammal; (g) an effective amount of a provided compound is administered topically (dermal) to the mammal; (h) an effective amount of a provided compound is administered by ophthalmic administration; or (i) an effective amount of a provided compound is administered rectally to the mammal.

In any of the aforementioned embodiments are some embodiments comprising single administrations of an effective amount of a provided compound, including some embodiments in which (i) a provided compound is administered once; (ii) a provided compound is administered to the mammal multiple times over the span of one day; (iii) continually; or (iv) continuously.

In any of the aforementioned embodiments are some embodiments comprising multiple administrations of an effective amount of a provided compound, including some embodiments in which (i) a provided compound is administered in a single dose; (ii) the time between multiple administrations is every 6 hours; (iii) a provided compound is administered to the mammal every 8 hours. In some embodiments, the method comprises a drug holiday, wherein the administration of the compound is temporarily suspended or the dose of the compound being administered is temporarily reduced; at the end of the drug holiday, dosing of the compound is resumed. The length of the drug holiday can vary from 2 days to 1 year.

In any of the aforementioned embodiments involving the treatment of proliferative disorders, including cancer, are some embodiments comprising administering at least one additional agent selected from the group consisting of alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase irreversible inhibitors such as irinotecan or topotecan, tyrosine kinase irreversible inhibitors such as gefinitinib or imatinib, or agents to treat signs or symptoms induced by such therapy including allopurinol, filgrastim, granisetron/ondansetron/palonosetron, dronabinol.

In some embodiments, the compounds of Formula (B-I), (I)—(XXVIIId) are irreversible inhibitors of KRas G12C activity. In certain embodiments, such irreversible inhibitors have an IC₅₀ below 10 microM in enzyme assay. In some embodiments, a KRas G12C inhibitor has an IC₅₀ of less than 1 microM, and in some embodiments, less than 0.25 microM.

Other objects, features, and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### DETAILED DESCRIPTION OF THE PREFERRED EMBODIMENTS

### Certain Terminology

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. Use of the term “including” as well as other forms, such as “include”, “includes,” and “included,” is not limiting. Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In some embodiments, an alkyl comprises five to fifteen carbon atoms (e.g., C₅-C₁₅ alkyl). In certain embodiments, an alkyl comprises five to eight carbon atoms (e.g., C₅-C₈ alkyl). The alkyl is attached to the rest of the molecule by a single bond, for example, methyl (Me), ethyl (Et), n-propyl (n-pr), 1-methylethyl (iso-propyl or i-Pr), n-butyl (n-Bu), n-pentyl, 1,1-dimethylethyl (t-butyl, ort-Bu), 3-methylhexyl, 2-methylhexyl, and the like. Unless stated otherwise specifically in the specification, an alkyl group is optionally substituted as defined and described below and herein.

The alkyl group could also be a “lower alkyl” having 1 to 6 carbon atoms.

As used herein, C₁-Cₓ includes C₁-C₂, C₁-C₃ . . . C₁-Cₓ.

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In some embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted as defined and described below and herein.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In some embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted as defined and described below and herein.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted as defined and described below and herein.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted as defined and described below and herein. “Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl (Ph), fluorenyl, and naphthyl. Unless stated otherwise specifically in the specification, the term “aryl” or the prefix “ar-” (such as in “aralkyl”) is meant to include aryl radicals optionally substituted as defined and described below and herein.

“Aralkyl” refers to a radical of the formula —R^{c}-aryl where R^{c} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In some embodiments, a carbocyclyl comprises five to seven carbon atoms. The carbocyclyl is attached to the rest of the molecule by a single bond. Carbocyclyl is optionally saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted as defined and described below and herein. “Halo” or “halogen” refers to bromo, chloro, fluoro or iodo substituents.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In some embodiments in which two or more hydrogen atoms are replaced with halogen atoms, the halogen atoms are not all the same as one another.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three to 14 ring atoms, such as three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

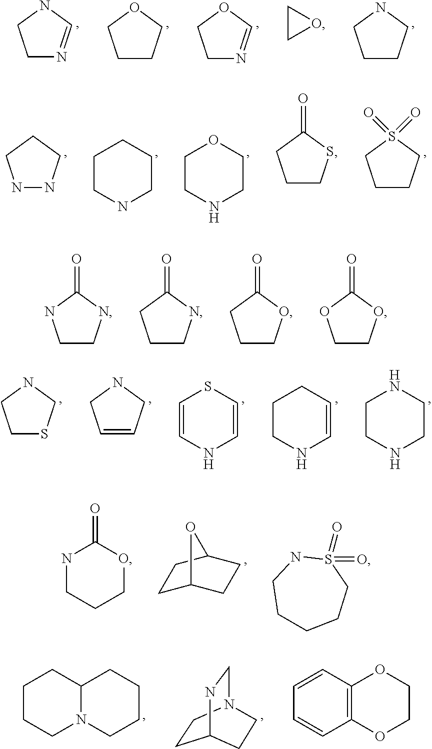

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. In some embodiments, heteroaryl rings have five, six, seven, eight, nine, or more than nine ring atoms. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl,isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl).

Unless stated otherwise specifically in the specification, the term “heteroaryl” is meant to include heteroaryl radicals as defined above which are optionally substituted as defined and described below and herein.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Sulfanyl” refers to the —S— radical.

“Sulfinyl” refers to the —S(═O)— radical.

“Sulfonyl” refers to the —S(═O)₂— radical.

“Amino” refers to the —NH₂ radical.

“Cyano” refers to the —CN radical.

“Nitro” refers to the —NO₂ radical.

“Oxa” refers to the —O— radical.

“Oxo” refers to the =O radical.

“Imino” refers to the =NH radical.

“Thioxo” refers to the =S radical.

An “alkoxy” group refers to a (alkyl)O— group, where alkyl is as defined herein.

An “aryloxy” group refers to an (aryl)O— group, where aryl is as defined herein.

“Carbocyclylalkyl” means an alkyl radical, as defined herein, substituted with a carbocyclyl group. “Cycloalkylalkyl” means an alkyl radical, as defined herein, substituted with a cycloalkyl group.

Non-limiting cycloalkylalkyl groups include cyclopropylmethyl, cyclobutylmethyl, cyclopentylmethyl, cyclohexylmethyl, and the like.

As used herein, the terms “heteroalkyl” “heteroalkenyl” and “heteroalkynyl” include optionally substituted alkyl, alkenyl and alkynyl radicals in which one or more skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond,” “direct bond” or “single bond” refers to a chemical bond between two atoms, or two moieties when the atoms joined by the bond are considered to be part of larger substructure.

An “isocyanato” group refers to a —NCO group.

An “isothiocyanato” group refers to a —NCS group.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “alkylthioalkyl” group refers to an alkyl group substituted with a —S-alkyl group.

As used herein, the term “acyloxy” refers to a group of formula RC(═O)O—.

“Carboxy” means a —C(O)OH radical.

As used herein, the term “acetyl” refers to a group of formula —C(═O)CH₃.

“Acyl” refers to the group —C(O)R.

As used herein, the term “trihalomethanesulfonyl” refers to a group of formula X₃CS(═O)₂— where X is a halogen.

“Cyanoalkyl” means an alkyl radical, as defined herein, substituted with at least one cyano group.

As used herein, the term “N-sulfonamido” or “sulfonylamino” refers to a group of formula RS(═O)₂NH—.

As used herein, the term “O-carbamyl” refers to a group of formula —OC(═O)NR₂.

As used herein, the term “N-carbamyl” refers to a group of formula ROC(═O)NH—.

As used herein, the term “O-thiocarbamyl” refers to a group of formula —OC(═S)NR₂.

As used herein, “N-thiocarbamyl” refers to a group of formula ROC(═S)NH—.

As used herein, the term “C-amido” refers to a group of formula —C(═O)NR₂.

“Aminocarbonyl” refers to a —CONH₂ radical.

As used herein, the term “N-amido” refers to a group of formula RC(═O)NH—.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to a (alkenyl)O— group, where alkenyl is as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3′^{d} Ed., John Wiley & Sons, New York, NY, 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

As described herein, compounds provided herein may be “optionally substituted”. In general, the term “substituted,” whether preceded by the term “optionally” or not, means that one or more hydrogens of a designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents provided herein are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “nucleophile” or “nucleophilic” refers to an electron rich compound, or moiety thereof.

The term “electrophile”, or “electrophilic” refers to an electron poor or electron deficient molecule, or moiety thereof. Examples of electrophiles include, but in no way are limited to, Michael acceptor moieties.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

As used herein, “amelioration” of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

“Bioavailability” refers to the percentage of the weight of compounds disclosed herein, such as, compounds of any of Formula (B-I), (I)—(XXVIIId) dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which compounds disclosed herein, such as, compounds of any of Formula (B-I), (I)—(XXVIIId) are absorbed into the general circulation when the pharmaceutical composition is taken orally as compared to intravenous injection.

“Blood plasma concentration” refers to the concentration of compounds disclosed herein, such as, compounds of any of Formula (B-I), (I)—(XXVIIId) in the plasma component of blood of a subject. It is understood that the plasma concentration of compounds of any of Formula (B-I), (I)—(XXVIIId) may vary significantly between subjects, due to variability with respect to metabolism and/or possible interactions with other therapeutic agents. In accordance with some embodiments disclosed herein, the blood plasma concentration of the compounds of any of Formula (B-I), (I)—(XXVIIId) may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound of any of Formula (B-I), (I)—(XXVIIId) may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound of any of Formula (B-I), (I)—(XXVIIId), age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The term “identical,” as used herein, refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence. By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75-100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75-100 nucleic acids in length, over a region that is about 50 nucleic acids in length, or, where not specified, across the entire sequence of a polynucleotide sequence.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “irreversible inhibitor,” as used herein, refers to a compound that, upon contact with a target protein (e.g., KRas G12C or KRas G12D) causes the formation of a new covalent bond with or within the protein, whereby one or more of the target protein's biological activities (e.g., phosphotransferase activity) is diminished or abolished notwithstanding the subsequent presence or absence of the irreversible inhibitor. In contrast, a reversible inhibitor compound upon contact with a target protein does not cause the formation of a new covalent bond with or within the protein and therefore can associate and dissociate from the target protein.

The term “irreversible inhibitor of KRas G12C protein-protein interaction” as used herein, refers to an inhibitor of KRas G12C that can form a covalent bond with an amino acid residue of KRas G12C. In one embodiment, the irreversible inhibitor of KRas G12C can form a covalent bond with a Cys residue of KRas G12C; in particular embodiments, the irreversible inhibitor can form a covalent bond with a Cys 12 residue (or a homolog thereof) of KRas G12C.

The term “prophylactically effective amount,” as used herein, refers that amount of a composition applied to a patient that will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

As used herein, the term “selective binding compound” refers to a compound that selectively binds to any portion of one or more target proteins.

As used herein, the term “selectively binds” refers to the ability of a selective binding compound to bind to a target protein, such as, for example, KRas G12C, with greater affinity than it binds to a non-target protein. In certain embodiments, specific binding refers to binding to a target with an affinity that is at least 10, 50, 100, 250, 500, 1000 or more times greater than the affinity for a non-target.

The term “irreversible inhibitor of KRas G12D protein-protein interaction” as used herein, refers to an inhibitor of KRas G12D that can form a covalent bond with an amino acid residue of KRas G12D. In one embodiment, the irreversible inhibitor of KRas G12D can form a covalent bond with a Cys residue of KRas G12D; in particular embodiments, the irreversible inhibitor can form a covalent bond with a Cys 12 residue (or a homolog thereof) of KRas G12D.

The term “prophylactically effective amount,” as used herein, refers that amount of a composition applied to a patient that will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

As used herein, the term “selective binding compound” refers to a compound that selectively binds to any portion of one or more target proteins.

As used herein, the term “selectively binds” refers to the ability of a selective binding compound to bind to a target protein, such as, for example, KRas G12D, with greater affinity than it binds to a non-target protein. In certain embodiments, specific binding refers to binding to a target with an affinity that is at least 10, 50, 100, 250, 500, 1000 or more times greater than the affinity for a non-target.

As used herein, the term “selective modulator” refers to a compound that selectively modulates a target activity relative to a non-target activity. In certain embodiments, specific modulator refers to modulating a target activity at least 10, 50, 100, 250, 500, 1000 times more than a non-target activity.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

The term “subject” or “patient” as used herein, refers to an animal which is the object of treatment, observation, or experiment. By way of example only, a subject may be, but is not limited to, a mammal including, but not limited to, a human.

As used herein, the term “target activity” refers to a biological activity capable of being modulated by a selective modulator. Certain exemplary target activities include, but are not limited to, binding affinity, signal transduction, enzymatic activity, tumor growth, inflammation, or inflammation-related processes, and amelioration of one or more symptoms associated with a disease or condition.

As used herein, the term “target protein” refers to a molecule or a portion of a protein capable of being bound by a selective binding compound. In certain embodiments, a target protein is KRas.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the IC₅₀ refers to an amount, concentration, or dosage of a particular test compound that achieves a 50% inhibition of a maximal response, such as inhibition of KRas G12C, in an assay that measures such response.

As used herein, EC₅₀ refers to a dosage, concentration, or amount of a particular test compound that elicits a dose-dependent response at 50% of maximal expression of a particular response that is induced, provoked or potentiated by the particular test compound.

Methods described herein include administering to a subject in need a composition containing a therapeutically effective amount of one or more KRas G12C inhibitor compounds described herein.

In some embodiments, methods described herein can be used to treat an autoimmune disease, which includes, but is not limited to, rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, and vulvodynia.

In some embodiments, methods described herein can be used to treat heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In some embodiments, methods described herein can be used to treat an inflammatory disease, which includes, but is not limited to asthma, inflammatory bowel disease, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In some embodiments, methods described herein can be used to treat a cancer, e.g., B-cell proliferative disorders, which include, but are not limited to diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, and lymphomatoid granulomatosis.

In some embodiments, methods described herein can be used to treat thromboembolic disorders, which include, but are not limited to myocardial infarct, angina pectoris (including unstable angina), reocclusions or restenoses after angioplasty or aortocoronary bypass, stroke, transitory ischemia, peripheral arterial occlusive disorders, pulmonary embolisms, and deep venous thromboses.

Symptoms, diagnostic tests, and prognostic tests for each of the above-mentioned conditions are known in the art. See, e.g., Harrison's Principles of Internal Medicine©,” 16th ed., 2004, The McGraw-Hill Companies, Inc. Dey et al. (2006), Cytojoumal 3(24), and the “Revised European American Lymphoma” (REAL) classification system (see, e.g., the website maintained by the National Cancer Institute).

A number of animal models of are useful for establishing a range of therapeutically effective doses of KRas G12C inhibitor compounds for treating any of the foregoing diseases.

For example, dosing of KRas G12C inhibitor compounds for treating an autoimmune disease can be assessed in a mouse model of rheumatoid arthritis. In this model, arthritis is induced in Balb/c mice by administering anti-collagen antibodies and lipopolysaccharide. See Nandakumar et al. (2003), Am. J. Pathol 163:1827-1837

In another example, dosing of KRas G12C irreversible inhibitors for the treatment of B-cell proliferative disorders can be examined in, e.g., a human-to-mouse xenograft model in which human B-cell lymphoma cells (e.g. Ramos cells) are implanted into immunodeficient mice (e.g., “nude” mice) as described in, e.g., Pagel et al. (2005), Clin Cancer Res 11(13):4857-4866

Animal models for treatment of thromboembolic disorders are also known.

The therapeutic efficacy of a provided compound for one of the foregoing diseases can be optimized during a course of treatment. For example, a subject being treated can undergo a diagnostic evaluation to correlate the relief of disease symptoms or pathologies to inhibition of in vivo KRas G12C activity achieved by administering a given dose of a KRas G12C inhibitor.

A number of animal models of are useful for establishing a range of therapeutically effective doses of KRas G12D inhibitor compounds for treating any of the foregoing diseases.

For example, dosing of KRas G12D inhibitor compounds for treating an autoimmune disease can be assessed in a mouse model of rheumatoid arthritis. In this model, arthritis is induced in Balb/c mice by administering anti-collagen antibodies and lipopolysaccharide. See Nandakumar et al. (2003), Am. J. Pathol 163:1827-1837.

In another example, dosing of KRas G12D irreversible inhibitors for the treatment of B-cell proliferative disorders can be examined in, e.g., a human-to-mouse xenograft model in which human B-cell lymphoma cells (e.g. Ramos cells) are implanted into immunodeficient mice (e.g., “nude” mice) as described in, e.g., Pagel et al. (2005), Clin Cancer Res 11(13):4857-4866

Animal models for treatment of thromboembolic disorders are also known.

The therapeutic efficacy of a provided compound for one of the foregoing diseases can be optimized during a course of treatment. For example, a subject being treated can undergo a diagnostic evaluation to correlate the relief of disease symptoms or pathologies to inhibition of in vivo KRas G12D activity achieved by administering a given dose of a KRas G12D inhibitor.

### Compounds

In the following description of KRas G12C inhibitor compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

KRas G12C inhibitor compounds can be used for the manufacture of a medicament for treating any of the foregoing conditions (e.g., autoimmune diseases, inflammatory diseases, allergy disorders, B-cell proliferative disorders, Myeloid cell proliferative disorder, Lymphoid cell proliferative disorder, or thromboembolic disorders).

In some embodiments, the KRas G12C inhibitor compound used for the methods described herein inhibits KRas G12C activity with an in vitro IC₅₀ of less than about 10 μM (e.g., less than about 1 M, less than about 0.5 μM, less than about 0.4 μM, less than about 0.3 μM, less than about 0.1 μM, less than about 0.08 μM, less than about 0.06 μM, less than about 0.05 μM, less than about 0.04 μM, less than about 0.03 μM, less than about 0.02 μM, less than about 0.01 μM, less than about 0.008 μM, less than about 0.006 μM, less than about 0.005 μM, less than about 0.004 μM, less than about 0.003 μM, less than about 0.002 μM, less than about 0.001 μM, less than about 0.00099 μM, less than about 0.00098 μM, less than about 0.00097 μM, less than about 0.00096 μM, less than about 0.00095 μM, less than about 0.00094 M, less than about 0.00093 μM, less than about 0.00092 μM, or less than about 0.00090 μM).

Also described herein are methods for synthesizing such irreversible inhibitors, methods for using such irreversible inhibitors in the treatment of diseases (including diseases wherein inhibition of KRas G12C provides therapeutic benefit to a patient having the disease). Further described are pharmaceutical compositions that include an inhibitor of KRas G12C.

Specifically described herein are irreversible inhibitors of KRas G12C that form a covalent bond with a cysteine residue on KRas G12C. Further described herein are irreversible inhibitors of KRas G12C that form a covalent bond with a Cys12 residue on KRas. Also described are pharmaceutical formulations that include an irreversible inhibitor of KRas G12C.

The inhibitor compounds described herein are selective for KRas having a cysteine residue in an amino acid sequence position of the KRas G12C protein that is homologous to the amino acid sequence position of cysteine 12 in KRas G12C. Irreversible inhibitor compounds described herein include a Michael acceptor moiety.

Generally, a reversible or irreversible inhibitor compound of KRas G12C used in the methods described herein is identified or characterized in an in vitro assay, e.g., an acellular biochemical assay or a cellular functional assay. Such assays are useful to determine an in vitro IC₅₀ for a reversible or irreversible KRas G12C inhibitor compound.

Further, covalent complex formation between KRas G12C and a candidate irreversible KRas G12C inhibitor is a useful indicator of irreversible inhibition of KRas G12C that can be readily determined by a number of methods known in the art (e.g., mass spectrometry). For example, some irreversible KRas G12C-inhibitor compounds can form a covalent bond with Cys 12 of KRas GC12 (e.g., via a Michael reaction). See S. Xu et al. Angewandte Chemie International Ed. 57(6), 1601-1605 (2017) (incorporated by reference in its entirety).

In the following description of KRas G12D inhibitor compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

KRas G12D inhibitor compounds can be used for the manufacture of a medicament for treating any of the foregoing conditions (e.g., autoimmune diseases, inflammatory diseases, allergy disorders, B-cell proliferative disorders, Myeloid cell proliferative disorder, Lymphoid cell proliferative disorder, or thromboembolic disorders).

In some embodiments, the KRas G12D inhibitor compound used for the methods described herein inhibits KRas G12D activity with an in vitro IC₅₀ of less than about 10 μM (e.g., less than about 1 M, less than about 0.5 μM, less than about 0.4 μM, less than about 0.3 μM, less than about 0.1 μM, less than about 0.08 μM, less than about 0.06 μM, less than about 0.05 μM, less than about 0.04 μM, less than about 0.03 μM, less than about 0.02 μM, less than about 0.01 μM, less than about 0.008 μM, less than about 0.006 μM, less than about 0.005 μM, less than about 0.004 μM, less than about 0.003 μM, less than about 0.002 μM, less than about 0.001 μM, less than about 0.00099 μM, less than about 0.00098 μM, less than about 0.00097 μM, less than about 0.00096 μM, less than about 0.00095 μM, less than about 0.00094 M, less than about 0.00093 μM, less than about 0.00092 μM, or less than about 0.00090 μM).

Also described herein are methods for synthesizing such irreversible inhibitors, methods for using such irreversible inhibitors in the treatment of diseases (including diseases wherein inhibition of KRas G12D provides therapeutic benefit to a patient having the disease). Further described are pharmaceutical compositions that include an inhibitor of KRas G12D.

Specifically described herein are irreversible inhibitors of KRas G12D that form a covalent bond with a cysteine residue on KRas G12D. Further described herein are irreversible inhibitors of KRas G12D that form a covalent bond with a Cys12 residue on KRas. Also described are pharmaceutical formulations that include an irreversible inhibitor of KRas G12D.

The inhibitor compounds described herein are selective for KRas having a cysteine residue in an amino acid sequence position of the KRas G12D protein that is homologous to the amino acid sequence position of cysteine 12 in KRas G12D. Irreversible inhibitor compounds described herein include a Michael acceptor moiety.

Generally, a reversible or irreversible inhibitor compound of KRas G12D used in the methods described herein is identified or characterized in an in vitro assay, e.g., an acellular biochemical assay or a cellular functional assay. Such assays are useful to determine an in vitro IC₅₀ for a reversible or irreversible KRas G12D inhibitor compound.

Further, covalent complex formation between KRas G12D and a candidate irreversible KRas G12D inhibitor is a useful indicator of irreversible inhibition of KRas G12D that can be readily determined by a number of methods known in the art (e.g., mass spectrometry). For example, some irreversible KRas G12D-inhibitor compounds can form a covalent bond with Cys 12 of KRas GC12 (e.g., via a Michael reaction). See S. Xu et al. Angewandte Chemie International Ed. 57(6), 1601-1605 (2017) (incorporated by reference in its entirety).

Described herein are compounds of any of Formulae (B-I), (I)-(XXVIIId). Also described herein are pharmaceutically acceptable salts, pharmaceutically acceptable solvates, pharmaceutically active metabolites, and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided. In some embodiments, when compounds disclosed herein contain an oxidizable nitrogen atom, the nitrogen atom can be converted to an N-oxide by methods well known in the art. In certain embodiments, isomers and chemically protected forms of compounds having a structure represented by any of Formula (B-I), (I)—(XXVIIId) are also provided.

In particular embodiments, provided herein are KRas G12C irreversible inhibitors according to compounds of formula (I).

Thus, in some embodiments, provided herein are compounds according to Formula (B-I) having the structure:

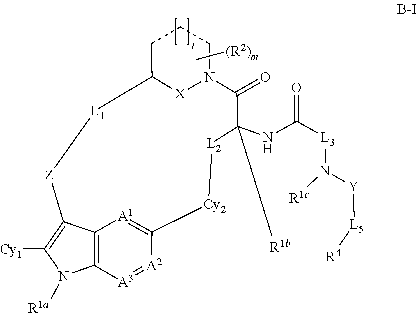

or a stereoisomer or a pharmaceutically acceptable salt thereof,
wherein:
each A¹, A², and A³ is independently —CR³=, or —N=; each R³ is independently H, halo, cyano, alkyl, haloalkyl, hydroxy, or alkoxy;
X is —CR^{1e}R^{1f}—, or —NR^{1g}—;
Z is absent or substituted or unsubstituted C₁-C₄ alkylene;
each Cy¹ and Cy² is independently substituted or unsubstituted aryl or heteroaryl;
Cy³ is substituted or unsubstituted cycloalkylene, or substituted or unsubstituted heterocycloalkyl;
L₁ is —C(O)—O—, —C(O)—N(R^{1d})—, or -Cy⁴-; Cy⁴ is substituted or unsubstituted heteroaryl; and —O— of —C(O)—O—, or —N(R^{1d})— of —C(O)—N(R^{1d})— is attached to Z; or —O— of —C(O)—O—, or —N(R^{1d})— of —C(O)—N(R^{1d})— is attached to the pyrrolo ring when Z is absent;
each L₂, and L₃ is independently substituted or unsubstituted C₁-C₄ alkylene;
Y is —C(O)—, —C(H)(CH₂F)—, —C(H)(CHF₂)—, or —C(H)(CF₃);
L₅ is substituted or unsubstituted C₁-C₆ alkylene, or Cy₃;
each R^{1a}, R^{1b}, R^{1c}, R^{1d}, R^{1e}, R^{1f}, and R^{1g} is independently H, or substituted or unsubstituted C₁-C₄ alkyl;
each R² is independently halo, cyano, alkyl, haloalkyl, or alkoxy; m is 0, 1, or 2;
R⁴ is any one of groups selected from i), and ii):
i) —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl;
ii) -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e},
L₄ is C(O), substituted or unsubstituted C₁-C₄ alkylene, or substituted or unsubstituted cycloalkyl;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, unsubstituted or substituted with one or more groups selected from substituted or unsubstituted amino, and substituted or unsubstituted heterocycloalkyl having 1-2 heteroatoms independently selected from nitrogen, oxygen, and sulfur;
R^{6d} is substituted or unsubstituted amino, substituted or unsubstituted heterocycloalkyl having 1-2 heteroatoms independently selected from nitrogen, oxygen, and sulfur, substituted or unsubstituted aryl, substituted or unsubstituted heteroaryl, or

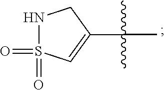 [SMILES: *C1=CS(=O)(=O)NC1]

R^{6e} is substituted or unsubstituted alkyl, substituted or unsubstituted aryl, substituted or unsubstituted heteroaryl, or

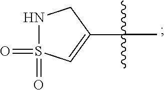 [SMILES: *C1=CS(=O)(=O)NC1]

each dotted bond is a single or a double bond; and the subscript t is 0, 1, or 2;
provided that when
i) X is —NR^{1g}—, R^{1g} is H, each dotted bond is a single bond, and t is 1; then R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

In some embodiments, L₅ is substituted or unsubstituted C₁-C₆ alkylene. In some embodiments, L₅ is substituted or unsubstituted Cy₃.

In some embodiments, t is 0. In some embodiments, t is 2. In some particular embodiments, t is 1

In some particular embodiments, provided herein are compounds according to Formula (I) having the structure:

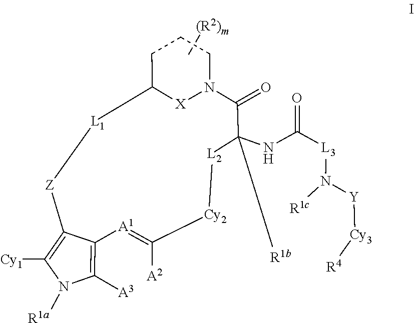

or a stereoisomer or a pharmaceutically acceptable salt thereof,
wherein:
each A¹, A², and A³ is independently —CR³=, or —N=; each R³ is independently H, halo, cyano, alkyl, haloalkyl, hydroxy, or alkoxy;
X is —CR^{1e}R^{1f}—, or —NR^{1g}—; Z is absent or substituted or unsubstituted C₁-C₄ alkylene;
each Cy¹ and Cy² is independently substituted or unsubstituted aryl or heteroaryl; Cy³ is substituted or unsubstituted heterocycloalkyl;
L¹ is —C(O)—O—, —C(O)—N(R^{1d})—, or -Cy⁴-; Cy⁴ is substituted or unsubstituted heteroaryl; and —O— of —C(O)—O—, or —N(R^{1d})— of —C(O)—N(R^{1d})— is attached to Z; or —O— of —C(O)—O—, or —N(R^{1d})— of —C(O)—N(R^{1d})— is attached to the pyrrolo ring when Z is absent;
each L², L³, and L⁴ is independently substituted or unsubstituted C₁-C₄ alkylene; Y is —C(O)—, or —C(H)CH₂F, or —C(H)(CF₃);
each R^{1a}, R^{1b}, R^{1c}, R^{1d}, R^{1e}, R^{1f}, and R^{1g} is independently H, or substituted or unsubstituted C₁-C₄ alkyl;
each R² is independently halo, cyano, alkyl, haloalkyl, or alkoxy; m is 0, 1, or 2;
R⁴ is any one of groups selected from i), and ii):
i) —C(O)—C(R^{6a})═C(R^{1b})(R^{6c}), —S(O)—C(R^{6a})═C(R^{1b})(R^{6c}), —S(O)₂—C(R^{6a})═C(R^{1b})(R^{6c}), or S(O)₂—CH₂—Cl;
ii) -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e},
L₄ is C(O), substituted or unsubstituted C₁-C₄ alkylene, or substituted or unsubstituted cycloalkyl;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, unsubstituted or substituted with one or more groups selected from substituted or unsubstituted amino, and substituted or unsubstituted heterocycloalkyl having 1-2 heteroatoms independently selected from nitrogen, oxygen, and sulfur;
R^{6d} is substituted or unsubstituted amino, substituted or unsubstituted heterocycloalkyl having 1-2 heteroatoms independently selected from nitrogen, oxygen, and sulfur, substituted or unsubstituted aryl, substituted or unsubstituted heteroaryl, or

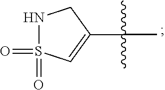 [SMILES: *C1=CS(=O)(=O)NC1]

R^{6e} is substituted or unsubstituted alkyl, substituted or unsubstituted aryl, substituted or unsubstituted heteroaryl, or

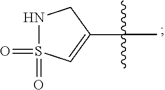 [SMILES: *C1=CS(=O)(=O)NC1]

and each dotted bond is a single or a double bond;
provided that when
i) X is —NR^{1g}—, R^{1g} is H, and each dotted bond is a single bond; then R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

In some embodiments, each A¹, A², and A³ is independently —CH═.

In some embodiments, L₁ is —C(O)—O—. In some embodiments, L₂ is —CH₂—, —C(Me)H—, or —C(Me)₂-.

In some embodiments, Cy₂ is substituted or unsubstituted heteroaryl. In some embodiments, Cy₂ is substituted or unsubstituted pyrrolyl, imidazolyl, thiazolyl, oxazolyl, or isoxazolyl. In some embodiments, Cy₂ is substituted or unsubstituted pyridyl, or pyrimidinyl. In some embodiments, Cy₂ is substituted or unsubstituted phenyl. In some embodiments, Cy₂ is unsubstituted phenyl.

In some embodiments, Z is substituted or unsubstituted C₃-C₄ alkylene. In some embodiments, Z is substituted or unsubstituted C₃ alkylene. In some embodiments, Z is C₃ alkylene, substituted with Me, Et, i-Pr, hydroxy, or hydroxymethyl. In some embodiments, Z is —CH₂—C(Me)₂-CH₂—.

In some embodiments, each dotted bond is a single bond.

In some embodiments, the compound is according to formula (IIa) or (IIb):

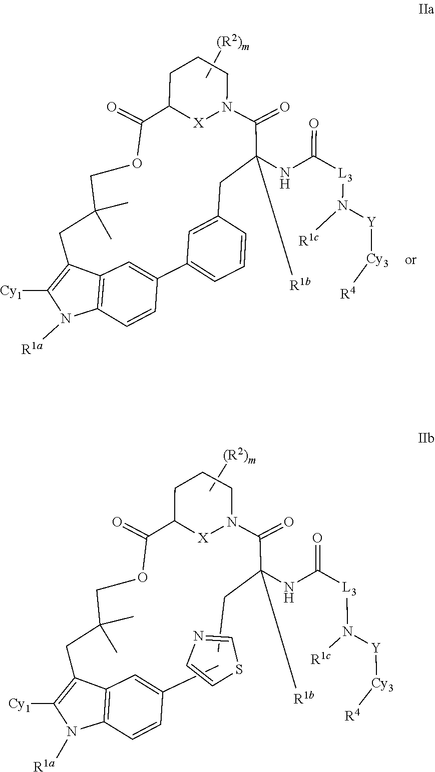

or a stereoisomer or a pharmaceutically acceptable salt thereof.

In some embodiments, L₃ is —CH₂—, —C(Me)H—, —C(Et)H—, —C(n-Pr)H—, —C(i-Pr)H—, or —C(Me)₂-. In some embodiments, L₃ is —C(i-Pr)H—.

In some embodiments, Y is —C(O)—.

In some embodiments, the compound is according to formula (IIIa) or (IIIb):

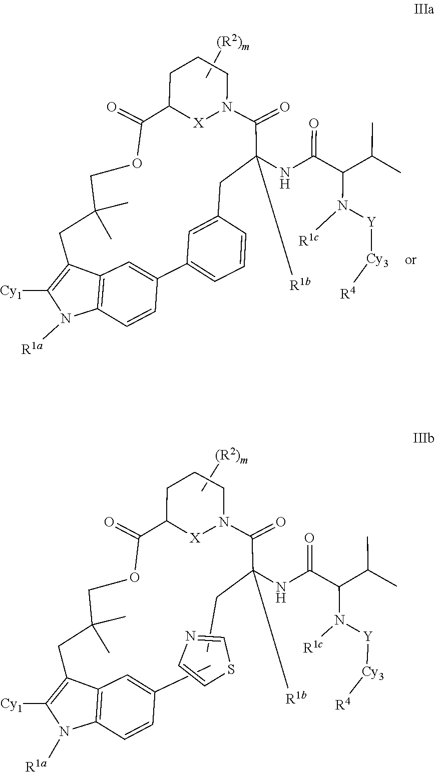

or a stereoisomer or a pharmaceutically acceptable salt thereof.

In some embodiments, R^{1b} is H, Me, or Et. In some embodiments, R^{1b} is H, or Me. In some embodiments, R^{1b} is H.

In some embodiments, R^{1c} is H, Me, or Et. In some embodiments, R^{1c} is H, or Me. In some embodiments, R^{1c} is Me.

In some embodiments, the compound is according to formula (IVa) or (IVb):

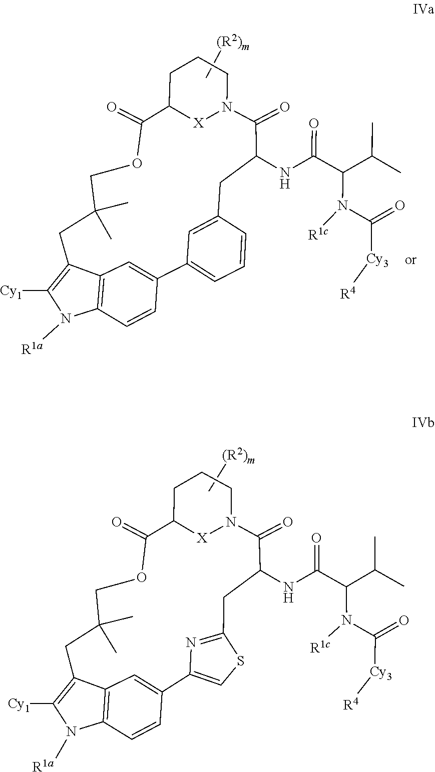

or a stereoisomer or a pharmaceutically acceptable salt thereof.

In some embodiments, R^{1a} is H, Me, Et, or i-Pr. In some embodiments, R^{1a} is H, Me, or Et. In some embodiments, R^{1a} is Et.

In some embodiments, m is 1, or 2. In some embodiments, m is 1, or 2; and each R² is independently alkyl or haloalkyl. In some embodiments, m is 1, or 2; and each R² is independently Me or CF₃. In some embodiments, m is 0

In some embodiments, the compound is according to formula (Va) or (Vb):

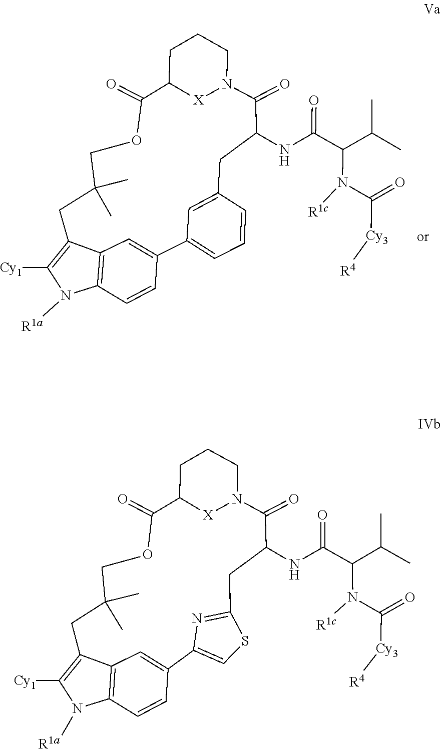

or a stereoisomer or a pharmaceutically acceptable salt thereof.

In some embodiments, Cy₁ is substituted or unsubstituted pyrrolyl, furanyl, imidazolyl, thiazolyl or thiadiazolyl. In some embodiments, Cy₁ is substituted or unsubstituted phenyl, pyridyl, naphthalenyl, pyrimidinyl, or pyrazinyl. In some embodiments, Cy₁ is substituted or unsubstituted phenyl, or pyridyl.

In some embodiments, Cy₁ is substituted or unsubstituted pyridyl. In some embodiments, Cy₁ is pyridyl, substituted with Me, Et, i-Pr, n-Pr, t-Bu, CF₃, OMe, OCF₃, CHF₂, CH₂OMe, CH₂—CH₂OMe, or C(OMe)(Me)H. In some embodiments, Cy₁ is pyridyl, substituted with C(OMe)(Me)H.

In some embodiments, the compound is according to formula (VIa) or (VIb):

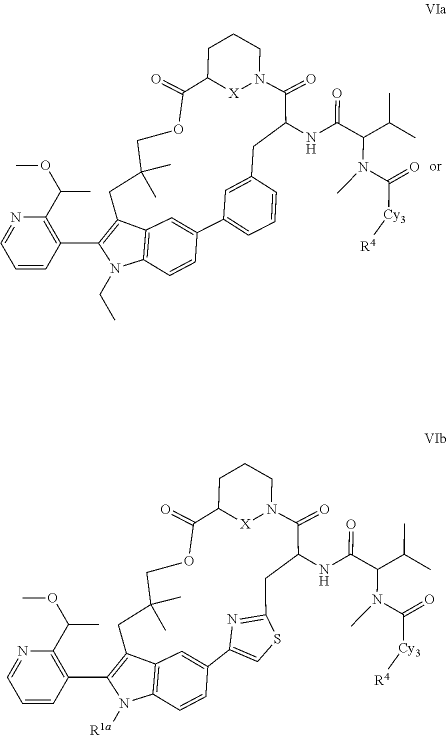

or a stereoisomer or a pharmaceutically acceptable salt thereof.

In some embodiments, X is NR^{1g}, and R^{1g} is H, Me, Et, or i-Pr. In some embodiments, X is NR^{1g}, and R^{1g} is H. In some embodiments, X is —NR^{1g}—, R^{1g} is H, each dotted bond is a single bond, t is 1; R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

In some embodiments, the compound is according to formula (VIIa) or (VIIb):

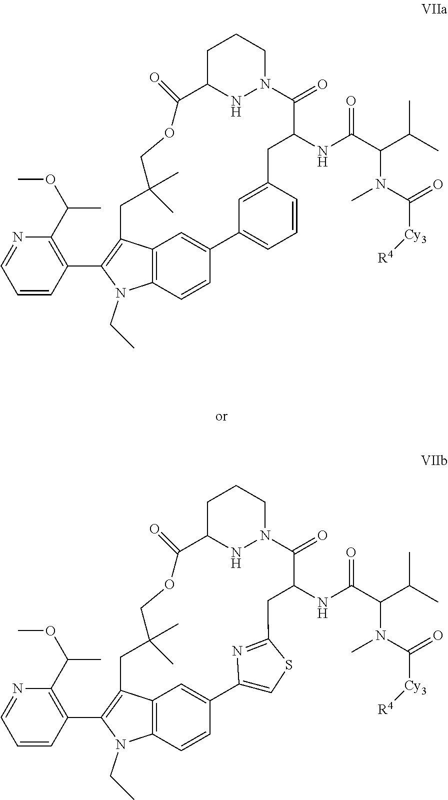

or a stereoisomer or a pharmaceutically acceptable salt thereof, and wherein R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

In some embodiments, X is CR^{1e}R^{1f}, and each R^{1e} and R^{1f} is independently H, Me, Et, or i-Pr. In some embodiments, X is CR^{1e}R^{1f}, and each R^{1e} and R^{1f} is H.

In some embodiments, the compound is according to formula (VIIIa) or (VIIIb):

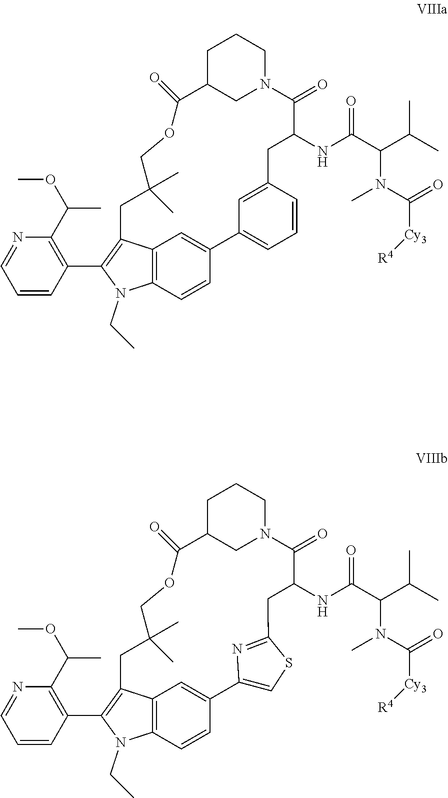

or a stereoisomer or a pharmaceutically acceptable salt thereof, and wherein R⁴ is —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl; -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

In some embodiments, Cy³ is substituted or unsubstituted cycloalkylene. In some embodiments, Cy³ is substituted or unsubstituted cyclopropyl, cyclobutyl, cyclopentyl or cyclohexyl. In some embodiments, Cy³ is substituted or unsubstituted N containing C₃-C₁₁ heterocycloalkyl. In some embodiments, Cy³ is substituted or unsubstituted N containing C₃-C₁₁ heterocycloalkyl; and R⁴ is attached to the N. In some embodiments, Cy³ is substituted or unsubstituted N containing C₃-C₁₁ heterocycloalkyl, and the heterocycloalkyl is a monocyclic, bicyclic, bridged, fused, or partially saturated heterocyclic ring.

In some embodiments, Cy³ is substituted or unsubstituted pyrrolidinyl, piperidinyl, piperazinyl, morpholinyl, aziridinyl, azetidinyl, azepinyl, diazepinyl,

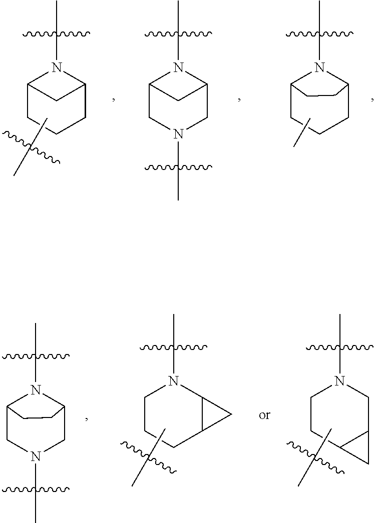

In some embodiments, Cy³ is substituted or unsubstituted pyrrolidinyl, piperidinyl, piperazinyl, or morpholinyl. In some embodiments, Cy³ is substituted or unsubstituted pyrrolidinyl.

In some embodiments, the compound is according to formula (IXa), (IXb), (IXc) or (IXd):

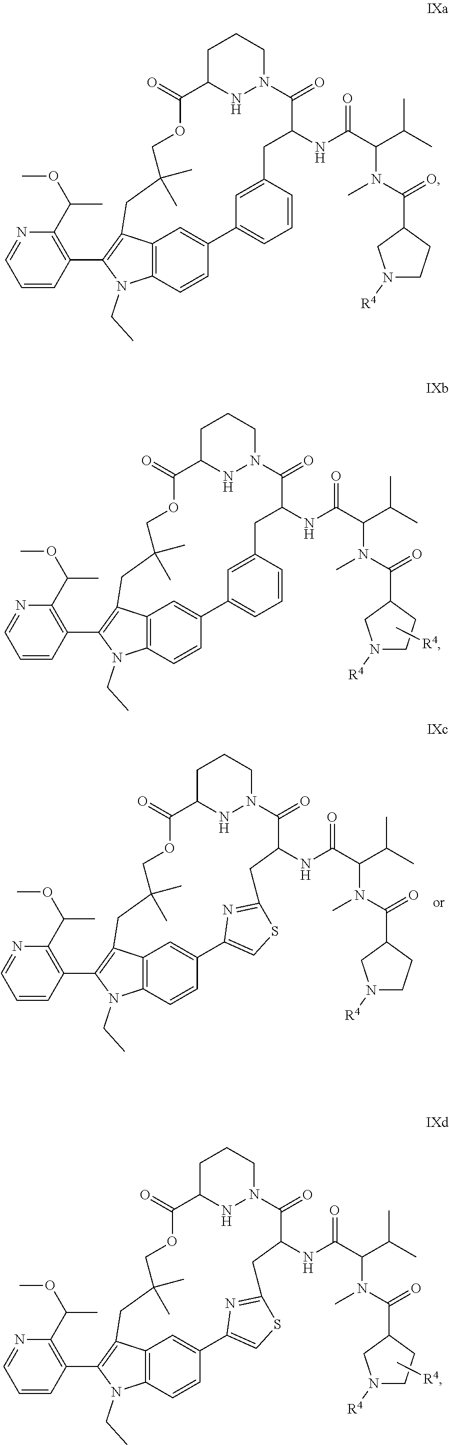

or a stereoisomer or a pharmaceutically acceptable salt thereof, and wherein R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or —C≡C—R^{6e}; and R7 is H, or C1-C4 alkyl.

In some embodiments, the compound is according to formula (Xa), (Xb), (Xc), or (Xd:

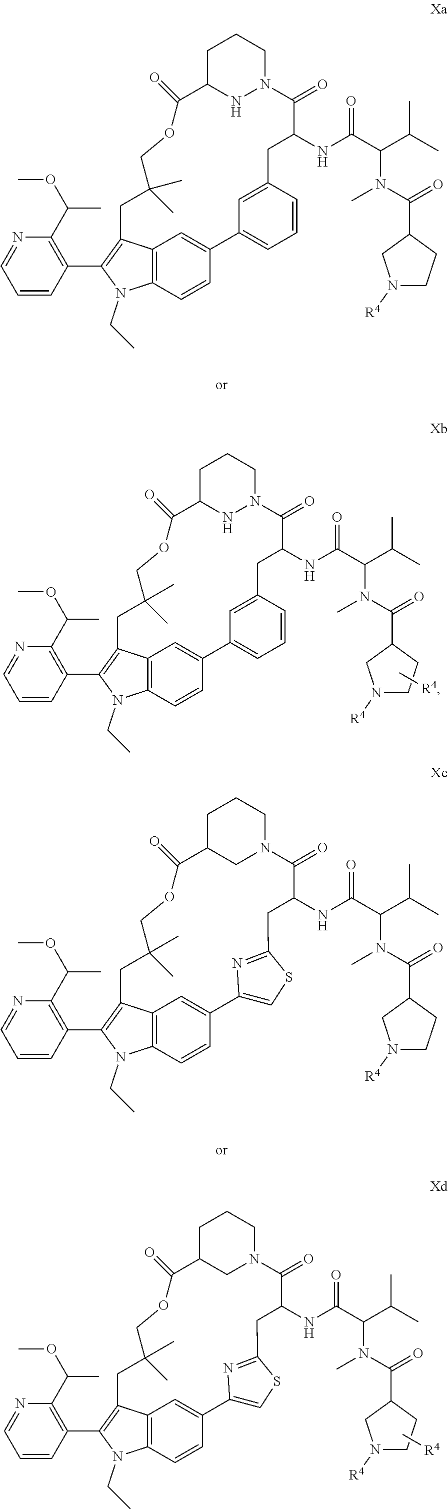

or a stereoisomer or a pharmaceutically acceptable salt thereof, and wherein R⁴ is —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl; -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or —C≡C—R^{6e}; and R7 is H, or C1-C4 alkyl.

In some embodiments, R⁷ is H, Me, Et, or i-Pr. In some embodiments, R⁷ is Me.

In some embodiments, R⁴ is -L₄-R^{6d}. In some embodiments, R⁴ is -L₄-C≡C—R^{6e}.

In some embodiments, L₄ is —CH₂—, —CH₂—CH₂—, —CH₂—CH₂—CH₂—, —C(O)—, —C(Me)H, —CMe₂-, or

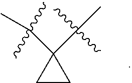 [SMILES: *C1(*)CC1]

In some embodiments, L₄ is —CH₂—. In some embodiments, L₄ is —C(O)—.

In some embodiments, the compound is according to formula (XIa), (XIb), or (XIc):

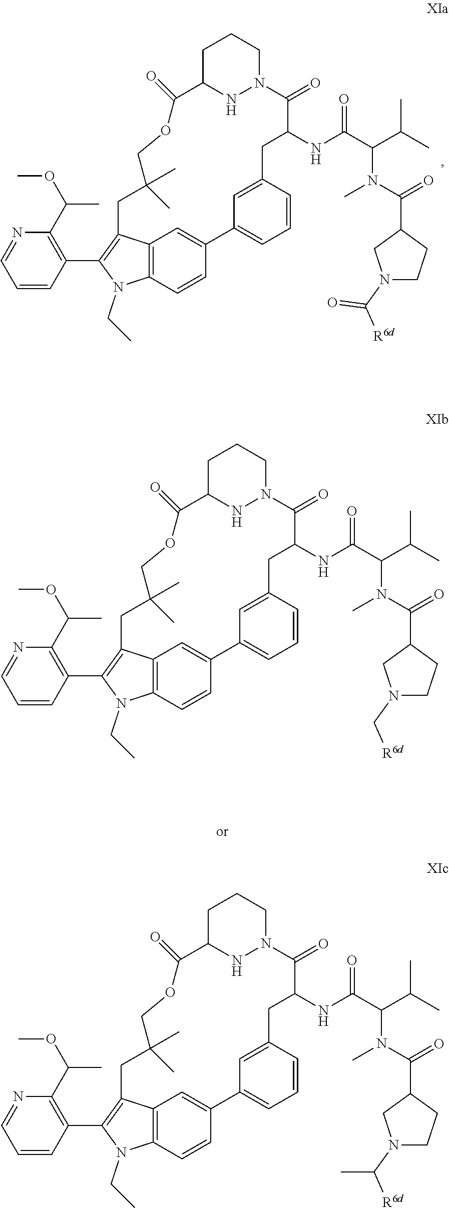

or a stereoisomer or a pharmaceutically acceptable salt thereof.

In some embodiments, the compound is according to formula (XIIa), (XIIb), or (XIIc):

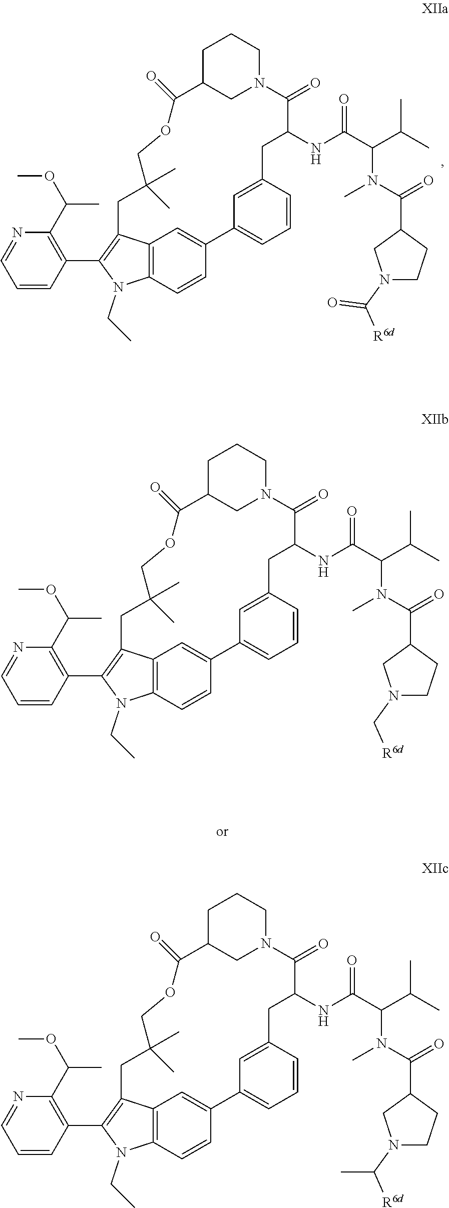

or a stereoisomer or a pharmaceutically acceptable salt thereof.

In some embodiments, R^{6d} is

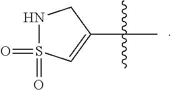 [SMILES: *C1=CS(=O)(=O)NC1]

In some embodiments, the compound is according to formula (XIIIa) or (XIIIb):

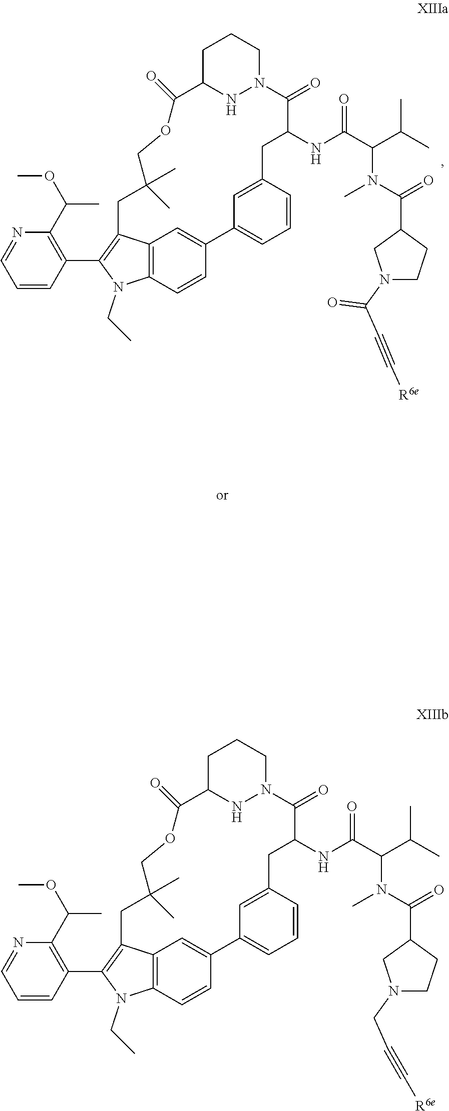

or a stereoisomer or a pharmaceutically acceptable salt thereof.

In some embodiments, the compound is according to formula (XIVa) or (XIVb):

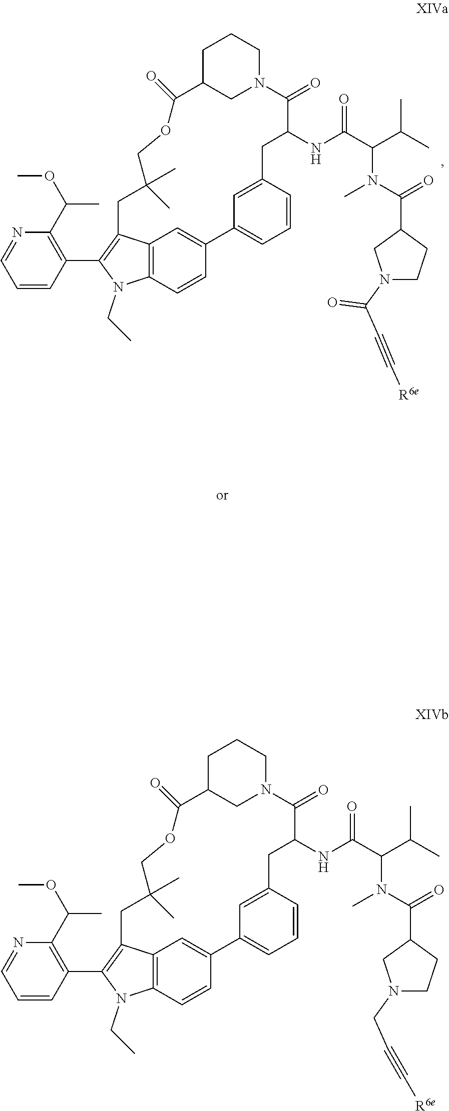

or a stereoisomer or a pharmaceutically acceptable salt thereof.

In some embodiments, R^{6e} is substituted or unsubstituted alkyl. In some embodiments, R^{6e} is Me, Et. In some embodiments, R^{6e} is substituted or unsubstituted aryl, substituted or unsubstituted heteroaryl.

In some embodiments, R^{6e} is substituted or unsubstituted heteroaryl. In some embodiments, R^{6e} is substituted or unsubstituted benzoxazolyl, benzthiazolyl, benzimidazolyl, indazolyl, or indolyl.

In some embodiments, R^{6e} is substituted or unsubstituted benzoxazolyl.

In some embodiments, R⁴ is -L₄-C(R^{6a})═C(R^{6b})—C(O)—R^{6d}, -L₄-C(R^{6a})═C(R^{6b})—S(O)—R^{6d}, or -L₄-C(R^{6a})═C(R^{6b})—S(O)₂—R^{6d}; and L₄ is —CH₂—, —CH₂—CH₂—, —CH₂—CH₂—CH₂—, —C(O)—, —C(Me)H, —CMe₂-, or

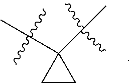 [SMILES: *C1(*)CC1]

In some embodiments, R⁴ is —CH₂—C(R^{6a})═C(R^{6b})—C(O)—R^{6d}, —CH₂—C(R^{6a})═C(R^{6b})—S(O)—R^{6d}, or —CH₂—C(R^{6a})═C(R^{6b})—S(O)₂—R^{6d}.

In some embodiments, R⁴ is —C(O)—C(R^{6a})═C(R^{6b})—C(O)—R^{6d}, —C(O)—C(R^{6a})═C(R^{6b})—S(O)—R^{6d}, or —C(O)—C(R^{6a})═C(R^{6b})—S(O)₂—R^{6d}.

In some embodiments, each of R^{6a}, and R^{6b} is H. In some embodiments, each of R^{6a} and R^{6b} is H or F. In some embodiments, one of R^{6a} and R^{6b} is CN. In some embodiments, R⁴ is —CH₂—CH═CH—C(O)—R^{6d}, —CH₂—CH═CH—S(O)—R^{6d}, or —CH₂—CH═CH—S(O)₂—R^{6d}. In some embodiments, R⁴ is —C(Me)H—CH═CH—C(O)—R^{6d}, —C(Me)H—CH═CH—S(O)—R^{6d}, or —C(Me)H—CH═CH—S(O)₂—R^{6d}. In some embodiments, R⁴ is —C(O)—CH═CH—C(O)—R^{6d}, —C(O)—CH═CH—S(O)—R^{6d}, or —C(O)—CH═CH—S(O)₂—R^{6d}.

In some embodiments, the compound is according to formula (XVa), (XVb), (XVc), or (XVd):

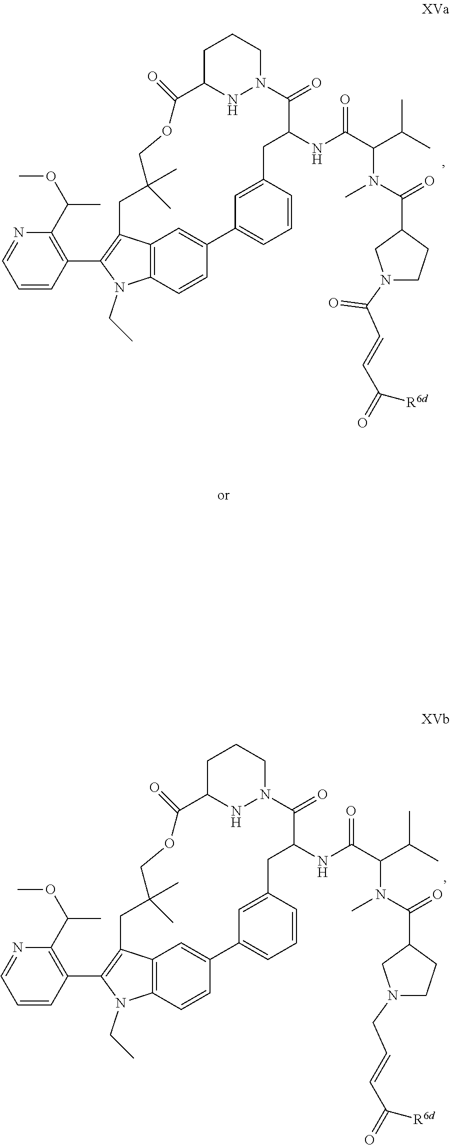

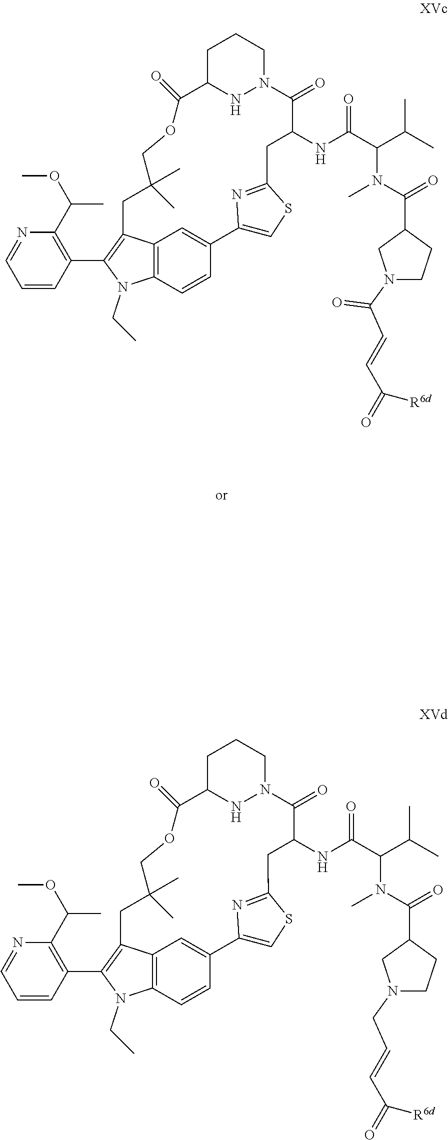

or a stereoisomer or a pharmaceutically acceptable salt thereof.

In some embodiments, the compound is according to formula (XVIa), (XVIb), (XVIc), or (XVId):

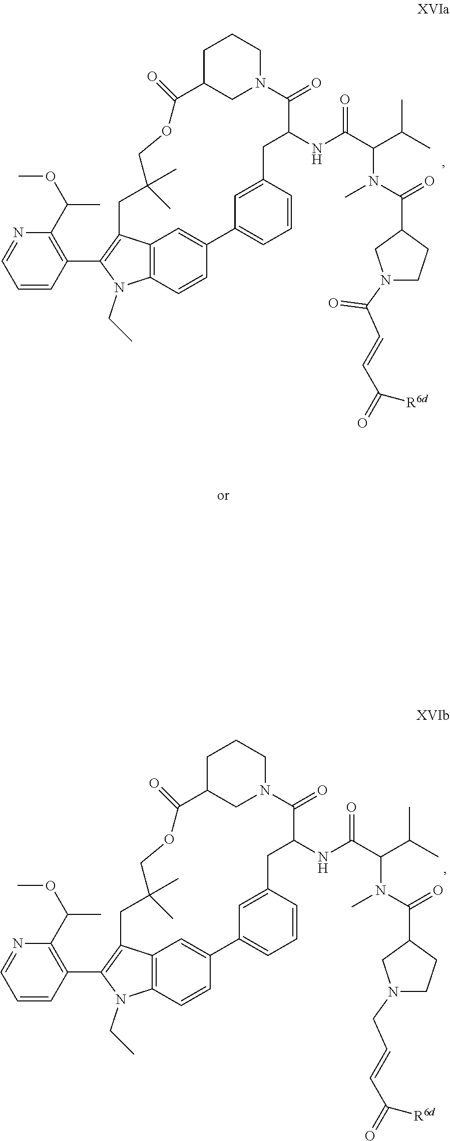

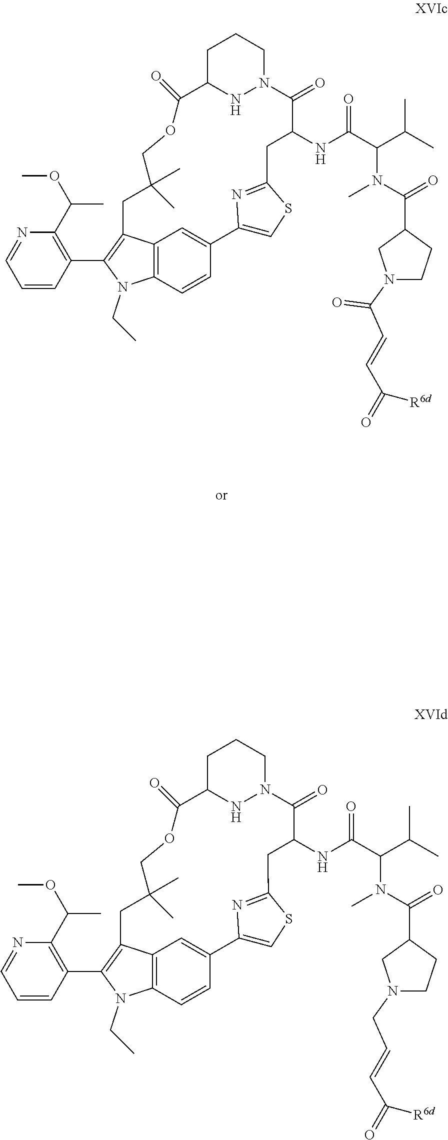

or a stereoisomer or a pharmaceutically acceptable salt thereof.

In some embodiments, R^{6a} and R^{6b} form a bond.

In some embodiments, R⁴ is —CH₂—C═C≡C(O)—R^{6d}, —CH₂—C≡C—S(O)—R^{6a}, or —CH₂—C≡C—S(O)₂—R^{6d}.

In some embodiments, R⁴ is —C(Me)H—C═C≡C(O)—R^{6d}, —C(Me)H—C≡C—S(O)—R^{6a}, or —C(Me)H—C≡C—S(O)₂—R^{6d}.

In some embodiments, R⁴ is —C(O)—C═C≡C(O)—R^{6d}, —C(O)—C≡C—S(O)—R^{6d}, or —C(O)—C≡C—S(O)₂—R^{6d}.

In some embodiments, the compound is according to formula (XVIIa), (XVIIb), (XVIIc), or (XVIId):

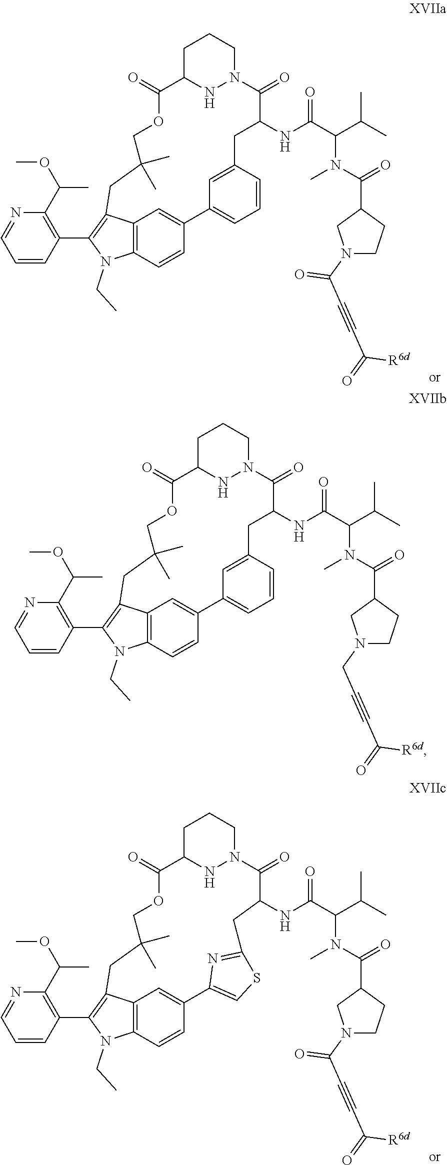

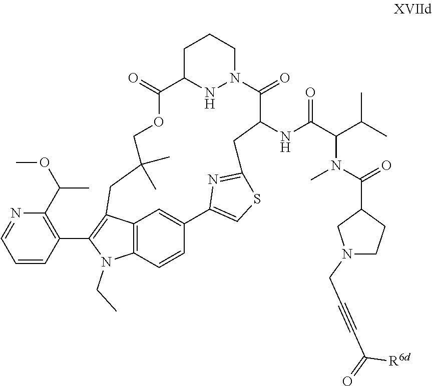

or a stereoisomer or a pharmaceutically acceptable salt thereof.
In some embodiments, the compound is according to formula (XVIIIa) or (XVIIIb):

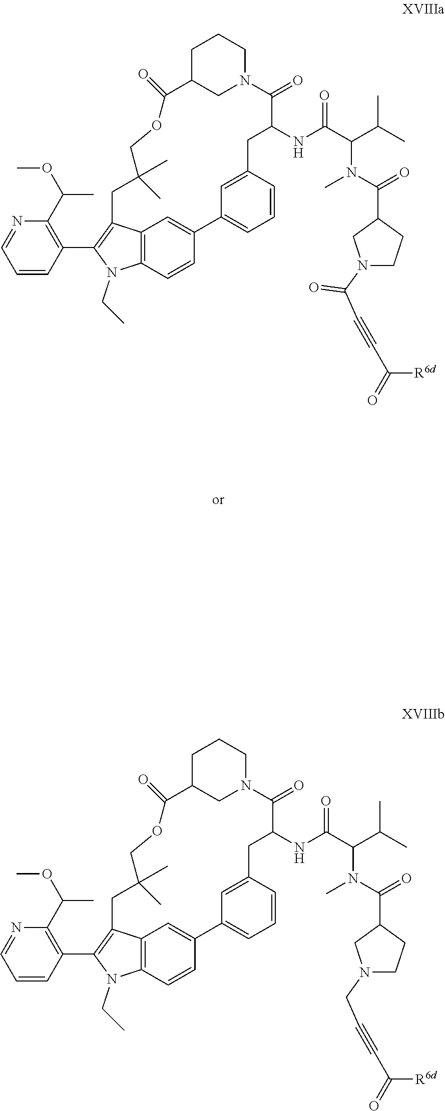

or a stereoisomer or a pharmaceutically acceptable salt thereof.

In some embodiments, R^{6d} is substituted or unsubstituted amino, substituted or unsubstituted heterocycloalkyl having 1-2 heteroatoms independently selected from nitrogen, oxygen, and sulfur, substituted or unsubstituted aryl, substituted or unsubstituted heteroaryl, or

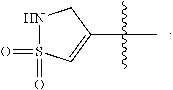 [SMILES: *C1=CS(=O)(=O)NC1]

In some embodiments, R^{6d} is

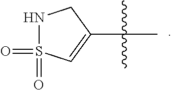 [SMILES: *C1=CS(=O)(=O)NC1]

In some embodiments, R^{6d} is substituted or unsubstituted amino.

In some embodiments, R^{6d} is dialkylamino.

In some embodiments, R^{6d} is dimethylamino.

In some embodiments, R^{6d} is substituted or unsubstituted heterocycloalkyl having 1-2 heteroatoms independently selected from nitrogen, oxygen, and sulfur.

In some embodiments, R^{6d} is substituted or unsubstituted pyrrolidinyl, piperidinyl, piperizinyl, or morpholinyl.

In some embodiments, R^{6d} is substituted or unsubstituted pyrrolidinyl.

In some embodiments, R^{6d} is substituted or unsubstituted morpholinyl.

In some embodiments, R^{6d} is substituted or unsubstituted piperidinyl.

In some embodiments, R^{6d} is substituted or unsubstituted aryl.

In some embodiments, R^{6d} is substituted or unsubstituted heteroaryl.

In some embodiments, the compound is according to formula (X):

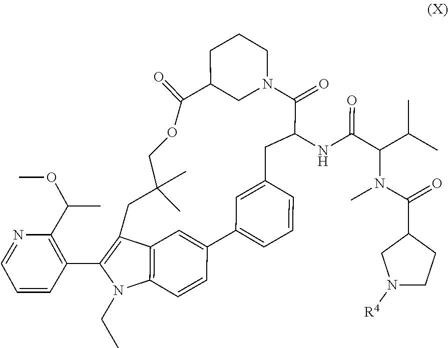

or a stereoisomer or a pharmaceutically acceptable salt thereof, and wherein R⁴ is —C(O)—C(R^{6a})═C(R^{6b})(R^{6c}), —S(O)—C(R^{6a})═C(R^{6b})(R^{6c}), —S(O)₂—C(R^{6a})═C(R b)(R^{6c}), or S(O)₂—CH₂—Cl.

In some embodiments, R⁴ is —C(O)—C(R^{6a})═C(R^{b})(R^{6c}).

In some embodiments, R⁴ is-S(O)₂—C(R^{6a})═C(R^{6b})(R^{6c}).

In some embodiments, R⁴ is S(O)₂—CH₂—Cl.

In some embodiments, the compound is according to formula (XXIa) or (XXIb):

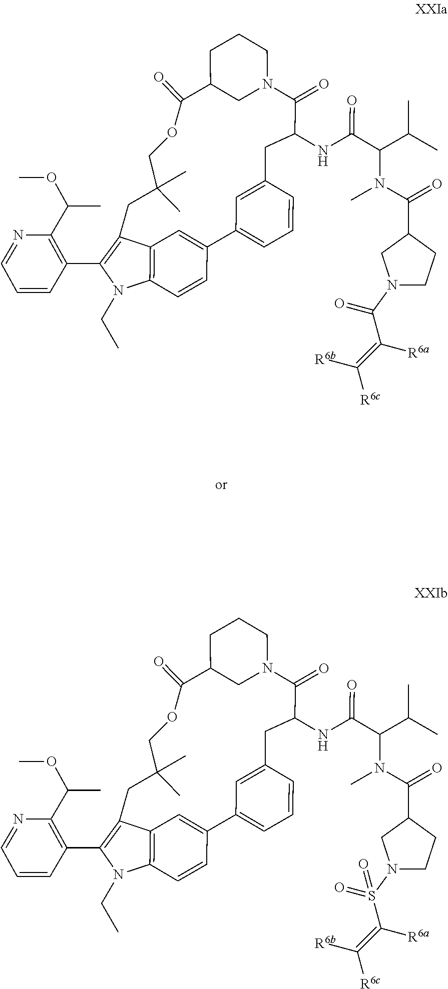

or a stereoisomer or a pharmaceutically acceptable salt thereof.

In some embodiments, each of R^{6a}, and R^{6b} is H.

In some embodiments, one of R^{6a}, and R^{6b} is F and the other is H.

In some embodiments, one of R^{6a}, and R^{6b} is alkyl and the other is H.

In some embodiments, one of R^{6a}, and R^{6b} is Me or Et, and the other is H

In some embodiments, one of R^{6a}, and R^{6b} is CN and the other is H.

In some embodiments, the compound is according to formula (XXIIa) or (XXIIb):

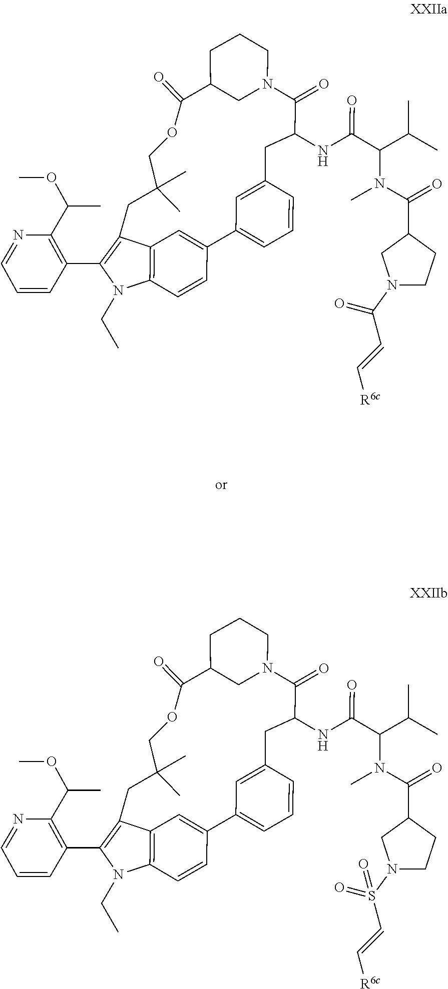

or a stereoisomer or a pharmaceutically acceptable salt thereof.

In some embodiments, R^{6a} and R^{6b} form a bond.

In some embodiments, the compound is according to formula (XXIIIa) or (XXIIIb):

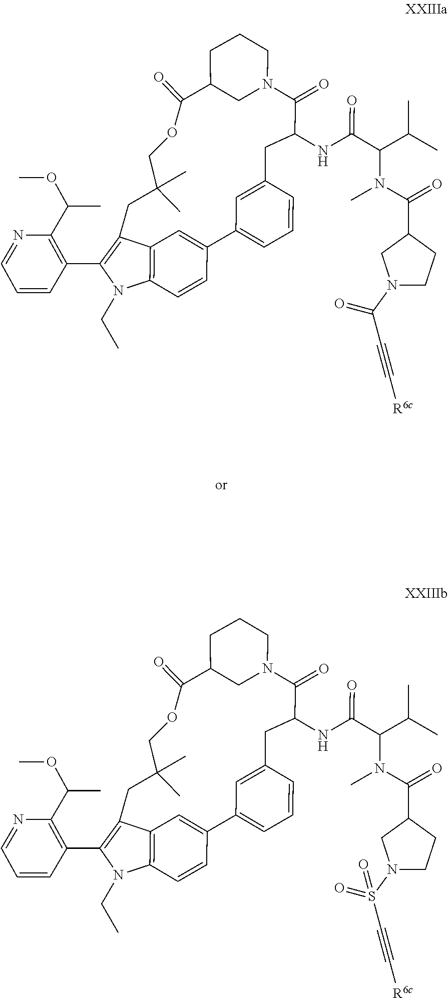

or a stereoisomer or a pharmaceutically acceptable salt thereof.

In some embodiments, R^{6c} is H.

In some embodiments, R^{6c} is substituted or unsubstituted alkyl.

In some embodiments, R^{6c} is H, or substituted or unsubstituted alkyl.

In some embodiments, R^{6c} is unsubstituted alkyl.

In some embodiments, R^{6c} is Me, or Et.

In some embodiments, R^{6c} is Me.

In some embodiments, R^{6c} is substituted alkyl.

In some embodiments, R^{6c} is alkyl substituted with amino, alkylamino or dialkylamino.

In some embodiments, R^{6c} is alkyl substituted with dimethylamino.

In some embodiments, R^{6c} is —CH₂NMe₂.

In some embodiments, R^{6c} is alkyl substituted with hydroxy, CN, or substituted or unsubstituted alkoxy.

In some embodiments, R^{6c} is alkyl substituted with OH, OMe, CN, or OCF3

In some embodiments, R^{6c} is alkyl substituted with cycloalkyl.

In some embodiments, R^{6c} is alkyl substituted with cyclopropyl, cyclobutyl, cyclopentyl or cyclohexyl.

In some embodiments, R^{6c} is alkyl substituted with heterocycloalkyl.

In some embodiments, R^{6c} is —(CH₂)_{q}-heterocycloalkyl; and q is 1, 2, 3, or 4

In some embodiments, R^{6c} is —(CH₂)_{q}-heterocycloalkyl; and q is 1

In some embodiments, R^{6c} is —(CH₂)_{q}-heterocycloalkyl; and q is 2

In some embodiments, R^{6c} is —(CH₂)_{q}-heterocycloalkyl; and q is 3

In some embodiments, R^{6c} is —CH₂-azetidin-1-yl, —CH₂-pyrrolidin-1-yl, —CH₂-piperidin-1-yl, or —CH₂-morpholin-1-yl.

In some embodiments, the compound is according to formula (XXIVa) or (XXIVb):

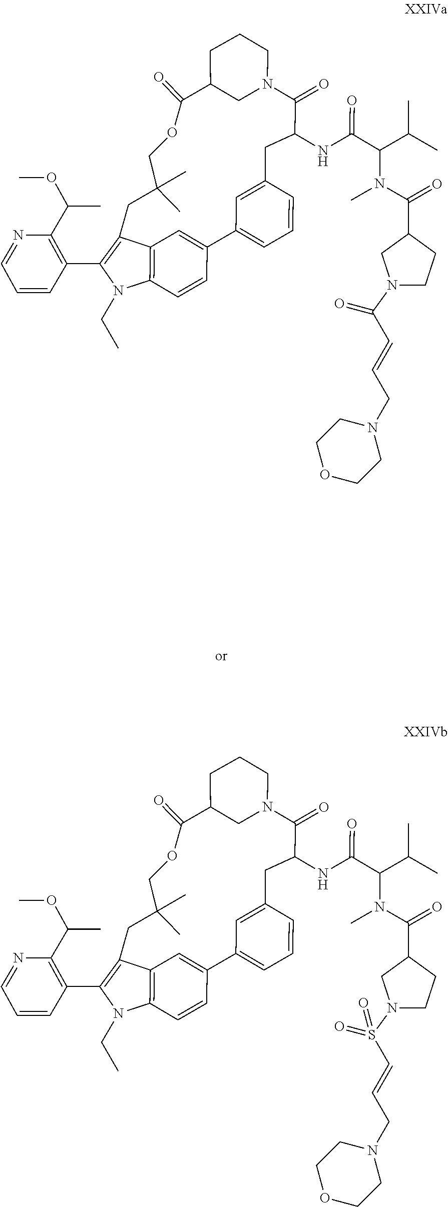 [SMILES: CCn1c(-c2cccnc2C(C)OC)c2c3cc(ccc31)-c1cccc(c1)CC(NC(=O)[C@@H](C(C)C)N(C)C(=O)[C@@H]1CCN(C(=O)/C=C/CN3CCOCC3)C1)C(=O)N1CCCC(C1)C(=O)OCC(C)(C)C2 ; CCn1c(-c2cccnc2C(C)OC)c2c3cc(ccc31)-c1cccc(c1)CC(NC(=O)[C@@H](C(C)C)N(C)C(=O)[C@@H]1CCN([S@TB17](=O)(=O)/C=C/CN3CCOCC3)C1)C(=O)N1CCCC(C1)C(=O)OCC(C)(C)C2]

or a stereoisomer or a pharmaceutically acceptable salt thereof.

In some embodiments, the compound is according to formula (XXVa) or (XXVb):

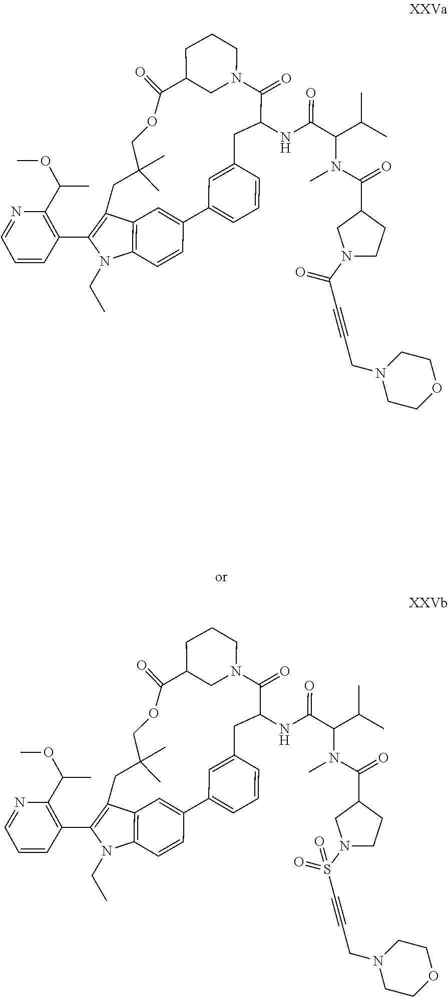 [SMILES: CCn1c(-c2cccnc2C(C)OC)c2c3cc(ccc31)-c1cccc(c1)CC(NC(=O)C(C(C)C)N(C)C(=O)[C@@H]1CCN(C(=O)C#CCN3CCOCC3)C1)C(=O)N1CCCC(C1)C(=O)OCC(C)(C)C2 ; CCn1c(-c2cccnc2C(C)OC)c2c3cc(ccc31)-c1cccc(c1)CC(NC(=O)C(C(C)C)N(C)C(=O)[C@@H]1CCN([S@TB17](=O)(=O)C#CCN3CCOCC3)C1)C(=O)N1CCCC(C1)C(=O)OCC(C)(C)C2]

or a stereoisomer or a pharmaceutically acceptable salt thereof.

In some embodiments, the compound is according to formula (XXVIa), (XXVIb), (XXVIc), or (XXVId):

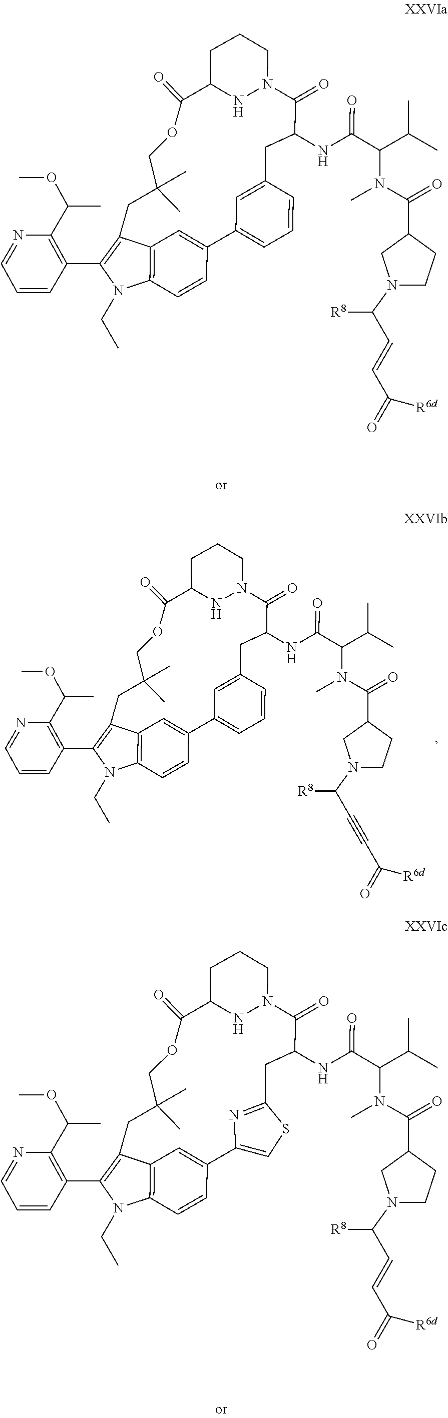

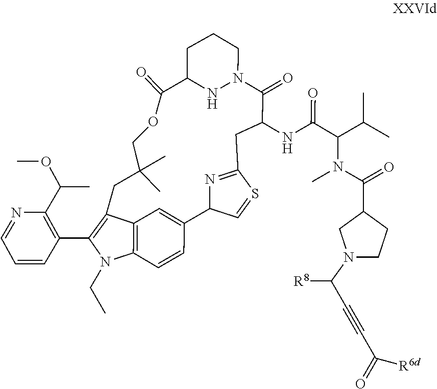

or a stereoisomer or a pharmaceutically acceptable salt thereof, wherein
R⁸ is H, or C₁-C₄ alkyl; and R^{6d} is alkylamino or dialkylamino, or a substituted or unsubstituted heterocycle.

In some embodiments, the compound is according to formula (XXVIIa), (XXVIIb), (XXVIIc), or (XXVIId):

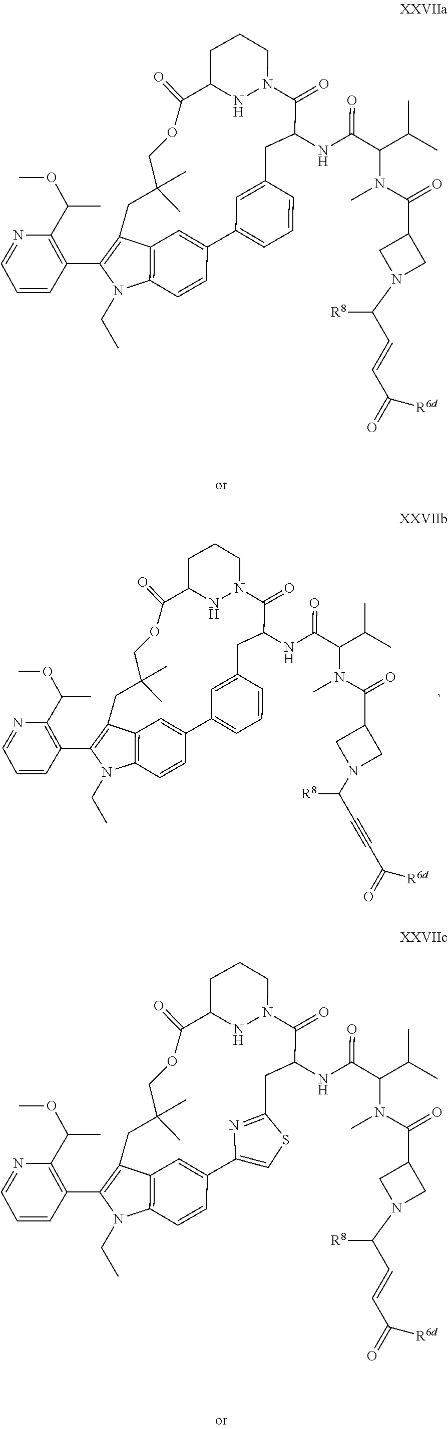

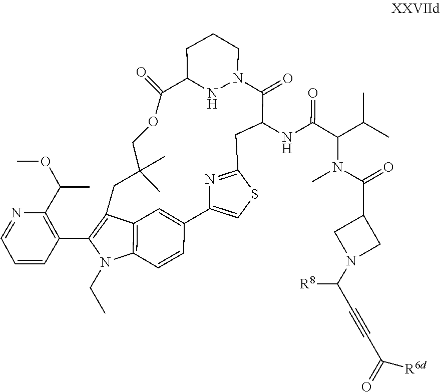

or a stereoisomer or a pharmaceutically acceptable salt thereof, wherein
R⁸ is H, or C₁-C₄ alkyl; and R^{6d} is alkylamino or dialkylamino, or a substituted or unsubstituted heterocycle.

In some embodiments, the compound is according to formula (XXVIIIa), (XXVIIIb), (XXVIIIc), or (XXVIIId):

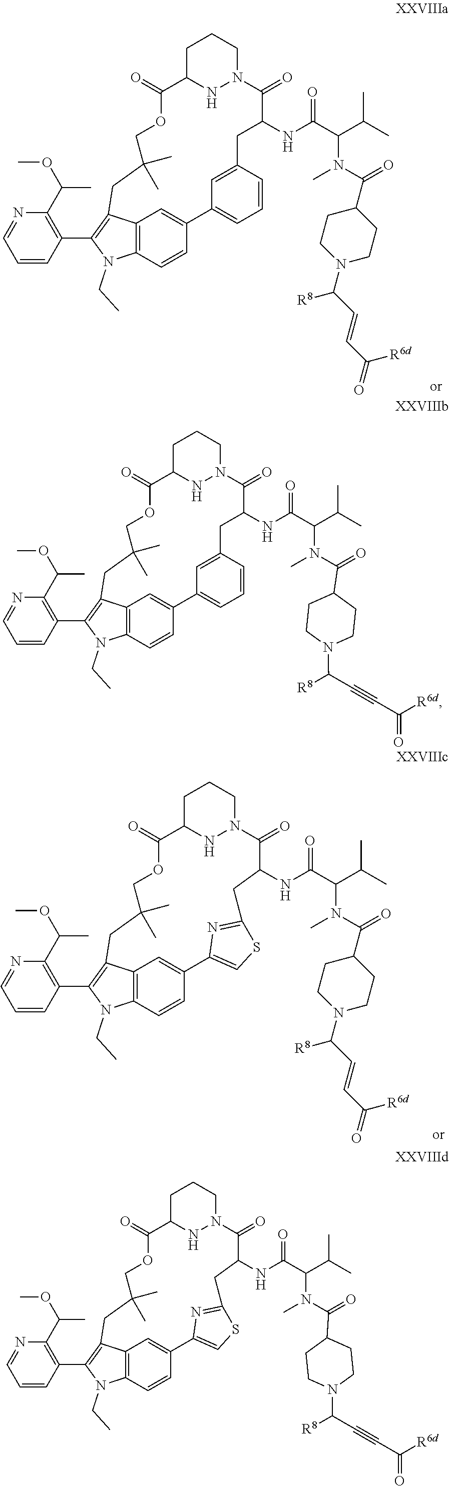

or a stereoisomer or a pharmaceutically acceptable salt thereof, wherein
R⁸ is H, or C₁-C₄ alkyl; and R^{6d} is alkylamino or dialkylamino, or a substituted or unsubstituted heterocycle.

In some embodiments, R^{6d} is NHMe, or NMe₂.

In some embodiments, R^{6d} is substituted or unsubstituted heterocycle.

In some embodiments, R^{6d} is substituted or unsubstituted azetidinyl.

In some embodiments, R^{6d} is azetidinyl, substituted with F or diF.

In some embodiments, R^{6d} is 3-fluoroazetidinyl, or 3,3-difluoro azetidinyl.

In some embodiments, R^{8d} is H.

In some embodiments, R^{8d} is Me.

In some particular embodiments, the compound is selected from any one of compounds listed in 1A and Table 1B.

In some particular embodiments, the compound is any compound selected from the following table, or a pharmaceutically acceptable salt or solvate or stereoisomer thereof:

| ID | Structure* | MW |
|---|---|---|
| 1 | 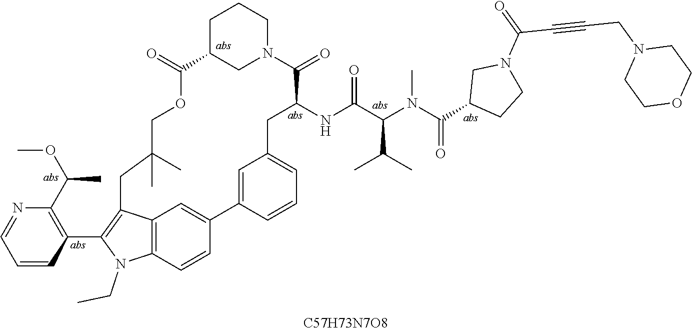 | 984.23 |
| 1.1 | 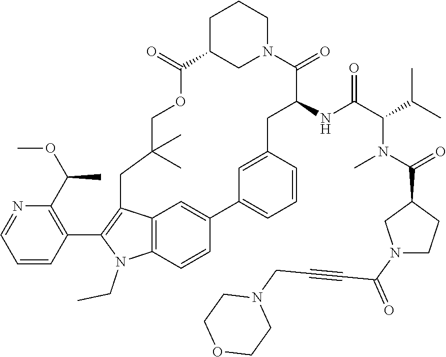 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#CCN3CCOCC3)C1)C(=O)N1CCC[C@H](C1)C(=O)OCC(C)(C)C2] | 984.23 |
| 2 | 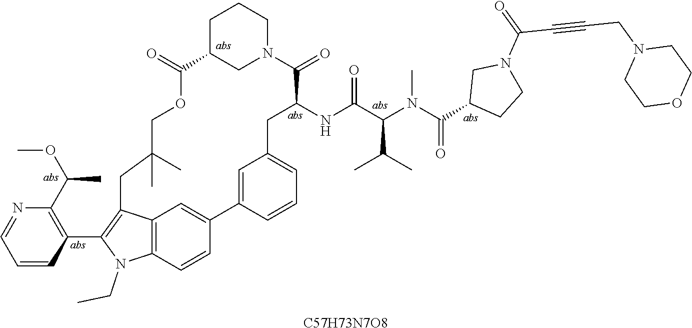 | 984.23 |
| 3 | 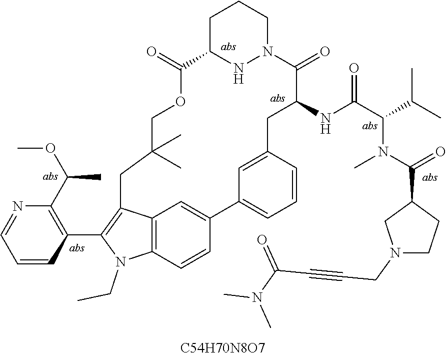 | 943.18 |
| 3.1 | 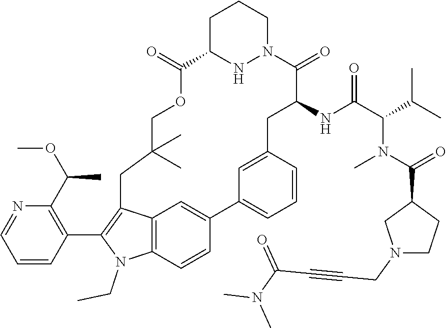 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(CC#CC(=O)N(C)C)C1)C(=O)N1CCC[C@H](N1)C(=O)OCC(C)(C)C2] | 943.18 |
| 3.2 | 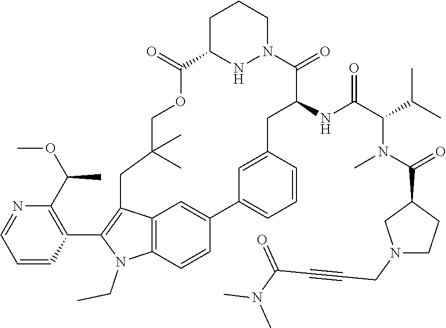 | 943.18 |
| 4 | 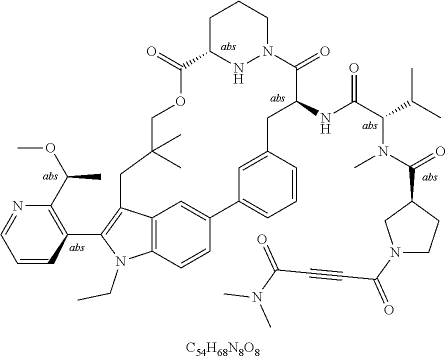 | 957.17 |
| 4.1 | 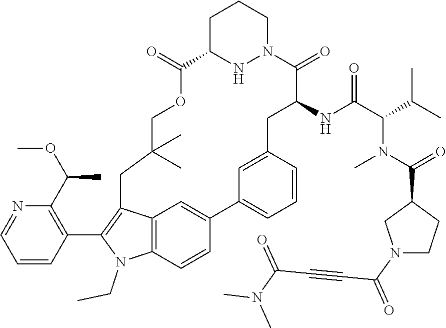 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#CC(=O)N(C)C)C1)C(=O)N1CCC[C@H](N1)C(=O)OCC(C)(C)C2] | 957.17 |
| 4.2 | 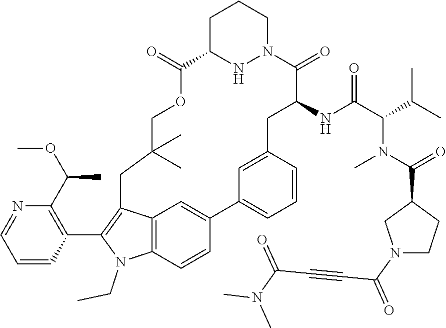 | 957.17 |
| 5 | 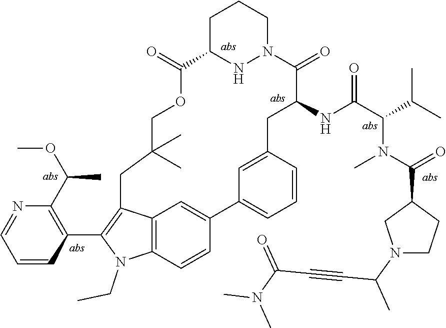 | 957.21 |
| 5.1 | 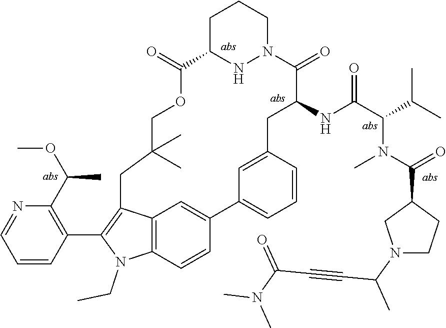 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(C)C#CC(=O)N(C)C)C1)C(=O)N1CCC[C@H](N1)C(=O)OCC(C)(C)C2] | 957.21 |
| 5.2 | 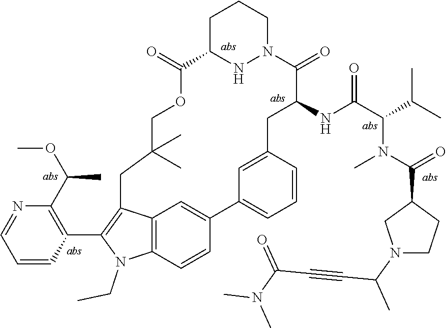 | 957.21 |

Certain compounds provided herein are depicted with particular isomers or conformational isomers on their pyridine rings. Each and every pyridine isomer or conformational isomer is a compound of the description herein.

Embodiments of the compounds of Formula (I) display improved potency against KRas G12C with IC₅₀ values of as low as less than 1 nM or less than 0.1 nM, and/or high occupancy of active site of KRas G12C (e.g., more than 50%, 70% or 90% occupancy) at low dosages of below 5 mg/kg (e.g., at or below 3 mg/kg) when administered in vivo (e.g., in rats).

In some embodiments, provided herein is a pharmaceutical composition comprising a compound according to formula (I).

In some embodiments, provided herein is a pharmaceutical composition comprising a therapeutically effective amount of a compound of formula (I), and a pharmaceutically acceptable excipient.

In some embodiments, the pharmaceutical composition is formulated for a route of administration selected from oral administration, parenteral administration, buccal administration, nasal administration, topical administration, or rectal administration.

In some embodiments, provided herein are methods for treating an autoimmune disease or condition comprising administering to a patient in need the pharmaceutical composition provided herein.

In some embodiments, provided herein are methods for treating a cancer comprising administering to a patient in need the pharmaceutical composition provided herein.

In some embodiments, the cancer is a B-cell proliferative disorder.

In some embodiments, the B-cell proliferative disorder is diffuse large B cell lymphoma, follicular lymphoma, or chronic lymphocytic leukemia. In some embodiments, the disorder is myeloid leukemia. In some embodiments, the disorder is acute myeloid leukemia (AML). In some embodiments, the B-cell proliferative disorder is lymphoid leukemia. In some embodiments, the disorder is acute lymphocytic leukemia (ALL). In some embodiments, the disorder is soft tissue tumors. In some embodiments, the tumor is glioblastoma. In some embodiments, the tumor is pancreatic tumor. In some embodiments, the disorder is renal cell cancer.

In some embodiments, provided herein are methods for treating mastocytosis comprising administering to a patient in need the pharmaceutical composition provided herein.

In some embodiments provided herein are methods for treating diffuse large B cell lymphoma, follicular lymphoma or chronic lymphocytic leukemia comprising administering to a subject in need thereof a composition containing a therapeutically effective amount of a compound of formula (I) that is inhibitor of the KRas G12C.

In some embodiments, provided herein are methods for treating mastocytosis, comprising administering to a subject in need thereof a composition containing a therapeutically effective amount of a compound of formula (I) that is inhibitor of KRas G12C.

In some embodiments, the subject in need is suffering from a cancer. In some embodiments, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenström macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds.

In some embodiments, provided herein is a method for treating an autoimmune disease by administering to a subject in need thereof a composition containing a therapeutically effective amount of at least one compound having the structure of Formula (B-I), (I)—(XXVIIId).

In some embodiments, provided herein is a method for treating a cancer by administering to a subject in need thereof a composition containing a therapeutically effective amount of at least one compound having the structure of Formula (B-I), (I)—(XXVIIId). In some embodiments, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis. In some embodiments, where the subject is suffering from a cancer, an anti-cancer agent is administered to the subject in addition to one of the above-mentioned compounds.

In some embodiments, the cancer is acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, or bladder cancer.

In some embodiments, the cancer is bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, or cardiac tumors, craniopharyngioma, cutaneous T-cell lymphoma, extrahepatic ductal carcinoma in situ (DCIS), embryonal tumors, Ewing sarcoma, extracranial germ cell tumor, extragonadal germ cell tumor, gastrointestinal carcinoid tumor, gastrointestinal stromal tumors (GIST), germ cell tumor, gestational trophoblastic tumor, Hodgkin's lymphoma, islet cell tumors, pancreatic neuroendocrine tumors, midline tract carcinoma, non-Hodgkin's lymphoma.

In some embodiments, the cancer is multiple endocrine neoplasia syndromes, multiple myeloma/plasma cell neoplasm, mycosis fungoides, myelodysplasia syndromes, myelodysplastic/myeloproliferative neoplasms, multiple myeloma.

In some embodiments, the cancer is Merkel cell carcinoma, malignant mesothelioma, malignant fibrous histiocytoma of bone and osteosarcoma, nasal cavity and paranasal sinus cancer, nasopharyngeal cancer, neuroblastoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, or primary central nervous system (CNS).

In some embodiments, the cancer is hairy cell leukemia, head and neck cancer, heart cancer, liver cancer, hypopharyngeal cancer, intraocular melanoma, kidney cancer, laryngeal cancer, lip and oral cavity cancer, liver cancer, lobular carcinoma in situ (LCIS), lung cancer, lymphoma, metastatic squamous neck cancer with occult primary, or mouth cancer.

In some embodiments, the cancer is multiple endocrine neoplasia syndromes, multiple myeloma/plasma cell neoplasm, mycosis fungoides, myelodysplasia syndromes, myelodysplastic/myeloproliferative neoplasms, multiple myeloma, lymphoma, prostate cancer, rectal cancer, or transitional cell cancer,

In some embodiments, the cancer is retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some embodiments, the cancer is chronic lymphocytic leukemia (CLL), chronic myelogenous leukemia (CML), chronic myleoproliferative disorders, colon cancer, colorectal cancer.

In some embodiments, the cancer is CNS cancer, endometrial cancer, ependymoma, esophageal cancer, esthesioneuroblastoma, eye cancer, fibrous histiocytoma of bone, gall bladder cancer, gastric cancer

In some embodiments are methods for treating inflammation comprising administering to the mammal at least once an effective amount of at least one compound having the structure of Formula (B-I), (I)—(XXVIIId).

In some embodiments, provided herein are methods for the treatment of cancer comprising administering to the mammal at least once an effective amount of at least one compound having the structure of Formula (B-I), (I)—(XXVIIId). The type of cancer may include, but is not limited to, pancreatic cancer and other solid or hematological tumors.

In certain embodiments, provided herein are methods for treating the following diseases or conditions comprising administering to the mammal a compound provided herein. In some embodiments, the disease or condition is ALL (Acute Lymphoblastic Lymphoma), DLBCL (Diffuse Large B-Cell Lymphoma), FL (Follicular Lymphoma), RCC (Renal Cell Carcinoma), Childhood Medulloblastoma, Glioblastoma, Pancreatic tumor or cancer, Liver cancer (Hepatocellular Carcinoma), Prostate Cancer (Myc), Triple Negative Breast (Myc), AML (Acute Myeloid Leukemia), or MDS (Myelo Dyslplastic Syndrome). In some embodiments, the disease or condition is Early-onset Dystonia. In yet some embodiments, the disease or condition is Kabuki Syndrome.

In some embodiments, the disease or condition is p53 driven tumor.

In some embodiments, provided herein are methods for treating osteoporosis or bone resorption disorders comprising administering to a subject in need thereof a composition containing a therapeutically effective amount of a compound of formula (I) that is inhibitor of KRas G12C.

In some embodiments, provided herein is a pharmaceutical composition comprising a pharmaceutically acceptable carrier and a pharmaceutically effective amount of a compound according to any one of the formulas described herein. In some embodiments, the compound is according to any one of Formula (B-I), (I)—(XXVIIId).

In some embodiments, the pharmaceutical composition is formulated for a route of administration selected from oral administration, parenteral administration, buccal administration, nasal administration, topical administration, or rectal administration.

In some embodiments, the carrier is a parenteral carrier.

In some embodiments, the carrier is an oral carrier.

In some embodiments, the carrier is a topical carrier.

Any combination of the groups described above for the various variables is contemplated herein.

It is understood that substituents and substitution patterns on the compounds provided herein can be selected by one of ordinary skill in the art to provide compounds that are chemically stable and that can be synthesized by techniques known in the art, as well as those set forth herein.

Further representative embodiments of compounds of Formula (I), include compounds listed in 1A and Table 1B, or a pharmaceutically acceptable salt, solvate, hydrate, or stereoisomer thereof.

Throughout the specification, groups and substituents thereof can be chosen by one skilled in the field to provide stable moieties and compounds.

In some embodiments, the compounds of Formula (B-I), (I)—(XXVIIId) inhibit KRas G12C. In some embodiments, the compounds of Formula (B-I), (I)—(XXVIIId) are used to treat patients suffering from KRas G12C-dependent or KRas G12C mediated conditions or diseases, including, but not limited to, cancer, and other autoimmune diseases.

In some embodiments, the compounds of Formula (B-I), (I)—(XXVIIId) inhibit KRas G12D. In some embodiments, the compounds of Formula (B-I), (I)—(XXVIIId) are used to treat patients suffering from KRas G12D-dependent or KRas G12D mediated conditions or diseases, including, but not limited to, cancer, and other diseases.

### Preparation of Compounds

Compounds of any of Formula (B-I), (I)—(XXVIIId) may be synthesized using standard synthetic reactions known to those of skill in the art or using methods known in the art. The reactions can be employed in a linear sequence to provide the compounds or they may be used to synthesize fragments which are subsequently joined by the methods known in the art. Exemplary methods are provided in the Examples herein.

Described herein are compounds that inhibit the activity of KRas G12C, and processes for their preparation. Also described herein are pharmaceutically acceptable salts, pharmaceutically acceptable solvates, pharmaceutically active metabolites, and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided.

The starting material used for the synthesis of the compounds described herein may be synthesized or can be obtained from commercial sources, such as, but not limited to, Aldrich Chemical Co. (Milwaukee, Wisconsin), Bachem (Torrance, California), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989). (all of which are incorporated by reference in their entirety). Additional methods for the synthesis of compounds described herein may be found in International Patent Publication No. WO 01/01982901, Arnold et al. Bioorganic & Medicinal Chemistry Letters 10 (2000) 2167-2170; Burchat et al. Bioorganic & Medicinal Chemistry Letters 12 (2002) 1687-1690. General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, crystallization, chromatography, and the like. Such materials may be characterized using conventional means, including physical constants and spectral data.

Compounds described herein may be prepared as a single isomer or a mixture of isomers.

In some embodiments, representative compounds of Formula (I) are prepared according to synthetic schemes depicted herein.

### Further Forms of Compounds

Compounds disclosed herein have a structure of Formula (B-I), (I)—(XXVIIId). It is understood that when reference is made to compounds described herein, it is meant to include compounds of any of Formula (B-I), (I)—(XXVIIId) as well as to all of the specific compounds that fall within the scope of these generic formulae, unless otherwise indicated.

Compounds described herein may possess one or more stereocenters and each center may exist in the R or S configuration. Compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

Diastereomeric mixtures can be separated into their individual diastereomers on the basis of their physical chemical differences by methods known, for example, by chromatography and/or fractional crystallization. In some embodiments, enantiomers can be separated by chiral chromatographic columns. In some embodiments, enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

Methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, compounds described herein can exist in unsolvated as well as solvated forms with pharmaceutically acceptable solvents such as water, ethanol, and the like. Solvated forms of compounds presented herein are also considered to be disclosed herein.

Compounds of any of Formula (B-I), (I)—(XXVIIId) in unoxidized form can be prepared from N-oxides of compounds of any of Formula (B-I), (I)—(XXVIIId) by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically, or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985).

Prodrug forms of the herein described compounds, wherein the prodrug is metabolized in vivo to produce a derivative as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. Prodrugs may be designed as reversible drug derivatives, for use as modifiers to enhance drug transport to site-specific tissues. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Sites on the aromatic ring portion of compounds of any of Formula (B-I), (I)—(XXVIIId) can be susceptible to various metabolic reactions, therefore incorporation of appropriate substituents on the aromatic ring structures, such as, by way of example only, halogens can reduce, minimize or eliminate this metabolic pathway.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

In additional or some embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like.

The corresponding counterions of the pharmaceutically acceptable salts may be analyzed and identified using various methods including, but not limited to, ion exchange chromatography, ion chromatography, capillary electrophoresis, inductively coupled plasma, atomic absorption spectroscopy, mass spectrometry, or any combination thereof.

The salts are recovered by using at least one of the following techniques: filtration, precipitation with a non-solvent followed by filtration, evaporation of the solvent, or, in the case of aqueous solutions, lyophilization.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

It should be understood that a reference to a salt includes the solvent addition forms or crystal forms thereof, particularly solvates or polymorphs. Solvates contain either stoichiometric or non-stoichiometric amounts of a solvent, and are often formed during the process of crystallization with pharmaceutically acceptable solvents such as water, ethanol, and the like. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

Compounds described herein may be in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The screening and characterization of the pharmaceutically acceptable salts, polymorphs, and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UVIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

Throughout the specification, groups and substituents thereof can be chosen by one skilled in the field to provide stable moieties and compounds.

### Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical composition, as used herein, refers to a mixture of a compound described herein, such as, for example, compounds of any of Formula (B-I), (I)—(XXVIIId) with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, compositions may also include one or more salts in an amount required to bring osmolality of the composition into an acceptable range. Such salts include those having sodium, potassium or ammonium cations, and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The pharmaceutical compositions described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical compositions described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein, such as, for example, a compound of any of Formula (B-I), (I)—(XXVIIId) as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite, and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

“Binders” impart cohesive qualities and include, e.g., alginic acid and salts thereof, cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of any of Formula (B-I), (I)—(XXVIIId) and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizcers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel®PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, g, or ng of therapeutic agent per ml, dl, or 1 of blood serum, absorbed into the bloodstream after administration. As used herein, measurable plasma concentrations are typically measured in ng/ml or g/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. The terms patient and subject may be used interchangeably.

Moreover, the pharmaceutical compositions described herein, which include a compound of any of Formula (B-I), (I)—(XXVIIId) can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In some embodiments, the pharmaceutical composition is in the form of a powder. In some embodiments, the pharmaceutical composition is in the form of a tablet, including but not limited to, a fast-melt tablet. Additionally, pharmaceutical compositions described herein may be administered as a single capsule or in multiple capsule dosage form. In some embodiments, the pharmaceutical composition is administered in two, or three, or four, capsules or tablets.

In some embodiments, solid dosage forms, e.g., tablets, effervescent tablets, and capsules, are prepared by mixing particles of a compound of any of Formula (B-I), (I)—(XXVIIId) with one or more pharmaceutical excipients to form a bulk blend composition. When referring to these bulk blend compositions as homogeneous, it is meant that the particles of the compound of any of Formula (B-I), (I)—(XXVIIId) are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some embodiments, using standard coating procedures, such as those described in Remington's Pharmaceutical Sciences, 20th Edition (2000), a film coating is provided around the formulation of the compound of any of Formula (B-I), (I)—(XXVIIId). In some embodiments, some or all of the particles of the compound of any of Formula (B-I), (I)—(XXVIIId) are coated. In some embodiments, some or all of the particles of the compound of any of Formula (B-I), (I)—(XXVIIId), are microencapsulated. In still some embodiments, the particles of the compound of any of Formula (B-I), (I)—(XXVIIId) are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of any of Formula (B-I), (I)—(XXVIIId) from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel®PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Agoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™ PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In some embodiments, one or more layers of the pharmaceutical composition are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In some embodiments, the compressed tablets will include a film surrounding the final compressed tablet. In some embodiments, the film coating can provide a delayed release of the compound of any of Formula (B-I), (I)—(XXVIIId) from the formulation. In some embodiments, the film coating aids in patient compliance (e.g., Opadry® coatings or sugar coating). Film coatings including Opadry® typically range from about 1% to about 3% of the tablet weight. In some embodiments, the compressed tablets include one or more excipients.

A capsule may be prepared, for example, by placing the bulk blend of the formulation of the compound of any of Formula (B-I), (I)—(XXVIIId), described above, inside of a capsule. In some embodiments, the formulations (non-aqueous suspensions and solutions) are placed in a soft gelatin capsule. In some embodiments, the formulations are placed in standard gelatin capsules or non-gelatin capsules such as capsules comprising HPMC. In some embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of the compound of any of Formula (B-I), (I)—(XXVIIId) and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In some embodiments, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with compounds of any of Formula (B-I), (I)—(XXVIIId) which sufficiently isolate the compound of any of Formula (B-I), (I)—(XXVIIId) from other non-compatible excipients. Materials compatible with compounds of any of Formula (B-I), (I)—(XXVIIId) are those that delay the release of the compounds of any of Formula (B-I), (I)—(XXVIIId), in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In some embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In some embodiments, the microencapsulating material useful for delaying the release of the pharmaceutical compositions is from the USP or the National Formulary (NF). In some embodiments, the microencapsulation material is Klucel. In some embodiments, the microencapsulation material is methocel.

Microencapsulated compounds of any of Formula (B-I), (I)—(XXVIIId) may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In some embodiments, the particles of compounds of any of Formula (B-I), (I)—(XXVIIId) are microencapsulated prior to being formulated into one of the above forms. In still some embodiments, some or most of the particles are coated prior to being further formulated by using standard coating procedures, such as those described in Remington's Pharmaceutical Sciences, 20th Edition (2000).

In some embodiments, the solid dosage formulations of the compounds of any of Formula (B-I), (I)—(XXVIIId) are plasticized (coated) with one or more layers. Illustratively, a plasticizer is generally a high boiling point solid or liquid. Suitable plasticizers can be added from about 0.010% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In some embodiments, a powder including the formulations with a compound of any of Formula (B-I), (I)—(XXVIIId), described herein, may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still some embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid, and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the formulations described herein, which include a compound of Formula (I), are solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734, each of which is specifically incorporated by reference. In some embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518, each of which is specifically incorporated by reference.

The pharmaceutical solid oral dosage forms including formulations described herein, which include a compound of any of Formula (B-I), (I)—(XXVIIId) can be further formulated to provide a controlled release of the compound of Formula (I). Controlled release refers to the release of the compound of any of Formula (B-I), (I)—(XXVIIId) from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In some embodiments, the polymers and compatible mixtures thereof, and some of their properties, include, but are not limited to:
Shellac, also called purified lac, a refined product obtained from the resinous secretion of an insect. This coating dissolves in media of pH>7;
Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS, and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;
Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles <1 m. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;
Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In some embodiments, the formulations described herein, which include a compound of Formula (I), are delivered using a pulsatile dosage form. A pulsatile dosage form is capable of providing one or more immediate release pulses at predetermined time points after a controlled lag time or at specific sites. Pulsatile dosage forms including the formulations described herein, which include a compound of any of Formula (B-I), (I)—(XXVIIId) may be administered using a variety of pulsatile formulations known in the art. For example, such formulations include, but are not limited to, those described in U.S. Pat. Nos. 5,011,692, 5,017,381, 5,229,135, and 5,840,329, each of which is specifically incorporated by reference. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284, all of which are specifically incorporated by reference. In some embodiments, the controlled release dosage form is pulsatile release solid oral dosage form including at least two groups of particles, (i.e. multiparticulate) each containing the formulation described herein. The first group of particles provides a substantially immediate dose of the compound of any of Formula (B-I), (I)—(XXVIIId) upon ingestion by a mammal. The first group of particles can be either uncoated or include a coating and/or sealant. The second group of particles includes coated particles, which includes from about 2% to about 75%, from about 2.5% to about 70%, or from about 40% to about 70%, by weight of the total dose of the compound of any of Formula (B-I), (I)—(XXVIIId) in said formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the formulation that includes a compound of any of Formula (I).

Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014, and 6,932,983, each of which is specifically incorporated by reference.

In some embodiments, pharmaceutical compositions are provided that include particles of the compounds of any of Formula (B-I), (I)—(XXVIIId), described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition to the particles of compound of Formula (I), the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In some embodiments, an aqueous suspension can be re-suspended into a homogenous suspension by physical agitation lasting less than 1 minute. In some embodiments, an aqueous suspension can be re-suspended into a homogenous suspension by physical agitation lasting less than 45 seconds. In yet some embodiments, an aqueous suspension can be re-suspended into a homogenous suspension by physical agitation lasting less than 30 seconds. In still some embodiments, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In some embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In some embodiments, the aqueous liquid dispersion can comprise a sweetening agent or flavoring agent in a concentration ranging from about 0.001% to about 1.0% the volume of the aqueous dispersion. In some embodiments, the aqueous liquid dispersion can comprise a sweetening agent or flavoring agent in a concentration ranging from about 0.005% to about 0.5% the volume of the aqueous dispersion. In yet some embodiments, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical compositions described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817, and 6,391,452, each of which is specifically incorporated by reference. Formulations that include a compound of any of Formula (B-I), (I)—(XXVIIId) which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation, the compounds of any of Formula (B-I), (I)—(XXVIIId), described herein may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations that include compounds of any of Formula (B-I), (I)—(XXVIIId) may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery of the compound of any of Formula (B-I), (I)—(XXVIIId), is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the compound of any of Formula (B-I), (I)—(XXVIIId), and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In some embodiments, the transdermal formulations described herein include at least three components: (1) a formulation of a compound of any of Formula (I); (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In some embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of the compounds of any of Formula (B-I), (I)—(XXVIIId). The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include a compound of any of Formula (B-I), (I)—(XXVIIId), suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions, or emulsions in oily or aqueous vehicles, and may contain formulatory agents such as suspending, stabilizing and/or dispersing agents. Pharmaceutical compositions for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Formulations

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams, or ointments. Such pharmaceutical compounds can contain solubilizers, stabilizers, tonicity enhancing agents, buffers, and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

The compounds described herein can be used in the preparation of medicaments for the inhibition of KRas or a homolog thereof, or for the treatment of diseases or conditions that would benefit, at least in part, from inhibition of KRas or a homolog thereof. In addition, a method for treating any of the diseases or conditions described herein in a subject in need of such treatment, involves administration of pharmaceutical compositions containing at least one compound of any of Formula (B-I), (I)—(XXVIIId), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Combination Treatments

The KRas G12C inhibitor compositions described herein can also be used in combination with other well known therapeutic reagents that are selected for their therapeutic value for the condition to be treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In certain instances, it may be appropriate to administer at least one KRas G12C inhibitor compound described herein in combination with another therapeutic agent. By way of example only, if one of the side effects experienced by a patient upon receiving one of the KRas G12C inhibitor compounds described herein is nausea, then it may be appropriate to administer an anti-nausea agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is a compound of Formula (B-I), (I)—(XXVIIId), described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life, and kinetic profile of the pharmaceutical agent. Circadian variation of the target molecule concentration may also determine the optimal dose interval.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

The compounds described herein and combination therapies can be administered before, during or after the occurrence of a disease or condition, and the timing of administering the composition containing a compound can vary. Thus, for example, the compounds can be used as a prophylactic and can be administered continuously to subjects with a propensity to develop conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, within the first 6 hours of the onset of the symptoms, or within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound should be administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, between about 1 month to about 5 years, or from about 1 month to about 3 years.

Exemplary Therapeutic Agents for Use in Combination with a KRas G12C Inhibitor or a KRas G12D Inhibitor Compound

Where the subject is suffering from or at risk of suffering from an autoimmune disease, an inflammatory disease, or an allergy disease, an KRas G12C inhibitor compound can be used with one or more of the following therapeutic agents in any combination: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific irreversible inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-3, interferon-γ, interleukin-2, allergy vaccines, antihistamines, antileukotrienes, beta-agonists, theophylline, or anticholinergics.

Where the subject is suffering from or at risk of suffering from a B-cell lymhoproliferative disorder (e.g., plasma cell myeloma), the subjected can be treated with a KRas G12C inhibitor compound in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Other anti-cancer agents that can be employed in combination with an KRas G12C inhibitor compound include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rIL2), interferon α-2a; interferon α-2b; interferon α-n1; interferon α-n3; interferon β-la; interferon γ-lb; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with an KRas G12C inhibitor compound include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis irreversible inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase irreversible inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase irreversible inhibitors; gemcitabine; glutathione irreversible inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin irreversible inhibitors; matrix metalloproteinase irreversible inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase irreversible inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome irreversible inhibitors; protein A-based immune modulator; protein kinase C inhibitor; protein kinase C irreversible inhibitors, microalgal; protein tyrosine phosphatase irreversible inhibitors; purine nucleoside phosphorylase irreversible inhibitors; purpurins; pyrazoloacridine; pyridoxylated hemoglobin polyoxyethylerie conjugate; raf antagonists; raltitrexed; ramosetron; ras farnesyl protein transferase irreversible inhibitors; ras irreversible inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction irreversible inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division irreversible inhibitors; stipiamide; stromelysin irreversible inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase irreversible inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation irreversible inhibitors; tretinoin; triacetyluridine; triciribine; trimetrexate; triptorelin; tropisetron; turosteride; tyrosine kinase irreversible inhibitors; tyrphostins; UBC irreversible inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with an KRas G12C inhibitor compound include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products useful in combination with an KRas G12C inhibitor compound include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination an KRas G12C inhibitor compound include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists useful in combination with an KRas G12C inhibitor compound include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with an KRas G12C inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, lnanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Where the subject is suffering from or at risk of suffering from a thromboembolic disorder (e.g., stroke), the subject can be treated with an KRas G12C inhibitor compound in any combination with one or more other anti-thromboembolic agents. Examples of anti-thromboembolic agents include, but are not limited to, any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa irreversible inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of KRas, or in which KRas is a mediator or contributor to the symptoms or cause.

For example, the container(s) can include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded, or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

The examples below as well as throughout the application, the following abbreviations have the following meanings. If not defined, the terms have their generally accepted meanings.
aq=aqueous
Boc=tert-butyloxycarbonyl
t-BuOH=tertiary butanol
DCE=1,2-dichloroethane
DCM=dichloromethane
DIAD=diisopropyl azodicarboxylate
DIEA or DIPEA=N,N-diisopropylethylamine
DMAP=dimethylaminopyridine
DMF=dimethylformamide
DMSO=dimethylsulfoxide
ESI=electron spray ionization
EA, EtOAc=ethyl acetate
Et₂O=diethyl ether
g=gram
HCl=hydrogen chloride
HPLC=high performance liquid chromatography
hr=hour
¹H NMR=proton nuclear magnetic resonance
IPA=isopropyl alcohol
KOAc=potassium acetate
LC-MS=liquid chromatography mass spectroscopy
M=molar
MeCN=acetonitrile
MeOH=methanol
mg=milligram
min=minute
ml=milliliter
mM=millimolar
mmol=millimole
m.p.=melting point
MS=mass spectrometry
m/z=mass-to-charge ratio
N=normal
NIS=N-iodosuccinimide
nM=nanomolar
nm=nanometer
Pd(dppf)Cl₂=[1,1′-Bis(diphenylphosphino)ferrocene]dichloropalladium(II)
PE=petroleum ether
PyBOP=benzotriazol-1-yl-oxytripyrrolidinophosphonium hexafluorophosphate
quant.=quantitative
RP=reverse phase
rt or r.t.=room temperature
Sat.=saturated
TEA=triethylamine
TFA=trifluoroacetic acid
THF=tetrahydrofuran
TLC=Thin layer chromatography
μL=microliter
μM=Micromolar

### SYNTHESIS OF INTERMEDIATES

### Preparation of Intermediate 64

### Synthesis of 4-(morpholin-4-yl)but-2-ynoic acid (64)

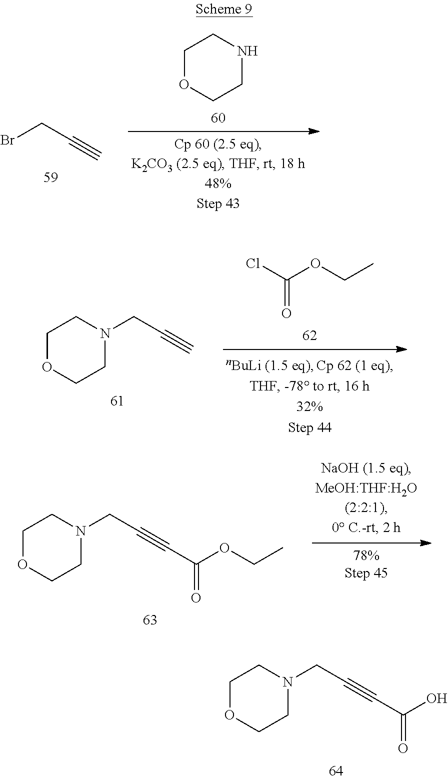

Synthesis of 4-(prop-2-yn-1-yl) morpholine: (61) To a stirred suspension of morpholine 60 (51 mL, 250 mmol, 2.5 eq) and K₂CO₃ (146 g, 250 mmol, 2.5 eq) in THF (500 mL) was added propargyl bromide 59 (35 mL, 80% in toluene, 15 g solution, 100 mmol, 1.0 eq) dropwise over 20 min at 25° C. The resulting mixture was stirred vigorously for 18 h, after which the reaction mixture was diluted with 200 mL EtOAc. The mixture was washed with water (100 mL) and brine (2×100 mL), and the combined aqueous layers were further extracted with DCM (2×500 mL). The combined organic layers were dried over Na₂SO₄, filtered, and concentrated in vacuo to give a yellow-orange oily residue which was purified by silica gel column chromatography (20% EtOAc in heptane) to afford 4-(prop-2-yn-1-yl)morpholine 61 as yellow liquid (15 g, 112 mmol, 48% yield).

### Step 44

Synthesis of ethyl 4-(morpholin-4-yl) but-2-ynoate (63): To a stirred solution of 4-(prop-2-yn-1-yl) morpholine 61 (14 g, 112 mmol) in THF (150 mL) was added ^{t}BuLi (44.7 mL, 2.5 M in hexane, 112 mmol, 1 eq) at −78° C. under N₂ atmosphere. The reaction mixture was stirred at the same temperature for 30 min. Subsequently to it was added methyl chloroformate 62 (18.2 g, 1.5 eq, 168 mmol) dropwise at same temperature. The reaction mixture was allowed to come to rt and stirred for next 16 h. After completion of the reaction (TLC monitoring), reaction mixture was cooled to 0° C. and was quenched with NH₄Cl (100 mL) and extracted with EtOAc (2×250 mL). The combined organic layers were washed with brine, dried over anhydrous Na₂SO₄, and filtered. After filtration, the filtrate was concentrated under reduced pressure and the residue was purified by silica gel column chromatography (30% EtOAc in heptane) to afford ethyl 4-(morpholin-4-yl)but-2-ynoate 63 (7 g, 32% yield) as a colorless liquid.

### Step 45

Synthesis of 4-(morpholin-4-yl)but-2-ynoic acid (64): To a stirred ice cold solution of ethyl 4-(morpholin-4-yl)but-2-ynoate 63 (6 g, 25.4 mmol) in a mixture of methanol (20 mL) and THF (20 mL) was added sodium hydroxide (1.52 g, 1.5 eq, 45.6 mmol) dissolved in water (10 mL) dropwise. The reaction mixture was allowed to come to rt and stirred for next 2 h. After completion of reaction (monitored by TLC), the solvent was evaporated under the reduced pressure. The pH of reaction mixture was then adjusted to ˜4 by using sodium hydrogen sulphate. Then reaction mixture was extracted with ether (2×50 mL). Aq. layer was lyophilized to get a crude, which was dissolved in methanol (10 mL) and subsequently filtered to remove any insoluble salt. The filtrate was evaporated and dried under reduced pressure to get 4-(morpholin-4-yl)but-2-ynoic acid 64 (3.2 g, 78% yield) as a yellowish semi-solid.

### Preparation of Intermediate 70

### Synthesis of N-methyl-N—((S)-1-(4-morpholinobut-2-ynoyl)pyrrolidine-3-carbonyl)-L-valine (70)

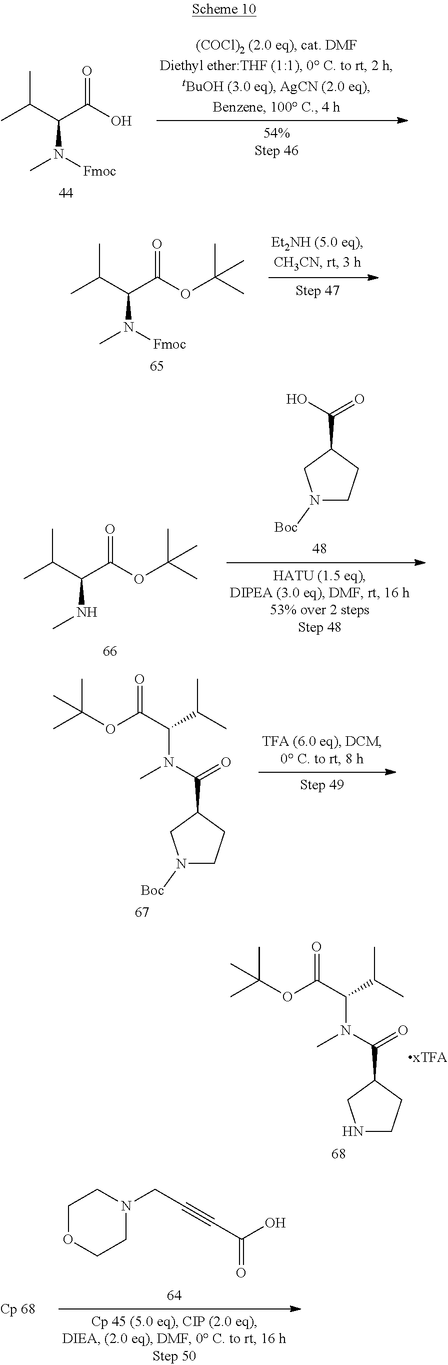

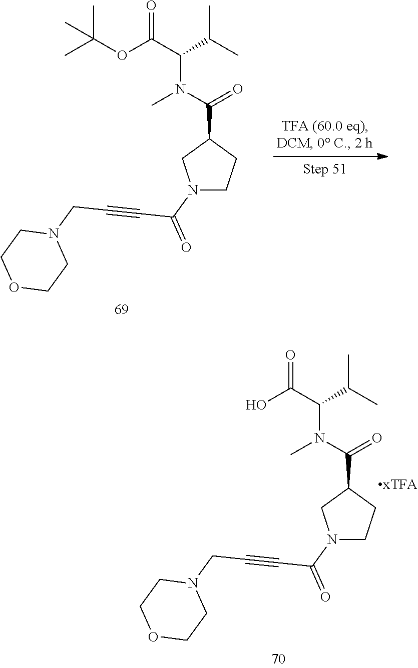 [SMILES: CC(C)[C@@H](C(=O)OC(C)(C)C)N(C)C(=O)[C@H]1CCN(C(=O)C#CCN2CCOCC2)C1 ; CC(C)[C@@H](C(=O)O)N(C)C(=O)[C@H]1CCN(C(=O)C#CCN2CCOCC2)C1]

### Step 46

Synthesis of tert-butyl N-(((9H-fluoren-9-yl)methoxy)carbonyl)-N-methyl-L-valinate (65): A suspension of N-(((9H-fluoren-9-yl)methoxy)carbonyl)-N-methyl-L-valine 44 (10 g, 28.3 mmol) in 100 mL of 20% THF in Et₂O was cooled in an ice-H₂O bath under N₂ and treated with oxalyl chloride (14.5 mL, 170 mmol), followed by a catalytic amount (10 drops) of DMF. The mixture was stirred for 2 h in the cold and then 45 min at ambient temperature, and the volatiles were removed in vacuo. The yellow, oily residue was taken up in benzene (100 mL), flushed with N₂, and treated with tert-butyl alcohol (13 mL, 141 mmol) followed by addition of AgCN (19.8 g, 84.9 mmol). The suspension was heated under reflux (100° C.) for 1.5 h, then cooled, diluted with EtOAc (300 mL), and filtered through a pad of Celite. The filtrate was concentrated under reduced pressure to give a yellow oil, which was purified by silica gel column chromatography (EtOAc/heptane), to afford tert-butyl N-(((9H-fluoren-9-yl)methoxy)carbonyl)-N-methyl-L-valinate 65 (7 g, 28.3 mmol, 54%) as a viscous, colorless oil.

### Step 47

Synthesis of tert-butyl methyl-L-valinate 66: To a stirred solution of tert-butyl N-(((9H-fluoren-9-yl)methoxy)carbonyl)-N-methyl-L-valinate 65 (7 g, 17.1 mmol) in acetonitrile (20 mL) at rt was added diethylamine (20 mL). After being stirred for 3 h, the reaction mixture was concentrated in vacuo. The residue was azeotroped twice with toluene to obtain the crude tert-butyl methyl-L-valinate 66 (7 g, crude) which was used in the next reaction without further purification.

### Step 48

Synthesis of tert-butyl (S)-3-(((S)-1-(tert-butoxy)-3-methyl-1-oxobutan-2-yl)(methyl)carbamoyl)pyrrolidine-1-carboxylate (67): To a stirred solution of tert-butyl methyl-L-valinate 66 (7 g crude as obtained in the previous step) and (S)-1-(tert-butoxycarbonyl)pyrrolidine-3-carboxylic acid 48 (3.6 g, 17.1 mmol) in DMF (50 mL) was added DIPEA (15.7 mL, 85.5 mmol) at rt. HATU (12.9 g, 34 mmol) was added and the mixture was stirred for 16 h. After completion of the reaction (TLC monitoring), reaction mixture was extracted with EtOAc (2×50 mL) and washed with H₂O (50 mL) and brine (50 mL). The combined organic layers were dried over anhydrous Na₂SO₄ and filtered. After filtration, the filtrate was concentrated under reduced pressure and the residue was purified by silica gel column chromatography (heptane/EtOAc) to afford tert-butyl (S)-3-(((S)-1-(tert-butoxy)-3-methyl-1-oxobutan-2-yl)(methyl)carbamoyl)pyrrolidine-1-carboxylate 67 (3.6 g, 53%, over last two steps) as a colorless oil.

### Step 49

Synthesis of tert-butyl N-methyl-N—((S)-pyrrolidine-3-carbonyl)-L-valinate (68): To a stirred ice cold solution of tert-butyl (S)-3-(((5)-1-(tert-butoxy)-3-methyl-1-oxobutan-2-yl)(methyl)carbamoyl)pyrrolidine-1-carboxylate 67 (3 g, 7.8 mmol) in dichloromethane (40 mL) was added trifluoroacetic acid (3.58 mL, 6 eq, 46.8 mmol). The reaction mixture was allowed to come to rt and stirred for next 8 h. After completion of the reaction (TLC monitoring), reaction mixture was concentrated under reduced pressure to get crude tert-butyl N-methyl-N—((S)-pyrrolidine-3-carbonyl)-L-valinate 68 (3 g, as TFA salt) as a thick liquid, which was used directly in the next step without further purification.

### Step 50

Synthesis of tert-butyl N-methyl-N—((S)-1-(4-morpholinobut-2-ynoyl)pyrrolidine-3-carbonyl)-L-valinate (69): To a stirred mixture of tert-butyl N-methyl-N—((S)-pyrrolidine-3-carbonyl)-L-valinate 68 (3 g, 7.53 mmol; crude as TFA salt, not quantified), and 4-morpholinobut-2-ynoic acid 64 (6.3 g, 37.6 mmol) in DMF (20 mL) was added DIPEA (2.8 mL, 37.6 mmol) followed by CIP (3.1 g, 11.3 mmol) and was stirred at 0° C. for 2 h. After completion of the reaction (TLC/LC monitoring), ice-cold water (50 mL) was added to the reaction mixture and extracted vigorously with EtOAc (3×200 mL). The organic layer was further washed with ice-cold brine (100 mL). The combined organic layers were dried over anhydrous Na₂SO₄, and filtered. After filtration, the filtrate was concentrated under reduced pressure to obtain the crude tert-butyl N-methyl-N—((S)-1-(4-morpholinobut-2-ynoyl)pyrrolidine-3-carbonyl)-L-valinate 69 (1.7 g) as thick liquid, which was used directly in the next step without further purification.

### Step 51

Synthesis of N-methyl-N—((S)-1-(4-morpholinobut-2-ynoyl)pyrrolidine-3-carbonyl)-L-valine (70): To a stirred ice cold solution of tert-butyl N-methyl-N—((S)-1-(4-morpholinobut-2-ynoyl)pyrrolidine-3-carbonyl)-L-valinate 69 (300 mg, 0.689 mmol; as crude) in dichloromethane (8 mL) was added excess of trifluoroacetic acid (3.1 mL, 41.3 mmol), and the reaction mixture was stirred at 0° C. for next 2 h. After completion of the reaction (LC monitoring), reaction mixture was concentrated under reduced pressure to afford N-methyl-N—((S)-1-(4-morpholinobut-2-ynoyl)pyrrolidine-3-carbonyl)-L-valine 70 (250 mg, crude as TFA salt) as an oil, which was used directly in the next step without further purification.

### Representative Synthesis of Compounds of Invention

### Example 1

### Compound 1

### Synthesis of (3S)—N-((2S)-1-(((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide (80) [Compound 1]

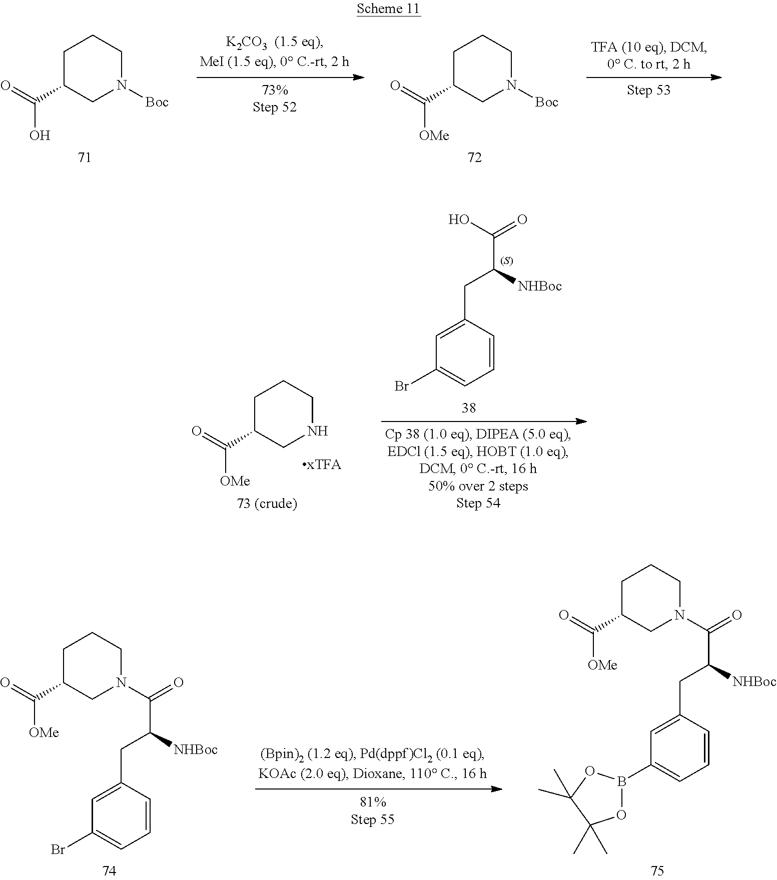

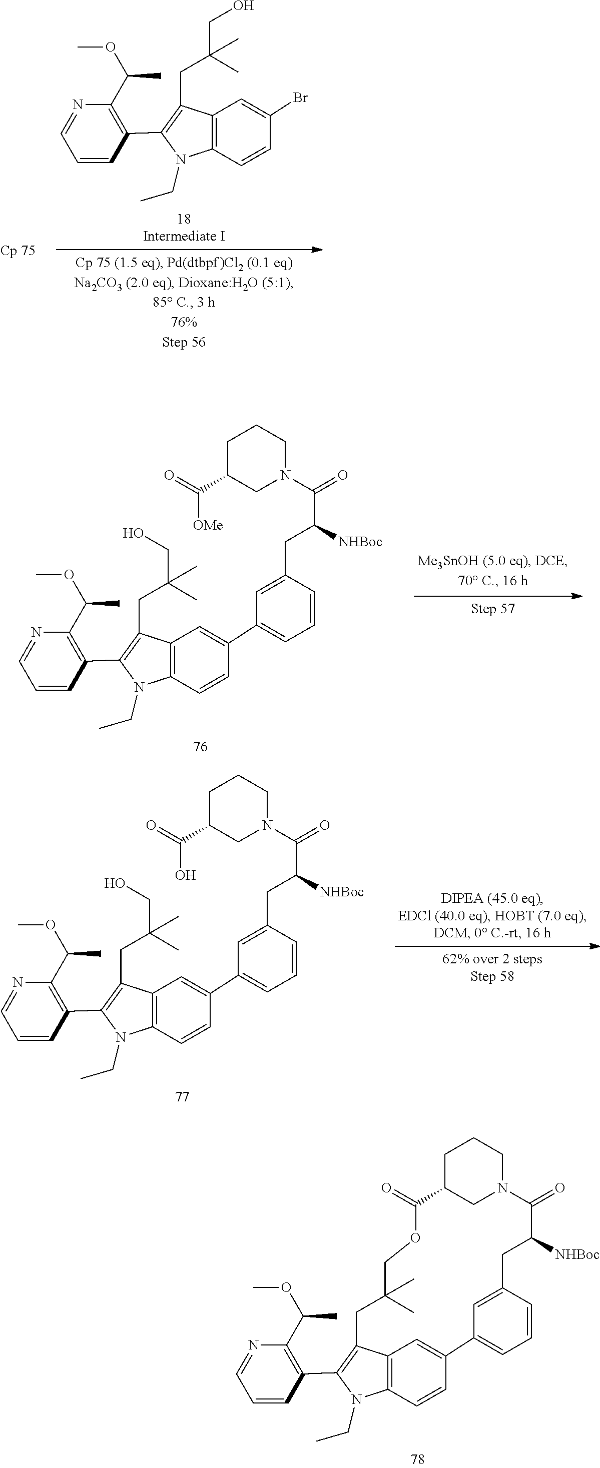

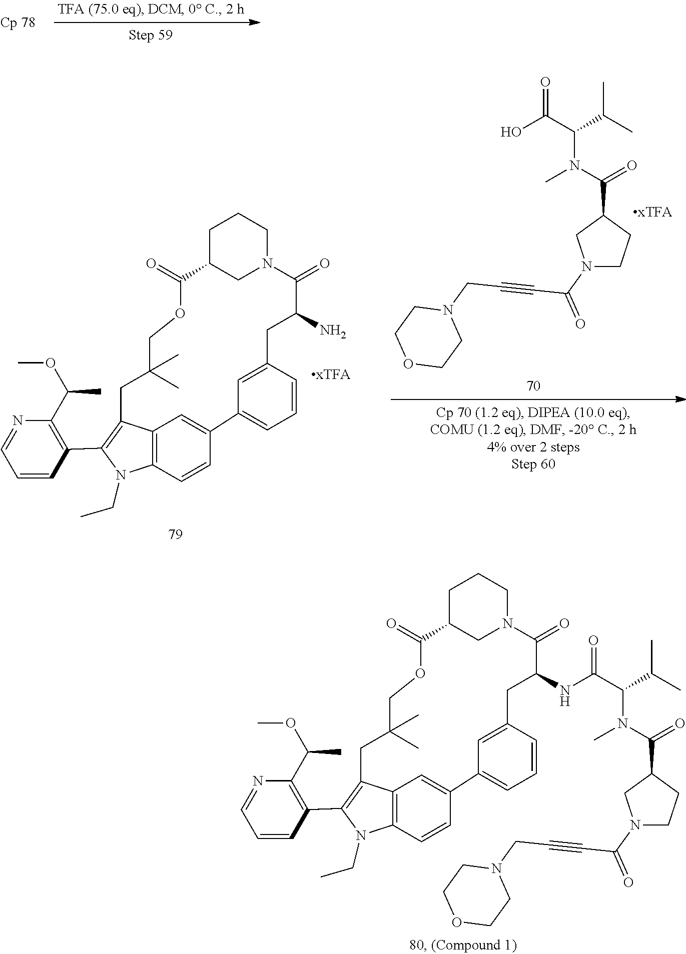

### Step 52

Synthesis of 1-(tert-butyl) 3-methyl (R)-piperidine-1,3-dicarboxylate (72): A mixture of (3R)-1-[(tert-butoxy)carbonyl]piperidine-3-carboxylic acid 71 (2.8 g, 12.2 mmol) and K₂C₃(2.53 g, 18.3 mmol) in dimethylformamide (20 mL) was stirred at rt for 10 min under nitrogen atmosphere. To it was then added iodomethane (2.6 g, 18.3 mmol) and stirred the reaction at rt for 2 h. After completion of reaction (TLC monitoring), the reaction mixture was diluted with EtOAc (200 mL) and washed with chilled brine solution (3×50 mL). The combined organic layer was dried over anhydrous Na₂SO₄, filtered and the filtrate thus obtained was concentrated under reduced pressure to get a crude which was purified by Combi-flash column chromatography over silica gel using 5-20% EtOAc in heptane to obtain 1-tert-butyl 3-methyl (3R)-piperidine-1,3-dicarboxylate 72 (2.4 g, 73% yield) as an oil.

### Step 53

Synthesis of methyl (3R)-piperidine-3-carboxylate (73): To a stirred ice cold solution of 1-tert-butyl 3-methyl (3R)-piperidine-1,3-dicarboxylate 72 (2 g, 5.2 mmol) in dichloromethane (30 mL) was added trifluoroacetic acid (4 mL, 52 mmol). The reaction mixture was allowed to come to rt and stirred for next 2 h. After completion of the reaction (TLC monitoring), reaction mixture was concentrated under reduced pressure to obtain methyl (3R)-piperidine-3-carboxylate 73 (2.6 g, crude as TFA salt), which was used directly in the next step without further purification.

### Step 54

Synthesis of methyl (R)-1-((S)-3-(3-bromophenyl)-2-((tert-butoxycarbonyl)amino)propanoyl)piperidine-3-carboxylate (74): To an ice cold mixture of (3R)-piperidine-3-carboxylate 73 (2.5 g, 10.5 mmol; as crude TFA salt) in DCM (20 mL) was added DIPEA (7.6 mL, 43.5 mmol) at 0° C. and stirred the reaction for 10 min. To it was then added (S)-3-(3-bromophenyl)-2-((tert-butoxycarbonyl)amino)propanoic acid 38 (3 g, 8.7 mmol) dissolved in DCM (40 mL), followed by HOBT (2.0 g, 13 mmol) and EDCI (3.3 g, 17.4 mmol), respectively. The mixture was warmed to rt and stirred for 16 h. After completion of reaction (TLC/LC monitoring), the reaction mass was diluted with DCM (200 mL) and washed with H₂O (3×100 mL). The organic layer was dried over anhydrous Na₂SO₄, filtered, and the filtrate was concentrated under reduced pressure to obtain a crude which was purified by Combi-flash column chromatography over silica gel using 5-20% EtOAc in heptane to afford methyl (R)-1-((S)-3-(3-bromophenyl)-2-((tert-butoxycarbonyl)amino)propanoyl)piperidine-3-carboxylate 74 (2 g, 50%) as white solid.

### Step 55

Synthesis of methyl (R)-1-((S)-2-((tert-butoxycarbonyl)amino)-3-(3-(4,4,5,5-tetramethyl-1,3,2-dioxaborolan-2-yl)phenyl)propanoyl)piperidine-3-carboxylate (75): To a stirred mixture of methyl (R)-1-((S)-3-(3-bromophenyl)-2-((tert-butoxycarbonyl)amino)propanoyl)piperidine-3-carboxylate 74 (1 g, 2.13 mmol) in 1,4-dioxane (20 mL) was added KOAc (627 mg, 6.39 mmol) under an atmosphere of Ar. The resulting mixture was purged with Argon for 10 min. Subsequently to it was added bis(pinacolato)diboron (649 mg, 2.56 mmol) followed by [1,1′-Bis(diphenylphosphino)ferrocene]palladium(II) dichloride] (156 mg, 0.213 mmol). The resulting reaction mixture was heated at 110° C. for next 16 h. After reaction monitoring (TLC/LC), the reaction mixture was cooled to rt, quenched with water (50 mL) and extracted with EtOAc (3×50 mL). Combined organic layer was washed with brine (20 mL), dried over anhydrous Na₂SO₄ and evaporated under reduced pressure to get the crude residue which was purified by Combi-flash column chromatography over silica gel using 20-50% EtOAc in heptane to get methyl (R)-1-((S)-2-((tert-butoxycarbonyl)amino)-3-(3-(4,4,5,5-tetramethyl-1,3,2-dioxaborolan-2-yl)phenyl)propanoyl)piperidine-3-carboxylate 75 (0.9 g, 81% yield) as a sticky liquid.

### Step 56

Synthesis of methyl (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylate (76): To a stirred mixture of (S)-3-(5-bromo-1-ethyl-2-(2-(1-methoxyethyl)pyridin-3-yl)-1H-indol-3-yl)-2,2-dimethylpropan-1-ol 18 (350 mg, 0.788 mmol) and methyl (R)-1-((S)-2-((tert-butoxycarbonyl)amino)-3-(3-(4,4,5,5-tetramethyl-1,3,2-dioxaborolan-2-yl)phenyl)propanoyl)piperidine-3-carboxylate 75 (610 mg, 1.18 mmol) in 1,4-dioxane (15 mL) and H₂O (3 mL) at rt under an atmosphere of Ar was added Na₂CO₃ (170 mg, 1.6 mmol) followed by [1,1′-Bis(di-tert-butylphosphino)ferrocene]dichloropalladium(II)] (51 mg, 0.078 mmol). The resulting mixture was purged with Argon for 10 min, and was then heated to 85° C. for next 3 h. After completion of the reaction (TLC/LC monitoring), the reaction mass was diluted with EtOAc (50 mL) and washed with H₂O (3×20 mL). The organic layer was dried over anhydrous Na₂SO₄, filtered and the filtrate was concentrated under reduced pressure to get a crude which was purified by Combi-flash column chromatography over silica gel using 30-70% EtOAc in heptane affording methyl (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylate 76 (400 mg, 67% yield) as a light yellow solid.

### Step 57

Synthesis of (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylic acid (77): To a stirred mixture of methyl (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylate 76 (400 mg, 0.53 mmol) in DCE (10 mL) at rt was added trimethyltin hydroxide (481 mg, 2.64 mmol) in portions. The mixture was heated to 70° C. for 16 h. After completion of the reaction (TLC/LC monitoring), the reaction mass was filtered and the filter cake was thoroughly washed with DCM (100 mL). The filtrate was concentrated under reduced pressure to give (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylic acid 77 (500 mg, crude), which was used directly in the next step without further purification.

### Step 58

Synthesis of tert-butyl ((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)carbamate (78): To a stirred mixture of (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylic acid 77 (500 mg, 0.675 mmol) in DCM (70 mL) at 0° C. under an atmosphere of N₂ was added DIPEA (5.29 mL, 30.4 mmol), followed by HOBT (719 mg, 4.72 mmol) and EDCI (5.17 g, 27 mmol) in portions. The mixture was warmed to rt and stirred for 16 h. After completion of the reaction (TLC/LC monitoring), the reaction mass was diluted with DCM (100 mL), washed with brine (3×50 mL), dried over anhydrous Na₂SO₄, and filtered. The filtrate was concentrated under reduced pressure and the residue was purified by Combi-flash column chromatography over silica gel using 20-70% EtOAc in heptane to give tert-butyl ((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)carbamate 78 (240 mg, 62% yield, over 2 steps) as a white solid.

### Step 59

### (6³R,4S)-4-amino-1¹-ethyl-1²-(2-((S)-1-methoxyethyl)pyridin-3-yl)-10,10-dimethyl-1¹H-8-oxa-1(5,3)-indola-6(1,3)-piperidina-2(1,3)-benzenacycloundecaphane-5,7-dione (79)

To a stirred ice cold solution of tert-butyl ((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)carbamate 78 (240 mg, 0.33 mmol) in DCM (10 mL) was added trifluoroacetic acid (1.9 mL, 24.9 mmol) and the reaction mixture was stirred at 0° C. for 2 h. After completion of reaction (TLC monitoring), reaction mass was concentrated under reduced pressure to give a crude which was further evaporated with toluene (20 mL; repeated thrice) eventually to give (6³R,4S)-4-amino-1¹-ethyl-1²-(2-((S)-1-methoxyethyl)pyridin-3-yl)-10,10-dimethyl-1¹H-8-oxa-1(5,3)-indola-6(1,3)-piperidina-2(1,3)-benzenacycloundecaphane-5,7-dione 79 (300 mg, crude as TFA salt), which was used directly in the next step without further purification.

### Step 60

Synthesis of (3S)—N-((2S)-1-(((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide (90) [Compound 1]: To a stirred mixture of (6³R,4S)-4-amino-1¹-ethyl-1²-(2-((S)-1-methoxyethyl)pyridin-3-yl)-10,10-dimethyl-1¹H-8-oxa-1(5,3)-indola-6(1,3)-piperidina-2(1,3)-benzenacycloundecaphane-5,7-dione 79 (240 mg, 0.326 mmol; crude as TFA salt) and N-methyl-N—((S)-1-(4-morpholinobut-2-ynoyl)pyrrolidine-3-carbonyl)-L-valine 70 (193 mg, 0.391 mmol; crude as TFA salt) in DMF (10 mL) at −20° C. under an atmosphere of N₂ was added DIPEA (0.5 mL, 3.26 mmol), followed by COMU (168 mg, 0.391 mmol) in portions. The mixture was stirred at −20° C. for 2 h. After completion of the reaction (TLC/LC monitoring), the reaction mixture was diluted with ice-cold brine (20 mL) and extracted with EtOAc (3×20 mL). The combined organic layer was dried over anhydrous Na₂SO₄, and filtered. The filtrate was concentrated under reduced pressure and the residue was purified by RP-HPLC to afford (3S)—N-((2S)-1-(((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide 80, Compound 1 (15 mg, 4% yield, over last two steps) as a white solid. ¹H NMR (400 MHz, DMSO-d₆): 8.75 (m, 1H), 7.99-7.96 (m, 1H), 7.90-7.86 (m, 1H), 7.79-7.73 (m, 1H), 7.65-7.58 (m, 1H), 7.57-7.48 (m, 3H), 7.34-7.25 (m, 1H), 7.04-6.99 (m, 1H), 5.03 (m, 1H), 4.34 (m, 1H), 4.23 (m, 1H), 4.02 (m, 3H), 3.81 (m, 2H), 3.69 (m, 1H), 3.51 (m, 12H), 3.02 (m, 9H), 2.81 (m, 3H), 2.13 (m, 4H), 1.91 (m, 2H), 1.72 (m, 3H), 1.54 (m, 1H), 1.42 (m, 3H), 1.22 (m, 2H), 0.94 (m, 6H), 0.72 (m, 6H), 0.49 (m, 3H) [along with minor peaks, arising presumably due to rotameric components, intensity of which reduced significantly in VT 1H-NMR]; LCMS=[M+H]⁺: 984.81, Purity=99.87%.

### Example 2

### Compound 2

### Synthesis of (3S)—N-((2S)-1-(((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide (85) [Compound 2]

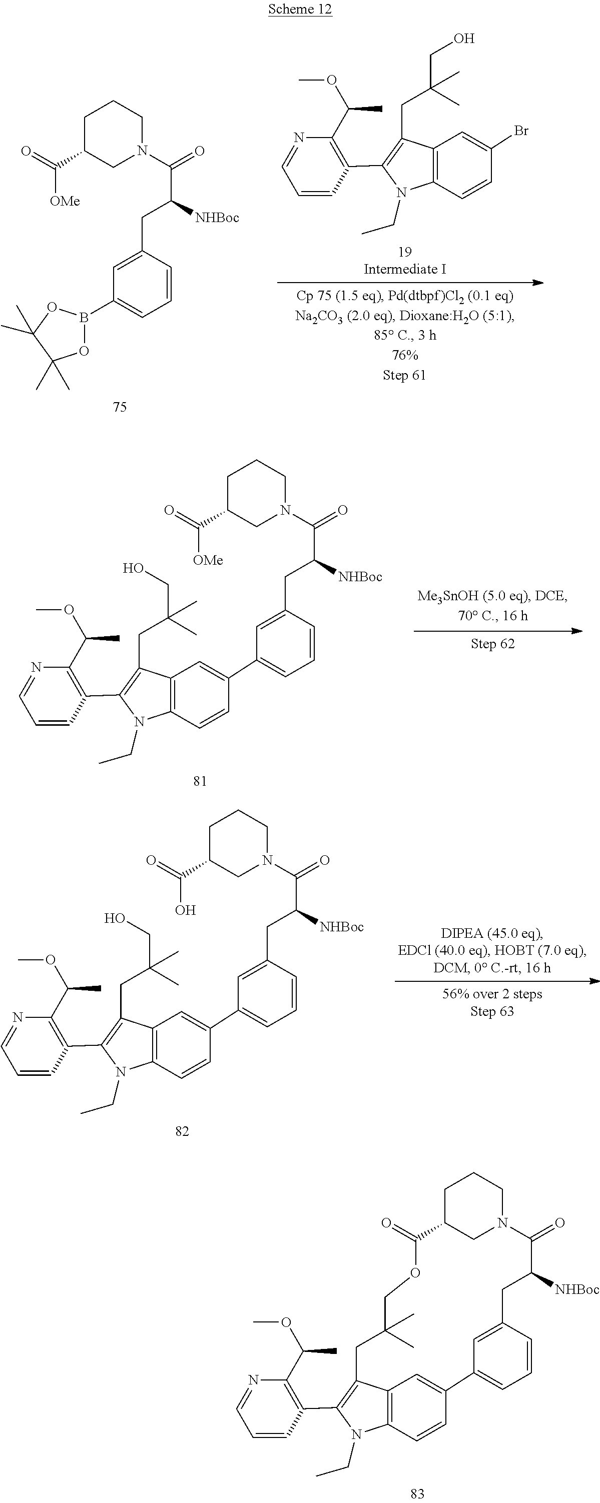

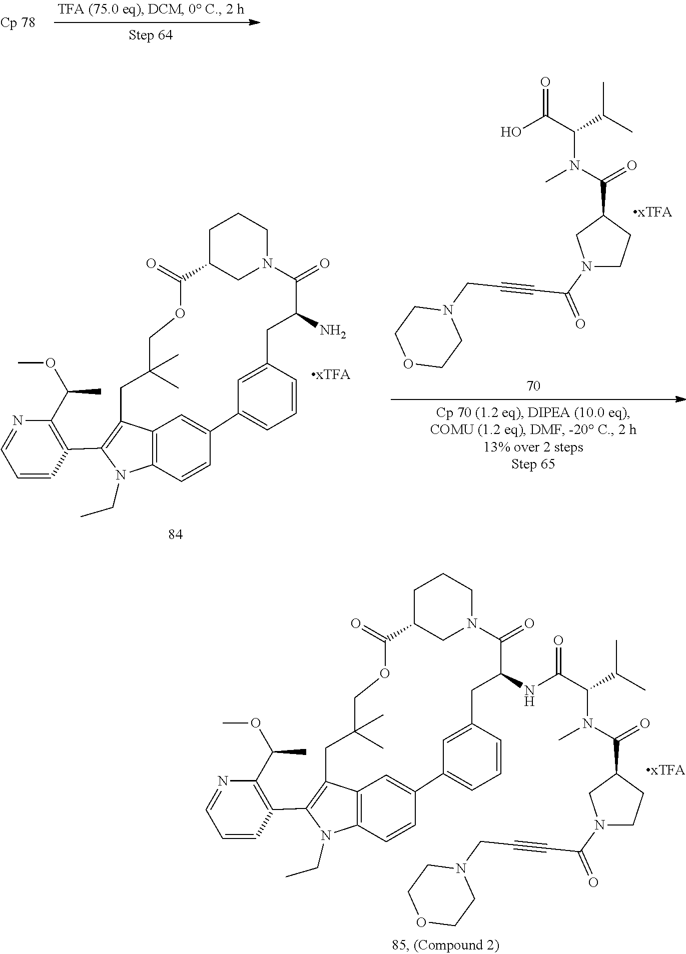

### Step 61

Synthesis of methyl (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylate (76): To a stirred mixture of (S)-3-(5-bromo-1-ethyl-2-(2-(1-methoxyethyl)pyridin-3-yl)-1H-indol-3-yl)-2,2-dimethylpropan-1-ol 19 (relatively polar atropisomer among the two atropisomers, 350 mg, 0.788 mmol) and methyl (R)-1-((S)-2-((tert-butoxycarbonyl)amino)-3-(3-(4,4,5,5-tetramethyl-1,3,2-dioxaborolan-2-yl)phenyl)propanoyl)piperidine-3-carboxylate 75 (610 mg, 1.18 mmol) in 1,4-dioxane (15 mL) and H₂O (3 mL) at rt under an atmosphere of Ar was added Na₂CO₃ (170 mg, 1.6 mmol) followed by [1,1′-Bis(di-tert-butylphosphino)ferrocene]dichloropalladium(II)] (51 mg, 0.078 mmol). The resulting mixture was purged with Argon for 10 min, and was then heated to 85° C. for next 3 h. After completion of the reaction (TLC/LC monitoring), the reaction mass was diluted with EtOAc (50 mL) and washed with H₂O (3×20 mL). The organic layer was dried over anhydrous Na₂SO₄, filtered and the filtrate was concentrated under reduced pressure to get a crude which was purified by Combi-flash column chromatography over silica gel using 30-70% EtOAc in heptane affording methyl (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylate 81 (450 mg, 76% yield) as a light yellow solid.

### Step 62

Synthesis of (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylic acid (82): To a stirred mixture of methyl (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylate 81 (450 mg, 0.596 mmol) in DCE (10 mL) at rt was added trimethyltin hydroxide (542 mg, 2.98 mmol) in portions. The mixture was heated to 70° C. for 16 h. After completion of the reaction (TLC/LC monitoring), the reaction mass was filtered and the filter cake was thoroughly washed with DCM (100 mL). The filtrate was concentrated under reduced pressure to give (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylic acid 82 (500 mg, crude), which was used directly in the next step without further purification.

### Step 63

Synthesis of tert-butyl ((6³R,4S)-1¹-ethyl-1²-(2-((5)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)carbamate (83): To a stirred mixture of (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)piperidine-3-carboxylic acid 82 (500 mg, crude, 0.596 mmol) in DCM (60 mL) at 0° C. under an atmosphere of N₂ was added DIPEA (4.7 mL, 27.0 mmol), followed by HOBT (635 mg, 4.17 mmol) and EDCI (4.6 g, 23.9 mmol) in portions. The mixture was warmed to rt and stirred for 16 h. After completion of the reaction (TLC/LC monitoring), the reaction mass was diluted with DCM (100 mL), washed with brine (3×50 mL), dried over anhydrous Na₂SO₄, and filtered. The filtrate was concentrated under reduced pressure and the residue was purified by Combi-flash column chromatography over silica gel using 20-70% EtOAc in heptane to give tert-butyl ((6³R,4S)-1¹-ethyl-1²-(2-(S))-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)carbamate 83 (240 mg, 56% yield, over 2 steps) as a white solid.

### Step 64

Synthesis of (6³R,4S)-4-amino-1¹-ethyl-1²-(2-((S)-1-methoxyethyl)pyridin-3-yl)-10,10-dimethyl-1¹H-8-oxa-1(5,3)-indola-6(1,3)-piperidina-2(1,3)-benzenacycloundecaphane-5,7-dione (84) To a stirred ice cold solution of tert-butyl ((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)carbamate 83 (240 mg, 0.33 mmol) in DCM (10 mL) was added trifluoroacetic acid (1.9 mL, 24.9 mmol) and the mixture was stirred at this temperature for 2 h. After completion of reaction (TLC monitoring), reaction mass was concentrated under reduced pressure to give a crude which was further evaporated with toluene (20 mL; repeated thrice) eventually to give (6³R,4S)-4-amino-1¹-ethyl-1²-(2-((S)-1-methoxyethyl)pyridin-3-yl)-10,10-dimethyl-1¹H-8-oxa-1(5,3)-indola-6(1,3)-piperidina-2(1,3)-benzenacycloundecaphane-5,7-dione 84 (300 mg, crude as TFA salt), which was used directly in the next step without further purification.

### Step 65

Synthesis of (3S)—N-((2S)-1-(((6³R,4S)-1¹-ethyl-1²-(2-((5)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide (95) [Compound 2]: To a stirred mixture of (6³R,4S)-4-amino-1¹-ethyl-1²-(2-((S)-1-methoxyethyl)pyridin-3-yl)-10,10-dimethyl-1¹H-8-oxa-1(5,3)-indola-6(1,3)-piperidina-2(1,3)-benzenacycloundecaphane-5,7-dione 84 (240 mg, 0.326 mmol; crude as TFA salt) and N-methyl-N—((S)-1-(4-morpholinobut-2-ynoyl)pyrrolidine-3-carbonyl)-L-valine 70 (193 mg, 0.391 mmol; crude as TFA salt) in DMF (10 mL) at −20° C. under an atmosphere of N₂ was added DIPEA (0.5 mL, 3.26 mmol), followed by COMU (168 mg, 0.391 mmol) in portions. The mixture was stirred at −20° C. for 2 h. After completion of the reaction (TLC/LC monitoring), the reaction mixture was diluted with ice-cold brine (20 mL) and extracted with EtOAc (3×20 mL). The combined organic layer was dried over anhydrous Na₂SO₄, and filtered. The filtrate was concentrated under reduced pressure and the residue was purified by RP-HPLC to afford (3S)—N-((2S)-1-(((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide 85, Compound 2 (41 mg, 13% yield, over last two steps) as a white solid. ¹H NMR (400 MHz, DMSO-d₆): 8.76 (d, J=4.4 Hz, 1H), 7.97-7.95 (m, 1H), 8.87 (d, J=7.2 Hz, 1H), 7.74-7.69 (m, 1H), 7.59-7.49 (m, 4H), 7.35-7.28 (m, 1H), 7.04-7.03 (m, 1H), 4.87-4.78 (m, 1H), 4.71-4.68 (m, 1H), 4.35 (m, 1H), 4.07-4.06 (m, 1H), 4.01-3.96 (m, 3H), 3.81-3.79 (m, 4H), 3.59-3.54 (m, 9H), 3.48-3.41 (m, 3H), 3.14 (s, 3H), 3.11-3.09 (m, 2H), 2.99-2.98 (m, 2H), 2.96-2.95 (m, 2H), 2.79 (m, 1H), 2.42-2.39 (m, 4H), 2.24 (m, 1H), 2.14-2.09 (m, 3H), 1.96-1.88 (m, 1H), 1.66-1.63 (m, 1H), 1.51-1.45 (m, 1H), 1.22 (d, J=6.4 Hz, 3H), 1.15-1.09 (m, 3H), 1.01-0.99 (m, 1H), 0.88-0.84 (m, 6H), 0.79-0.73 (m, 3H), 0.50 (m, 3H) [along with minor peaks, arising presumably due to rotameric components, intensity of which reduced significantly in VT 1H-NMR]; LCMS=[M+H]⁺: 984.85, Purity=99.19%.

### Example 3

### Compound 3

### Synthesis of (3S)-1-(4-(dimethylamino)-4-oxobut-2-yn-1-yl)-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide 58, Compound 3

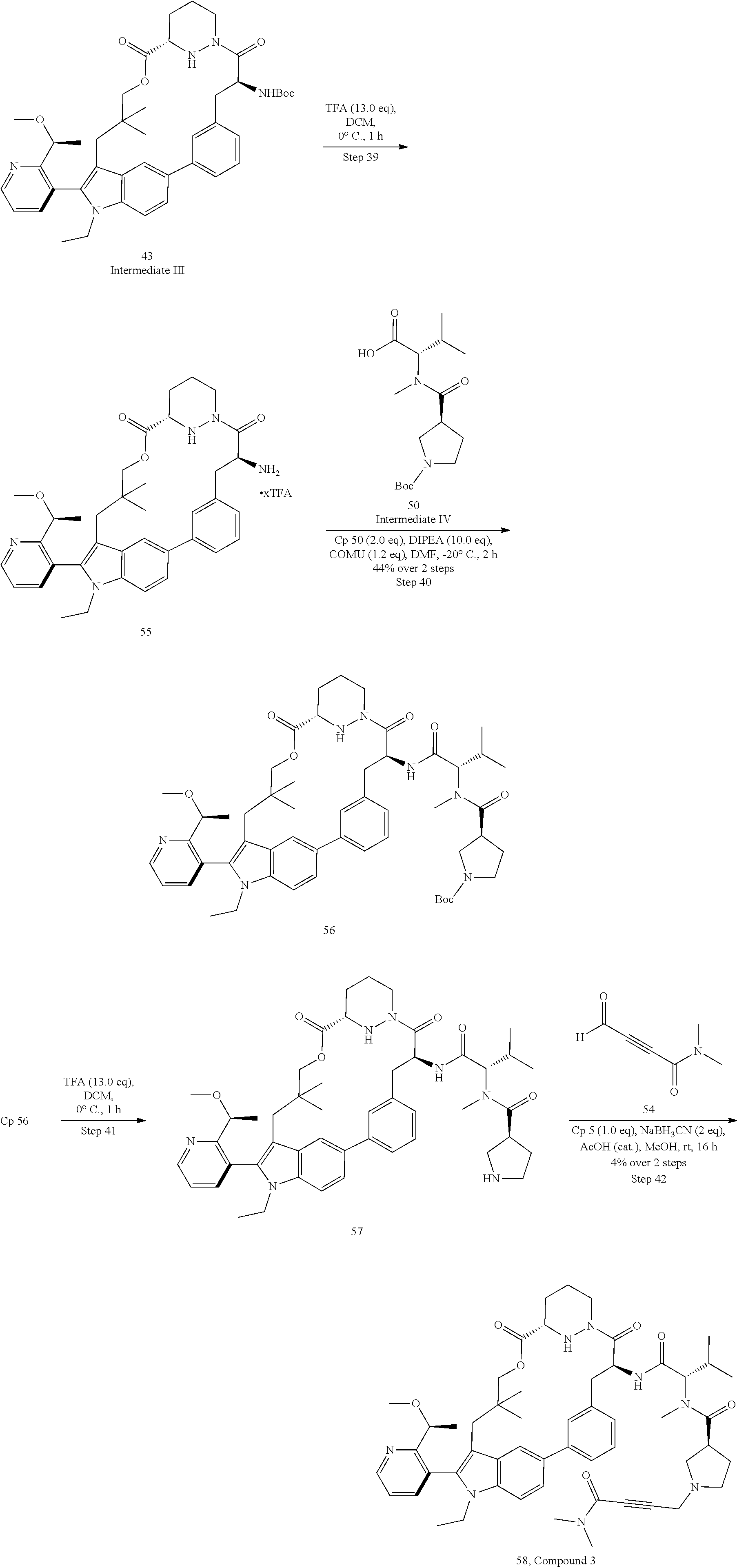

### Step 39

Synthesis of tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (55): To a stirred ice cold solution of tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate 43 (US20210130303, 700 mg, 0.967 mmol) in DCM (10 mL) was added trifluoroacetic acid (5.5 mL, 72.6 mmol) and the mixture was stirred at this temperature for 1 h. After completion of reaction (TLC monitoring), reaction mass was concentrated under reduced pressure to give a crude which was further evaporated with toluene (20 mL; repeated thrice) eventually to give (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione 55 (as TFA salt, 750 mg crude) which was used directly in the next step without further purification.

### Step 40

Synthesis of tert-butyl (3S)-3-(((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)pyrrolidine-1-carboxylate (56): To a stirred solution of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane- 5,7-dione 55 (US20210130303, 750 mg; crude as TFA salt, 1.2 mmol) in DMF (10 mL) at −20° C. under an atmosphere of N₂ was added DIPEA (2 mL, 12 mmol), followed by (2S)-3-methyl-2-[N-methyl-1-[(3S)-1-(prop-2-enoyl)pyrrolidin-3-yl]formamido]butanoic acid 50 (787 mg, 2.4 mmol) and COMU (616 mg, 1.44 mmol) respectively. The mixture was stirred at −20° C. for 2 h. After completion of reaction (TLC monitoring), the reaction mass was diluted with brine (50 mL) and extracted with EtOAc (3×50 mL). The combined organic layers were washed with brine (2×50 mL), dried over anhydrous Na₂SO₄, and filtered. The filtrate was concentrated under reduced pressure and the residue thus obtained was purified by silica gel column chromatography to give tert-butyl (3S)-3-(((2S)-1-(((6³S,4S)-1-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)pyrrolidine-1-carboxylate 56 (400 mg, 44% yield, over last two steps) as white solid.

### Step 41

Synthesis of (3S)—N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (57): To a stirred ice cold solution of tert-butyl (3S)-3-(((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)pyrrolidine-1-carboxylate 56 (400 mg, 0.428 mmol) in DCM (10 mL) was added trifluoroacetic acid (0.328 mL, 4.28 mmol) and the reaction mixture was stirred at 0° C. for 1 h. After completion of reaction (TLC monitoring), reaction mass was concentrated under reduced pressure to give a crude which was further evaporated with toluene (20 mL; repeated thrice) eventually to give the crude TFA salt. It was then basified using aq. NH₄OH and extracted with 20% IPA in CHCl₃ (4×50 mL) to get (3S)—N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide 57 (280 mg, crude), which was used directly in the next step without further purification.

### Step 42

Synthesis of (3S)-1-(4-(dimethylamino)-4-oxobut-2-yn-1-yl)-N-((2S)-1-(((6³S,4S)-1¹-ethyl-1²-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (58): To a stirred mixture (3S)—N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide 57 (100 mg, 0.120 mmol) and N,N-dimethyl-4-oxobut-2-ynamide 54 (22.5 mg, 0.18 mmol) in MeOH (5 mL) was added Acetic acid (catalytic; 0.1 mL) at 0° C. The reaction mixture was stirred at 0° C. for 30 min. Sodium cyanoborohydride (16 mg, 0.24 mmol) was then added to the reaction mixture, it was allowed to come to rt and stirred for next 16 h. After completion of reaction (monitored by TLC), reaction mass was concentrated under reduced pressure to get a crude which was purified by RP-HPLC to afford (3S)-1-(4-(dimethylamino)-4-oxobut-2-yn-1-yl)-N-((2S)-1-(((6³S,4S)-11-ethyl-1²-(2-((S)-1-methoxyethyl)pyridin-3-yl)-10,10-dimethyl-5,7-dioxo-6¹,6²,6³,6⁴,6⁵,6⁶-hexahydro-¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide 58, Compound 3 (5 mg, 4.3% yield, over last two steps) as a white solid.

¹H NMR (400 MHz, DMSO-d₆): 8.75 (d, J=4.0 Hz, 1H), 7.98 (m, 1H), 7.84-7.81 (m, 2H), 7.73-7.59 (m, 3H), 7.54-7.51 (m, 1H), 7.28-7.19 (m, 1H), 7.06 (d, J=8.8 Hz, 1H), 5.37-5.28 (m, 2H), 4.26-4.24 (m, 3H), 4.06-3.95 (m, 3H), 3.17-3.13 (m, 9H), 2.86-2.74 (m, 12H), 2.56 (m, 1H), 2.42-2.38 (m, 1H), 2.08-1.96 (m, 6H), 1.84-1.81 (m, 2H), 1.67 (m, 1H), 1.58-1.53 (m, 1H), 1.39 (d, J=6.0 Hz, 3H), 1.23 (s, 2H), 0.97-0.87 (m, 7H), 0.76-0.71 (m, 7H) [along with minor peaks, arising presumably due to rotameric components, intensity of which reduced significantly in VT 1H-NMR]; LCMS=[M+H]⁺: 943.84, Purity=96.77%.

### Example 4

### Compound 4

### Synthesis of Compound 4

Compound 4 was prepared or can be prepared following the synthetic schemes depicted below and using the general methods and procedures described herein.

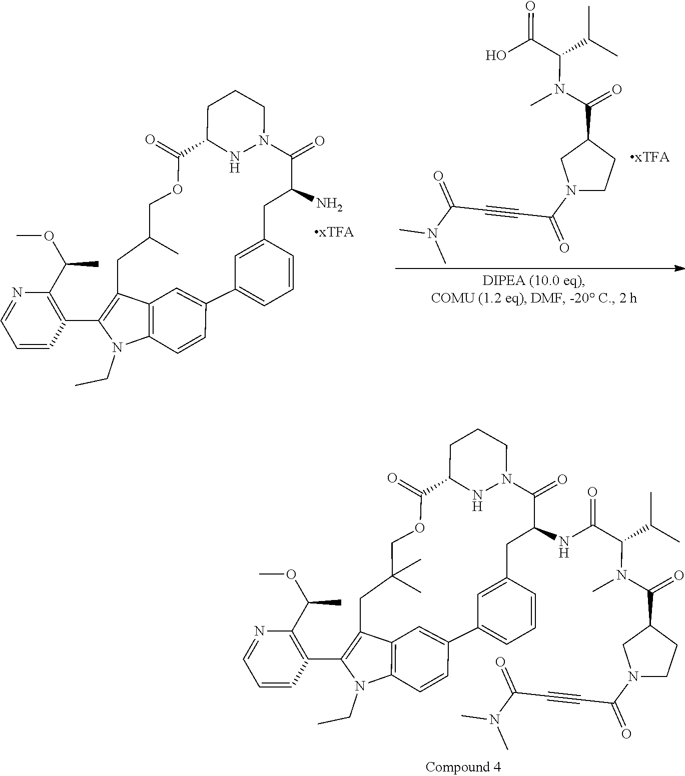

### Alternate Synthesis of Compound 4

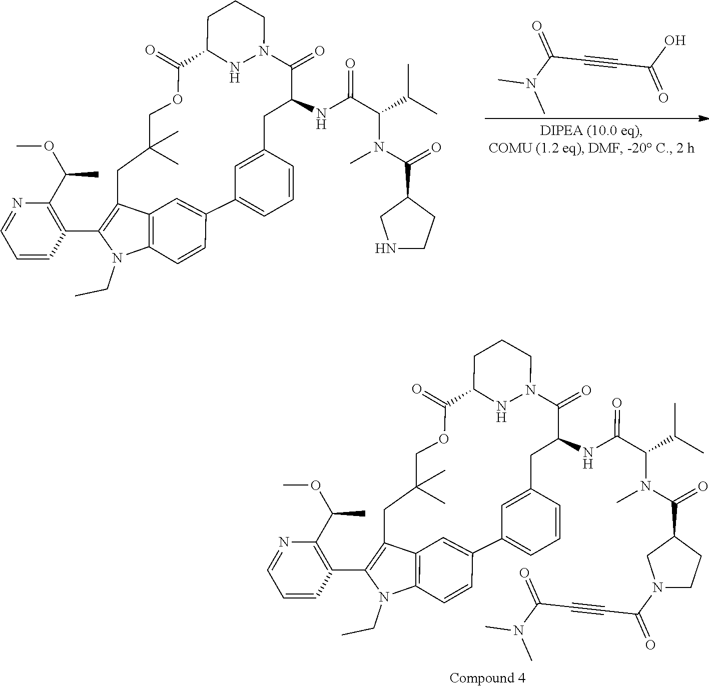

### Synthesis of Compound 5

Compound 5 was prepared or can be prepared following the synthetic schemes depicted below and using the general methods and procedures described herein.

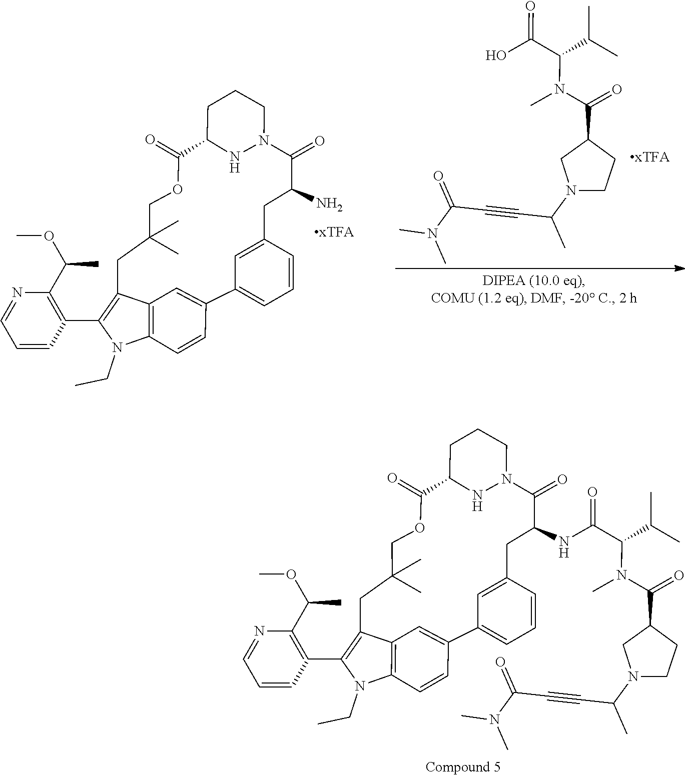

### Alternate Synthesis of Compound 5

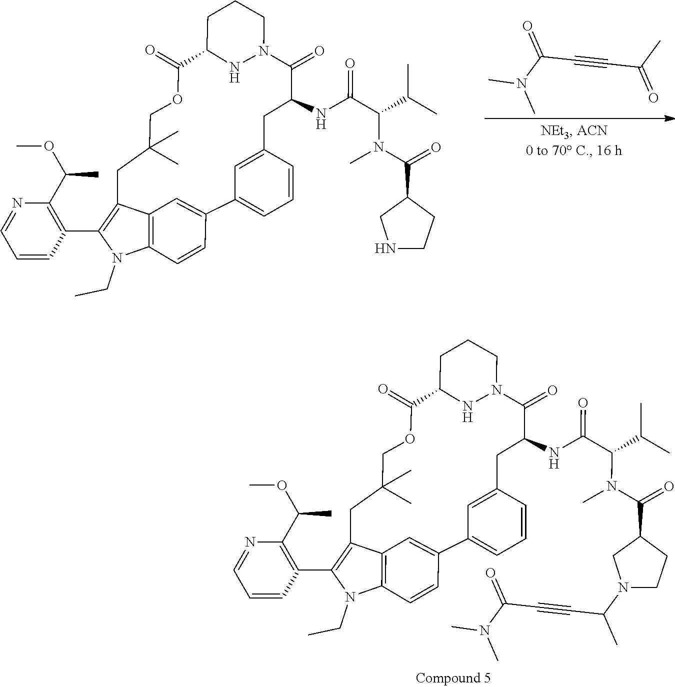

### Example 6

### Compound 101

### Synthesis of (3S)-1-((E)-4-(dimethylamino)-4-oxobut-2-en-1-yl)-N-((2S)-1-(((6³S,4S)-1¹-ethyl-1²-(2((S 1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (Compound 101)

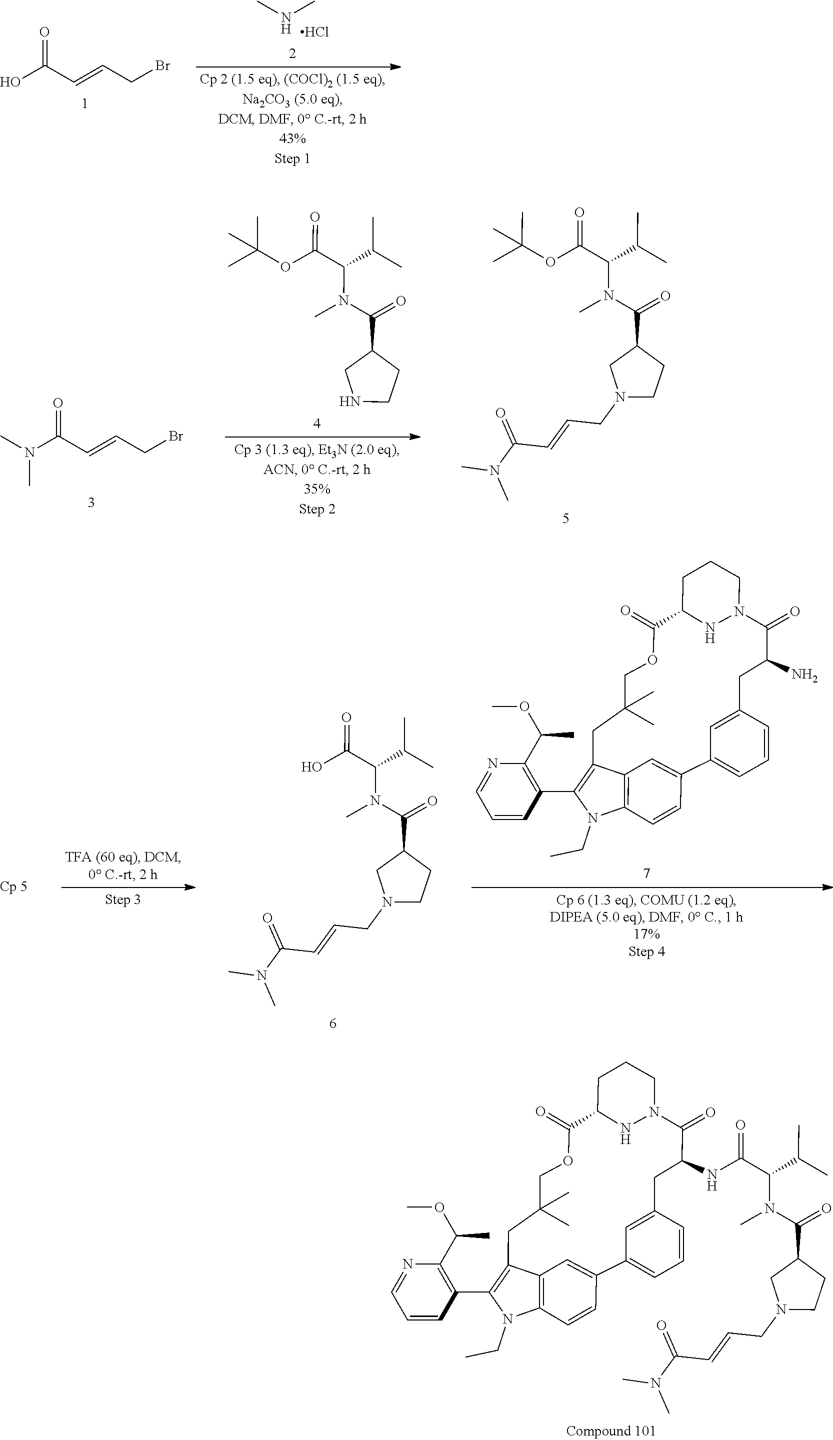

### Step 1

Synthesis of (2E)-4-bromo-N,N-dimethylbut-2-enamide (3): To a stirred solution of (2E)-4-bromobut-2-enoic acid 1 (30 g, 181.1 mmol) in DCM (500 mL) at 0° C. under N₂ was added oxalyl chloride (23.4 mL, 1.5 eq, 273 mmol) in a drop-wise fashion followed by catalytic amount of DMF (1.5 mL, 0.01 eq, 19.38 mmol). The resulting solution was stirred at 0° C. for next 1 h, and subsequently to it was added Na₂CO₃ (96.6 g, 5 eq, 750 mmol) followed by dimethylamine hydrochloride 2 (22.26 g, 1.5 eq, 273 mmol) under an atmosphere of N₂. The resulting reaction mixture was then allowed to come to rt and stirred for next 2 h. After completion of reaction (TLC monitoring), the reaction mixture was quenched with water, extracted with DCM (3×200 mL), and washed with brine. The combined organic layer was dried over anhydrous Na₂SO₄, filtered, the filtrate concentrated under reduced pressure and the crude residue was purified by Combi-flash column chromatography over silica gel (30% EtOAc in n-Heptane) to afford (2E)-4-bromo-N,N-dimethylbut-2-enamide 3 (15 g, 43%) as a yellow solid. ¹H NMR: (CDCl₃, 400 MHz) δ 6.87-6.94 (m, 1H), 6.48 (d, J=14.8, 1H), 4.035 (d, 2H, J=8.8 Hz), 3.037 (s, 6H); LCMS (ESI): m/z [M+H]⁺ calc'd for C₆H₁₁BrNO 190.00; found 190.12.

### Step 2

Synthesis of tert-butyl (2S)-2-{1-[(3S)-1-[(2E)-3-(dimethylcarbamoyl)prop-2-en-1-yl]pyrrolidin-3-yl]-N-methylformamido}-3-methylbutanoate (5): To a stirred solution of tert-butyl (2S)-3-methyl-2-{N-methyl-1-[(3S)-pyrrolidin-3-yl]formamido}butanoate 4 (13.8 g, 48.6 mmol) and (2E)-4-bromo-N,N-dimethylbut-2-enamide 3 (12.12 g, 1.3 eq, 63 mmol) in acetronitrile (120 mL) at 0° C. under an atmosphere of N₂, was added triethylamine (13.65 mL, 2 eq, 96.9 mmol). The reaction mixture was allowed to come to rt and stirred for next 2 h. After completion of the reaction (TLC monitoring), the reaction mixture was concentrated under reduced pressure to get the crude which was purified by Combi-flash column chromatography over silica gel to afford tert-butyl (2S)-2-{1-[(3S)-1-[(2E)-3-(dimethylcarbamoyl)prop-2-en-1-yl]pyrrolidin-3-yl]-N-methylformamido}-3-methylbutanoate 5 (6 g, 35%) as a brown semi-solid. LCMS (ESI): m/z [M+H]⁺ calc'd for C₂₁H₃₈N₃O₄ 396.29; found 396.51.

### Step 3

Synthesis of N—((S)-1-((E)-4-(dimethylamino)-4-oxobut-2-en-1-yl)pyrrolidine-3-carbonyl)-N-methyl-L-valine (6): To a stirred solution of tert-butyl (2S)-2-{1-[(3S)-1-[(2E)-3-(dimethylcarbamoyl)prop-2-en-1-yl]pyrrolidin-3-yl]-N-methylformamido}-3-methylbutanoate 5 (5 g, 12.65 mmol) in DCM (100 mL) under an atmosphere of N₂ was added trifluoroacetic acid (58 mL, 60 eq, 760 mmol) at 0° C. The reaction mixture was allowed to come to rt and stirred for next 2 h. After completion of the reaction (TLC monitoring), the reaction mixture was concentrated under reduced pressure to get the crude N—((S)-1-((E)-4-(dimethylamino)-4-oxobut-2-en-1-yl)pyrrolidine-3-carbonyl)-N-methyl-L-valine 6 (5 g, as TFA salt) as a thick liquid, which was used directly in the next step without further purification. LCMS (ESI): m/z [M+H]⁺ calc'd for C₁₇H₃₀N₃O₄ 340.22; found 340.24.

### Step 4

Synthesis of (3S)-1-((E)-4-(dimethylamino)-4-oxobut-2-en-1-yl)-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (Compound 101): To a stirred solution of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione 7 (2 g, 3.21 mmol) and N—((S)-1-((E)-4-(dimethylamino)-4-oxobut-2-en-1-yl)pyrrolidine-3-carbonyl)-N-methyl-L-valine 6 (1.89 g, 1.3 eq, 4.17 mmol, crude as TFA salt, its preparation is described in another section) in DMF (30 mL) at 0° C. under an atmosphere of N₂ was added DIPEA (2.85 mL, 5 eq, 16 mmol) followed by COMU (1.65 g, 1.2 eq, 3.85 mmol) and the reaction mixture was stirred at 0° C. for 1 h. After completion of the reaction (TLC/LC monitoring), the reaction mixture was diluted with ice-cold brine (20 mL) and extracted with EtOAc (3×100 mL). The combined organic layer was dried over anhydrous Na₂SO₄, and filtered. The filtrate was concentrated under reduced pressure and the residue was purified by RP-HPLC to afford (3S)-1-((E)-4-(dimethylamino)-4-oxobut-2-en-1-yl)-N-((2S)-1-(((6³S,4S)-1¹-ethyl-1²-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide, Compound 101 (500 mg, 17% w.r.t Cp 7) as a white solid. ¹H NMR (400 MHz, DMSO-d₆): δ 8.77-8.75 (m, 1H), 7.99 (m, 1H), 7.84-7.81 (m, 2H), 7.73-7.60 (m, 3H), 7.54-7.51 (m, 1H), 7.27-7.17 (m, 1H), 7.10 (m, 1H), 6.62-6.60 (m, 2H), 5.53-5.29 (m, 2H), 4.30-4.23 (m, 3H), 4.07-3.95 (m, 3H), 3.69-3.59 (m, 2H), 3.24-3.23 (m, 2H), 3.13 (m, 3H), 3.05-3.01 (m, 3H), 2.97-2.94 (m, 1H), 2.88-2.80 (m, 6H), 2.78-2.74 (m, 4H), 2.71-2.63 (m, 2H), 2.46-2.37 (m, 1H), 2.10-1.81 (m, 6H), 1.69-1.52 (m, 2H), 1.38 (d, J=6.0 Hz, 3H), 0.97-0.88 (m, 6H), 0.77-0.70 (m, 6H), 0.51 (m, 3H); LCMS (ESI): m/z [M+H]⁺ calc'd for C₅₄H₇₃N₈O₇ 945.56; found 945.48,

### Example 7

### Compound 102

### Synthesis of (3S)-1-(4-(dimethylamino)but-2-ynoyl)-N-((2S)-1-(((6³S,4S)-1¹-ethyl-1²-(2-((S)-1-methoxyethyl)pyridin-3-yl)-10,10-dimethyl-5,7-dioxo-1 ¹H-8-oxa-1(5,3)-indola-6(1,3)-pyrrolidina-2(1,3)-benzenacycloundecaphane-4-yl)amino)-3-methyl-1-oxobutan-2-yl)-N-methylpyrrolidine-3-carboxamide (Compound 102)

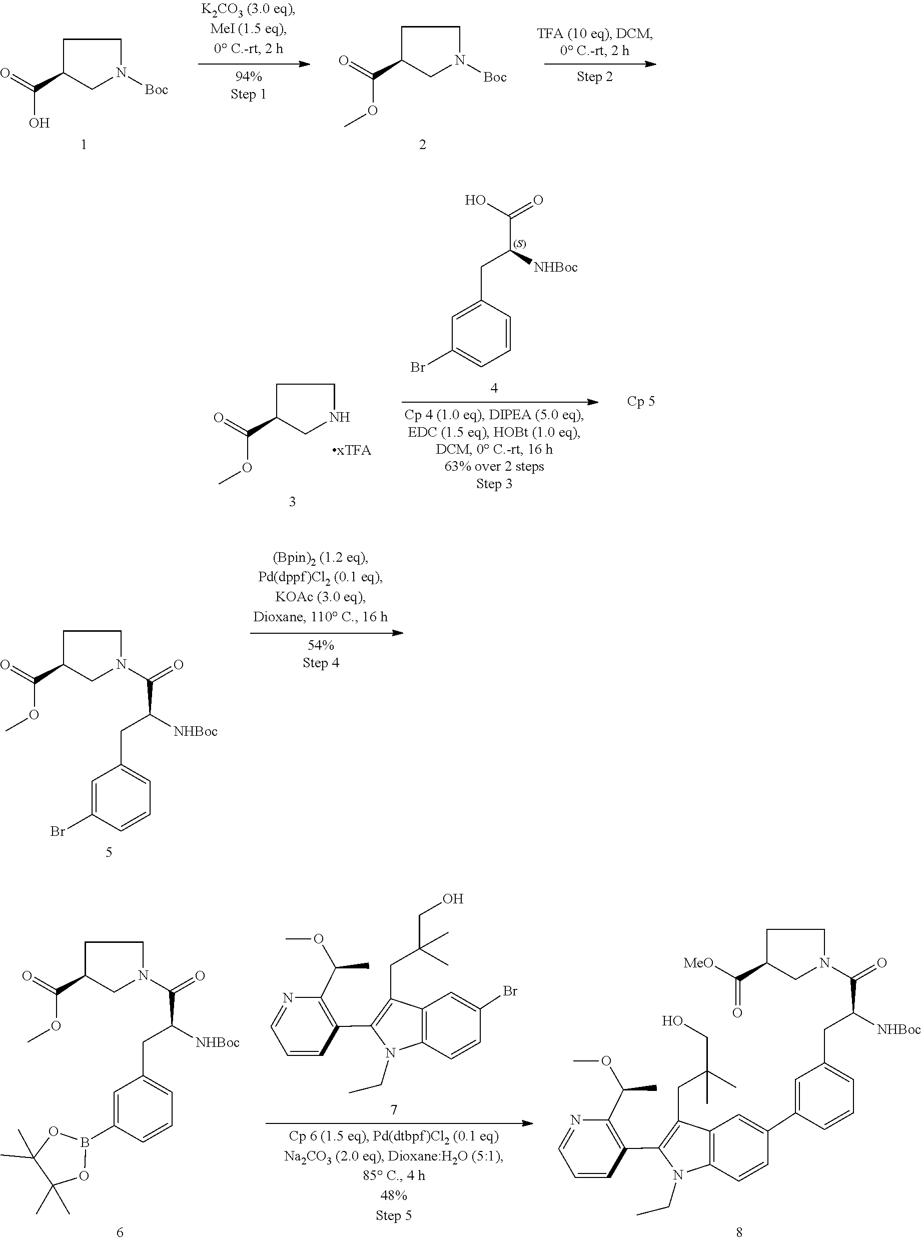

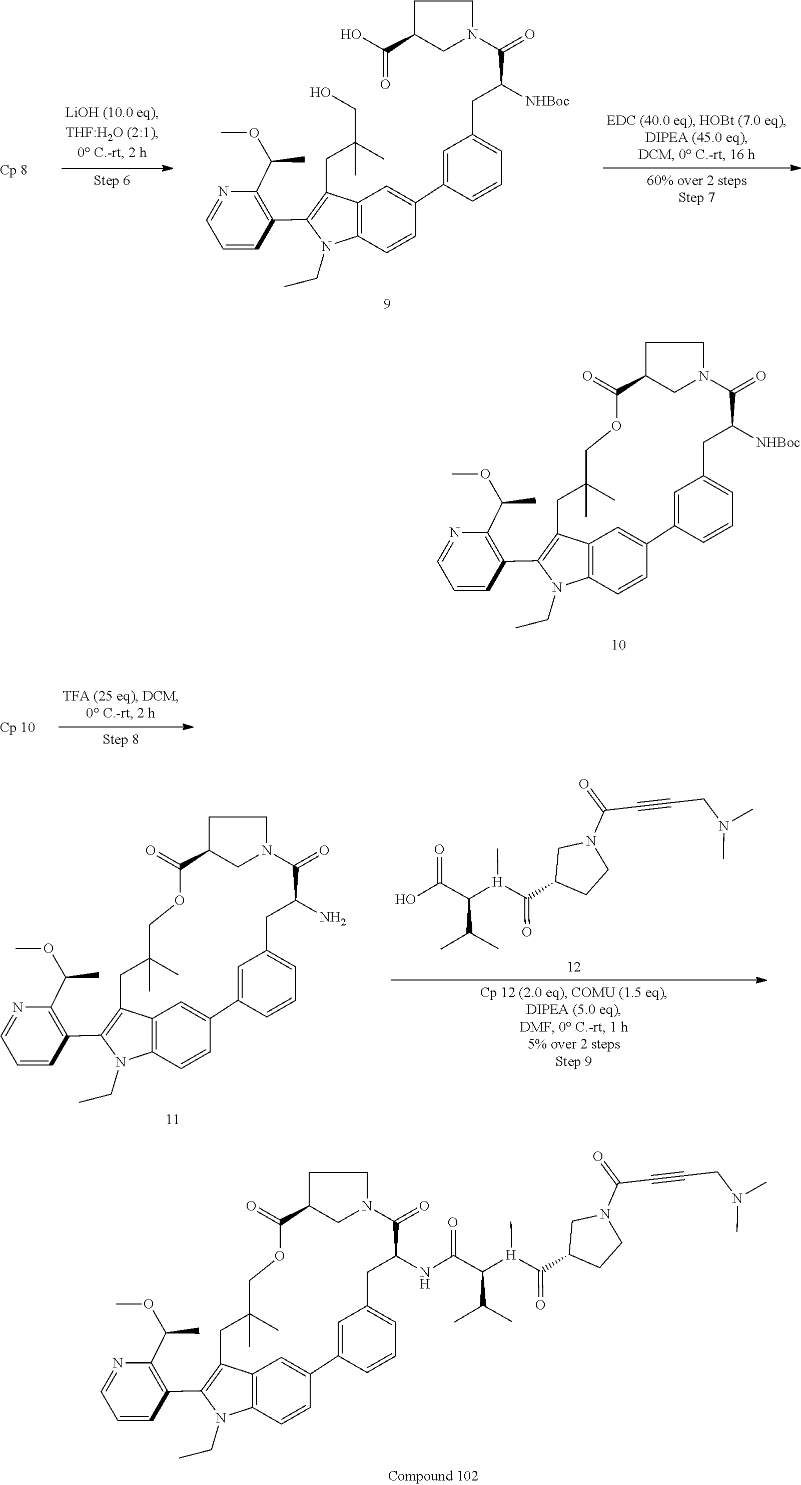

### Step 1

Synthesis of 1-(tert-butyl) 3-methyl (S)-pyrrolidine-1,3-dicarboxylate (2): A mixture of (S)-1-(tert-butoxycarbonyl)pyrrolidine-3-carboxylic acid 1 (5 g, 23.2 mmol) and K₂CO₃ (9.63 g, 3 eq, 69.7 mmol) in DMF (50 mL) was stirred at rt for 10 min under nitrogen atmosphere. To it was then added iodomethane (2.17 mL, 1.5 eq, 34.8 mmol) at 0° C. and stirred the reaction at rt for 2 h. After completion of reaction (TLC monitoring), the reaction mixture was diluted with EtOAc (200 mL) and washed with chilled brine solution (3×50 mL). The combined organic layer was dried over anhydrous Na₂SO₄, filtered and the filtrate thus obtained was concentrated under reduced pressure to get a crude which was purified by Combi-flash column chromatography over silica gel using 5-20% EtOAc in n-heptane to obtain 1-(tert-butyl) 3-methyl (S)-pyrrolidine-1,3-dicarboxylate 2 (5 g, 93%) as an oil. ¹H NMR (400 MHz, DMSO-d₆): δ 3.62 (s, 3H), 3.45-3.43 (m, 1H), 3.36-3.34 (m, 1H), 3.29-3.22 (m, 2H), 3.208-3.11 (m, 1H), 2.08-2.00 (m, 1H), 1.98-1.95 (m, 1H), 1.38 (s, 9H).

### Step 2

Synthesis of methyl (S)-pyrrolidine-3-carboxylate (3): To a stirred ice cold solution of 1-(tert-butyl) 3-methyl (S)-pyrrolidine-1,3-dicarboxylate 2 (5 g, 21.8 mmol) in DCM (50 mL) was added trifluoroacetic acid (12 mL, 10 eq, 218 mmol). The reaction mixture was allowed to come to rt and stirred for next 2 h. After completion of the reaction (TLC monitoring), reaction mixture was concentrated under reduced pressure to obtain methyl (S)-pyrrolidine-3-carboxylate 3 (5.3 g, crude as TFA salt), which was used in the next step without further purification.

### Step 3

Synthesis of methyl (S)-1-((S)-3-(3-bromophenyl)-2-((tert-butoxycarbonyl)amino)propanoyl)pyrrolidine-3-carboxylate (5): To an ice cold solution of methyl (S)-pyrrolidine-3-carboxylate 3 (5.3 g, 21.8 mmol, crude as TFA salt) in DCM (80 mL) was added DIPEA (20 mL, 5 eq, 109 mmol) under N₂ atmosphere. The resulting reaction mixture was stirred for 5 min, and to it were subsequently added HOBt (3.32 g, 1 eq, 21.8 mmol) and EDC (10.44 g, 1.5 eq, 32.7 mmol) followed by (S)-3-(3-bromophenyl)-2-((tert-butoxycarbonyl)amino)propanoic acid 4 (7.5 g, 21.8 mmol).

The reaction mixture was allowed to come to rt and stirred for next 16 h. After reaction monitoring (TLC), the reaction mixture was diluted with DCM (100 mL) and washed with saturated aq. NH₄Cl solution (3×50 mL). The organic layer was dried over anhydrous Na₂SO₄, filtered and evaporated under reduced pressure to get the crude residue which was purified by Combi-flash column chromatography over silica gel using 20-70% EtOAc in heptane to afford methyl (S)-1-((S)-3-(3-bromophenyl)-2-((tert-butoxycarbonyl)amino)propanoyl)pyrrolidine-3-carboxylate 5 (6.3 g, 63% over 2 steps) as a light brown solid. ¹H NMR (400 MHz, DMSO-d₆): δ 7.38 (s, 2H), 7.18 (d, J=4.8 Hz, 2H), 5.40-5.34 (m, 1H), 4.56 (s, 1H), 3.56 (s, 3H), 3.52-3.48 (m, 2H), 3.08-2.96 (m, 4H), 2.12 (m, 1H), 2.00-1.95 (m, 1H), 1.92-1.76 (m, 1H), 1.44 (s, 9H).

### Step 4

Synthesis of methyl (S)-1-((S)-2-((tert-butoxycarbonyl)amino)-3-(3-(4,4,5,5-tetramethyl-1,3,2-dioxaborolan-2-yl)phenyl)propanoyl)pyrrolidine-3-carboxylate (6): To a stirred mixture of methyl (S)-1-((S)-3-(3-bromophenyl)-2-((tert-butoxycarbonyl)amino)propanoyl)pyrrolidine-3-carboxylate 5 (3 g, 6.59 mmol) in 1,4-dioxane (60 mL) was added KOAc (1.94 g, 3 eq, 19.8 mmol) under an atmosphere of Argon. The resulting mixture was purged with Argon for 10 min. Subsequently to it was added bis(pinacolato)diboron (2.01 g, 1.2 eq, 7.6 mmol) followed by [1,1′-Bis(diphenylphosphino)ferrocene]dichloropalladium(II)] (482 mg, 0.1 eq, 0.659 mmol). The resulting reaction mixture was heated at 110° C. for next 16 h. After reaction monitoring (TLC), the reaction mixture was filtered through celite bed and washed with EtOAc. The filtrate was evaporated under reduced pressure to get the crude residue which was purified by Combi-flash column chromatography over silica gel using 60-70% EtOAc in n-heptane to give methyl (S)-1-((S)-2-((tert-butoxycarbonyl)amino)-3-(3-(4,4,5,5-tetramethyl-1,3,2-dioxaborolan-2-yl)phenyl)propanoyl)pyrrolidine-3-carboxylate 6 (1.8 g, 54%) as a semi-solid. ¹H NMR (400 MHz, DMSO-d₄): δ 7.62 (m, 2H), 7.28-7.26 (m, 1H), 5.43-5.41 (m, 1H), 4.56-4.52 (m, 1H), 3.72 (s, 3H), 3.63-3.58 (m, 1H), 3.56-3.54 (m, 1H), 3.01-2.92 (m, 2H), 2.76-2.71 (m, 1H), 2.03-1.96 (m, 1H), 1.41 (s, 9H), 1.32 (s, 12H), 1.26-1.23 (d, J=12.8 Hz, 4H).

### Step 5

Synthesis of methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylate (8): To a stirred mixture of (S)-3-(5-bromo-1-ethyl-2-(2-(1-methoxyethyl)pyridin-3-yl)-1H-indol-3-yl)-2,2-dimethylpropan-1-ol 7 (1 g, 2.22 mmol) and methyl (S)-1-((S)-2-((tert-butoxycarbonyl)amino)-3-(3-(4,4,5,5-tetramethyl-1,3,2-dioxaborolan-2-yl)phenyl)propanoyl)pyrrolidine-3-carboxylate 6 (1.67 g, 1.5 eq, 3.32 mmol) in 1,4-dioxane (50 mL) at rt under an atmosphere of Argon, was added sequentially Na₂CO₃ (470 mg, 2 eq, 4.44 mmol), [1,1′-Bis(di-tert-butylphosphino)ferrocene]dichloropalladium(II) (145 mg, 0.1 eq, 0.22 mmol), and H₂O (10 mL). The reaction mixture was then purged with Argon gas for 10 min and heated to 85° C. for next 4 h. After reaction monitoring (TLC), the reaction mixture was cooled to rt, diluted with H₂O (20 mL) and extracted with EtOAc (3×100 mL). The combined organic layers were washed with brine (2×50 mL), dried over anhydrous Na₂SO₄, and filtered. The filtrate was concentrated under reduced pressure and the residue was purified by Combi-flash column chromatography over silica gel using 50-100% EtOAc in heptane to afford methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylate 8 (800 mg, 48%) as a solid. LCMS (ESI): m/z [M+H]⁺ calc'd for C₄₃H₅₇N₄O₇ 741.42; found 741.29.

### Step 6

Synthesis of (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylic acid (9): To a stirred mixture of methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylate 8 (1.4 g, 1.88 mmol) in THF (20 mL) and water (10 mL) in 2:1 ratio at 0° C. under N₂ atmosphere, was added LiOH (792 mg, 10 eq, 18.88 mmol) in portions. The reaction mixture was allowed to come to rt and stirred for next 2 h. After completion of reaction (monitored by TLC and LCMS), THF was evaporated under reduced pressure, the aqueous phase acidified with 1N HCl to pH ˜5 and extracted with 25% IPA in CHCl₃ (3×50 mL). The combined organic layer was dried over anhydrous Na₂SO₄, filtered and concentrated under reduced pressure to get the crude (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylic acid 9 (2 g, crude), which was used in the next step without further purification. LCMS (ESI): m/z [M+H]⁺ calc'd for C₄₂H₅₅N₄O₇ 727.40; found 727.57.

### Step 7

Synthesis of tert-butyl ((6³S,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)carbamate (10): To a stirred mixture of (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylic acid 9 (700 mg, 0.944 mmol) in DCM (20 mL) at 0° C. under an atmosphere of N₂ was added DIPEA (7.5 mL, 45 eq, 42.6 mmol), HOBT (895 mg, 7 eq, 6.62 mmol) and EDC (7.24 mg, 40 eq, 37.8 mmol) in portions. The mixture was warmed to rt and stirred for 16 h. After reaction monitoring (TLC), the reaction mass was diluted with DCM (100 mL), and washed with brine (3×50 mL). The organic layer was dried over anhydrous Na₂SO₄, and filtered. The filtrate was concentrated under reduced pressure and the residue was purified by Combi-flash column chromatography over silica gel using 30-80% EtOAc in heptane to afford tert-butyl ((6³S,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)carbamate 10 (400 mg, 60% yield, over last two steps) as a solid. LCMS (ESI): m/z [M+H]⁺ calc'd for C₄₂H₅₃N₄O₆ 709.40; found 709.43.

### Step 8

Synthesis of (6³S,4S)-4-amino-1¹-ethyl-1²-(2-((S)-1-methoxyethyl)pyridin-3-yl)-10,10-dimethyl-1¹H-8-oxa-1(5,3)-indola-6(1,3)-pyrrolidina-2(1,3)-benzenacycloundecaphane-5,7-dione (11): To an ice cold stirred solution of tert-butyl ((6³S,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)carbamate 10 (200 mg, 0.282 mmol) in DCM (5 mL) was added TFA (0.5 mL, 25 eq, 7.05 mmol) at 0° C. The reaction mixture was gradually warmed to rt and stirred for next 2 h. After completion of reaction (TLC monitoring), the reaction mass was concentrated under reduced pressure to give a crude which was further basified using aq. NaHCO₃ to pH ˜10, extracted with 20% IPA in CHCl₃ (3×50 mL), and evaporated in vacuo eventually to obtain (6³S,4S)-4-amino-1¹-ethyl-1²-(2-((S)-1-methoxyethyl)pyridin-3-yl)-10,10-dimethyl-1¹H-8-oxa-1(5,3)-indola-6(1,3)-pyrrolidina-2(1,3)-benzenacycloundecaphane-5,7-dione 11 (120 mg) as a brown solid, which was used in the next step without further purification. LCMS (ESI): m/z [M+H]⁺ calc'd for C₃₇H₄₅N₄O₄ 609.34; found 609.39.

### Step 9

Synthesis of (3S)-1-(4-(dimethylamino)but-2-ynoyl)-N-((2S)-1-(((6³S,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)amino)-3-methyl-1-oxobutan-2-yl)-N-methylpyrrolidine-3-carboxamide (Compound 102): To an ice cold stirred solution of (6³S,4S)-4-amino-1¹-ethyl-1²-(2-((S)-1-methoxyethyl)pyridin-3-yl)-10,10-dimethyl-1¹H-8-oxa-1(5,3)-indola-6(1,3)-pyrrolidina-2(1,3)-benzenacycloundecaphane-5,7-dione 11 (180 mg, 0.296 mmol) and N—((S)-1-(4-(dimethylamino)but-2-ynoyl)pyrrolidine-3-carbonyl)-N-methyl-L-valine 12 (200 mg, 2 eq, 0.592 mmol, crude as TFA salt) in DMF (10 mL) at 0° C. under an atmosphere of N₂ was added DIPEA (0.25 ml, 5 eq, 1.478 mmol), and stirred at this temperature for 5 min. Subsequently, COMU (190 mg, 1.5 eq, 0.444 mmol) was added, the reaction mixture was gradually warmed to rt, and stirred for next 1 h. After completion of reaction (TLC/LCMS monitoring), the reaction mixture was quenched with chilled saturated aq.NH₄Cl (50 mL) and extracted with EtOAc (3×50 mL). The combined organic layer was washed with chilled brine (2×50 mL), dried over anhydrous Na₂SO₄, and filtered. The filtrate was concentrated under reduced pressure and the residue thus obtained was purified by RP-HPLC to afford (3S)-1-(4-(dimethylamino)but-2-ynoyl)-N-((2S)-1-(((6³S,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)amino)-3-methyl-1-oxobutan-2-yl)-N-methylpyrrolidine-3-carboxamide (Compound 102) (11 mg, 4.8%, 98.64% purity) as a white solid. ¹H NMR (400 MHz, DMSO-d₆): δ 8.76 (d, J=4.8 Hz, 1H), 7.95 (dd, J=1.6, 7.6 Hz, 1H), 7.83 (d, J=11.6 Hz, 1H), 7.75 (m, 1H), 7.57 (d, J=8.4 Hz, 1H), 7.54-7.51 (m, 2H), 7.48-7.43 (m, 1H), 7.32-7.27 (m, 1H), 7.03 (t, J=8.2 Hz, 1H), 4.88-4.78 (m, 1H), 4.71 (d, J=9.6 Hz, 1H), 4.46-4.42 (m, 1H), 4.35-4.31 (m, 1H), 4.17-4.13 (m, 1H), 4.08-3.92 (m, 2H), 3.84-3.77 (m, 2H), 3.73-3.58 (m, 4H), 3.53-3.50 (m, 1H), 3.48-3.39 (m, 3H), 3.26-3.21 (m, 2H), 3.19-3.16 (m, 2H), 2.97 (d, J=6.4 Hz, 3H), 2.83 (s, 2H), 2.76 (d, J=4.4 Hz, 3H), 2.21 (t, J=3.6 Hz, 3H), 2.13 (s, 3H), 2.12-2.10 (m, 1H), 2.07-2.01 (m, 2H), 1.99-1.90 (m, 2H), 1.65-1.46 (m, 1H), 1.43 (dd, J=2, 6.2 Hz, 3H), 1.22 (t, J=7.2 Hz, 3H), 0.92 (m, 3H), 0.78 (s, 3H), 0.62 (s, 3H), 0.46 (d, J=14 Hz, 3H). LCMS (ESI): m/z [M+H]⁺ calc'd for C₅₄H₇₀N₇O₇ 928.53; found 928.25.

### Example 8

### Compound 103

### Synthesis of (3S)-1-(4-(dimethylamino)but-2-ynoyl)-N-((2S)-1-(((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)amino)-3-methyl-1-oxobutan-2-yl)-N-methylpyrrolidine-3-carboxamide (Compound 103)

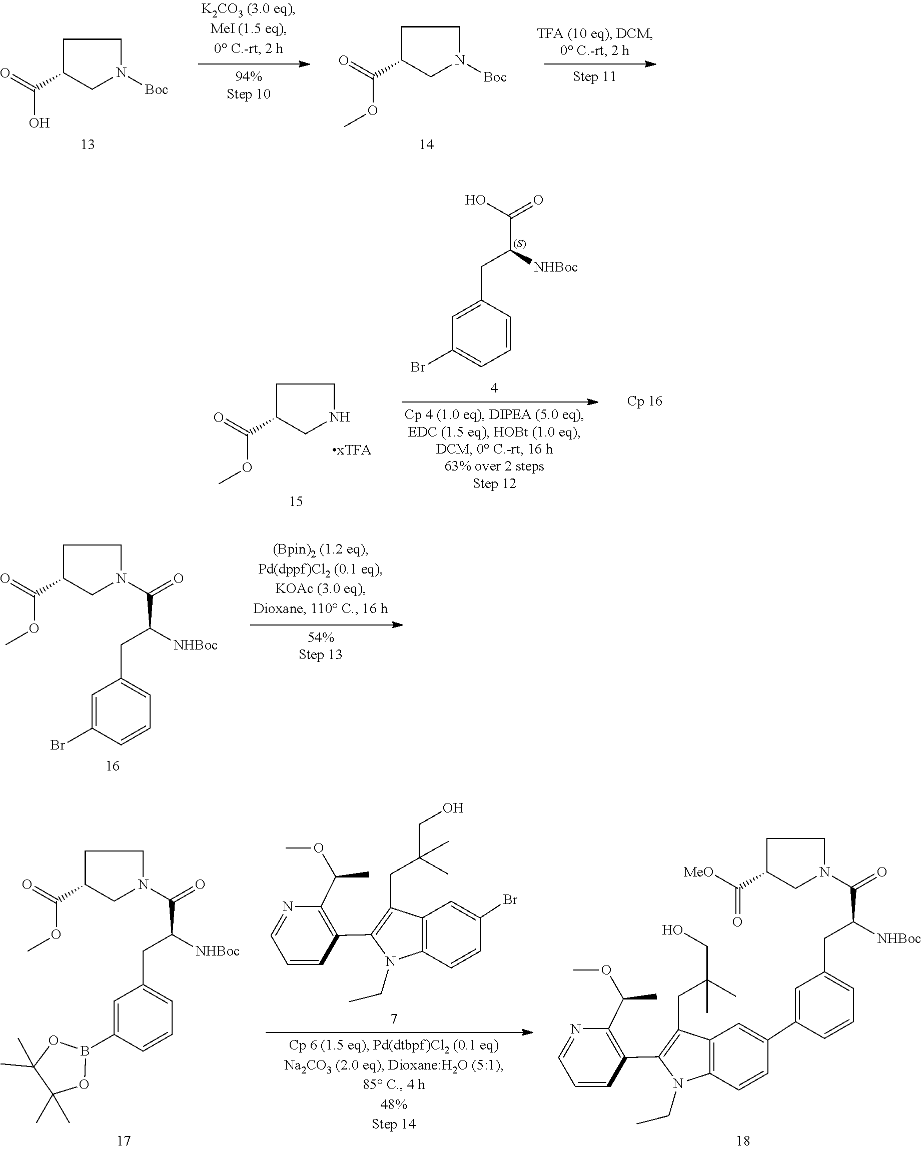

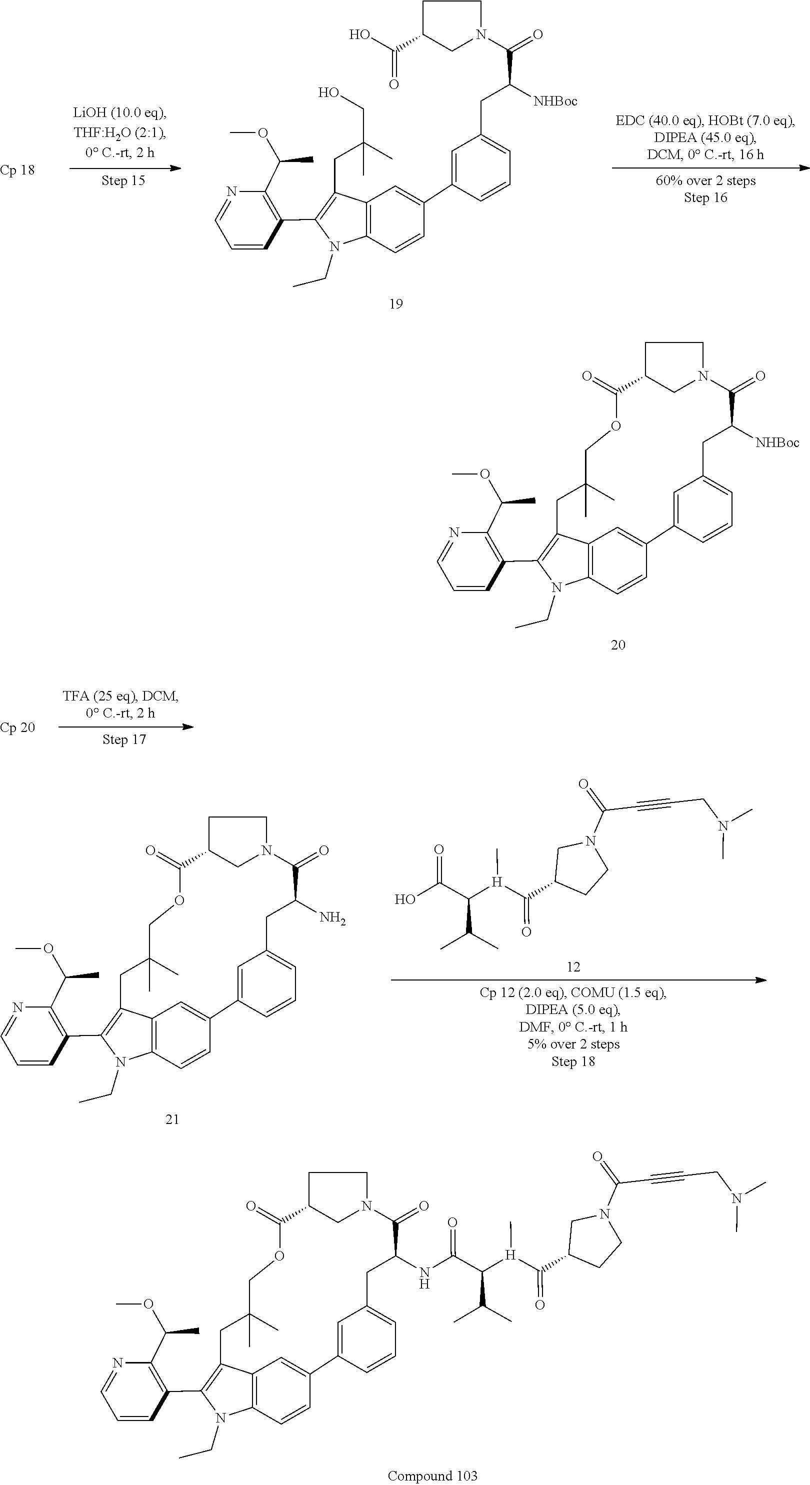

### Step 10

Synthesis of 1-(tert-butyl) 3-methyl (R)-pyrrolidine-1,3-dicarboxylate (14): Following the procedure as described in Scheme 1, Step 1 (vide supra) using (R)-1-(tert-butoxycarbonyl)pyrrolidine-3-carboxylic acid 13, 1-(tert-butyl) 3-methyl (R)-pyrrolidine-1,3-dicarboxylate 14 was prepared. ¹H NMR (400 MHz, DMSO-d₄): δ 3.62 (s, 3H), 3.45-3.43 (m, 1H), 3.36-3.34 (m, 1H), 3.29-3.22 (m, 2H), 3.18-3.11 (m, 1H), 2.08-2.05 (m, 1H), 1.98-1.95 (m, 1H), 1.38 (s, 9H).

### Step 11

Synthesis of methyl (R)-pyrrolidine-3-carboxylate (15): Following the procedure as described in Scheme 1, Step 2 (vide supra) using 1-(tert-butyl) 3-methyl (R)-pyrrolidine-1,3-dicarboxylate 14, methyl (R)-pyrrolidine-3-carboxylate 15 (crude, as TFA salt) was prepared, which was used in the next step without further purification.

### Step 12

Synthesis of methyl (R)-1-((S)-3-(3-bromophenyl)-2-((tert-butoxycarbonyl)amino)propanoyl)pyrrolidine-3-carboxylate (16): Following the procedure as described in Scheme 1, Step 3 (vide supra) using methyl (R)-pyrrolidine-3-carboxylate 15 (crude, as TFA salt) and (S)-3-(3-bromophenyl)-2-((tert-butoxycarbonyl)amino)propanoic acid 4, methyl (R)-1-((S)-3-(3-bromophenyl)-2-((tert-butoxycarbonyl)amino)propanoyl)pyrrolidine-3-carboxylate 16 was prepared. LCMS (ESI): m/z [M+H]⁺ calc'd for C₂₀H₂₈BrN₂O₅ 455.11; found 455.09.

### Step 13

Synthesis of methyl (R)-1-((S)-2-((tert-butoxycarbonyl)amino)-3-(3-(4,4,5,5-tetramethyl-1,3,2-dioxaborolan-2-yl)phenyl)propanoyl)pyrrolidine-3-carboxylate (17): Following the procedure as described in Scheme 1, Step 4 (vide supra) using methyl (R)-1-((S)-3-(3-bromophenyl)-2-((tert-butoxycarbonyl)amino)propanoyl)pyrrolidine-3-carboxylate 16, methyl (R)-1-((S)-2-((tert-butoxycarbonyl)amino)-3-(3-(4,4,5,5-tetramethyl-1,3,2-dioxaborolan-2-yl)phenyl)propanoyl)pyrrolidine-3-carboxylate 17 was prepared. ¹H NMR (400 MHz, DMSO-d₆): δ 7.67 (m, 2H), 7.32-7.27 (m, 3H), 5.49 (q, J=5.6, 1H), 5.28 (d, J=8.4, 1H), 4.06-4.00 (m, 2H), 3.70 (s, 3H), 2.98-2.88 (m, 2H), 2.11-2.05 (m, 1H), 2.05 (brs, 3H), 1.41 (s, 9H), 1.27 (s, 12H).

### Step 14

Synthesis of methyl (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylate (18): Following the procedure as described in Scheme 1, Step 5 (vide supra) using (S)-3-(5-bromo-1-ethyl-2-(2-(1-methoxyethyl)pyridin-3-yl)-1H-indol-3-yl)-2,2-dimethylpropan-1-ol 7 and methyl (R)-1-((S)-2-((tert-butoxycarbonyl)amino)-3-(3-(4,4,5,5-tetramethyl-1,3,2-dioxaborolan-2-yl)phenyl)propanoyl)pyrrolidine-3-carboxylate 17, methyl (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylate 18 was prepared. LCMS (ESI): m/z [M+H]⁺ calc'd for C₄₃H₅₇N₄O₇ 741.42; found 741.29.

### Step 15

Synthesis of (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylic acid (19): Following the procedure as described in Scheme 1, Step 6 (vide supra) using methyl (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylate 18, (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylic acid 19 was prepared. LCMS (ESI): m/z [M+H]⁺ calc'd for C₄₂H₅₅N₄O₇ 727.40; found 727.59.

### Step 16

Synthesis of tert-butyl ((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)carbamate (20): Following the procedure as described in Scheme 1, Step 7 (vide supra) using (R)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)phenyl)propanoyl)pyrrolidine-3-carboxylic acid 19, tert-butyl ((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)carbamate 20 was prepared. LCMS (ESI): m/z [M+H]⁺ calc'd for C₄₂H₅₃N₄O₆ 709.39; found 709.45.

### Step 17

Synthesis of (6³R,4S)-4-amino-1¹-ethyl-1²-(2-((S)-1-methoxyethyl)pyridin-3-yl)-10,10-dimethyl-1H-8-oxa-1(5,3)-indola-6(1,3)-pyrrolidina-2(1,3)-benzenacycloundecaphane-5,7-dione (21): Following the procedure as described in Scheme 1, Step 8 (vide supra) using tert-butyl ((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)carbamate 20, (6³R,4S)-4-amino-1¹-ethyl-1²-(2-((S)-1-methoxyethyl)pyridin-3-yl)-10,10-dimethyl-1¹H-8-oxa-1(5,3)-indola-6(1,3)-pyrrolidina-2(1,3)-benzenacycloundecaphane-5,7-dione 21 was prepared. LCMS (ESI): m/z [M+H]⁺ calc'd for C₃₇H₄₅N₄O₄ 609.34; found 609.39.

### Step 18

Synthesis of (3S)-1-(4-(dimethylamino)but-2-ynoyl)-N-((2S)-1-(((6³R,4S)-1¹-ethyl-1²-(2-((S)-1-methoxyethyl)pyridin-3-yl)-10,10-dimethyl-5,7-dioxo-1H-8-oxa-1(5,3)-indola-6(1,3)-pyrrolidina-2(1,3)-benzenacycloundecaphane-4-yl)amino)-3-methyl-1-oxobutan-2-yl)-N-methylpyrrolidine-3-carboxamide (Compound 103): Following the procedure as described in Scheme 1, Step 9 (vide supra) using (6³R,4S)-4-amino-1¹-ethyl-1²-(2-((S)-1-methoxyethyl)pyridin-3-yl)-10,10-dimethyl-1¹H-8-oxa-1(5,3)-indola-6(1,3)-pyrrolidina-2(1,3)-benzenacycloundecaphane-5,7-dione 21 and N—((S)-1-(4-(dimethylamino)but-2-ynoyl)pyrrolidine-3-carbonyl)-N-methyl-L-valine 12, after RP-HPLC (3S)-1-(4-(dimethylamino)but-2-ynoyl)-N-((2S)-1-(((6³R,4S)-1¹-ethyl-1²-(2-((S)-1-methoxyethyl)pyridin-3-yl)-10,10-dimethyl-5,7-dioxo-1¹H-8-oxa-1(5,3)-indola-6(1,3)-pyrrolidina-2(1,3)-benzenacycloundecaphane-4-yl)amino)-3-methyl-1-oxobutan-2-yl)-N-methylpyrrolidine-3-carboxamide (Compound 103) (18 mg, 5%, 97.53% purity) was obtained as a white solid. ¹H NMR (400 MHz, DMSO-d₆): δ 8.75 (d, 4.4 Hz, 1H), 8.03-7.94 (m, 1H), 7.92-7.83 (m, 1H), 7.63-7.61 (m, 1H), 7.58-7.56 (m, 1H), 7.52-7.50 (m, 2H), 7.46-7.37 (m, 1H), 7.33-7.27 (m, 1H), 7.11-7.03 (m, 1H), 4.82-4.76 (m, 1H), 4.72-4.70 (m, 1H), 4.36-4.19 (m, 2H), 4.10-3.96 (m, 2H), 3.92-3.89 (m, 1H), 3.87-3.81 (m, 1H), 3.79-3.78 (m, 2H), 3.67-3.63 (m, 2H), 3.59-3.53 (m, 2H), 3.50-3.45 (m, 3H), 3.42 (d, J=5.6 Hz, 1H), 3.41-3.37 (m, 1H), 3.18-3.14 (m, 2H), 3.09 (d, 2.8 Hz, 1H), 3.00-2.88 (m, 6H), 2.86-2.82 (m, 1H), 2.60-2.58 (m, 1H), 2.23-2.21 (m, 1H), 2.20-2.15 (m, 6H), 2.07-2.05 (m, 1H), 1.94-1.93 (m, 1H), 1.87-1.75 (m, 1H), 1.45-1.38 (m, 3H), 1.12 (q, 7.6 Hz, 2H), 0.92-0.85 (m, 5H), 0.80-0.73 (m, 6H), 0.40-0.32 (m, 3H). LCMS (ESI): m/z [M+H]⁺ calc'd for C₅₄H₇₀N₇O₇ 928.53; found 928.25.

### Example 9

### Compound 104

### Synthesis of (3S)-1-acryloyl-N-((2S)-1-(((6³S,8S,Z)-21-ethyl-2²-(2-((S)-1-methoxyethyl)pyridin-3-yl)-4,4-dimethyl-7-oxo-6¹,6²,6³,6⁴,6⁵,6⁶-hexahydro-2¹H,5¹H-2(5,3)-indola-6(3,1)-pyridazina-5(1,4)-triazola-1(1,3)-benzenacyclononaphane-8-yl)amino)-3-methyl-1-oxobutan-2-yl)-N-methylpyrrolidine-3-carboxamide, Compound 104

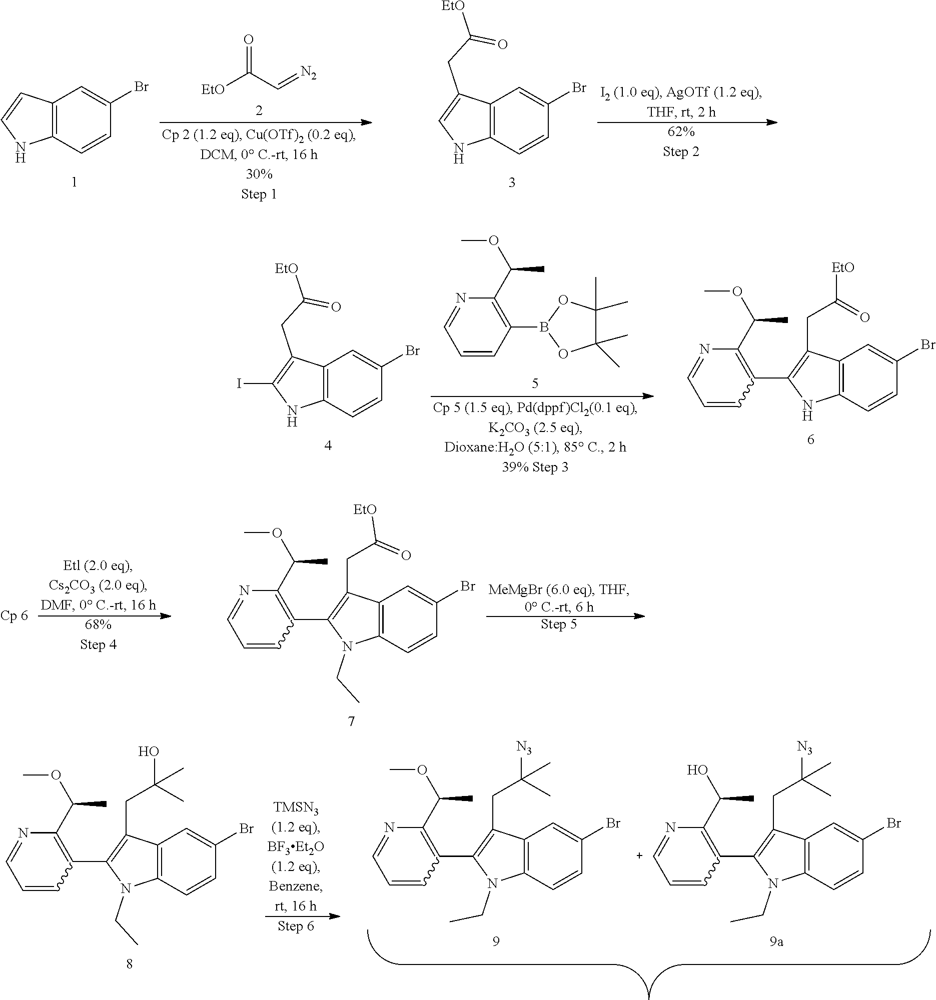

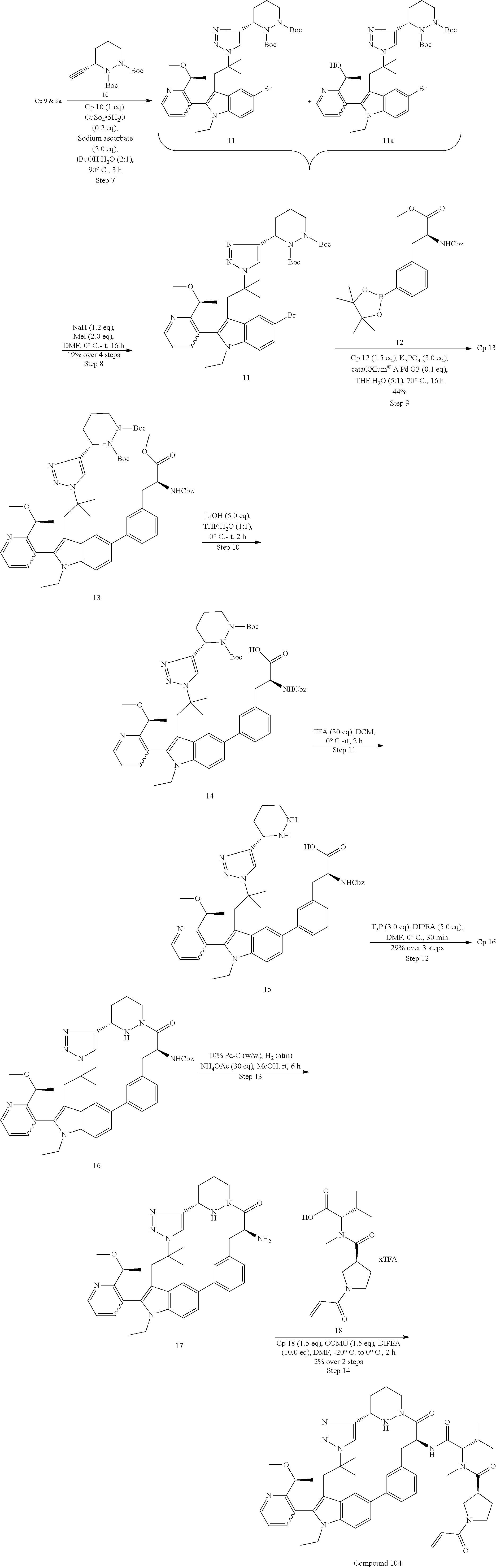

### Step 1

ethyl 2-(5-bromo-1H-indol-3-yl)acetate (3): To a stirred solution of 5-bromo-1H-indole 1 (50 g, 255 mmol) in dichloromethane (0.5 L) at 0° C. was added Cu(OTf)₂ (16.9 g, 0.2 eq, 51 mmol), followed by ethyl 2-diazoacetate (43.7 g, 1.2 eq, 306 mmol) dropwise. The reaction mixture was allowed to warm to rt and stirred for next 16 h. After completion of reaction (TLC monitoring), solvent was evaporated under reduced pressure to obtain a residue, which was purified by Combi-flash column chromatography over silica gel using 25% EtOAc in n-heptane to afford ethyl 2-(5-bromo-1H-indol-3-yl)acetate 3 (22 g, 30%) as a brown sticky liquid. ¹H NMR (400 MHz, DMSO-d₆): δ 8.23 (s, 1H), 7.74 (s, 1H), 7.27 (dd, J=7.2 Hz, 1.6 Hz, 1H), 7.18 (d, J=7.2 Hz, 1H), 7.11 (d, J=1.6 Hz, 1H), 4.16 (q, J=3.6 Hz, 2H), 3.71 (s, 2H), 0.85 (t, J=2.6 Hz, 3H); LCMS (ESI): m/z [M+H]⁺ calc'd for C₁₂H₁₃BrNO₂ 282.01 found 282.14.

### Step 2

ethyl 2-(5-bromo-2-iodo-1H-indol-3-yl)acetate (4): To stirred solution of ethyl 2-(5-bromo-1H-indol-3-yl)acetate 3 (22 g, 77.88 mmol) in THF (200 mL) was added I₂ (11.88 g, 1eq, 78.88 mmol) followed by AgOTf (23.98 g, 1.2 eq, 93.5 mmol) at 0° C. The mixture was allowed to warm to rt and stirred for next 2 h. After completion of reaction (TLC monitoring), saturated aq. solution of Na₂S203 (200 mL) was added to the reaction mixture, and extracted with EtOAc (3×500 mL). The combined organic layer was dried over anhydrous Na₂SO₄, filtered, and concentrated under reduced pressure and the residue was purified by Combi-flash column chromatography over silica gel to provide ethyl 2-(5-bromo-2-iodo-1H-indol-3-yl)acetate 4 (19.8 g, 62%) as a brown solid. ¹H NMR (400 MHz, DMSO-d₆): δ 8.21 (s, 1H), 7.67 (s, 1H), 7.13 (d, J=8.4 Hz, 1H), 7.07 (d, J=8.4 Hz, 1H), 4.17 (q, J=3.6 Hz, 2H), 3.66 (s, 2H), 1.26 (t, J=2.6 Hz, 3H); LCMS (ESI): m/z [M+H]⁺ calc'd for C₁₂H₁₂BrINO₂ 407.70 found 407.55.

### Step 3

ethyl (S)-2-(5-bromo-2-(2-(1-methoxyethyl)pyridin-3-yl)-1H-indol-3-yl)acetate (6): To a stirred solution of ethyl 2-(5-bromo-2-iodo-1H-indol-3-yl)acetate 4 (19.8 g, 48.62 mmol) and 2-[(1S)-1-methoxyethyl]-3-(4,4,5,5-tetramethyl-1,3,2-dioxaborolan-2-yl)pyridine 5 (12.77 g, 1.5 eq, 73.15 mmol) in 1,4-dioxane (120 mL) in a seal tube at rt was added sequentially K₂CO₃ (16.77 g, 2.5 eq, 121.27 mmol) and water (24 mL), and the reaction mixture was purged with Ar for 10 min. Subsequently, PdCl₂(dtbpf) (3.6 g, 0.1 eq, 4.86 mmol) was added, the reaction mixture was purged with Argon for another 5 min and heated to 85° C. for next 2 h. After reaction monitoring (TLC/LC-MS), the reaction mixture was cooled to rt, filtered over a Celite pad and thoroughly washed with EtOAc. The aqueous layer was further extracted with EtOAc (3×100 mL). The combined organic layer was washed with brine, dried over anhydrous Na₂SO₄, and filtered. The filtrate was concentrated under reduced pressure and the residue was purified by Combi-flash column chromatography over silica gel using 35% EtOAc in n-heptane to afford ethyl (S)-2-(5-bromo-2-(2-(1-methoxyethyl)pyridin-3-yl)-1H-indol-3-yl)acetate 6 (7.92 g, 39%) as a brown solid. ¹H NMR (400 MHz, DMSO-d₆): δ 9.69 (s, 1H), 8.66 (d, J=3.2 Hz, 1H), 8.03 (d, J=6.8 Hz, 1H), 7.82 (s, 1H), 7.39 (dd, J=6.8 Hz, 3.2 Hz, 2H), 7.27 (d, J=6.8 Hz, 2H), 4.62 (q, J=3.6 Hz, 1H), 4.15 (q, 2H), 3.36 (s, 3H), 1.38 (d, J=6.8, 3H), 1.28 (t, J=2.6, 3H); LCMS (ESI): m/z [M+H]⁺ calc'd for C₂₀H₂₂BrN₂O₃ 417.07 found 417.25.

### Step 4

ethyl 2-(5-bromo-2-{2-[(1S)-1-methoxyethyl]pyridin-3-yl}-1H-indol-3-yl)acetate (7): To an ice cold solution of ethyl (S)-2-(5-bromo-2-(2-(1-methoxyethyl)pyridin-3-yl)-1H-indol-3-yl)acetate 6 (7.92 g, 19.03 mmol) in DMF (25 mL) under an atmosphere of N₂ was added Cs₂CO₃ (12.37 g, 2 eq, 38.07 mmol) and Etl (5.91 g, 2.0 eq, 38.07 mmol) in portions. The mixture was warmed to rt and stirred for next 16 h. After completion of reaction (monitored by TLC), H₂O (150 mL) was then added to the reaction mixture and it was extracted with EtOAc (3×250 mL). The combined organic layer was washed with brine (3×300 mL), dried over anhydrous Na₂SO₄, and filtered. The filtrate was concentrated under reduced pressure and the residue was purified by Combi-flash column chromatography over silica gel to give ethyl 2-(5-bromo-2-{2-[(1S)-1-methoxyethyl]pyridin-3-yl}-1H-indol-3-yl)acetate 7 (5.76 g, 68%) as a brown sticky liquid. LCMS (ESI): m/z [M+H]⁺ calc'd for C₂₂H₂₆BrN₂O₃ 445.10 found 445.60.

### Step 5

(S)-1-(5-bromo-1-ethyl-2-(2-(1-methoxyethyl)pyridin-3-yl)-1H-indol-3-yl)-2-methylpropan-2-ol (8): To a stirred solution of ethyl 2-(5-bromo-1-ethyl-2-{2-[(1S)-1-methoxyethyl]pyridin-3-yl}-1H-indol-3-yl)acetate 7 (5.76 g, 12.97 mmol) in tetrahydrofuran (48 mL) was added methylmagnesium bromide (3M in Et₂O) (43.2 ml, 6 eq, 77.83 mmol) at 0° C. The reaction mixture was allowed to warm to rt and stirred for next 6 h. After completion of reaction (monitored by TLC), the reaction mixture was quenched by saturated aq. solution of NH₄Cl (100 mL) and extracted with ethyl acetate (3×250 mL). The combined organic layer was washed with brine (100 mL), dried over anhydrous Na₂SO₄, and filtered. The filtrate was concentrated under reduced pressure to get the crude (S)-1-(5-bromo-1-ethyl-2-(2-(1-methoxyethyl)pyridin-3-yl)-1H-indol-3-yl)-2-methylpropan-2-ol 8 (5.25 g) as a blackish viscous liquid which was used in the next step without further purification. LCMS (ESI): m/z [M+H] calc'd for C₂₂H₂₈BrN₂O₂ 431.13 found 431.26.

### Step 6

Synthesis of (S)-3-(2-azido-2-methylpropyl)-5-bromo-1-ethyl-2-(2-(1-methoxyethyl)pyridin-3-yl)-1H-indole 9 and (S)-1-(3-(3-(2-azido-2-methylpropyl)-5-bromo-1-ethyl-1H-indol-2-yl)pyridin-2-yl)ethan-1-ol (9a): To a stirred solution of (S)-1-(5-bromo-1-ethyl-2-(2-(1-methoxyethyl)pyridin-3-yl)-1H-indol-3-yl)-2-methylpropan-2-ol 8 (5.25 g, 12.20) in benzene (350 mL) at 0° C. was added azidotrimethylsilane (4.61 g, 1.2 eq, 14.64 mmol), followed by BF₃·Et₂O (5.67 g, 1.2 eq, 14.64 mmol) in a dropwise fashion at the same temperature. The reaction mixture was allowed to warm to rt and stirred for next 16 h. After completion of reaction (monitored by TLC), the reaction mixture was quenched by saturated aq. solution of NaHCO₃ (50 mL) and extracted with ethyl acetate (2×100 mL). The combined organic layer was washed with brine (50 mL), dried over anhydrous Na₂SO₄, and filtered. The filtrate was concentrated under reduce pressure to get a crude mixture of (S)-3-(2-azido-2-methylpropyl)-5-bromo-1-ethyl-2-(2-(1-methoxyethyl)pyridin-3-yl)-1H-indole 9 and (S)-1-(3-(3-(2-azido-2-methylpropyl)-5-bromo-1-ethyl-1H-indol-2-yl)pyridin-2-yl)ethan-1-ol 9a (5.54 g) as a brown solid. It was used in the next step as a mixture of Cp 9 and 9a without further purification.

### Step 7 & 8

### Synthesis of di-tert-butyl (S)-3-(1-(1-(5-bromo-1-ethyl-2-(2-((S)-1-methoxyethyl)pyridin-3-yl)-1H-indol-3-yl)-2-methylpropan-2-yl)-1H-1,2,3-triazol-4-yl)tetrahydropyridazine-1,2-dicarboxylate (11)

Step 7: To a stirred mixture of (S)-3-(2-azido-2-methylpropyl)-5-bromo-1-ethyl-2-(2-(1-methoxyethyl)pyridin-3-yl)-1H-indole 9 and (S)-1-(3-(3-(2-azido-2-methylpropyl)-5-bromo-1-ethyl-1H-indol-2-yl)pyridin-2-yl)ethan-1-ol 9a (5.54 g, 12.17 mmol as obtained in step 6, as a mixture), and 1,2-di-tert-butyl (3S)-3-ethynyl-1,2-diazinane-1,2-dicarboxylate 10 (3.88 g, 12.17 mmol; for its preparation, see step 16, Scheme 2, vide infra) in ^{t}BuOH (30 mL) and water (15 mL) was added sequentially CuSO₄·5H₂O (625 mg, 0.2 eq, 2.43 mmol) and sodium ascorbate (2.47 g, 2 eq, 24.34 mmol) at rt and the reaction mixture was stirred at 90° C. for next 3 hr. After completion of reaction (monitored by TLC), the reaction mixture was diluted with water (100 mL) and extracted with DCM (2×500 ml). The combined organic layer was washed with brine (2×100 mL), dried over anhydrous Na₂SO₄, filtered, and concentrated under reduced pressure to afford a crude residue containing a mixture of di-tert-butyl (S)-3-(1-(1-(5-bromo-1-ethyl-2-(2-((S)-1-methoxyethyl)pyridin-3-yl)-1H-indol-3-yl)-2-methylpropan-2-yl)-1H-1,2,3-triazol-4-yl)tetrahydropyridazine-1,2-dicarboxylate 11 and di-tert-butyl (S)-3-(1-(1-(5-bromo-1-ethyl-2-(2-((S)-1-hydroxyethyl)pyridin-3-yl)-1H-indol-3-yl)-2-methylpropan-2-yl)-1H-1,2,3-triazol-4-yl)tetrahydropyridazine-1,2-dicarboxylate 11a (5.54 g), which was used in the next step without further purification.

Step 8. To an ice cold solution of a mixture of di-tert-butyl (S)-3-(1-(1-(5-bromo-1-ethyl-2-(2-((S)-1-methoxyethyl)pyridin-3-yl)-1H-indol-3-yl)-2-methylpropan-2-yl)-1H-1,2,3-triazol-4-yl)tetrahydropyridazine-1,2-dicarboxylate 11 and di-tert-butyl (S)-3-(1-(1-(5-bromo-1-ethyl-2-(2-((S)-1-hydroxyethyl)pyridin-3-yl)-1H-indol-3-yl)-2-methylpropan-2-yl)-1H-1,2,3-triazol-4-yl)tetrahydropyridazine-1,2-dicarboxylate 11a (5.54 g, 7.37 mmol), in DMF (30 mL) was added NaH, (600 mg, 1.2 eq, 8.44 mmol, 60% dispersion in oil) in portions, and the mixture was stirred at this temperature for 1 h. Subsequently, Mel (3.55 g, 2 eq, 14.4 mmol) was added dropwise, the mixture was allowed to warm to rt and stirred for next 16 h. After complete conversion of Cp 11a to 11 (TLC monitoring), the reaction mixture was cooled to 0° C., saturated aq. solution of NH₄Cl (250 mL) was added, and extracted with EtOAc (3×500 mL). The combined organic layer was washed with brine (50 mL), dried over anhydrous Na₂SO₄, and filtered. The filtrate was concentrated under reduced pressure and the residue was purified by Combi-flash column chromatography over silica gel using 50% EtOAc in n-heptane to afford di-tert-butyl (S)-3-(1-(1-(5-bromo-1-ethyl-2-(2-((S)-1-methoxyethyl)pyridin-3-yl)-1H-indol-3-yl)-2-methylpropan-2-yl)-1H-1,2,3-triazol-4-yl)tetrahydropyridazine-1,2-dicarboxylate 11 (1.93 g, 19% over four steps) as a viscous liquid. LCMS (ESI): m/z [M+H]⁺ calc'd for C₃₈H₅₃BrN₇O₅ 766.32 found 766.26

### Step 9

di-tert-butyl(S)-3-(1-(1-(5-(3-((S)-2-(((benzyloxy)carbonyl)amino)-3-methoxy-3-oxopropyl)phenyl)-1-ethyl-2-(2-((S)-1-methoxyethyl)pyridin-3-yl)-1H-indol-3-yl)-2-methylpropan-2-yl)-1H-1,2,3-triazol-4-yl)tetrahydropyridazine-1,2-dicarboxylate (13): To a stirred solution of di-tert-butyl (S)-3-(1-(1-(5-bromo-1-ethyl-2-(2-((S)-1-methoxyethyl)pyridin-3-yl)-1H-indol-3-yl)-2-methylpropan-2-yl)-1H-1,2,3-triazol-4-yl)tetrahydropyridazine-1,2-dicarboxylate 11 (1.93 g, 2.52 mmol) and methyl (2S)-2-{[(benzyloxy)carbonyl]amino}-3-[3-(4,4,5,5-tetramethyl-1,3,2-dioxaborolan-2-yl)phenyl]propanoate 12 (1.65 g, 1.5 eq, 3.78 mmol) in THF (15 mL) at rt was added sequentially K₃PO₄ (1.07 g, 3 eq, 7.56 mmol) and water (3 mL), and the reaction mixture was purged with Ar for 10 min. Subsequently, cataCXium® A Pd G3 (186 mg, 0.1 eq, 0.252 mmol) was added, the reaction mixture was purged with Ar for another 5 min and heated to 60° C. for next 16 h. After reaction monitoring (TLC/LC-MS), the reaction mixture was cooled to rt, filtered over a Celite pad and thoroughly washed with EtOAc. The organic layer was diluted with water (25 mL) and extracted with EtOAc (2×100 mL). The combined organic layer was washed with brine (50 mL), dried over anhydrous Na₂SO₄, and filtered. The filtrate was concentrated under reduced pressure and the residue was purified by Combi-flash column chromatography over silica gel using 50% EtOAc in n-heptane to afford di-tert-butyl(S)-3-(1-(1-(5-(3-((S)-2-(((benzyloxy)carbonyl)amino)-3-methoxy-3-oxopropyl)phenyl)-1-ethyl-2-(2-((S)-1-methoxyethyl)pyridin-3-yl)-1H-indol-3-yl)-2-methylpropan-2-yl)-1H-1,2,3-triazol-4-yl)tetrahydropyridazine-1,2-dicarboxylate 13 (1.10 g, 44%) as a yellow solid. LCMS (ESI): m/z [M+H]⁺ calc'd for C₅₆H₇₁N₈O₉ 999.53 found 999.25.

### Step 10

(S)-2-(((benzyloxy)carbonyl)amino)-3-(3-(3-(2-(4-((S)-1,2-bis(tert-butoxycarbonyl)hexahydropyridazin-3-yl)-1H-1,2,3-triazol-1-yl)-2-methylpropyl)-1-ethyl-2-(2-((S)-1-methoxyethyl)pyridin-3-yl)-1H-indol-5-yl)phenyl)propanoic acid (14): To a stirred solution of di-tert-butyl(S)-3-(1-(1-(5-(3-((S)-2-(((benzyloxy)carbonyl)amino)-3-methoxy-3-oxopropyl)phenyl)-1-ethyl-2-(2-((S)-1-methoxyethyl)pyridin-3-yl)-1H-indol-3-yl)-2-methylpropan-2-yl)-1H-1,2,3-triazol-4-yl)tetrahydropyridazine-1,2-dicarboxylate 13 (1.10 g, 1.104 mmol) in THF (10 mL) was added an aqueous solution of LiOH·H₂O (132 mg, 5 eq, 5.52 mmol), dissolved in water (10 mL). The reaction mixture was allowed to warm to rt and stirred for next 2 h. After completion of reaction (monitored by TLC and LCMS), THF was evaporated under reduced pressure, the aqueous phase acidified by 1N HCl to pH ˜5 and extracted with 10% MeOH in DCM (3×50 mL). The combined organic layer was dried over anhydrous Na₂SO₄, filtered and concentrated under reduced pressure to get the crude (S)-2-(((benzyloxy)carbonyl)amino)-3-(3-(3-(2-(4-((S)-1,2-bis(tert-butoxycarbonyl)hexahydropyridazin-3-yl)-1H-1,2,3-triazol-1-yl)-2-methylpropyl)-1-ethyl-2-(2-((S)-1-methoxyethyl)pyridin-3-yl)-1H-indol-5-yl)phenyl)propanoic acid 14 (939 mg) as a light brown solid, which was used in the next step without further purification. LCMS (ESI): m/z [M+H]⁺ calc'd for C₅₅H₆₉N₈O₉ 985.51 found 985.68.

### Step 11

(S)-2-(((benzyloxy)carbonyl)amino)-3-(3-(1-ethyl-3-(2-(4-((S)-hexahydropyridazin-3-yl)-1H-1,2,3-triazol-1-yl)-2-methylpropyl)-2-(2-((S)-1-methoxyethyl)pyridin-3-yl)-1H-indol-5-yl)phenyl)propanoic acid (15): To an ice cold stirred solution of (S)-2-(((benzyloxy)carbonyl)amino)-3-(3-(3-(2-(4-((S)-1,2-bis(tert-butoxycarbonyl)hexahydropyridazin-3-yl)-1H-1,2,3-triazol-1-yl)-2-methylpropyl)-1-ethyl-2-(2-((S)-1-methoxyethyl)pyridin-3-yl)-1H-indol-5-yl)phenyl)propanoic acid 14 (939 mg, 0.95 mmol) in DCM (10 mL) was added TFA (1.82 ml, 30 eq, 28.62 mmol) dropwise at 0° C.

The reaction mixture was allowed to come to rt and stirred for next 2 h. After completion of reaction (TLC/LCMS monitoring), the resulting reaction mass was concentrated under reduced pressure to give a crude which was further basified using aq. solution of NH₄OH (pH ˜10), extracted with 20% IPA in CHCl₃ (3×50 mL), and evaporated in vacuo eventually to get the crude (S)-2-(((benzyloxy)carbonyl)amino)-3-(3-(1-ethyl-3-(2-(4-((S)-hexahydropyridazin-3-yl)-1H-1,2,3-triazol-1-yl)-2-methylpropyl)-2-(2-((S)-1-methoxyethyl)pyridin-3-yl)-1H-indol-5-yl)phenyl)propanoic acid 15 (720 mg) as a brown solid. It was used in the next reaction without further purification. LCMS (ESI): m/z [M+H]⁺ calc'd for C₄₅H₅₃N₈O₅ 785.41 found 785.68.

### Step 12

benzyl ((6³S,8S,Z)-21-ethyl-2²-(2-((S)-1-methoxyethyl)pyridin-3-yl)-4,4-dimethyl-7-oxo-6¹,6²,6³,6⁴,6⁵,6⁶-hexahydro-2¹H,5¹H-2(5,3)-indola-6(3,1)-pyridazina-5(1,4)-triazola-1(1,3)-benzenacyclononaphane-8-yl)carbamate (16): To a stirred solution of (2S)-2-{[(benzyloxy)carbonyl]amino}-3-{3-[3-(2-{4-[(3S)-1,2-diazinan-3-yl]-1H-1,2,3-triazol-1-yl}-2-methylpropyl)-1-ethyl-2-{2-[(1S)-1-methoxyethyl]pyridin-3-yl}-1H-indol-5-yl]phenyl}propanoic acid (720 mg, 0.91 mmol) in DMF (8 mL) at 0° C. was added DIPEA (0.8 mL, 5eq, 4.59 mmol). Subsequently, T₃P (1.64 mL, 50% in EtOAc, 3 eq, 2.76 mmol) was added and the reaction mixture was stirred at −20° C. for next 30 min. After completion of reaction (TLC/LC-MS monitoring), the reaction mixture was diluted with ice-cold brine solution (20 mL) and extracted with EtOAc (3×50 mL). The combined organic layer was dried over anhydrous Na₂SO₄, filtered, and concentrated under reduced pressure to get a crude, which was further purified by Combi-flash column chromatography over silica gel using 70% EtOAc in n-Heptane to eventually obtain benzyl ((6³S,8S,Z)-21-ethyl-2²-(2-((S)-1-methoxyethyl)pyridin-3-yl)-4,4-dimethyl-7-oxo-6¹,6²,6³,6⁴,6⁵,6⁶-hexahydro-2¹H,5¹H-2(5,3)-indola-6(3,1)-pyridazina-5(1,4)-triazola-1(1,3)-benzenacyclononaphane-8-yl)carbamate 16 (250 mg, 29% over three steps) as a viscous liquid. LCMS (ESI): m/z [M+H]⁺ calc'd for C₄₅H₅₁N₈O₄ 767.40 found 767.55.

### Step 13

(6³S,8S,Z)-8-amino-21-ethyl-2²-(2-((S)-1-methoxyethyl)pyridin-3-yl)-4,4-dimethyl-6¹,6²,6³,6⁴,6⁵,6⁶-hexahydro-2¹H,5¹H-2(5,3)-indola-6(3,1)-pyridazina-5(1,4)-triazola-1(1,3)-benzenacyclononaphan-7-one (17): To a stirred solution of benzyl ((6³S,8S,Z)-21-ethyl-2²-(2-((S)-1-methoxyethyl)pyridin-3-yl)-4,4-dimethyl-7-oxo-6¹,6²,6³,6⁴,6⁵,6⁶-hexahydro-2¹H,5¹H-2(5,3)-indola-6(3,1)-pyridazina-5(1,4)-triazola-1(1,3)-benzenacyclononaphane-8-yl)carbamate 16 (250 mg, 0.326 mmol, in MeOH (5 mL) was added 10% Pd/C (250 mg w/w) followed by NH₄OAc (754 mg, 30 eq, 9.79 mmol) and the reaction mixture was stirred under H₂ balloon atmosphere at rt for 6 h. After completion of reaction (TLC/LC-MS monitoring), the reaction mass was filtered through a Celite pad, thoroughly washed with EtOAC and evaporated under reduced pressure to get the crude (6³S,8S,Z)-8-amino-21-ethyl-2²-(2-((S)-1-methoxyethyl)pyridin-3-yl)-4,4-dimethyl-6¹,6²,6³,6⁴,6⁵,6⁶-hexahydro-2¹H,5¹H-2(5,3)-indola-6(3,1)-pyridazina-5(1,4)-triazola-1(1,3)-benzenacyclononaphan-7-one 17 (108 mg) as a brown sticky solid, which was used in the next reaction without further purification. LCMS (ESI): m/z [M+H]⁺ calc'd for C₃₇H₄₅N₈O₂ 633.36 found 633.48.

### Step 14

(3S)-1-acryloyl-N-((2S)-1-(((6³S,8S,Z)-21-ethyl-2²-(2-((S)-1-methoxyethyl)pyridin-3-yl)-4,4-dimethyl-7-oxo-6¹,6²,6³,6⁴,6⁵,6⁶-hexahydro-21H,51H-2(5,3)-indola-6(3,1)-pyridazina-5(1,4)-triazola-1(1,3)-benzenacyclononaphane-8-yl)amino)-3-methyl-1-oxobutan-2-yl)-N-methylpyrrolidine-3-carboxamide (Compound 104): To stirred solution of (6³S,8S,Z)-8-amino-21-ethyl-2²-(2-((S)-1-methoxyethyl)pyridin-3-yl)-4,4-dimethyl-6¹,6²,6³,6⁴,6⁵,6⁶-hexahydro-21H,51H-2(5,3)-indola-6(3,1)-pyridazina-5(1,4)-triazola-1(1,3)-benzenacyclononaphan-7-one 17 (108 mg, 0.11 mmol, crude) and N—((S)-1-acryloylpyrrolidine-3-carbonyl)-N-methyl-L-valine 18 (20 mg, 1.5 eq, 0.071 mmol, crude as TFA salt) in DMF (3 mL) was added DIPEA (0.2 mL, 10 eq, 1.1 mmol) at −20° C. under an atmosphere of N₂, followed by COMU (125 mg, 1.5 eq, 0.29 mmol). The reaction mixture was gradually warmed to 0° C. and stirred at this temperature for next 2 h. After completion of the reaction (TLC/LC-MS monitoring), the reaction mixture was diluted with ice-cold brine (20 mL) and extracted with EtOAc (3×50 mL). The combined organic layer was dried over anhydrous Na₂SO₄, and filtered. The filtrate was concentrated under reduced pressure and the residue was purified by RP-HPLC eventually to afford (3S)-1-acryloyl-N-((2S)-1-(((6³S,8S,Z)-21-ethyl-2²-(2-((S)-1-methoxyethyl)pyridin-3-yl)-4,4-dimethyl-7-oxo-6¹,6²,6³,6⁴,6⁵,6⁶-hexahydro-21H,51H-2(5,3)-indola-6(3,1)-pyridazina-5(1,4)-triazola-1(1,3)-benzenacyclononaphane-8-yl)amino)-3-methyl-1-oxobutan-2-yl)-N-methylpyrrolidine-3-carboxamide, Compound 104 (6 mg, 2% over last two steps, 81.49% LCMS purity, as a mixture of two atropisomers) as a white solid. ¹H NMR (400 MHz, DMSO-d4): δ 8.86-8.81 (m, 1H), 8.86-8.81 (m, 1H), 8.35-8.28 (m, 1H), 8.06-7.99 (m, 1H), 7.64-7.51 (m, 6H), 7.42-7.18 (m, 4H), 6.79-6.59 (m, 1H), 6.19-6.15 (m, 1H), 5.74-5.64 (m, 1H), 5.48-5.32 (m, 1H), 5.25-5.18 (m, 1H), 4.68-4.63 (m, 1H), 4.34-3.91 (m, 4H), 3.83-3.56 (m, 5H), 3.10-3.08 (m, 3H), 2.99-2.67 (m, 7H), 1.89-1.74 (m, 4H), 1.65-1.44 (m, 4H), 1.49-1.27 (m, 17H) [plausible assignments are done here; due to presence of atropisomeric components, analogous peaks overlap each other causing unambiguous assignment of peaks and corresponding proton counts beyond the scope of current data]; LCMS (ESI): m/z [M+H]⁺ calc'd for C₅₁H₆₅N₁₀O₅ 897.51; found 897.62.

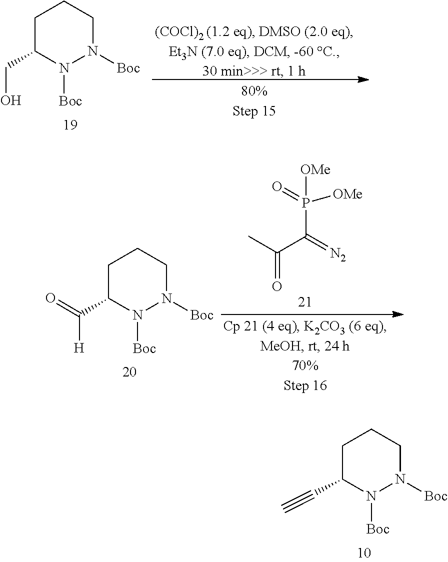

### Step 15

Synthesis of di-tert-butyl (S)-3-formyltetrahydropyridazine-1,2-dicarboxylate (20): A solution of DMSO (4.4 mL, 2.0 eq, 63.2 mmol) in DCM (55 mL) was added to a solution of (COCl)₂ (3.2 mL, 1.2 eq, 37.9 mmol) in DCM (5 mL) at −60° C. After 5 min, a solution of 1,2-di-tert-butyl (3S)-3-(hydroxymethyl)-1,2-diazinane-1,2-dicarboxylate 19 (10 g, 31.6 mmol), dissolved in DCM (55 mL), was added in the reaction mixture. After 30 min, triethylamine (31 mL, 7 eq, 221.2 mmol) was added, the reaction mixture allowed to warm to rt and stirred for next 1 h. After completion of reaction (TLC monitoring), the reaction mixture was quenched with water at 0° C. and extracted with DCM (3×100 mL). The combined organic layer was washed sequentially with brine, 1% aq. solution of HCl, and 5% aq. solution of NaHCO₃. The combined organic layer was dried over anhydrous Na₂SO₄ and concentrated under reduced pressure to get a crude residue, which was purified by Combi-flash column chromatography over silica gel using 20% EtOAc in n-heptane to afford di-tert-butyl (S)-3-formyltetrahydropyridazine-1,2-dicarboxylate 20 (8 g, 80%) as a colorless liquid.

### Step 16

Synthesis of di-tert-butyl (S)-3-ethynyltetrahydropyridazine-1,2-dicarboxylate (10): To a stirred solution of 1,2-di-tert-butyl (3S)-3-formyl-1,2-diazinane-1,2-dicarboxylate 20 (8 g, 25.4 mmol) in MeOH (160 mL) was added K₂CO₃ (21 g, 6 eq, 152.4 mmol) at rt under N₂ atmosphere, and the reaction mixture was stirred for 5 min. Subsequently, dimethyl (1-diazo-2-oxopropyl) phosphonate 21 (15.2 mL, 4 eq, 101.6 mmol) was added slowly, and the resulting reaction mixture was stirred for next 24 h. After reaction monitoring (TLC), the reaction mixture was quenched with water and extracted with DCM (3×100 mL). The combined organic layer was dried over anhydrous Na₂SO₄ and concentrated under reduced pressure to get a crude, which was purified by Combi-flash column chromatography over silica gel using 30% EtOAc in n-heptane to obtain 1,2-di-tert-butyl (3S)-3-ethynyl-1,2-diazinane-1,2-dicarboxylate 10 (5.5 g, 70%) as a white solid.

### Example 10

### Compound 105

### Synthesis of (2R,6R)-4-(4-(dimethylamino)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N,2,6-trimethylpiperazine-1-carboxamide (Compound 105)

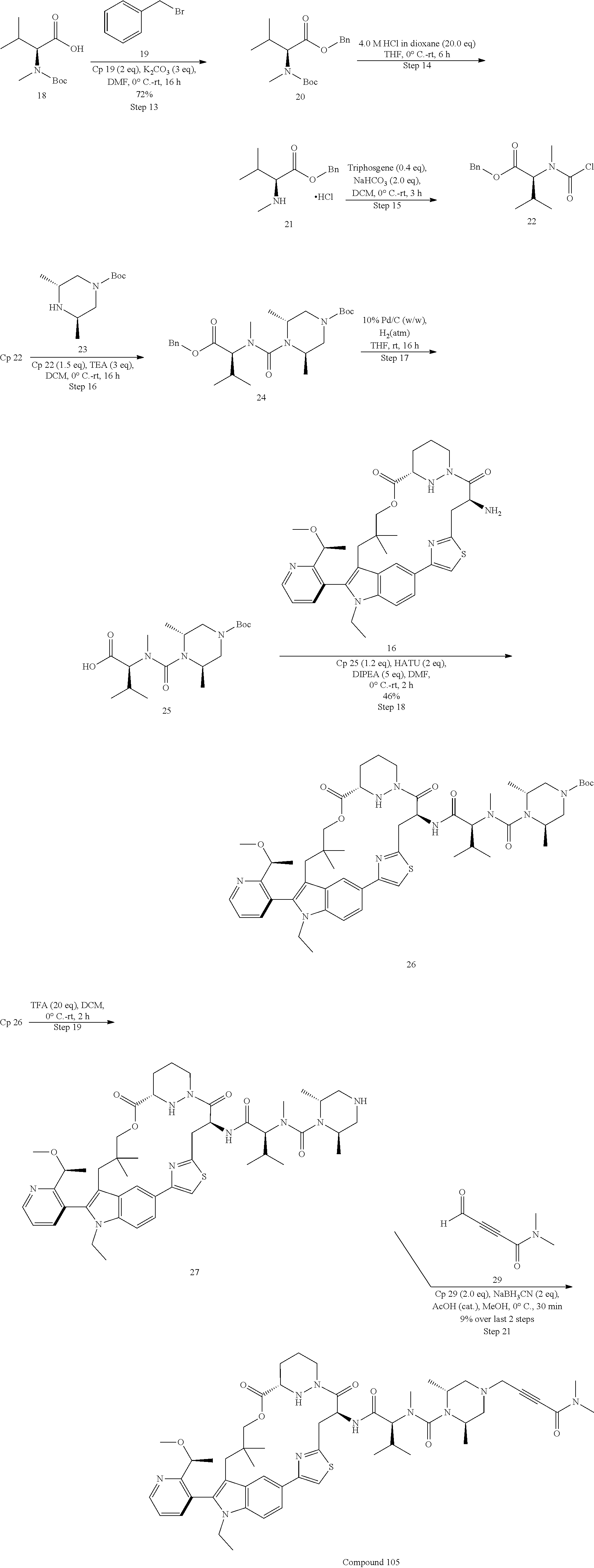

### Step 13

Synthesis of benzyl N-(tert-butoxycarbonyl)-N-methyl-L-valinate (20): To a stirred solution of (2S)-2-{[(tert-butoxy)carbonyl](methyl)amino}-3-methylbutanoic acid 18 (5 g, 21.6 mmol) in DMF (50 mL) at 0° C. was added K₂CO₃ (8.96 g, 3 eq, 64.9 mmol) and the reaction mixture was stirred at this temperature for 15 min before benzylbromide 19 (5.28 mL, 2 eq, 43.2 mmol) was added to it. The resulting reaction mixture was allowed to warm to rt and stirred for next 16 h. After completion of the reaction (monitored by TLC), the reaction mixture was quenched with ice cold water (200 mL) and extracted with EtOAc (3×100 mL). The combined organic layer was washed with ice cold brine (2×100 mL), dried over anhydrous Na₂SO₄, filtered and evaporated under reduced pressure to obtain a crude residue, which was purified by Combi-flash column chromatography over silica gel using 5-10% EtOAc in n-heptane to get benzyl N-(tert-butoxycarbonyl)-N-methyl-L-valinate 20 (5 g, 72%) as a colourless liquid. LCMS (ESI): m/z [M+H]⁺ calc'd for C₁₈H₂₈NO₄ 322.20; found 322.20.

### Step 14

Synthesis of benzyl methyl-L-valinate hydrochloride (21): To a stirred solution of benzyl N-(tert-butoxycarbonyl)-N-methyl-L-valinate 20 (2.5 g, 7.78 mmol) in THF (50 mL) was added 4.0 M HCl in dioxane (40 mL, 20 eq, 156 mmol) at 0° C. The resulting reaction mixture was allowed to warm to rt and stirred for next 6 h. After completion of the reaction (monitored by TLC), the reaction mixture was evaporated under reduced pressure to obtain the crude benzyl methyl-L-valinate hydrochloride 21 (1.8 g) as a white solid, which was used in the next step without further purification. LCMS (ESI): m/z [M+H]⁺ calc'd for C₁₃H₂₀NO₂ 222.15; found 222.16.

### Step 15

Synthesis of benzyl N-(chlorocarbonyl)-N-methyl-L-valinate (22): To a stirred solution of benzyl methyl-L-valinate hydrochloride 21 (1.16 g, 4.52 mmol) in DCM (20 mL) at 0° C. was added NaHCO₃ (652 mg, 2 eq, 7.76 mmol), followed by triphosgene (460 mg, 0.4 eq, 1.55 mmol) at the same temperature. The reaction mixture was allowed to warm to rt and stirred for next 3 h. After completion of reaction (monitored by TLC), the reaction mixture was filtered through a cotton plug and concentrated under reduced pressure to get the crude benzyl N-(chlorocarbonyl)-N-methyl-L-valinate 22 (800 mg) as a colorless oil, which was used in the next step without further purification.

### Step 16

Synthesis of tert-butyl (3R,5R)-4-(((S)-1-(benzyloxy)-3-methyl-1-oxobutan-2-yl)(methyl)carbamoyl)-3,5-dimethylpiperazine-1-carboxylate (24): To a stirred solution of tert-butyl (3R,5R)-3,5-dimethylpiperazine-1-carboxylate 23 (0.5 g, 2.33 mmol) in DCM (20 mL) at 0° C. was added Et₃N (1 mL, 3 eq, 7 mmol) and stirred for 10 min. Subsequently, benzyl N-(chlorocarbonyl)-N-methyl-L-valinate 22 (993 mg, 1.5 eq, 3.5 mmol), dissolved in DCM (10 mL), was added dropwise to the reaction mixture at 0° C. The reaction mixture was allowed to warm to rt and stirred for next 16 h. After completion of reaction (monitored by TLC), the reaction mixture was evaporated under reduced pressure to get the crude tert-butyl (3R,5R)-4-(((S)-1-(benzyloxy)-3-methyl-1-oxobutan-2-yl)(methyl)carbamoyl)-3,5-dimethylpiperazine-1-carboxylate 24 (600 mg) as a colorless oil, which was used in the next step without further purification. LCMS (ESI): m/z [M+H]⁺ calc'd for C₂₅H₄₀N₃O₅ 462.30; found 462.40.

### Step 17

Synthesis of N-((2R,6R)-4-(tert-butoxycarbonyl)-2,6-dimethylpiperazine-1-carbonyl)-N-methyl-L-valine (25): To a stirred solution of tert-butyl (3R,5R)-4-(((S)-1-(benzyloxy)-3-methyl-1-oxobutan-2-yl)(methyl)carbamoyl)-3,5-dimethylpiperazine-1-carboxylate 24 (600 mg, 1.30 mmol) in THF (20 mL) was added 10% Pd/C (600 mg) and the reaction mixture was stirred under a H₂ balloon atmosphere for next 16 h at rt. After completion of reaction (monitored by TLC), the resulting reaction mixture was filtered through a celite pad and washed thoroughly with EtOAc (100 mL). The combined filtrate was evaporated under reduced pressure to get the crude N-((2R,6R)-4-(tert-butoxycarbonyl)-2,6-dimethylpiperazine-1-carbonyl)-N-methyl-L-valine 25 (400 mg) as a colorless oil, which was used in the next step without further purification. LCMS (ESI): m/z [M+H]⁺ calc'd for C₁₈H₃₄N₃O₅ 372.25; found 372.18.

### Step 18

Synthesis of tert-butyl (3R,5R)-4-(((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)(methyl)carbamoyl)-3,5-dimethylpiperazine-1-carboxylate (26): To a stirred solution of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione 16 (250 mg, 0.396 mmol, its preparation is described in Scheme 1, step 11, vide supra) and N-((2R,6R)-4-(tert-butoxycarbonyl)-2,6-dimethylpiperazine-1-carbonyl)-N-methyl-L-valine 25 (180 mg, 1.2 eq, 0.476 mmol) in DMF (10 mL) at 0° C. was added DIPEA (0.3 mL, 5 eq, 1.98 mmol), followed by HATU (305 mg, 2 eq, 0.8 mmol). The reaction mixture was allowed to warm to rt and stirred for next 2 h. After completion of reaction (monitored by TLC and LCMS), the reaction mixture was diluted with ice cold water (50 mL) and extracted with EtOAc (3×100 mL). The combined organic layer was then dried over anhydrous Na₂SO₄, filtered and concentrate under the reduced pressure to get the crude, which was purified by Combi-flash column chromatography over silica gel using 50-80% EtOAc in n-heptane to get tert-butyl (3R,5R)-4-(((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)(methyl)carbamoyl)-3,5-dimethylpiperazine-1-carboxylate 26 (180 mg, 46%) as a brown sticky oil. LCMS (ESI): m/z [M+H]⁺ calc'd for C₅₂H₇₄N₉O₈S 984.54; found 984.50.

### Step 19

Synthesis of (2R,6R)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)-N,2,6-trimethylpiperazine-1-carboxamide (27): To a stirred solution of tert-butyl (3R,5R)-4-(((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)(methyl)carbamoyl)-3,5-dimethylpiperazine-1-carboxylate 26 (400 mg, 0.406 mmol) in DCM (10 mL) was added TFA (0.63 mL, 20 eq, 8.13 mmol) at 0° C. The reaction mixture was allowed to warm to rt and stirred for next 2 h. After completion of reaction (monitored by TLC and LCMS), the resulting reaction mixture was concentrated under reduced pressure to get a crude residue, which was basified by saturated aq. solution of NaHCO₃ to pH ˜10 and extracted with 25% IPA in CHCl₃ (3×50 mL). The combined organic layer was dried over anhydrous Na₂SO₄, filtered, and the filtrate concentrated under reduced pressure to get the crude (2R,6R)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)-N,2,6-trimethylpiperazine-1-carboxamide 27 (300 mg, as free amine) as off white semi-solid which was used in the next step without further purification. LCMS (ESI): m/z [M+H]⁺ calc'd for C₄₇H₆₆N₉O₆S 884.49; found 884.47.

### Step 21

Synthesis of (2R,6R)-4-(4-(dimethylamino)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N,2,6-trimethylpiperazine-1-carboxamide (Compound 105): To a stirred mixture (2R,6R)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N,2,6-trimethylpiperazine-1-carboxamide 27 (140 mg, 0.158 mmol) and N,N-dimethyl-4-oxobut-2-ynamide 29 (40 mg, 2 eq, 0.316 mmol) in MeOH (10 mL) was added acetic acid (catalytic, 10 μL) at 0° C. Sodium cyanoborohydride (20 mg, 2 eq, 0.316 mmol) was then added to the reaction mixture and it was stirred for 30 min. After completion of reaction (monitored by LCMS/TLC), the reaction mass was quenched with water (10 mL) and extracted with DCM (3×50 mL). The combined organic layer was dried over anhydrous Na₂SO₄, filtered and concentrated under reduced pressure to get a crude which was purified by RP-HPLC to afford (2R,6R)-4-(4-(dimethylamino)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N,2,6-trimethylpiperazine-1-carboxamide, Compound 105 (14 mg, 9% over last 2 steps, 99.64% purity) as a white solid. ¹H NMR (400 MHz, DMSO-d₆): δ 8.74 (dd, J=4.8, 2.6 Hz, 1H), 8.59 (d, J=8 Hz, 1H), 8.46 (s, 1H), 7.84 (s, 1H), 7.79-7.77 (m, 1H), 7.75-7.73 (m, 1H), 7.57 (d, J=8.8 Hz, 1H), 7.51 (q, J=4.8 Hz, 1H), 5.42 (t, J=8 Hz, 1H), 5.06 (d, J=12.4 Hz, 1H), 4.48 (d, J=11.2 Hz, 1H), 4.36-4.30 (m, 2H), 4.28-4.18 (m, 2H), 4.17-4.09 (m, 1H), 3.59-3.51 (m, 4H), 3.39-3.45 (m, 2H), 3.25 (s, 4H), 3.23-3.20 (m, 1H), 3.16 (s, 3H), 2.96 (d, J=14 Hz, 1H), 2.89 (s, 3H), 2.85 (s, 3H), 2.76-2.72 (m, 2H), 2.40-2.36 (m, 2H), 2.17-2.12 (m, 2H), 2.10-1.95 (m, 1H), 1.81-1.77 (m, 2H), 1.56-1.49 (m, 1H), 1.36 (d, J=6 Hz, 3H), 1.23 (brs, 1H), 1.16-1.01 (m, 6H), 0.93-0.90 (m, 6H), 0.83 (t, J=6.8 Hz, 3H), 0.79 (d, J=6.8 Hz, 3H), 0.30 (brs, 3H); LCMS (ESI): m/z [M+H]⁺ calc'd for C₅₃H₇₃N₁₀O₇S 993.54; found 993.59.

### Example 11

### Compound 106 and 107

### Synthesis of (3S)—N-((2S)-1-(((6³S,4S)-1¹-ethyl-1²-(2-((S)-1-methoxyethyl)pyridin-3-yl)-10,10-dimethyl-5,8-dioxo-1¹H-9-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide (Compound 106)

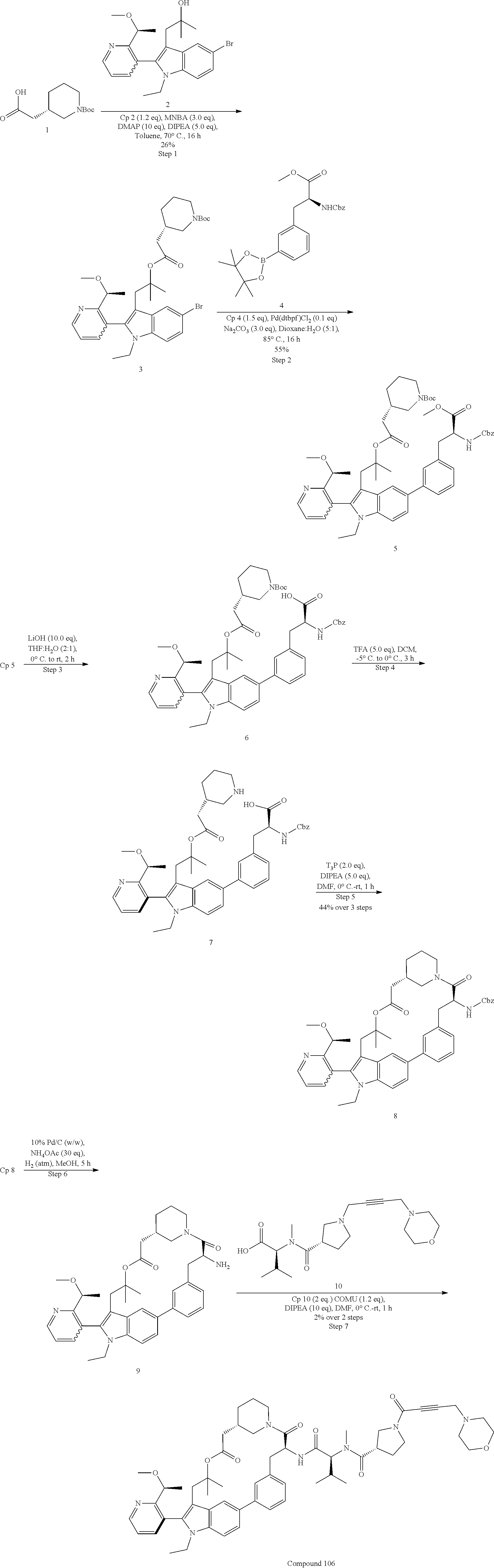

### Step 1

Synthesis of tert-butyl (S)-3-(2-((1-(5-bromo-1-ethyl-2-(2-((S)-1-methoxyethyl)pyridin-3-yl)-1H-indol-3-yl)-2-methylpropan-2-yl)oxy)-2-oxoethyl)piperidine-1-carboxylate (3): To a stirred solution (S)-2-(1-(tert-butoxycarbonyl)piperidin-3-yl)acetic acid 1 (2 g, 8.22 mmol) and (S)-1-(5-bromo-1-ethyl-2-(2-(1-methoxyethyl)pyridin-3-yl)-1H-indol-3-yl)-2-methylpropan-2-ol 2 (4.26 g, 1.2 eq, 9.86 mmol) in toluene (0.4 L) at 0° C. under nitrogen atmosphere was added DMAP (10.1 g, 10 eq, 82.2 mmol) and DIPEA (7.16 mL, 5 eq, 41.1 mmol) followed by MNBA (8.49 g, 3 eq, 24.7 mmol). The reaction mixture was stirred at 70° C. for 16 h. After completion of reaction (monitored by TLC), the reaction mixture was quenched with water (100 mL) and extracted with ethyl acetate (2×250 mL). The combined organic layer was washed with brine (100 mL), dried over anhydrous Na₂SO₄, filtered and evaporated under reduced pressure to obtain a crude residue, which was purified by Combi-flash column chromatography over silica-gel using 40-50% EtOAc in n-heptane to afford tert-butyl (S)-3-(2-((1-(5-bromo-1-ethyl-2-(2-((S)-1-methoxyethyl)pyridin-3-yl)-1H-indol-3-yl)-2-methylpropan-2-yl)oxy)-2-oxoethyl)piperidine-1-carboxylate 3 (1.4 g, 26%) as a brown semi-solid. LCMS (ESI): m/z [M+H]⁺ calc'd for C₃₄H₄₇BrN₃O₅ 656.27; found 656.43.

### Step 2

Synthesis of tert-butyl (S)-3-(2-((1-(5-(3-((S)-2-(((benzyloxy)carbonyl)amino)-3-methoxy-3-oxopropyl)phenyl)-1-ethyl-2-(2-((S)-1-methoxyethyl)pyridin-3-yl)-1H-indol-3-yl)-2-methylpropan-2-yl)oxy)-2-oxoethyl)piperidine-1-carboxylate (5): To a stirred solution of tert-butyl (3S)-3-(2-{[1-(5-bromo-1-ethyl-2-{2-[(1S)-1-methoxyethyl]pyridin-3-yl}-1H-indol-3-yl)-2-methylpropan-2-yl]oxy}-2-oxoethyl)piperidine-1-carboxylate 3 (600 mg, 0.914 mmol) and methyl (2S)-2-{[(benzyloxy)carbonyl]amino}-3-[3-(4,4,5,5-tetramethyl-1,3,2-dioxaborolan-2-yl)phenyl]propanoate 4 (602 mg, 1.5 eq, 1.37 mmol) in 1,4-dioxane (15 mL) in a sealed RB at rt was added sequentially Na₂CO₃ (291 mg, 3 eq, 2.74 mmol) and H₂O (3 mL) and the reaction mixture was purged with N₂ for 15 min. Subsequently, PdCl₂(dtbpf) (59.5 mg, 0.1 eq, 0.091 mmol) was added, and the reaction mixture was stirred at 85° C. for next 16 h. After reaction monitoring (TLC), the reaction mixture was cooled to rt, diluted with H₂O (10 mL) and extracted with EtOAc (3×50 mL). The combined organic layer was washed with brine (100 mL), dried over anhydrous Na₂SO₄, and filtered. The filtrate was concentrated under reduced pressure and the residue was purified by Combi-flash column chromatography over silica gel using 70% EtOAc in n-heptane to afford tert-butyl (S)-3-(2-((1-(5-(3-((S)-2-(((benzyloxy)carbonyl)amino)-3-methoxy-3-oxopropyl)phenyl)-1-ethyl-2-(2-((S)-1-methoxyethyl)pyridin-3-yl)-1H-indol-3-yl)-2-methylpropan-2-yl)oxy)-2-oxoethyl)piperidine-1-carboxylate 5 (450 mg, 55%) as a brown solid. LCMS (ESI): m/z [M+H]⁺ calc'd for C₅₂H₆₅N₄O₉ 889.47; found 889.76.

### Step 3

Synthesis of (S)-2-(((benzyloxy)carbonyl)amino)-3-(3-(3-(2-(2-((S)-1-(tert-butoxycarbonyl)piperidin-3-yl)acetoxy)-2-methylpropyl)-1-ethyl-2-(2-((S)-1-methoxyethyl)pyridin-3-yl)-1H-indol-5-yl)phenyl)propanoic acid (6): To a stirred solution of tert-butyl (S)-3-(2-((1-(5-(3-((S)-2-(((benzyloxy)carbonyl)amino)-3-methoxy-3-oxopropyl)phenyl)-1-ethyl-2-(2-((S)-1-methoxyethyl)pyridin-3-yl)-1H-indol-3-yl)-2-methylpropan-2-yl)oxy)-2-oxoethyl)piperidine-1-carboxylate 5 (400 mg, 0.45 mmol) in THF (10 mL) and water (5 mL) at 0° C. was added LiOH·H₂O (108 mg, 10 eq, 4.5 mmol). The reaction mixture was allowed to warm to rt and stirred for next 2 h. After completion of reaction (monitored by TLC and LCMS), THF was evaporated under reduced pressure, the aqueous phase acidified by 1N HCl to pH ˜5 and extracted with 10% MeOH in DCM (3×50 mL). The combined organic layer was dried over anhy. Na₂SO₄, filtered and concentrated under reduced pressure to get the crude (S)-2-(((benzyloxy)carbonyl)amino)-3-(3-(3-(2-(2-((S)-1-(tert-butoxycarbonyl)piperidin-3-yl)acetoxy)-2-methylpropyl)-1-ethyl-2-(2-((S)-1-methoxyethyl)pyridin-3-yl)-1H-indol-5-yl)phenyl)propanoic acid 6 (300 mg) as a brown solid, which was used in the next step without further purification. LCMS (ESI): m/z [M+H]⁺ calc'd for C₅₁H₆₃N₄O₉ 875.45; found 875.23.

### Step 4

Synthesis of (S)-2-(((benzyloxy)carbonyl)amino)-3-(3-(1-ethyl-2-(2-((S)-1-methoxyethyl)pyridin-3-yl)-3-(2-methyl-2-(2-((S)-piperidin-3-yl)acetoxy)propyl)-1H-indol-5-yl)phenyl)propanoic acid (7): To a stirred solution of (S)-2-(((benzyloxy)carbonyl)amino)-3-(3-(3-(2-(2-((5)-1-(tert-butoxycarbonyl)piperidin-3-yl)acetoxy)-2-methylpropyl)-1-ethyl-2-(2-((S)-1-methoxyethyl)pyridin-3-yl)-1H-indol-5-yl)phenyl)propanoic acid 6 (300 mg, 0.343 mmol) in DCM (10 mL) was added TFA (0.13 mL, 5 eq, 1.71 mmol) at −5° C. under N₂ atmosphere. The reaction mixture was gradually warmed to 0° C. and stirred for next 3 h. After completion of reaction (TLC monitoring), the reaction mixture was evaporated under reduced pressure to get the crude (S)-2-(((benzyloxy)carbonyl)amino)-3-(3-(1-ethyl-2-(2-((S)-1-methoxyethyl)pyridin-3-yl)-3-(2-methyl-2-(2-((S)-piperidin-3-yl)acetoxy)propyl)-1H-indol-5-yl)phenyl)propanoic acid 7 (200 mg) as a brown semi-solid, which was used in the next step without further purification. LCMS (ESI): m/z [M+H]⁺ calc'd for C₄₆H₅₅N₄O₇ 775.40; found 775.10.

### Step 5

Synthesis of benzyl ((6³S,4S)-1¹-ethyl-1²-(2-((S)-1-methoxyethyl)pyridin-3-yl)-10,10-dimethyl-5,8-dioxo-1¹H-9-oxa-1(5,3)-indola-6(1,3)-piperidina-2(1,3)-benzenacycloundecaphane-4-yl)carbamate (8): To a stirred solution of (S)-2-(((benzyloxy)carbonyl)amino)-3-(3-(1-ethyl-2-(2-((S)-1-methoxyethyl)pyridin-3-yl)-3-(2-methyl-2-(2-((S)-piperidin-3-yl)acetoxy)propyl)-1H-indol-5-yl)phenyl)propanoic acid 7 (200 mg, 0.258 mmol) in DCM (8 mL) was added DIPEA (0.22 mL, 5 eq, 1.29 mmol) at 0° C. under Argon atmosphere. Subsequently, T₃P (0.3 mL, 50% in EtOAc, 2 eq, 0.516 mmol) was added and the reaction mixture was stirred at rt for 1 h. After completion of reaction (TLC/LCMS monitoring), the reaction mixture was diluted with ice-cold brine (20 mL) and extracted with EtOAc (3×50 mL). The combined organic layer was dried over anhydrous Na₂SO₄, filtered, and the filtrate was concentrated under reduced pressure to get the crude residue which was purified by Combi-flash column chromatography over silica gel using 5% MeOH in DCM to afford benzyl ((6³S,4S)-1¹-ethyl-1²-(2-((S)-1-methoxyethyl)pyridin-3-yl)-10,10-dimethyl-5,8-dioxo-1¹H-9-oxa-1(5,3)-indola-6(1,3)-piperidina-2(1,3)-benzenacycloundecaphane-4-yl)carbamate 8 (150 mg, 44% over 3 steps) as a yellow solid. LCMS (ESI): m/z [M+H]⁺ calc'd for C₄₆H₅₃N₄O₆ 757.39; found 757.23.

### Step 6

Synthesis of (6³S,4S)-4-amino-1¹-ethyl-1²-(2-((S)-1-methoxyethyl)pyridin-3-yl)-10,10-dimethyl-1¹H-9-oxa-1(5,3)-indola-6(1,3)-piperidina-2(1,3)-benzenacycloundecaphane-5,8-dione (9): To a stirred solution of benzyl ((6³S,4S)-1¹-ethyl-1²-(2-((S)-1-methoxyethyl)pyridin-3-yl)-10,10-dimethyl-5,8-dioxo-1¹H-9-oxa-1(5,3)-indola-6(1,3)-piperidina-2(1,3)-benzenacycloundecaphane-4-yl)carbamate 8 (300 mg, 0.39 mmol) in MeOH (30 mL) was added 10% Palladium on Carbon (300 mg) followed by NH₄OAc (416 mg, 30 eq, 11.9 mmol). The reaction mixture was then stirred under Hydrogen balloon atmosphere at rt for 5 h. After completion of reaction (TLC/LC-MS monitoring), the reaction mass was filtered through a Celite bed, thoroughly washed with EtOAc and evaporated under reduced pressure to get the crude (6³S,4S)-4-amino-1¹-ethyl-1²-(2-((S)-1-methoxyethyl)pyridin-3-yl)-10,10-dimethyl-1H-9-oxa-1(5,3)-indola-6(1,3)-piperidina-2(1,3)-benzenacycloundecaphane-5,8-dione 9 (0.2 g) as a brown solid, which was used in the next step without further purification. LCMS (ESI): m/z [M+H]⁺ calc'd for C₃₈H₄₇N₄O₄ 623.35; found 623.75.

### Step 7

Synthesis of (3S)—N-((2S)-1-(((6³S,4S)-1¹-ethyl-1²-(2-((S)-1-methoxyethyl)pyridin-3-yl)-10,10-dimethyl-5,8-dioxo-1¹H-9-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide (Compound 106): To a stirred solution of (6³S,4S)-4-amino-1¹-ethyl-1²-(2-((S)-1-methoxyethyl)pyridin-3-yl)-10,10-dimethyl-1¹H-9-oxa-1(5,3)-indola-6(1,3)-piperidina-2(1,3)-benzenacycloundecaphane-5,8-dione 9 (150 mg, 0.241 mmol) and N—((S)-1-acryloylpyrrolidine-3-carbonyl)-N-methyl-L-valine 10 (183 mg, 2 eq, 0.482 mmol) in DMF (5 mL) was added DIPEA (0.42 mL, 10 eq, 2.41 mmol) at 0° C. under N₂ atmosphere, followed by COMU (124 mg, 1.2 eq, 0.289 mmol) was added and the reaction mixture was stirred at rt for 1 h. After completion of the reaction (TLC/LC-MS monitoring), the reaction mixture was diluted with ice-cold brine (20 mL) and extracted with EtOAc (3×50 mL). The combined organic layer was dried over anhydrous Na₂SO₄, and filtered. The filtrate was concentrated under reduced pressure and the residue was purified by RP-HPLC to afford a separable mixture of two diastereomers, Compound 106 (5 mg, 2% over 2 steps, 92.23% LCMS purity), and Compound 107 (5 mg, 2% over 2 steps, 96.02% LCMS purity) both as a white solid, respectively.

Data for Compound 106: LCMS (ESI): m/z [M+H]⁺ calc'd for C₅₇H₇₄N₇O₈ 984.55; found 984.52

Data for Compound 107: LCMS (ESI): m/z [M+H]⁺ calc'd for C₅₇H₇₄N₇O₈ 984.55; found 984.48.

### Example 12

### Compound 108

### Synthesis of (3S)—N-((2S)-1-(((6³R,4S)-1¹-ethyl-1²-(2-((S)-1-methoxyethyl)pyridin-3-yl)-10,10-dimethyl-5,8-dioxo-1¹H-9-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide (Compound 108)

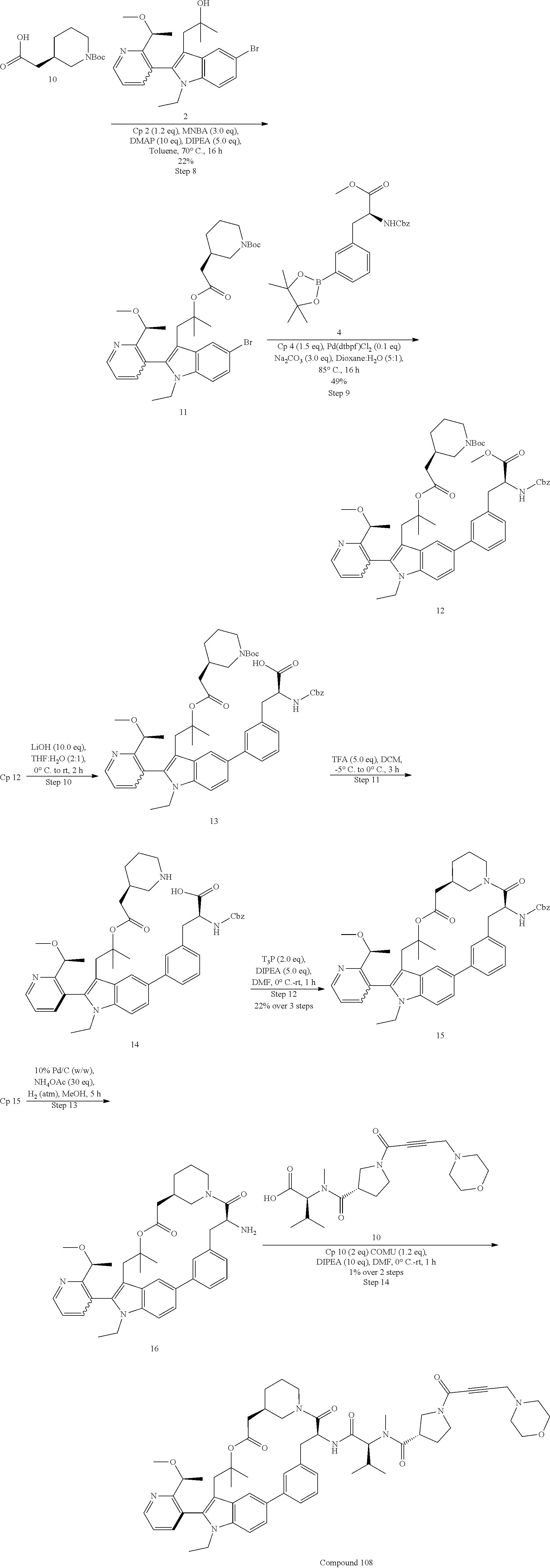

### Step 8

Synthesis of tert-butyl (R)-3-(2-((1-(5-bromo-1-ethyl-2-(2-((S)-1-methoxyethyl)pyridin-3-yl)-1H-indol-3-yl)-2-methylpropan-2-yl)oxy)-2-oxoethyl)piperidine-1-carboxylate (11): Following the procedure as described in Step 1, Scheme 1 (vide supra) using (R)-2-(1-(tert-butoxycarbonyl)piperidin-3-yl)acetic acid 10 and Cp 2, (R)-3-(2-((1-(5-bromo-1-ethyl-2-(2-((S)-1-methoxyethyl)pyridin-3-yl)-1H-indol-3-yl)-2-methylpropan-2-yl)oxy)-2-oxoethyl)piperidine-1-carboxylate 11 was obtained as a brown semi-solid.

### Step 9

Synthesis of tert-butyl (R)-3-(2-((1-(5-(3-((S)-2-(((benzyloxy)carbonyl)amino)-3-methoxy-3-oxopropyl)phenyl)-1-ethyl-2-(2-((S)-1-methoxyethyl)pyridin-3-yl)-1H-indol-3-yl)-2-methylpropan-2-yl)oxy)-2-oxoethyl)piperidine-1-carboxylate (12): Following the procedure as described in Step 2, Scheme 1 (vide supra) using Cp 11 and Cp 4, tert-butyl (R)-3-(2-((1-(5-(3-((S)-2-(((benzyloxy)carbonyl)amino)-3-methoxy-3-oxopropyl)phenyl)-1-ethyl-2-(2-((S)-1-methoxyethyl)pyridin-3-yl)-1H-indol-3-yl)-2-methylpropan-2-yl)oxy)-2-oxoethyl)piperidine-1-carboxylate 12 was obtained as a brown semi-solid.

### Step 10

Synthesis of (S)-2-(((benzyloxy)carbonyl)amino)-3-(3-(3-(2-(2-((R)-1-(tert-butoxycarbonyl)piperidin-3-yl)acetoxy)-2-methylpropyl)-1-ethyl-2-(2-((S)-1-methoxyethyl)pyridin-3-yl)-1H-indol-5-yl)phenyl)propanoic acid (13): Following the procedure as described in Step 3, Scheme 1 (vide supra) using Cp 12, (S)-2-(((benzyloxy)carbonyl)amino)-3-(3-(3-(2-(2-((R)-1-(tert-butoxycarbonyl)piperidin-3-yl)acetoxy)-2-methylpropyl)-1-ethyl-2-(2-((S)-1-methoxyethyl)pyridin-3-yl)-1H-indol-5-yl)phenyl)propanoic acid 13 was obtained as a brown solid.

### Step 11

Synthesis of (S)-2-(((benzyloxy)carbonyl)amino)-3-(3-(1-ethyl-2-(2-((S)-1-methoxyethyl)pyridin-3-yl)-3-(2-methyl-2-(2-((R)-piperidin-3-yl)acetoxy)propyl)-1H-indol-5-yl)phenyl)propanoic acid (14): Following the procedure as described in Step 4, Scheme 1 (vide supra) using Cp 13, (S)-2-(((benzyloxy)carbonyl)amino)-3-(3-(1-ethyl-2-(2-((S)-1-methoxyethyl)pyridin-3-yl)-3-(2-methyl-2-(2-((R)-piperidin-3-yl)acetoxy)propyl)-1H-indol-5-yl)phenyl)propanoic acid 14 was obtained as a brown semi-solid.

### Step 12

Synthesis of benzyl ((6³R,4S)-1¹-ethyl-1²-(2-((S)-1-methoxyethyl)pyridin-3-yl)-10,10-dimethyl-5,8-dioxo-1¹H-9-oxa-1(5,3)-indola-6(1,3)-piperidina-2(1,3)-benzenacycloundecaphane-4-yl)carbamate (15): Following the procedure as described in Step 5, Scheme 1 (vide supra) using Cp 14, benzyl ((6³R,4S)-1¹-ethyl-1²-(2-((S)-1-methoxyethyl)pyridin-3-yl)-10,10-dimethyl-5,8-dioxo-1H-9-oxa-1(5,3)-indola-6(1,3)-piperidina-2(1,3)-benzenacycloundecaphane-4-yl)carbamate 15 was obtained as yellow solid.

### Step 13

Synthesis of (6³R,4S)-4-amino-1¹-ethyl-1²-(2-((S)-1-methoxyethyl)pyridin-3-yl)-10,10-dimethyl-1¹H-9-oxa-1(5,3)-indola-6(1,3)-piperidina-2(1,3)-benzenacycloundecaphane-5,8-dione (16): Following the procedure as described in Step 6, Scheme 1 (vide supra) using Cp 15, (6³R,4S)-4-amino-1¹-ethyl-1²-(2-((S)-1-methoxyethyl)pyridin-3-yl)-10,10-dimethyl-1¹H-9-oxa-1(5,3)-indola-6(1,3)-piperidina-2(1,3)-benzenacycloundecaphane-5,8-dione 16 was obtained as a brown solid.

### Step 14

Synthesis of (3S)—N-((2S)-1-(((6³R,4S)-1¹-ethyl-1²-(2-((S)-1-methoxyethyl)pyridin-3-yl)-10,10-dimethyl-5,8-dioxo-1¹H-9-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide (Compound 108): Following the procedure as described in Step 7, Scheme 1 (vide supra) using Cp 16 and Cp 10, after RP-HPLC purification (3S)—N-((2S)-1-(((6³R,4S)-1¹-ethyl-1²-(2-((S)-1-methoxyethyl)pyridin-3-yl)-10,10-dimethyl-5,8-dioxo-1¹H-9-oxa-1(5,3)-indola-6(1,3)-piperidina-2(1,3)-benzenacycloundecaphane-4-yl)amino)-3-methyl-1-oxobutan-2-yl)-N-methyl-1-(4-morpholinobut-2-ynoyl)pyrrolidine-3-carboxamide Compound 108 (1 mg, 1% over 2 steps, 92.77% LCMS purity) was obtained as a white solid. LCMS (ESI): m/z [M+H]⁺ calc'd for C₅₇H₇₄N₅O₈ 984.55; found 984.18.

### Example 13

### Compound 109

### Synthesis of 1-(4-(dimethylamino)-4-oxobut-2-yn-1-yl)-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpiperidine-4-carboxamide (Compound 109)

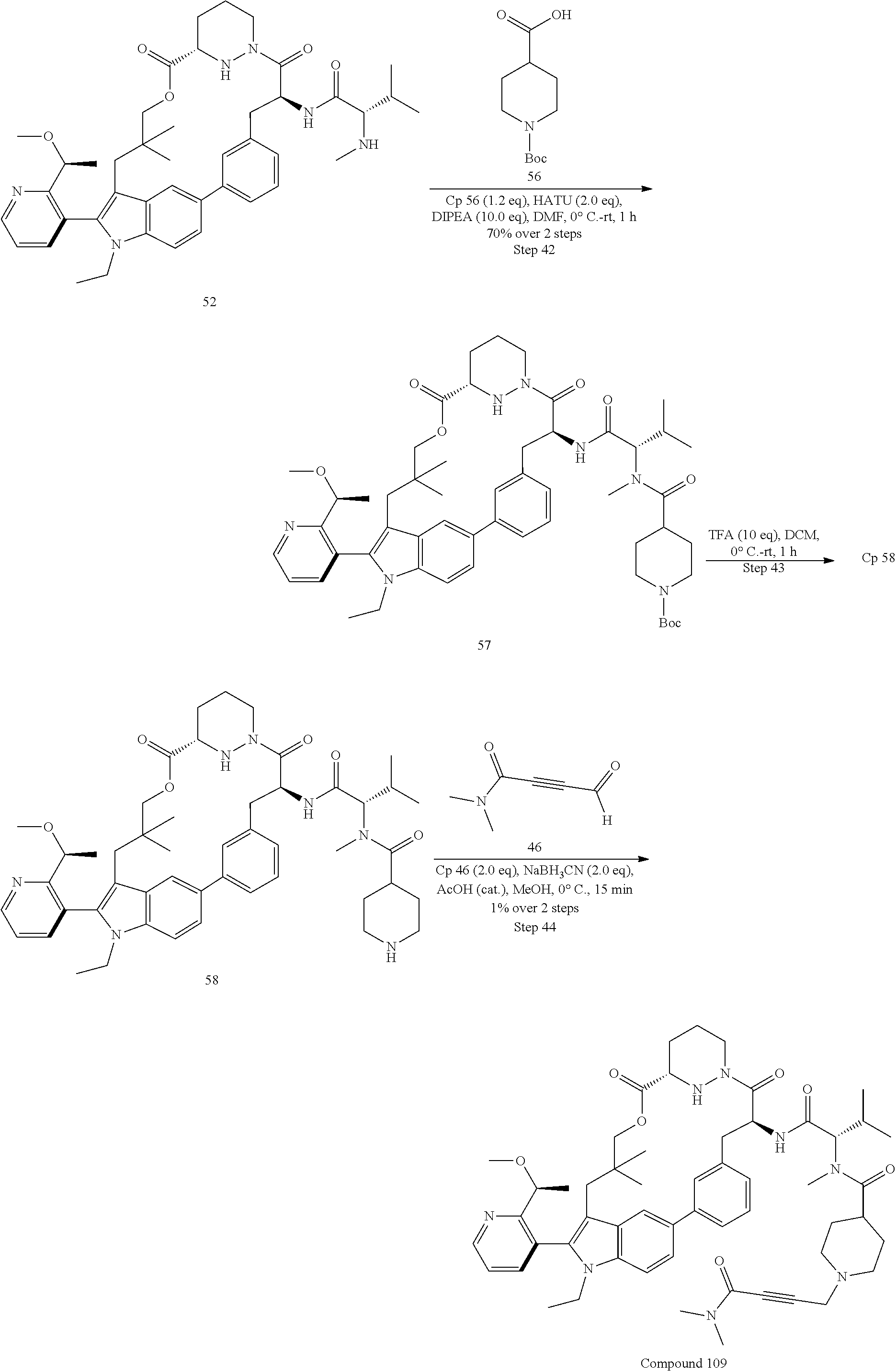

### Step 42

Synthesis of tert-butyl 4-(((2S)-1-(((6³S,4S)-1¹-ethyl-1²-(2-((S)-1-methoxyethyl)pyridin-3-yl)-10,10-dimethyl-5,7-dioxo-6′,6²,6³,6⁴,6⁵,6⁶-hexahydro-1¹H-8-oxa-1(5,3)-indola-6(1,3)-pyridazina-2(1,3)-benzenacycloundecaphane- 4-yl)amino)-3-methyl-1-oxobutan-2-yl)(methyl)carbamoyl)piperidine-1-carboxylate (57): To a stirred solution of (2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide 52 (1.2 g, 1.63 mmol) and 1-(tert-butoxycarbonyl)piperidine-4-carboxylic acid 56 (337 mg, 1.2 eq, 1.47 mmol) in DMF (20 mL) at 0° C. under an atmosphere of N₂ was added DIPEA (2.8 mL, 10 eq, 16.3 mmol), followed by HATU (1.2 g, 2 eq, 3.26 mmol). The reaction mixture was allowed to warm to rt and stirred for next 1 h. After completion of reaction (TLC monitoring), the reaction mixture was diluted with chilled brine (20 mL) and extracted with EtOAc (3×50 mL). The combined organic layer was dried over anhydrous Na₂SO₄, filtered, and concentrate under reduced pressure to get a crude, which was further purified by Combi-flash column chromatography over silica gel using 70% EtOAc in n-Heptane eventually to afford tert-butyl 4-(((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)piperidine-1-carboxylate 57 (0.9 g, 70% over 2 steps) as a brown solid. LCMS (ESI): m/z [M+H]⁺ calc'd for C₅₄H₇₄N₇O₈ 948.56; found 948.91.

### Step 43

Synthesis of N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)piperidine-1-carboxylate (58): To a stirred solution of tert-butyl 4-(((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)piperidine-1-carboxylate 57 (0.9 g, 0.950 mmol) in DCM (30 mL) at 0° C. was added TFA (0.7 mL, 10 eq, 9.50 mmol) under nitrogen atmosphere. The reaction mixture was gradually warmed to rt and stirred for next 1 h. After completion of reaction (TLC monitoring), the reaction mass was concentrated under reduced pressure to give a crude which was further basified using aq. solution of NaHCO₃ and extracted with 15% MeOH in DCM (3×50 mL). The combined organic layer was dried over anhydrous Na₂SO₄, filtered, and concentrated under reduced pressure eventually to obtain the crude N-((2S)-1-(((6³S,4S)-1¹-ethyl-1²-(2-((S)-1-methoxyethyl)pyridin-3-yl)-10,10-dimethyl-5,7-dioxo-6¹,6²,63, 64, 65, 6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)piperidine-1-carboxylate 58 (0.6 g) as a brown solid, which was used in the next step without further purification. LCMS (ESI): m/z [M+H]⁺ calc'd for C₄₉H₆₆N₇O₆ 848.51; found 848.39.

### Step 44

Synthesis of 1-(4-(dimethylamino)-4-oxobut-2-yn-1-yl)-N—((S)-1-(((4S,8S)-1¹-ethyl-1² (2-((S)-1-methoxyethyl)pyridin-3-yl)-6,8,12,12-tetramethyl-5,9-dioxo-1¹H-10-oxa-6,7-diaza-1(5,3)-indola-2(1,3)-benzenacyclotridecaphane-4-yl)amino)-3-methyl-1-oxobutan-2-yl)-N-methylpiperidine-4-carboxamide (Compound 109): To a stirred solution of N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)piperidine-1-carboxylate 58 (0.3 g, 0.354 mmol) and N,N-dimethyl-4-oxobut-2-ynamide 46 (67 mg, 2 eq, 0.531 mmol) in MeOH (20 mL) was added acetic acid (catalytic, 10 μL) at 0° C. Sodium cyanoborohydride (45 mg, 2 eq, 0.708 mmol) was then added and the reaction mixture was stirred for 15 min. After completion of reaction (monitored by LCMS/TLC), the reaction mass was quenched with water (10 mL) and extracted with 10% MeOH in DCM (2×50 mL). The combined organic layer was dried over anhydrous Na₂SO₄ and concentrated under reduced pressure to get a crude which was purified by RP-HPLC to afford 1-(4-(dimethylamino)-4-oxobut-2-yn-1-yl)-N—((S)-1-(((4S,8S)-1¹-ethyl-1²-(2-((S)-1-methoxyethyl)pyridin-3-yl)-6,8,12,12-tetramethyl-5,9-dioxo-1¹H-10-oxa-6,7-diaza-1(5,3)-indola-2(1,3)-benzenacyclotridecaphane-4-yl)amino)-3-methyl-1-oxobutan-2-yl)-N-methylpiperidine-4-carboxamide, Compound 109 (4.5 mg, 1% over 2 steps, 95.11% purity by LCMS) as a white solid. ¹H NMR (400 MHz, DMSO-d₆): δ 8.76 (dd, J=3.6, 9.6 Hz, 2H), 7.98 (s, 1H), 7.86-7.81 (m, 2H), 7.731-7.66 (m, 2H), 7.61 (dd, J=8.8, 6.8 Hz, 1H), 7.54-7.50 (m, 1H), 7.24-7.16 (m, 1H), 7.07 (d, J=7.6 Hz, 1H), 5.40-5.28 (m, 2H), 4.68 (d, J=10.8 Hz, 1H), 4.32-4.22 (m, 3H), 4.09-3.95 (m, 3H), 3.67 (d, J=11.2 Hz, 1H), 3.61-3.54 (m, 3H), 3.17 (s, 3H), 3.12 (d, J=3.6 Hz, 3H), 2.98-2.95 (m, 1H), 2.91-2.90 (m, 3H), 2.85 (d, J=3.2 Hz, 3H), 2.80 (brs, 3H), 2.73 (brs, 3H), 2.28-2.22 (m, 1H), 2.11-2.00 (m, 2H), 1.83-1.80 (m, 1H), 1.71-1.65 (m, 3H), 1.59-1.47 (m, 3H), 1.38 (d, J=6.4 Hz, 3H), 0.99-0.86 (m, 6H), 0.79-0.70 (m, 6H), 0.51 (brs, 3H) [along with minor peaks, arising presumably due to rotameric components, intensity of which reduced in VT ¹H-NMR]; LCMS (ESI): m/z [M+H]⁺ calc'd for C₅₄H₇₃N₈O₇ 957.56; found 957.50.

### Example 14

### Compound 110

### Synthesis of (3S)—N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-1-((E)-4-(3-fluoroazetidin-1-yl)-4-oxobut-2-en-1-yl)-N-methylpyrrolidine-3-carboxamide (Compound 110)

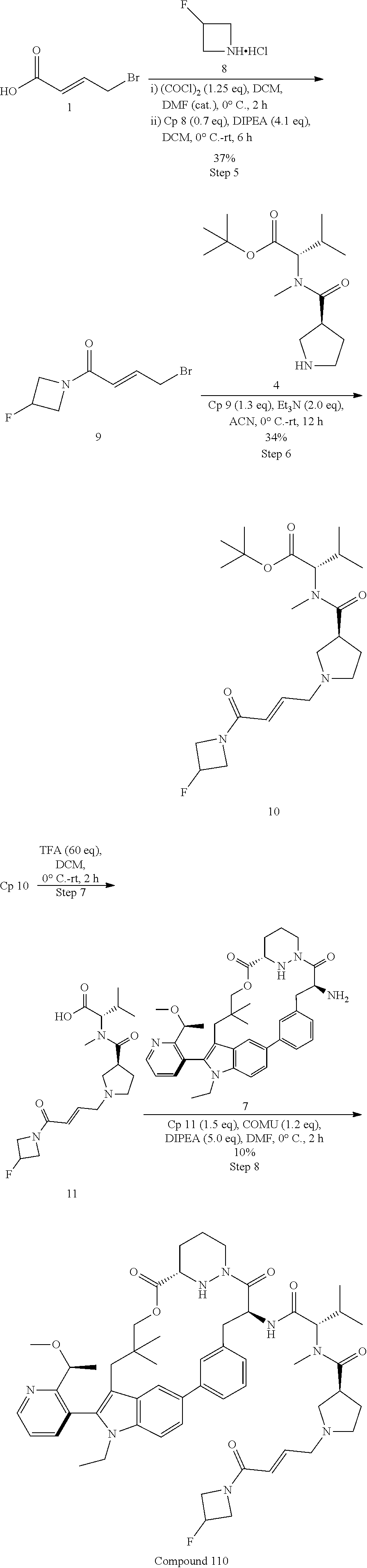

### Step 5

Synthesis of (E)-4-bromo-1-(3-fluoroazetidin-1-yl)but-2-en-1-one(3): To a stirred solution of (E)-4-bromobut-2-enoic acid 1 (1.2 g, 7.27 mmol) in DCM (10 mL) at 0° C. under an atmosphere of N₂, was added oxalyl chloride (0.78 mL, 1.25 eq, 9.09 mmol) in a drop-wise fashion followed by catalytic amount of DMF (0.05 mL, 0.07 eq, 0.646 mmol). The resulting solution was stirred at 0° C. for next 2 h, and subsequently to it was added DIPEA (5.26 mL, 4.1 eq, 30.3 mmol), followed by 3-fluoroazetidine hydrochloride 8 (676 mg, 0.7 eq, 6.06 mmol). The resulting reaction mixture was then allowed to come to rt and stirred for next 6 h. After completion of reaction (TLC monitoring), the reaction mixture was quenched with water (50 mL), extracted with DCM (3×100 mL), and washed with brine. The combined organic layer was dried over anhydrous Na₂SO₄, and filtered. After filtration, the filtrate was concentrated under reduced pressure and the crude residue was purified by Combi-flash column chromatography over silica gel using 50-70% EtOAc in n-heptane to afford (E)-4-bromo-1-(3-fluoroazetidin-1-yl)but-2-en-1-one 9 (600 mg, 37%) as a brown solid. ¹H NMR (400 MHz, DMSO-d₆): δ 6.71-6.64 (m, 1H), 6.277 (d, J=15.2, 1H), 4.78-4.62 (m, 2H), 4.33 (d, J=12, 2H), 4.306-4.242 (m, 2H).

### Step 6

Synthesis of tert-butyl N—((S)-1-((E)-4-(3-fluoroazetidin-1-yl)-4-oxobut-2-en-1-yl)pyrrolidine-3-carbonyl)-N-methyl-L-valinate(10): To a stirred solution of (E)-4-bromo-1-(3-fluoroazetidin-1-yl)but-2-en-1-one 9 (406 mg, 1.3 eq, 1.828 mmol) and tert-butyl N-methyl-N—((S)-pyrrolidine-3-carbonyl)-L-valinate 4 (400 mg, 1.408 mmol) in acetonitrile (5 mL) under an atmosphere of N₂, was added triethylamine (0.4 mL, 2 eq, 2.82 mmol) at 0° C. The reaction mixture was allowed to come to rt and stirred for next 12 h. After completion of reaction (TLC monitoring), the reaction mixture was concentrated under reduced pressure to get the crude which was purified by Combi-flash column chromatography over silica gel to afford tert-butyl N—((S)-1-((E)-4-(3-fluoroazetidin-1-yl)-4-oxobut-2-en-1-yl)pyrrolidine-3-carbonyl)-N-methyl-L-valinate 10 (0.2 g, 34%) as a yellow sticky solid. LCMS (ESI): m/z [M+H]⁺ calc'd for C₂₂H₃₇FN₃O₄ 426.28; found 426.30.

### Step 7

Synthesis of N—((S)-1-((E)-4-(3-fluoroazetidin-1-yl)-4-oxobut-2-en-1-yl)pyrrolidine-3-carbonyl)-N-methyl-L-valine (11): To a stirred solution of tert-butyl N—((S)-1-((E)-4-(3-fluoroazetidin-1-yl)-4-oxobut-2-en-1-yl)pyrrolidine-3-carbonyl)-N-methyl-L-valinate 10 (0.2 g, 0.47 mmol) in DCM (5 mL) under an atmosphere of N₂, was added trifluoroacetic acid (2.1 mL, 60 eq, 28.2 mmol) at 0° C. The reaction mixture was allowed to come to rt and stirred for next 2 h. After completion of reaction (TLC monitoring), the reaction mixture was concentrated under reduced pressure to get the crude N—((S)-1-((E)-4-(3-fluoroazetidin-1-yl)-4-oxobut-2-en-1-yl)pyrrolidine-3-carbonyl)-N-methyl-L-valine 11 (120 mg, as TFA salt) as a thick liquid, which was used in the next step without further purification. LCMS (ESI): m/z [M+H]⁺ calc'd for C₁₈H₂₉FN₃O₄ 370.21; found 370.24.

### Step 8

Synthesis of (3S)—N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-1-((E)-4-(3-fluoroazetidin-1-yl)-4-oxobut-2-en-1-yl)-N-methylpyrrolidine-3-carboxamide (Compound 110):

To stirred solution of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione 7 (0.2 g, 0.321 mmol) and (2S)-2-{1-[(3S)-1-[(2E)-4-(3-fluoroazetidin-1-yl)-4-oxobut-2-en-1-yl]pyrrolidin-3-yl]-N-methylformamido}-3-methylbutanoic acid 11 (233 mg, 1.5 eq, 0.481 mmol, crude as TFA salt, its preparation is described in another section) in DMF (3 mL) at 0° C. under an atmosphere of N₂ was added DIPEA (0.285 mL, 5 eq, 1.6 mmol) followed by COMU (165 mg, 1.2 eq, 0.385 mmol) and the reaction mixture was stirred at 0° C. for 2 h. After completion of reaction (TLC/LC monitoring), the reaction mixture was diluted with ice cold brine (20 mL) and extracted with ethyl acetate (3×50 mL). The Combined organic layer was dried over anhydrous Na₂SO₄, and filtered. The filtrate was concentrated under reduced pressure and the residue was purified by RP-HPLC to afford (3S)—N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-1-((E)-4-(3-fluoroazetidin-1-yl)-4-oxobut-2-en-1-yl)-N-methylpyrrolidine-3-carboxamide, Compound 110 (30 mg, 10% w.r.t Cp 7) as a white solid. ¹H NMR (400 MHz, DMSO-d₆): δ 8.61-8.07 (m, 1H), 8.75 (d, J=1.2 Hz, 1H), 7.98 (s, 1H), 7.82 (dd, J=1.6, 7.6 Hz, 2H), 7.11 (d, J=8.8 Hz, 1H), 7.65-7.64 (m, 1H), 7.61 (d, J=8.4 Hz, 1H), 7.54-7.51 (m, 1H), 7.22 (q, J=7.6 Hz, 1H), 7.06 (d, J=7.6 Hz, 1H), 7.70-6.64 (m, 1H), 6.15 (d, J=14 Hz, 1H), 5.31 (t, J=8.8 Hz, 2H), 5.48-5.01 (m, 1H), 4.68 (d, J=10.8 Hz, 1H), 4.54-4.47 (m, 1H), 4.27-4.24 (m, 5H), 4.06-3.92 (m, 3H), 3.67 (d, J=10.8 Hz, 1H), 3.60 (d, J=11.2 Hz, 1H), 3.55-3.51 (m, 1H), 3.02-2.95 (m, 1H), 2.93-2.84 (m, 1H), 3.26-3.23 (m, 2H), 3.13-3.09 (m, 3H), 2.86-2.80 (m, 2H), 2.78-2.76 (m, 2H), 2.74-2.72 (m, 2H), 2.70-2.68 (m, 1H), 2.60-2.58 (m, 1H), 2.40-2.35 (m, 1H), 2.05-1.89 (m, 4H), 1.87-1.80 (m, 1H), 1.71-1.68 (m, 1H), 1.61-1.58 (m, 1H), 1.56-1.50 (m, 1H), 1.38 (d, J=6.4 Hz, 3H), 0.97-0.85 (m, 6H), 0.76-0.71 (m, 6H), 0.429 (m, 3H); LCMS (ESI): m/z [M+H]⁺ calc'd for C₅₅H₇₂FN₈O₇ 975.55; found 975.39.

### Example 15

### Compound 111

### Synthesis of (3S)-1-((E)-4-(dimethylamino)-4-oxobut-2-en-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 111)

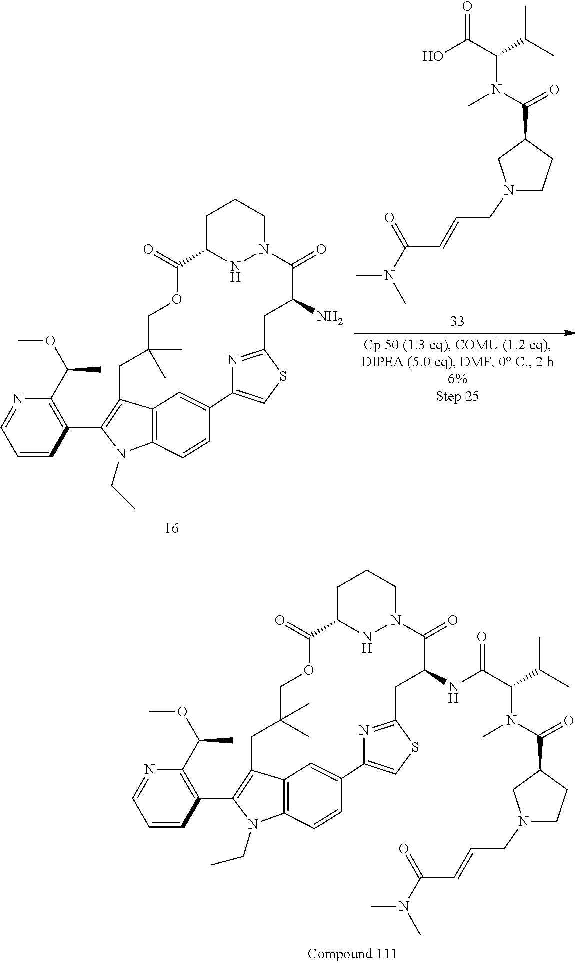

### Step 25

Synthesis of (3S)-1-((E)-4-(dimethylamino)-4-oxobut-2-en-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 111): To a stirred solution of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione 16 (0.2 g, 0.317 mmol) and N—((S)-1-((E)-4-(dimethylamino)-4-oxobut-2-en-1-yl)pyrrolidine-3-carbonyl)-N-methyl-L-valine 5 (140 mg, 1.3 eq, 0.412 mmol) in DMF (10 mL) at 0° C. under an atmosphere of N₂ was added DIPEA (0.3 mL, 5 eq, 1.59 mmol) followed by COMU (163 mg, 1.2 eq, 0.38 mmol) and the reaction mixture was stirred at 0° C. for 2 h. After completion of reaction (TLC and LCMS monitoring), the reaction mixture was diluted with ice-cold brine (50 mL) and extracted with EtOAc (3×100 mL). The combined organic layer was dried over anhydrous Na₂SO₄, filtered, and concentrated under reduced pressure to get a crude, which was purified by RP-HPLC to eventually afford (3S)-1-((E)-4-(dimethylamino)-4-oxobut-2-en-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide, Compound 111 (18 mg, 6%, 98.67% purity) as a white solid. ¹H NMR (400 MHz, DMSO-d₆): δ 8.74 (d, J=3.2, 1.2 Hz, 1H), 8.48 (d, J=4.8 Hz, 1H), 7.85-7.72 (m, 2H), 7.56 (d, J=8.8 Hz, 1H), 7.51 (q, J=4.8 Hz, 1H), 6.69-6.56 (m, 2H), 5.51-5.41 (m, 1H), 5.12-5.04 (m, 1H), 4.75 (d, J=11.2 Hz, 1H), 4.35-4.29 (m, 2H), 4.28-4.26 (m, 2H), 4.20-4.00 (m, 1H), 3.62-3.59 (m, 3H), 3.22 (s, 6H), 3.18 (d, J=7.2 Hz, 1H), 2.97 (s, 3H), 2.87 (s, 3H), 2.85-2.83 (m, 5H), 2.79-2.64 (m, 3H), 2.42-2.37 (m, 2H), 2.17-1.95 (m, 5H), 1.79 (brs, 2H), 1.56-1.46 (m, 1H), 1.35 (d, J=6 Hz, 3H), 1.23 (brs, 1H), 0.96-0.89 (m, 6H), 0.85-0.73 (m, 6H), 0.30 (brs, 3H) [along with minor peaks, arising presumably due to rotameric components, intensity of which reduced in VT ¹H NMR]; LCMS (ESI): m/z [M+H]⁺ calc'd for C₅₁H₇₀N₉O₇S 952.51; found 952.55.

### Example 16

### Compound 112

### Synthesis of 1-(4-(dimethylamino)-4-oxobut-2-yn-1-yl)-N-((2S)-1-(((6³S,4S)-1¹-ethyl-1²-(2((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylazetidine-3-carboxamide (Compound 112)

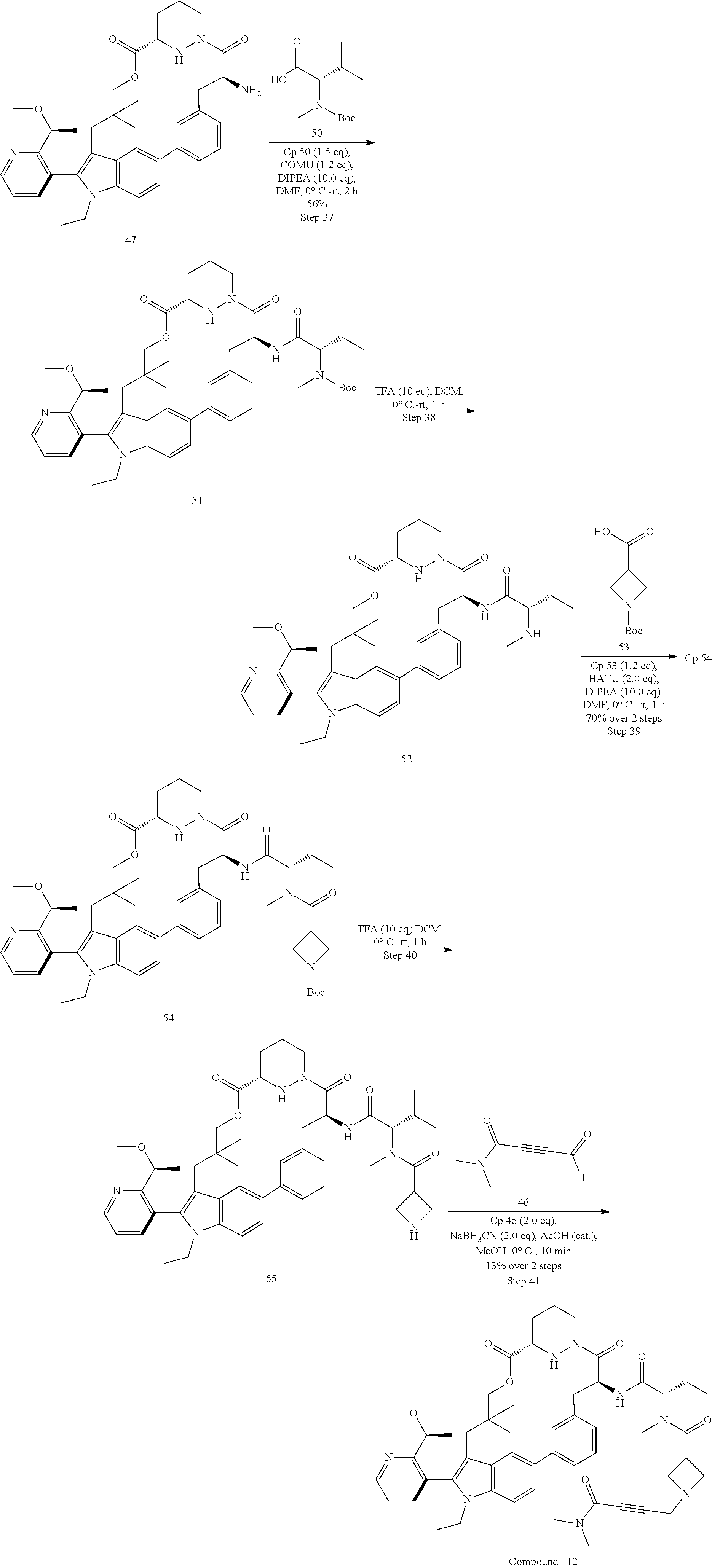

### Step 37

Synthesis of tert-butyl ((2S)-1-(((6³S,4S)-1¹-ethyl-1²-(2-((S)-1-methoxyethyl)pyridin-3-yl)-10,10-dimethyl-5,7-dioxo-6¹,6²,6′,6′,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (51): To a stirred solution of (6³S,4S)-4-amino-1¹-ethyl-1²-(2-((S)-1-methoxyethyl)pyridin-3-yl)-10,10-dimethyl-6¹,6²,63, 64, 65, 6⁶-hexahydro-1¹H-8-oxa-1(5,3)-indola-6(1,3)-pyridazina-2(1,3)-benzenacycloundecaphane-5,7-dione 47 (1.2 g, 1.92 mmol) in DMF (20 mL) at 0° C. under an atmosphere of N₂ was added DIPEA (3.42 mL, 10 eq, 19.2 mmol) followed by N-(tert-butoxycarbonyl)-N-methyl-L-valine 50 (667 mg, 1.5 eq, 2.89 mmol) and COMU (989 mg, 1.2 eq, 2.31 mmol), respectively. The reaction mixture was allowed to warm to rt and stirred for next 2 h. After completion of reaction (TLC monitoring), the reaction mixture was diluted with chilled brine (50 mL) and extracted with EtOAc (3×50 mL). The combined organic layer was washed with brine (50 mL), dried over anhydrous Na₂SO₄ and filtered. The filtrate was concentrated under reduced pressure and the residue thus obtained was purified by Combi-flash column chromatography over silica gel using 50-60% EtOAc in n-Heptane to afford tert-butyl ((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate 51 (900 mg, 56%) as a reddish solid. LCMS (ESI): m/z [M+H]⁺ calc'd for C₄₈H₆₅N₆O₇ 837.49; found 837.33.

### Step 38

Synthesis of (2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide (52): To a stirred solution of tert-butyl ((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate 51 (900 mg, 1.08 mmol) in DCM (10 mL) at 0° C. was added TFA (0.82 mL, 10 eq, 10.8 mmol) under nitrogen atmosphere. The reaction mixture was gradually warmed to rt and stirred for next 1 h. After completion of reaction (TLC monitoring), the reaction mass was concentrated under reduced pressure to give a crude which was further basified using saturated aq. solution of NaHCO₃ and extracted with 20% IPA in CHCl₃ (4×100 mL). The combined organic layer was dried over anhydrous Na₂SO₄, filtered and concentrated under reduced pressure eventually to obtain the crude (2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide 52 (800 mg) as a brown solid, which was used in the next step without further purification. LCMS (ESI): m/z [M+H]⁺ calc'd for C₄₃H₅₇N₆O₅ 737.44; found 737.55.

### Step 39

Synthesis of tert-butyl 3-(((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)(methyl)carbamoyl)azetidine-1-carboxylate (54): To a stirred solution of (2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide 52 (900 mg, 1.22 mmol) and 1-(tert-butoxycarbonyl)azetidine-3-carboxylic acid 53 (295 mg, 1.2 eq, 1.47 mmol) in DMF (10 mL) at 0° C. under an atmosphere of N₂ was added DIPEA (2.17 mL, 10 eq, 12.2 mmol), followed by HATU (575 mg, 2 eq, 2.44 mmol). The reaction mixture was allowed to warm to rt and stirred for next 1 h. After completion of reaction (TLC monitoring), the reaction mixture was diluted with chilled brine (20 mL) and extracted with EtOAc (3×50 mL). The combined organic layer was dried over anhydrous Na₂SO₄, filtered, and concentrate under reduced pressure to get a crude, which was further purified by Combi-flash column chromatography over silica gel using 80% EtOAc in n-Heptane eventually to afford tert-butyl 3-(((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)azetidine-1-carboxylate 54 (0.7 g, 70% over 2 steps) as a brown solid. LCMS (ESI): m/z [M+H]⁺ calc'd for C₅₂H₇₀N₇O₈ 920.53; found 920.33.

### Step 40

Synthesis of N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylazetidine-3-carboxamide (55): To a stirred solution of tert-butyl 3-(((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)azetidine-1-carboxylate 54 (0.7 g, 0.761 mmol) in DCM (20 mL) at 0° C. was added TFA (0.58 mL, 10 eq, 7.61 mmol) under nitrogen atmosphere. The reaction mixture was gradually warmed to rt and stirred for next 1 h.

After completion of reaction (TLC monitoring), the reaction mass was concentrated under reduced pressure to give a crude which was further basified using aq. solution of NaHCO₃ and extracted with 15% MeOH in DCM (3×50 mL). The combined organic layer was dried over anhydrous Na₂SO₄, filtered, and concentrated under reduced pressure eventually to obtain the crude N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylazetidine-3-carboxamide 55 (0.6 g, crude) as a brown solid. LCMS (ESI): m/z [M+H]⁺ calc'd for C₄₇H₆₂N₇O₆ 820.48; found 820.39.

### Step 41

Synthesis of 1-(4-(dimethylamino)-4-oxobut-2-yn-1-yl)-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylazetidine-3-carboxamide (Compound 112): To a stirred solution of N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylazetidine-3-carboxamide 55 (0.2 g, 0.244 mmol) and N,N-dimethyl-4-oxobut-2-ynamide 46 (61 mg, 2 eq, 0.488 mmol) in MeOH (20 mL) was added acetic acid (catalytic, 10 μL) at 0° C. Sodium cyanoborohydride (30.7 mg, 2 eq, 0.488 mmol) was then added and the reaction mixture was stirred for 10 min. After completion of reaction (monitored by LCMS/TLC), the reaction mass was quenched with water (10 mL) and extracted with 10% MeOH in DCM (2×50 mL). The combined organic layer was dried over anhydrous Na₂SO₄ and concentrated under reduced pressure to get a crude which was purified by RP-HPLC to afford 1-(4-(dimethylamino)-4-oxobut-2-yn-1-yl)-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylazetidine-3-carboxamide, Compound 112 (40 mg, 13% over 2 steps, 90.36% purity by LCMS) as a white solid. ¹H NMR (400 MHz, DMSO-d₆): 8.76 (d, J=3.6 Hz, 1H), 8.56-8.09 (m, 1H), 7.98 (s, 1H), 7.79-7.88 (m, 2H), 7.71-7.59 (m, 2H), 7.54-7.49 (m, 1H), 7.30-7.21 (m, 1H), 7.08 (d, J=5.6 Hz, 1H), 5.42-5.30 (m, 2H), 4.65 (d, J=10.8 Hz, 1H), 4.29-4.22 (m, 3H), 4.04-3.96 (m, 2H), 3.68-3.56 (m, 5H), 3.50 (s, 2H), 3.19-3.09 (m, 6H), 2.98-2.91 (m, 1H), 2.84-2.77 (m, 6H), 2.75-2.61 (m, 5H), 2.08-1.99 (m, 2H), 1.83-1.79 (m, 1H), 1.69-1.55 (m, 2H), 1.38 (d, J=6 Hz, 3H), 0.96-0.88 (m, 6H), 0.72 (q, J=7.6 Hz, 6H), 0.54 (d, J=9.6 Hz, 3H) [along with minor peaks, arising presumably due to rotameric components, intensity of which reduced in VT ¹H NMR]; LCMS (ESI): m/z [M+H]⁺ calc'd for C₅₃H₆₉N₈O₇ 929.53; found 929.77.

### Example 17

### Compound 113

### Synthesis of (3S)-1-(4-(dimethylamino)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 113)

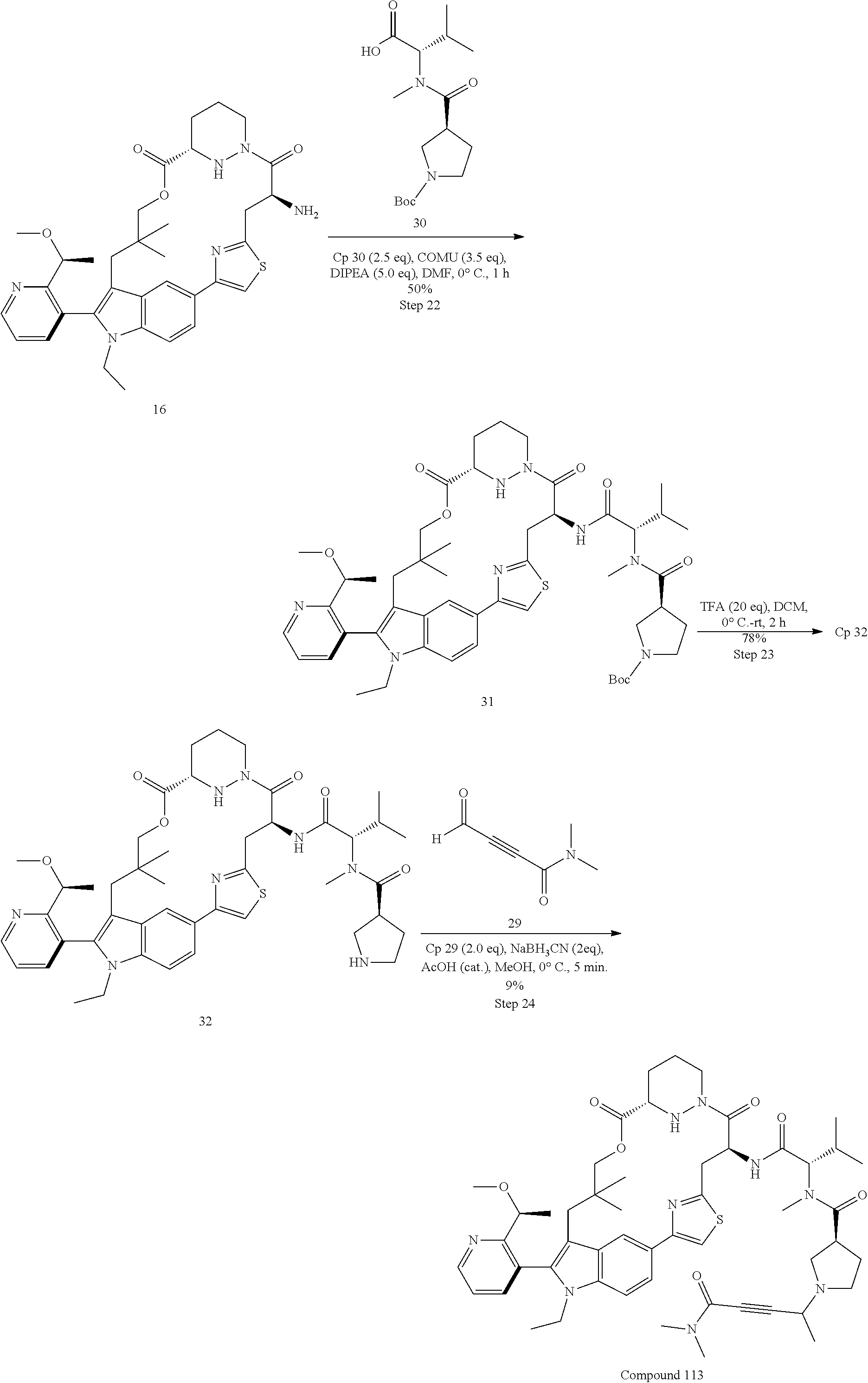

### Step 22

Synthesis of tert-butyl (3S)-3-(((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)(methyl)carbamoyl)pyrrolidine-1-carboxylate (31): To a stirred solution of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane- 5,7-dione 16 (2 g, 3.16 mmol) and N—((S)-1-(tert-butoxycarbonyl)pyrrolidine-3-carbonyl)-N-methyl-L-valine 30 (2.6 g, 2.5 eq, 7.92 mmol) in DMF (20 mL) at 0° C. was added DIPEA (3.0 mL, 5 eq, 15.8 mmol) and stirred for 5 min. Subsequently, COMU (4.7 g, 3.5 eq, 11 mmol) was added and the reaction mixture was stirred at 0° C. for 1 h. After completion of the reaction (TLC/LCMS monitoring), the reaction mixture was diluted with ice-cold brine (50 mL) and extracted with EtOAc (3×100 mL). The combined organic layer was dried over anhydrous Na₂SO₄, filtered and concentrated under reduced pressure to get a crude residue, which was purified by Combi-flash column chromatography over silica gel using 70% EtOAc in n-heptane to afford tert-butyl (3S)-3-(((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)(methyl)carbamoyl)pyrrolidine-1-carboxylate 31 (1.5 g, 50% w.r.t Cp 16) as an off white solid. LCMS (ESI): m/z [M+H]⁺ calc'd for C₅₀H₆₉N₈O₈ 941.50; found 941.48.

### Step 23

Synthesis of (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (32): To a stirred solution of tert-butyl (3S)-3-(((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)(methyl)carbamoyl)pyrrolidine-1-carboxylate 31 (1 g, 1.06 mmol) in DCM (20 mL) was added TFA (1.63 mL, 20 eq, 21.2 mmol) at 0° C. The reaction mixture was allowed to come to rt and stirred for next 2 h. After completion of reaction (TLC monitoring), the reaction mixture was concentrated under reduced pressure to get a crude, which was further basified by aq. solution of NaHCO₃ to pH ˜ 10. The aqueous phase was extracted with 25% IPA in CHCl₃ (3×100 mL), the combined organic layer was dried over anhydrous Na₂SO₄, and filtered. The filtrate was concentrated under reduced pressure to get the crude (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide 32 (700 mg, 78%) as an off white solid, which was used in the next step without further purification. LCMS (ESI): m/z [M+H]⁺ calc'd for C₄₅H₆₁N₈O₆S 841.44; found 841.47.

### Step 24

Synthesis of (3S)-1-(4-(dimethylamino)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 113): To a stirred solution of (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide 32 (400 mg, 0.475 mmol) and N,N-dimethyl-4-oxobut-2-ynamide 29 (120 mg, 2 eq, 952 mmol) in MeOH (10 mL) at 0° C. was added catalytic AcOH (catalytic, 10 mL) followed by NaBH₃CN (62 mg, 2 eq, 0.952 mmol), and the reaction mixture was stirred at the same temperature for 5 min. After completion of reaction (monitored by LCMS/TLC), the reaction mixture was quenched with water (10 mL) and extracted with 10% MeOH in DCM (3×50 mL). The combined organic layer was dried over anhydrous Na₂SO₄, filtered, and concentrated under reduced pressure to get a crude, which was purified by RP-HPLC to afford (3S)-1-(4-(dimethylamino)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide, Compound 113 (40 mg, 9%, 98.65% purity) as a white solid. ¹H NMR (400 MHz, DMSO-d): δ 8.75 (d, J=3.6 Hz, 1H), 8.48 (d, J=4.8 Hz, 1H), 7.85 (s, 1H), 7.86-7.72 (m, 2H), 7.57 (d, J=8.8 Hz, 1H), 7.51 (q, J=4.8 Hz, 1H), 5.52-5.42 (m, 1H), 5.08 (q, J=12 Hz, 1H), 4.76 (d, J=11.2 Hz, 1H), 4.37-4.30 (m, 2H), 4.28-4.11 (m, 3H), 4.01 (d, J=10.8 Hz, 1H) 3.69-3.64 (m, 2H), 3.59-3.55 (m, 2H), 3.35 (brs, 1H), 3.26 (s, 3H), 3.15 (d, J=6.4 Hz, 3H), 2.96 (s, 3H), 2.86 (s, 3H), 2.83 (s, 3H), 2.80-2.72 (m, 2H), 2.63-2.58 (m, 2H), 2.44-2.37 (m, 2H), 2.23-1.92 (m, 4H), 1.79 (brs, 2H), 1.59-1.40 (m, 1H), 1.36 (t, J=6 Hz, 3H), 0.96-0.89 (m, 6H), 0.85-0.75 (m, 6H).[along with minor peaks, arising presumably due to rotameric components, intensity of which reduced in VT ¹H NMR]; LCMS (ESI): m/z [M+H]⁺ calc'd for C₅₁H₆₈N₉O₇S 950.50; found 950.76.

### Example 18

### Compound 114

### Synthesis of (3S)-1-(5-(dimethylamino)-5-oxopent-3-yn-2-yl)-N-((2S)-1-(((63S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 114)

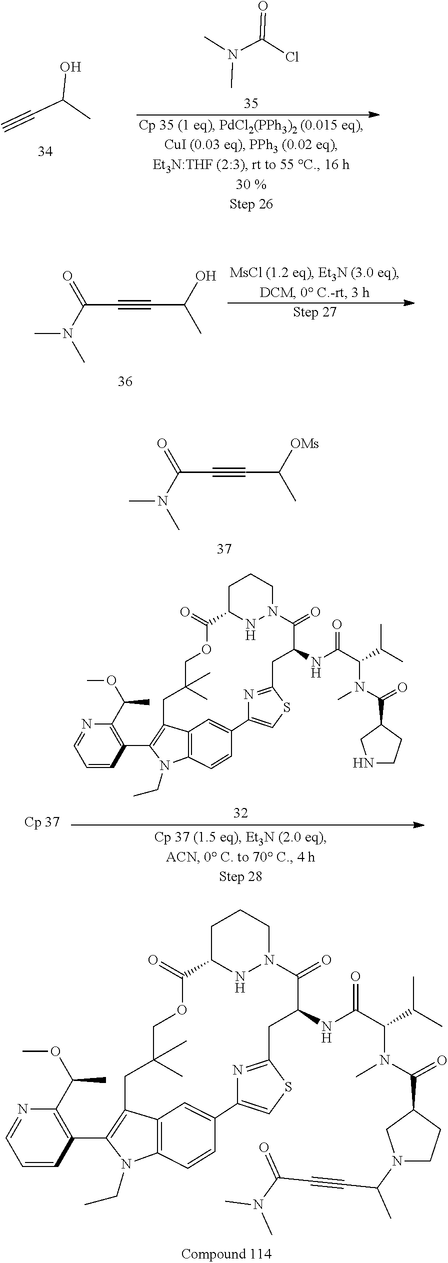

### Step 26

Synthesis of 4-hydroxy-N,N-dimethylpent-2-ynamide (36): To a stirred solution of but-3-yn-2-ol 34 (2 g, 28.5 mmol) in THF (12 mL) was added Et₃N (8 mL) followed by N,N-dimethylcarbamoyl chloride 35 (3.07 g, 1.0 eq, 28.5 mmol), and the reaction mixture was purged with N2 for 10 min. Subsequently, to it were added PdCl2(PPh3)2 (0.3 g, 0.015 eq, 0.428 mmol), CuI (163 mg, 0.03 eq, 0.856 mmol) and PPh3 (172 mg, 0.02 eq, 0.656 mmol) sequentially, and the reaction mixture was heated at 55° C. for next 16 h. After completion of reaction (monitored by TLC and LCMS), the reaction mixture was diluted with water (50 mL) and extracted with EtOAc (3×100 mL). The combined organic layer was dried over anhydrous Na2SO4, filtered, and concentrated under reduced pressure to get a crude, which was further purified by Combi-flash column chromatography over silica gel using 40-70% EtOAc in n-heptane to eventually obtain 4-hydroxy-N,N-dimethylpent-2-ynamide 36 (1.2 g, 30%) as a brown oil. 1H NMR (CDCl3, 400 MHz): δ 4.68 (q, J=6.4 Hz, 1H), 3.20 (s, 3H), 3.02 (s, 3H), 1.52 (d, J=8.8 Hz, 3H); LCMS (ESI): m/z [M+H]+ calc'd for C7H12NO2 142.09; found 142.36.

### Step 27

Synthesis of 5-(dimethylamino)-5-oxopent-3-yn-2-yl methanesulfonate (37): To a stirred solution of 4-hydroxy-N,N-dimethylpent-2-ynamide 36 (0.3 g, 2.13 mmol) in DCM (10 mL) at 0° C. under nitrogen atmosphere was added Et3N (0.87 mL, 3 eq, 6.38 mmol) and stirred for 5 min. Subsequently, MsCl (0.18 mL, 1.2 eq, 2.55 mmol) was added, the reaction mixture gradually warmed to rt and stirred for next 3 h. After completion of reaction (monitored by TLC and LCMS), water (10 mL) was added to the reaction mixture and extracted with DCM (3×20 mL). The combined organic layer was dried over anhydrous Na2SO4, filtered and concentrated under reduced pressure to get the crude 5-(dimethylamino)-5-oxopent-3-yn-2-yl methanesulfonate 37 (250 mg) as a yellow oil, which was used in the next step without father purification.

### Step 28

Synthesis of (3S)-1-(5-(dimethylamino)-5-oxopent-3-yn-2-yl)-N-((2S)-1-(((63S,4S,Z)-11-ethyl-12-(2-((S)-1-methoxyethyl)pyridin-3-yl)-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 114): To a stirred solution of (3S)—N-((2S)-1-(((63S,4S,Z)-11-ethyl-12-(2-((S)-1-methoxyethyl)pyridin-3-yl)-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide 32 (250 mg, 0.297 mmol) in ACN (10 mL) at rt under N2 atmosphere was added Et₃N (0.1 mL, 2 eq, 0.594 mmol) followed by 5-(dimethylamino)-5-oxopent-3-yn-2-yl methanesulfonate 37 (97.8 mg, 1.5 eq, 0.446 mmol), and the resulting reaction mixture was heated at 70° C. for next 4 h. After completion of reaction (monitored by TLC and LCMS), the reaction mixture was concentrated under reduced pressure to get a crude, which was purified by RP-HPLC purification to eventually afford (3S)-1-(5-(dimethylamino)-5-oxopent-3-yn-2-yl)-N-((2S)-1-(((63S,4S,Z)-11-ethyl-12-(2-((S)-1-methoxyethyl)pyridin-3-yl)-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide, Compound 114 (21 mg, 97.33% purity) as a white solid. 1H NMR (400 MHz, DMSO-d6): δ 8.75 (dd, J=4.8, 1.6 Hz, 1H), 8.48 (d, J=5.2 Hz, 1H), 7.86 (s, 1H), 7.85-7.72 (m, 2H), 7.57 (d, J=8.8 Hz, 1H), 7.51 (q, J=4.8 Hz, 1H), 5.51-5.43 (m, 1H), 5.14-5.06 (m, 1H), 4.76 (d, J=10.8 Hz, 1H), 4.36-4.28 (m, 2H), 4.23-4.09 (m, 3H), 4.05-3.88 (m, 2H), 3.68-3.65 (m, 1H), 3.59-3.52 (m, 2H), 3.26 (d, J=2.0 Hz, 3H), 3.26 (d, J=2.0 Hz, 3H), 3.15 (d, J=2.0 Hz, 3H), 2.96 (s, 3H), 2.83 (s, 3H), 2.79-2.70 (m, 2H), 2.38 (d, J=15.2 Hz, 2H), 2.21-2.01 (m, 4H), 1.93-1.90 (m, 1H), 1.79 (brs, 2H), 1.56-1.48 (m, 1H), 1.36-1.33 (m, 6H), 0.96-0.84 (m, 6H), 0.80-0.75 (m, 6H), 0.31 (d, J=2.4 Hz, 3H) [along with minor peaks, arising presumably due to rotameric components, intensity of which reduced in VT 1H NMR]; LCMS (ESI): m/z [M+H]+ calc'd for C52H70N9O7S 964.51; found 964.58.

### Example 19

### Compound 115

### Synthesis of (3S)-1-((E)-4-(3,3-difluoroazetidin-1-yl)-4-oxobut-2-en-1-yl)-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1 ¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (Compound 115)

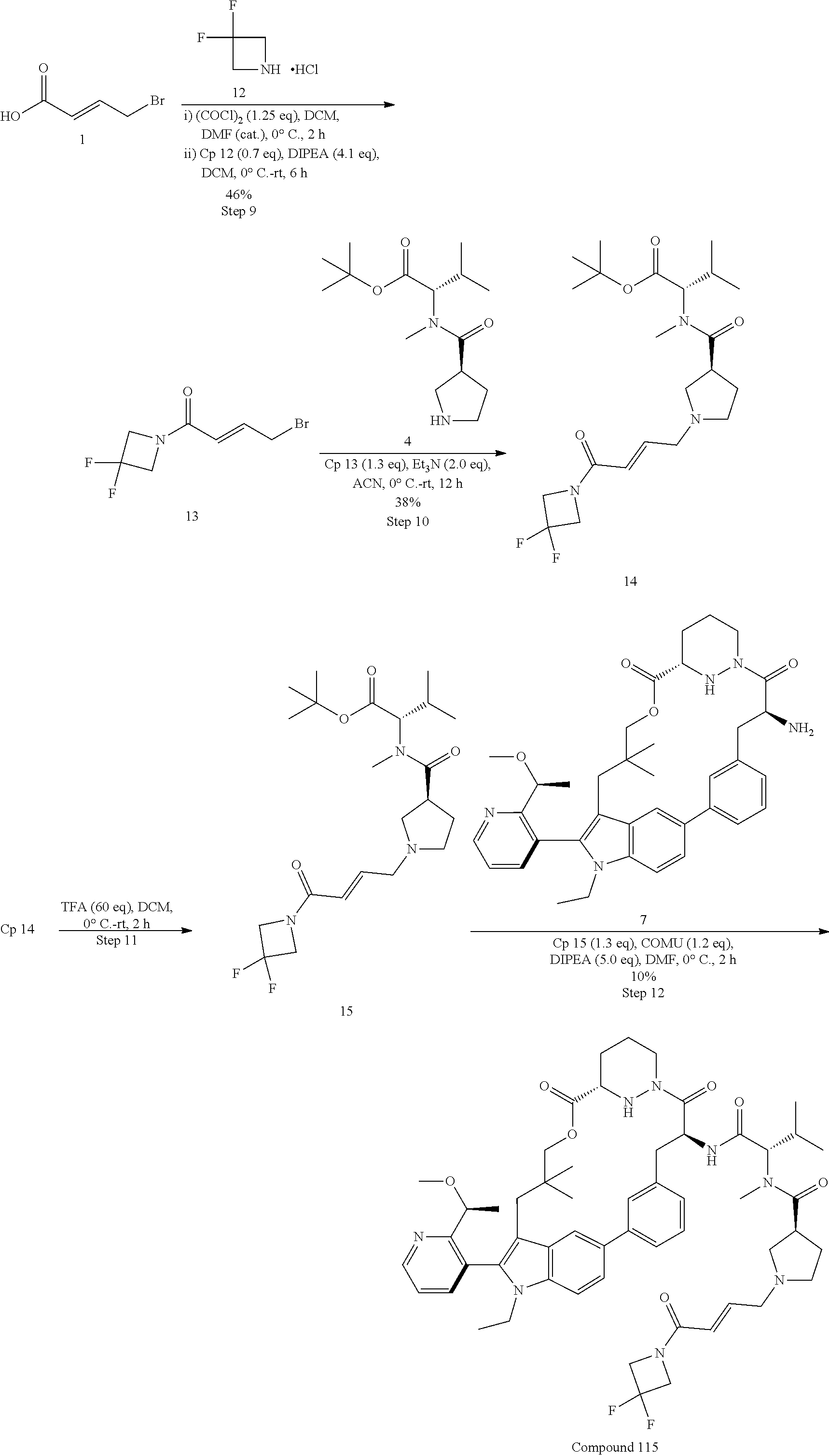

### Step 9

Synthesis of (E)-4-bromo-1-(3,3-difluoroazetidin-1-yl)but-2-en-1-one (13): To a stirred solution of (E)-4-bromobut-2-enoic acid 1 (1.2 g, 7.27 mmol) in DCM (10 mL) at 0° C. under an atmosphere of N₂, was added oxalyl chloride (0.78 mL, 1.25 eq, 9.09 mmol) in a drop-wise fashion followed by catalytic amount of DMF (0.05 mL, 0.07 eq, 0.646 mmol). The resulting solution was stirred at 0° C. for next 2 h, and subsequently to it was added DIPEA (5.26 mL, 4.1 eq, 30.3 mmol) followed by 3,3-difluoroazetidine hydrochloride 12 (785 mg, 0.7 eq, 6.06 mmol). The resulting reaction mixture was allowed to come to rt and stirred for next 6 h. After completion of reaction (TLC monitoring), the reaction mixture was quenched with water (20 mL), extracted with DCM (3×50 mL), and washed with brine. The combined organic layer was dried over anhydrous Na₂CO₃, and filtered. After filtration, the filtrate was concentrated under reduced pressure and the crude residue was purified by Combi-flash column chromatography over silica gel (50-70% EtOAc in n-heptane) to afford (E)-4-bromo-1-(3,3-difluoroazetidin-1-yl)but-2-en-1-one 13 (800 mg, 46%) as a brown solid. ¹H NMR (400 MHz, DMSO-d₄): δ 6.740-6.675 (m, 1H), 6.28 (d, J=18 Hz, 1H), 4.804-4.721 (m, 2H), 4.69-4.61 (m, 2H), 4.41 (d, J=13.2 Hz, 2H).

### Step 10

tert-butyl N—((S)-1-((E)-4-(3,3-difluoroazetidin-1-yl)-4-oxobut-2-en-1-yl)pyrrolidine-3-carbonyl)-N-methyl-L-valinate (14): To a stirred solution of tert-butyl N-methyl-N—((S)-pyrrolidine-3-carbonyl)-L-valinate 4 (600 mg, 2.10 mmol) and (2E)-4-bromo-1-(3,3-difluoroazetidin-1-yl)but-2-en-1-one 13 (650 mg, 1.3 eq, 2.10 mmol) in acetronitrile (15 mL) under an atmosphere of N₂, was added triethylamine (0.6 mL, 2 eq, 4.23 mmol) at 0° C. The reaction mixture was allowed to come to rt and stirred for next 12 h. After completion of the reaction (TLC monitoring), the reaction mixture was concentrated under reduced pressure to get the crude which was purified by Combi-flash column chromatography to afford tert-butyl N—((S)-1-((E)-4-(3,3-difluoroazetidin-1-yl)-4-oxobut-2-en-1-yl)pyrrolidine-3-carbonyl)-N-methyl-L-valinate 14 (300 mg, 38%) as a brown semi-solid. LCMS (ESI): m/z [M+H]⁺ calc'd for C₂₂H₃₆F₂N₃O₄ 444.27; found 444.12.

### Step 11

N—((S)-1-((E)-4-(3,3-difluoroazetidin-1-yl)-4-oxobut-2-en-1-yl)pyrrolidine-3-carbonyl)-N-methyl-L-valine (15): To a stirred solution of tert-butyl (2S)-2-{1-[(3S)-1-[(2E)-4-(3,3-difluoroazetidin-1-yl)-4-oxobut-2-en-1-yl]pyrrolidin-3-yl]-N-methylformamido}-3-methylbutanoate 14 (400 g, 0.902 mmol) in DCM (10 mL) under an atmosphere of N₂, was added trifluoroacetic acid (4.5 mL, 60 eq, 54.1 mmol) at 0° C. The reaction mixture was allowed to come to rt and stirred for next 2 h. After completion of reaction (TLC monitoring), the reaction mixture was concentrated under reduced pressure to get the crude N—((S)-1-((E)-4-(3,3-difluoroazetidin-1-yl)-4-oxobut-2-en-1-yl)pyrrolidine-3-carbonyl)-N-methyl-L-valine 15 (300 mg, crude as TFA salt) as a thick liquid, which was used in the next step without further purification. LCMS (ESI): m/z [M+H]⁺ calc'd for C₁₈H₂₈F₂N₃O₄ 388.20; found 388.25.

### Step 12

Synthesis of (3S)-1-((E)-4-(3,3-difluoroazetidin-1-yl)-4-oxobut-2-en-1-yl)-N-((2S)-1-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide (Compound 115): To a stirred solution of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione 7 (0.2 g, 0.321 mmol) and N—((S)-1-((E)-4-(3,3-difluoroazetidin-1-yl)-4-oxobut-2-en-1-yl)pyrrolidine-3-carbonyl)-N-methyl-L-valine 15 (209 mg, 1.3 eq, 0.417 mmol, crude as TFA salt, its preparation is described in another section) in DMF (5 mL) at 0° C. under an atmosphere of N₂ was added DIPEA (207 mg, 5 eq, 1.6 mmol) followed by COMU (165 mg, 1.2 eq, 0.385 mmol) and the reaction mixture was stirred at 0° C. for 2 h. After completion of the reaction (TLC/LC monitoring), the reaction mixture was diluted with ice cold brine (20 mL) and extracted with ethyl acetate (3×50 mL). The Combined organic layer was dried over anhydrous Na₂SO₄, and filtered. The filtrate was concentrated under reduced pressure and the crude residue was purified by RP-HPLC to afford (3S)-1-((E)-4-(3,3-difluoroazetidin-1-yl)-4-oxobut-2-en-1-yl)-N-((2S)-1-(((6³S,4S)-1¹-ethyl-1²(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylpyrrolidine-3-carboxamide, Compound 115 (30 mg, 10%) as a white solid. LCMS (ESI): m/z [M+H]⁺ calc'd for C₅₅H₇₁F₂N₈O₇ 993.54; found 993.52. ¹H NMR (400 MHz, DMSO-d₆): δ 8.76 (d, J=2.6 Hz, 1H), 8.66-8.10 (m, 1H), 7.98 (s, 1H), 7.84-7.80 (m, 2H), 7.72-7.69 (m, 1H), 7.67-7.63 (m, 1H), 7.61 (d, J=8.4 Hz, 1H), 7.53-7.50 (m, 1H), 7.20 (q, J=7.6 Hz, 1H), 7.08 (d, J=8.2 Hz, 1H), 6.762-6.68 (m, 1H), 6.20-615 (m, 1H), 5.31 (t, J=8.8 Hz, 2H), 4.69-4.60 (m, J=1.2 Hz, 2H), 4.30-4.22 (m, 5H), 4.06-3.99 (m, 3H), 3.68-3.49 (m, 3H), 3.22 (d, J=9.2 Hz, 3H), 3.13 (d, J=3.2 Hz, 3H), 3.04-2.90 (m, 1H), 2.86-2.82 (m, 2H), 2.78-2.74 (m, 4H), 2.70-2.69 (m, 2H), 2.66-2.60 (m, 2H), 2.45-2.35 (m, 1H), 2.05-1.88 (m, 4H), 1.87-1.80 (m, 1H), 1.71-1.61 (m, 1H), 1.38 (d, J=6 Hz, 3H), 0.97-0.87 (m, 6H), 0.79-0.70 (m, 6H), 0.59-0.49 (m, 3H); LCMS (ESI): m/z [M+H]⁺ calc'd for C₅₅H₇₁F₂N₈O₇ 993.54; found 993.52.

### Example 20

### Compound 116

### Synthesis of (3S)-1-(4-(azetidin-1-yl)-4-oxobut-2-yn-1-yl)-N-((2S)-1-(((6³S,4S Z)-1-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 116)

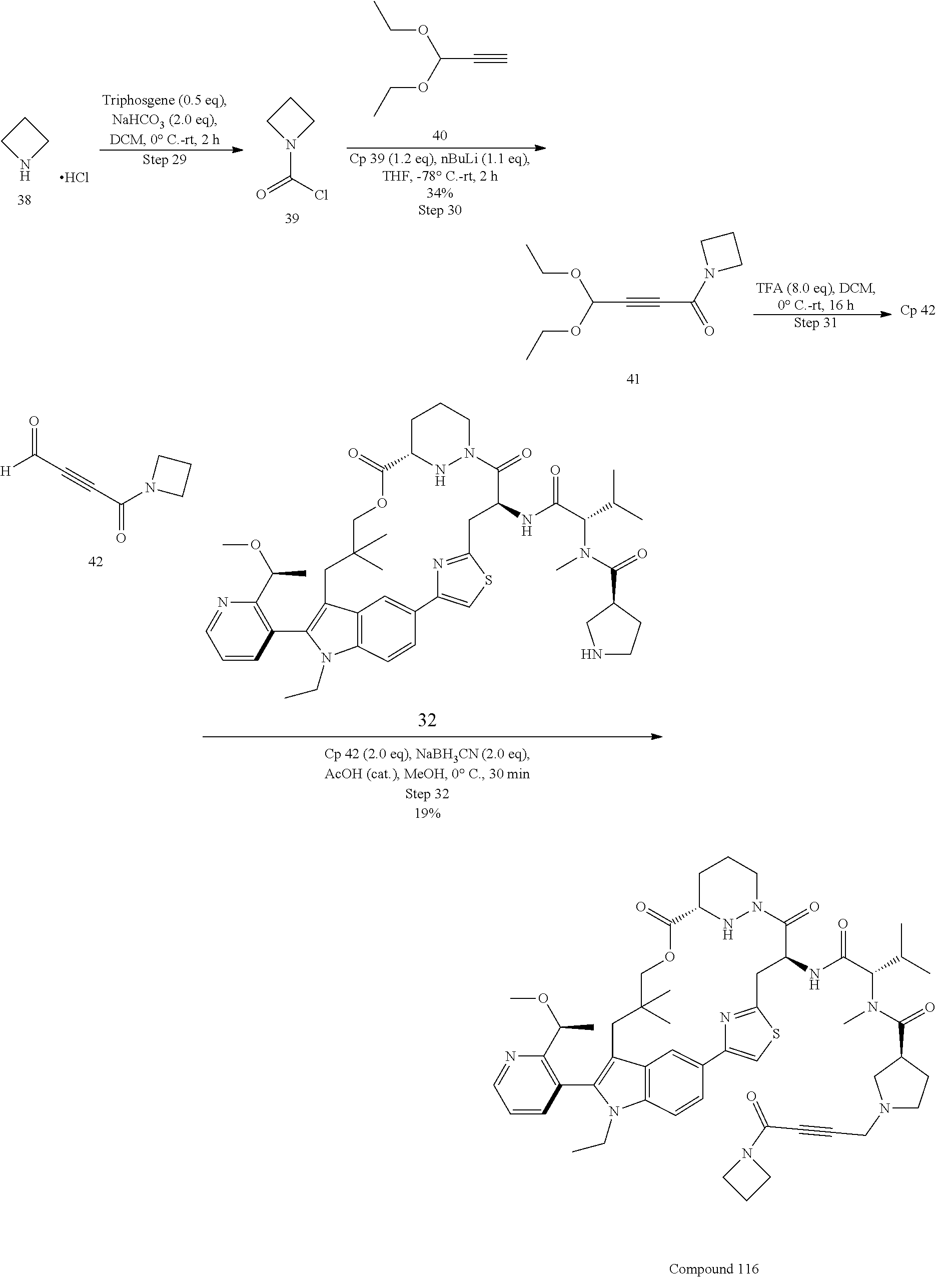

### Step 29

Synthesis of azetidine-1-carbonyl chloride (39): To a stirred solution of azetidine hydrochloride 38 (2.5 g, 26.7 mmol) in DCM (50 mL) at 0° C. was added NaHCO₃ (4.49 g, 2 eq, 80.1 mmol) followed by triphosgene (3.96 g, 0.5 eq, 13.4 mmol). The reaction mixture was allowed to warm to rt and stirred for next 2 h. After completion of reaction (monitoring by TLC), the reaction mixture was filtered through a cotton plug, thoroughly washed with DCM, and concentrated under reduced pressure to get the crude azetidine-1-carbonyl chloride 39 (2 g) as a colorless liquid, which was used in the next step without further purification.

### Step 30

Synthesis of 1-(azetidin-1-yl)-4,4-diethoxybut-2-yn-1-one (41): To a stirred solution of 3,3-diethoxyprop-1-yne 40 (1.8 g, 14 mmol) in THF (50 mL) was added ^{t}BuLi (6.1 mL, 2.5 M in hexane, 1.1 eq, 15.4 mmol) at −78° C. under nitrogen atmosphere, and the reaction mixture was stirred at the same temperature for 30 min. Subsequently, azetidine-1-carbonyl chloride 39 (2 g, 1.2 eq, 16.9 mmol), dissolved in THF (10 mL), was added dropwise to the reaction mixture at −78° C. The reaction mixture was gradually warmed to rt and stirred for next 2 h. After completion of reaction (monitored by TLC), the reaction mixture was cooled to 0° C., quenched with saturated solution of NH₄Cl (100 mL) and extracted with EtOAc (3×100 mL). The combined organic layer was dried over anhydrous Na₂SO₄, filtered and evaporated under reduced pressure to get a crude, which was further purified by Combi-flash column chromatography over silica gel to afford 1-(azetidin-1-yl)-4,4-diethoxybut-2-yn-1-one 41 (1 g, 34%) as a colorless liquid. ¹H NMR (400 MHz, DMSO-d₆): (5.5 (s, 1H), 4.13 (t, J=7.6 Hz, 2H), 3.90 (t, J=8 Hz, 2H), 3.66-3.60 (m, 2H), 3.58-3.52 (m, 2H), 2.27-2.19 (m, 2H), 1.14 (t, J=6.8 Hz, 6H); LCMS (ESI): m/z [M+H]⁺ calc'd for C₁₁H₁₈NO₃ 212.26; found 212.54.

### Step 31

Synthesis of 4-(azetidin-1-yl)-4-oxobut-2-ynal (42): To a stirred solution of 1-(azetidin-1-yl)-4,4-diethoxybut-2-yn-1-one 41 (0.3 g, 1.42 mmol) in DCM (30 mL) at 0° C. was added TFA (0.87 mL, 8 eq, 11.4 mmol) dropwise. The reaction mixture was gradually warmed to rt and stirred for next 16 h. After completion of reaction (monitored by TLC), evaporation of DCM was done via N₂ flashing followed by lyophilization of the resultant residue to evaporate the excess TFA eventually to obtain the crude 4-(azetidin-1-yl)-4-oxobut-2-ynal 42 (180 mg) as a brown sticky oil, which was used in the next step without further purification.

### Step 32

Synthesis of (3S)-1-(4-(azetidin-1-yl)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 116): To a stirred mixture of (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide 32 (250 mg, 0.297 mmol) and 4-(azetidin-1-yl)-4-oxobut-2-ynal 42 (81.5 mg, 2 eq, 0.594 mmol) in MeOH (10 mL) was added AcOH (catalytic, 10 L) at 0° C. Subsequently, NaBH₃CN (37.4 mg, 2 eq, 0.594 mmol) was added to the reaction mixture and stirred for 30 min. After completion of reaction (monitoring by TLC), the reaction mixture was quenched with water (10 mL) and extracted with DCM (3×50 mL). The combined organic layer was dried over anhydrous Na₂SO₄, filtered and concentrated under reduced pressure to get a crude which was purified by RP-HPLC to afford (3S)-1-(4-(azetidin-1-yl)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide, Compound 116 (55 mg, 19%, 98.69% purity) as a white solid. ¹H NMR (400 MHz, DMSO-d₆): δ 8.75 (dd, J=4.8, 1.6 Hz, 1H), 8.48 (d, J=4 Hz, 1H), 7.86 (s, 1H), 7.85-7.73 (m, 2H), 7.57 (d, J=8.4 Hz, 1H), 7.51 (q, J=4.8 Hz, 1H), 5.51-5.41 (m, 1H), 5.14-5.06 (m, 1H), 4.76 (d, J=11.2 Hz, 1H), 4.36-4.20 (m, 4H), 4.13 (t, J=7.6 Hz, 3H), 4.10 (d, J=8 Hz, 1H), 3.91-3.86 (m, 2H), 3.70-3.67 (m, 3H), 3.59-3.52 (m, 2H), 3.39-3.3 (m, 1H), 3.26 (s, 4H), 3.18-3.08 (m, 1H), 2.96 (s, 3H), 2.83-2.66 (m, 4H), 2.58-2.56 (m, 2H), 2.42-2.36 (m, 2H), 2.27-2.15 (m, 3H), 2.14-2.07 (m, 3H), 1.95-1.91 (m, 1H), 1.79 (brs, 2H), 1.56-1.48 (m, 1H), 1.36 (d, J=6 Hz, 3H), 0.96-0.90 (m, 6H), 0.85-0.75 (m, 6H) [along with minor peaks, arising presumably due to rotameric components, intensity of which reduced in VT ¹H NMR]. LCMS (ESI): m/z [M+H]⁺ calc'd for C₅₂H₆₈N₉O₇S 962.49; found 962.76.

### Example 21

### Compound 117

### Synthesis of (3S)-1-(4-(3,3-difluoroazetidin-1-yl)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 117)

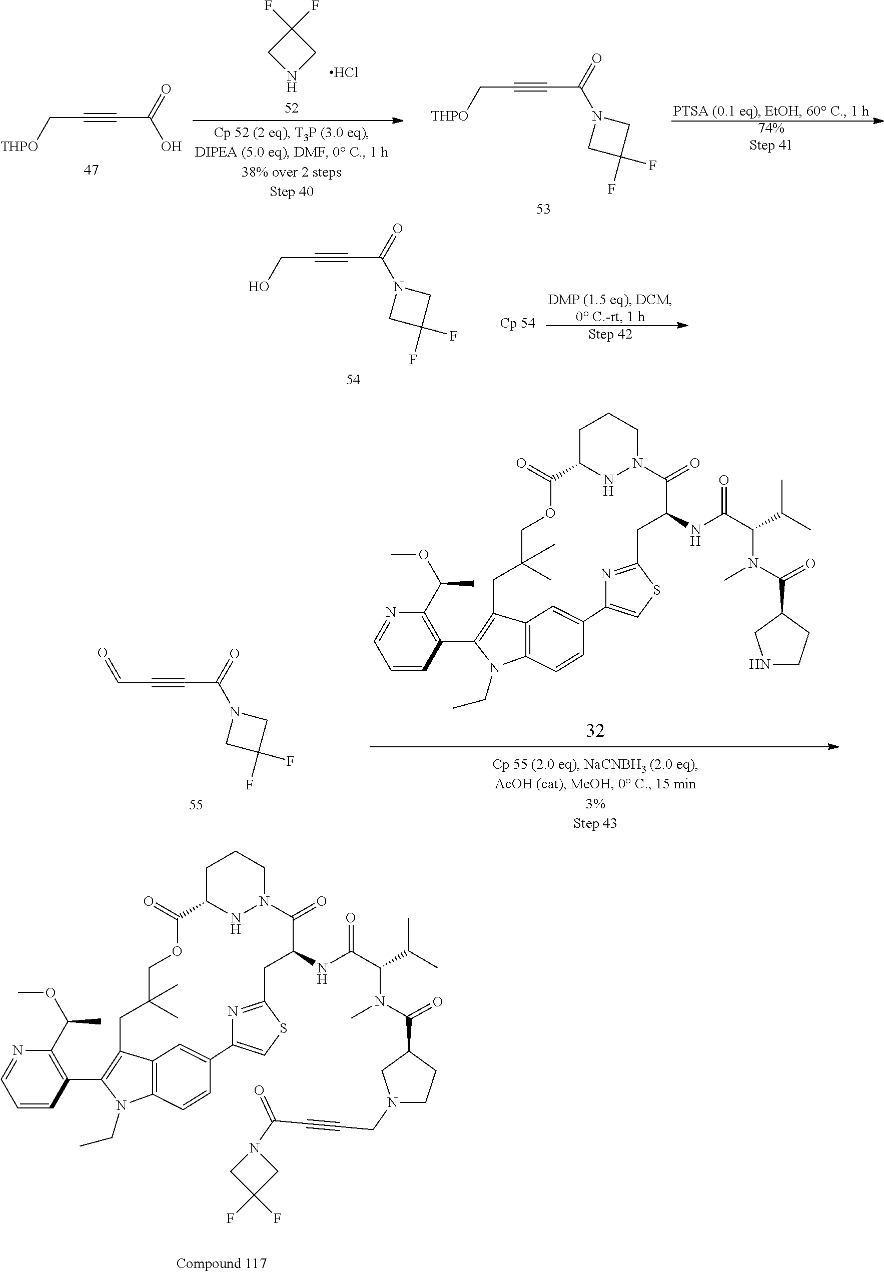

### Step 40

Synthesis of 1-(3,3-difluoroazetidin-1-yl)-4-((tetrahydro-2H-pyran-2-yl)oxy)but-2-yn-1-one (53): To a stirred solution of 3,3-difluoroazetidine hydrogen chloride 52 (1.40 g, 2 eq, 10.9 mmol) in DMF (20 mL) at 0° C. under Ar atmosphere was added DIPEA (4.8 mL, 5 eq, 27.2 mmol) and stirred for 5 min. Subsequently, 4-(oxan-2-yloxy)but-2-ynoic acid 47 (3.25 g, 5.45 mmol) was added followed by T₃P (50% in ethyl acetate, 33.5 mL, 3 eq, 16.3 mmol) and the reaction mixture was stirred for 1 h at 0° C. After completion of the reaction (monitoring by TLC), the reaction mixture was diluted with EtOAc (100 mL) and washed with chilled brine solution (3×100 mL). The combined organic layer was dried over anhydrous Na₂SO₄, filtered and evaporated under reduced pressure to get a crude residue, which was purified by Combi-flash column chromatography over silica gel using 5% MeOH in DCM eventually to obtain 1-(3,3-difluoroazetidin-1-yl)-4-((tetrahydro-2H-pyran-2-yl)oxy)but-2-yn-1-one 53 (2 g, 38% over 2 steps) as a colorless liquid. LCMS (ESI): m/z [M+H]⁺ calc'd for C₁₂H₁₆F₂NO₃ 260.11; found 260.11.

### Step 41

Synthesis of 1-(3,3-difluoroazetidin-1-yl)-4-hydroxybut-2-yn-1-one (45): To a stirred solution of 1-(3,3-difluoroazetidin-1-yl)-4-(oxan-2-yloxy)but-2-yn-1-one 53 (1 g, 3.86 mmol) in EtOH (50 mL) was added PTSA (67 mg, 0.1 eq, 0.386 mmol), and the reaction mixture was stirred at 60° C. for 1 h. After completion of the reaction (monitoring by TLC), the reaction mixture was evaporated under reduced pressure and diluted with EtOAc (100 mL). The organic layer was washed with saturated aq. solution of NaHCO₃ (3×50 mL) followed by brine (2×50 mL). The combined organic layer was dried over anhydrous Na₂SO₄, filtered and concentrated under reduced pressure to get a crude, which was purified by Combi-flash column chromatography over silica gel using 30-50% EtOAc in n-heptane to obtain 1-(3,3-difluoroazetidin-1-yl)-4-hydroxybut-2-yn-1-one 54 (500 mg, 74%) as a colorless liquid. ¹H NMR (400 MHz, CDCl₃): δ 5.52 (t, J=6 Hz, 1H), 4.24 (d, J=6 Hz, 2H), 4.60 (t, J=12 Hz, 2H), 4.37 (t, J=12.4 Hz, 2H).

### Step 42

Synthesis of 4-(3,3-difluoroazetidin-1-yl)-4-oxobut-2-ynal (55): To a stirred solution of 1-(3-fluoroazetidin-1-yl)-4-hydroxybut-2-yn-1-one 54 (500 mg, 2.85 mmol) in DCM (10 mL) at 0° C. was added DMP (1.8 g, 1.5 eq, 4.27 mmol) portion wise. The reaction mixture was allowed to warm to rt and stirred for 1 h. After completion of reaction (monitoring by TLC), the reaction mixture was diluted with DCM (100 mL), filtered through a Celite pad, and washed thoroughly with DCM. The combined filtrates were concentrated under reduced pressure to get the crude 4-(3,3-difluoroazetidin-1-yl)-4-oxobut-2-ynal 55 (300 mg, contaminated with DMP impurities) as a brown sticky liquid, which was used in the next step without further purification.

### Step 43

Synthesis of (3S)-1-(4-(3,3-difluoroazetidin-1-yl)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (Compound 117): To a stirred solution of (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide 32 (250 mg, 0.297 mmol) and 4-(3,3-difluoroazetidin-1-yl)-4-oxobut-2-ynal 55 (103 mg, 2 eq, 0.594 mmol) in methanol (5 mL) was added AcOH (catalytic, 10 μL) at 0° C. Subsequently, NaCNBH₃ (37.4 mg, 2 eq, 0.594 mmol) was added and the reaction mixture was stirred for 15 min. After completion of reaction (monitored by TLC and LCMS), the reaction mixture was diluted with water (10 mL) and extracted with 10% MeOH in DCM (2×50 mL). The combined organic layer was dried over anhydrous Na₂SO₄, filtered and concentrated under reduced pressure to get a crude which was purified by RP-HPLC to eventually afford (3S)-1-(4-(3,3-difluoroazetidin-1-yl)-4-oxobut-2-yn-1-yl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide, Compound 117 (9.8 mg, 3% w.r.t Cp 32, 97.03% purity) as a white solid. ¹H NMR (400 MHz, DMSO-d₆): δ 8.74 (dd, J=4.4, 1.2 Hz, 1H), 8.48 (d, J=4.8 Hz, 1H), 7.85 (s, 1H), 7.83-7.72 (m, 2H), 7.57 (d, J=8.4 Hz, 1H), 7.51 (q, J=4.8 Hz, 1H), 5.52-5.42 (m, 1H), 5.14-5.05 (m, 1H), 4.76 (d, J=10.8 Hz, 1H), 4.62 (q, J=12 Hz, 2H), 4.41-4.28 (m, 4H), 4.28-4.26 (m, 2H), 4.01 (d, J=10.4 Hz, 1H), 3.71-3.66 (m, 3H), 3.65-3.59 (m, 3H), 3.26 (d, J=2.4 Hz, 3H), 2.95 (s, 3H), 2.86 (s, 3H), 2.83-2.72 (m, 1H), 2.62-2.59 (m, 2H), 2.49-2.36 (m, 2H), 2.22-2.08 (m, 3H), 2.07-1.98 (m, 1H), 1.97-1.88 (m, 1H), 1.79 (brs, 2H), 1.52-1.45 (m, 1H), 1.35 (d, J=6 Hz, 3H), 1.28 (s, 3H), 0.96-0.89 (m, 6H), 0.85-0.75 (m, 6H) [along with minor peaks, arising presumably due to rotameric components, intensity of which reduced in VT ¹H NMR]; LCMS (ESI): m/z [M+H]⁺ calc'd for C₅₂H₆₆F₂N₉O₇S 998.48; found 998.61.

### Example 22

### Compound 118

### Synthesis of (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-1-(4-(3-fluoroazetidin-1-yl)-4-oxobut-2-yn-1-yl)-N-methylpyrrolidine-3-carboxamide (Compound 118)

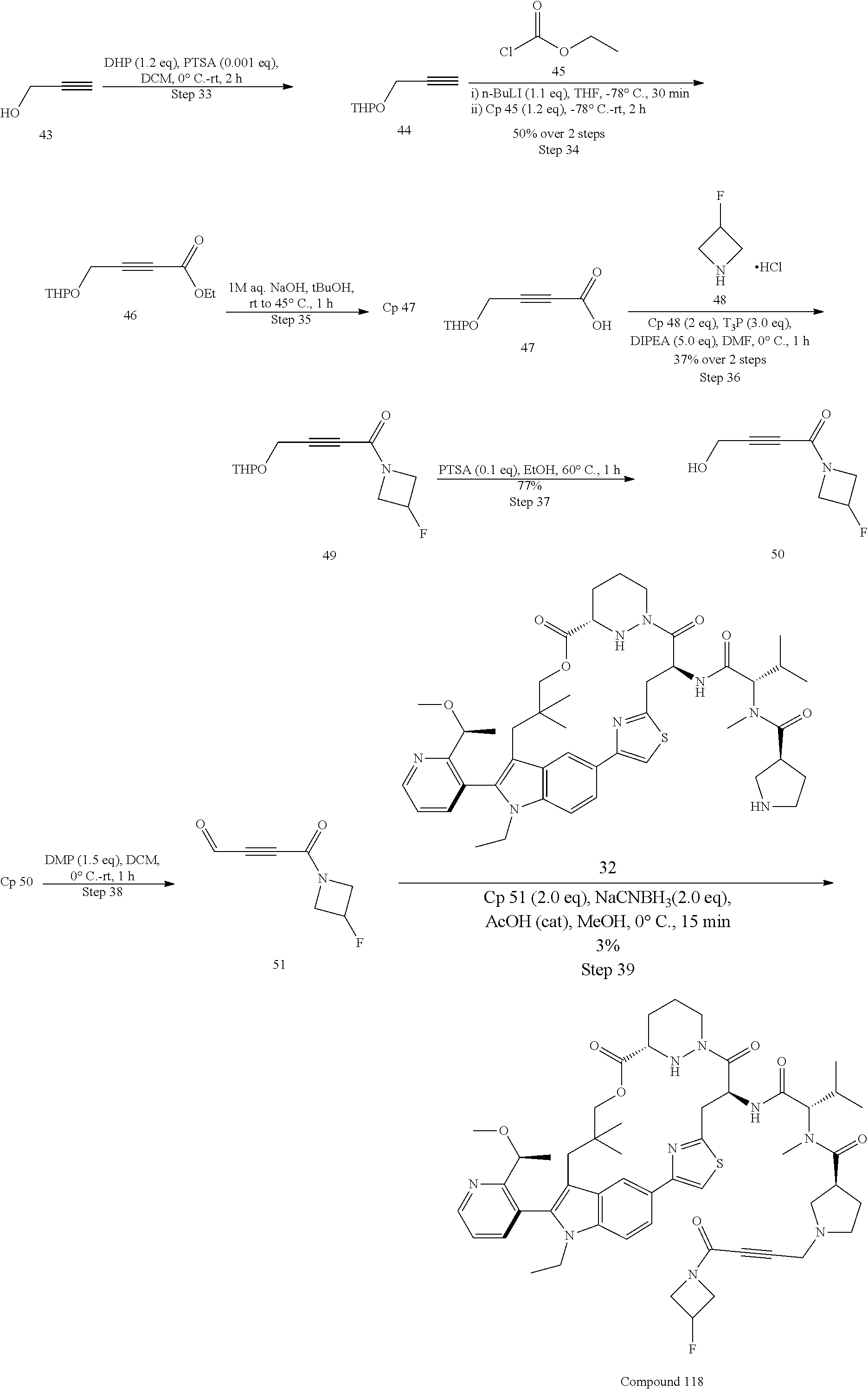

### Step 33

Synthesis of 2-(prop-2-yn-1-yloxy)tetrahydro-2H-pyran (44): To a stirred solution of 3-butyn-1-ol 43 (4.2 mL, 71.4 mmol) in DCM (100 mL) at 0° C., was added PTSA (150 mg, 0.001 eq, 0.7 mmol) followed by DHP (7.2 mL, 1.2 eq, 78.5 mmol) and the reaction mixture was stirred at this temperature for 10 min. Subsequently, the reaction mixture was warmed to rt and stirred for next 2 h. After completion of reaction (monitoring by TLC), the reaction mixture was diluted with saturated aq. solution of NaHCO₃ (30 mL) and extracted with DCM (2×50 mL). The combined organic layer was dried over anhydrous Na₂SO₄, filtered and concentrated in vacuo to get the crude 2-(prop-2-yn-1-yloxy)tetrahydro-2H-pyran 44 (9.5 g) as a yellow liquid, which was used in the next step without further purification.

### Step 34

Synthesis of ethyl 4-((tetrahydro-2H-pyran-2-yl)oxy)but-2-ynoate (46): To a stirred solution of 2-(prop-2-yn-1-yloxy)tetrahydro-2H-pyran 44 (7.5 g, 53.5 mmol) in THF (150 mL) was added ^{t}BuLi (24 mL, 2.5 M in hexane, 1.1 eq, 58.5 mmol) at −78° C. under nitrogen atmosphere, and the reaction mixture was stirred at the same temperature for 30 min. Subsequently, ethyl chloroformate 45 (6.2 mL, 1.2 eq, 64.2 mmol) was added dropwise at −78° C., the reaction mixture gradually warmed to rt and stirred for next 2 h. After completion of reaction (monitoring by TLC), the reaction mixture was cooled to 0° C., quenched with saturated aq. solution of NH₄Cl (100 mL) and extracted with EtOAc (3×100 mL). The combined organic layer was dried over anhydrous Na₂SO₄, filtered and evaporated under reduced pressure to get a crude which was purified by Combi-flash column chromatography over silica gel using 30-50% EtOAc in n-hexane to eventually obtain ethyl 4-((tetrahydro-2H-pyran-2-yl)oxy)but-2-ynoate 46 (6.3 g, 50% over 2 steps) as a colorless liquid. ¹H NMR (400 MHz, DMSO-d₆): δ 1.68-1.46 (m, 6H), 3.71-3.70 (m, 2H), 4.62-4.61 (m, 1H), 4.11 (s, 2H), 4.46 (q, J=16.8 Hz, 2H), 1.21 (t, J=7.2 Hz, 3H).

### Step 35

Synthesis of 4-((tetrahydro-2H-pyran-2-yl)oxy)but-2-ynoic acid (47): To stirred a solution of ethyl 4-((tetrahydro-2H-pyran-2-yl)oxy)but-2-ynoate 46 (6 g, 5.65 mmol) in ^{t}BuOH (100 mL) was added 1M aq. solution of NaOH (60 mL) at rt and the reaction mixture was stirred at 45° C. for 1 h. After completion of reaction (monitoring by TLC), the reaction mixture was concentrated under reduced pressure to get a crude, which was acidified by NaHSO₄ to pH ˜ 4 and extracted with 25% IPA in CHCl₃ (2×100 mL). The combined organic layer was dried over anhydrous Na₂SO₄, filtered and concentrated under reduced pressure to get the crude 4-((tetrahydro-2H-pyran-2-yl)oxy)but-2-ynoic acid 47 (4.5 g) as a light yellow liquid, which was used in the next step without further purification.

### Step 36

Synthesis of 1-(3-fluoroazetidin-1-yl)-4-((tetrahydro-2H-pyran-2-yl)oxy)but-2-yn-1-one (49): To a stirred solution of 3-fluoroazetidine hydrochloride 48 (3 g, 2 eq, 27 mmol) in DMF (30 mL) at 0° C. under Ar atmosphere was added DIPEA (11.5 mL, 5 eq, 67.5 mmol) and stirred for 5 min. Subsequently, 4-(oxan-2-yloxy)but-2-ynoic acid 47 (2.5 g, 13.5 mmol) was added followed by T₃P (26 mL, 50% in ethyl acetate, 3 eq, 40.5 mmol) and the reaction mixture was stirred for 1 h at 0° C. After completion of the reaction (monitoring by TLC), the reaction mixture was diluted with EtOAc (100 mL) and washed with chilled brine solution (3×100 mL). The combined organic layer was dried over anhydrous Na₂SO₄, filtered and evaporated under reduced pressure to get a crude residue, which was purified by Combi-flash column chromatography over silica gel using 5% MeOH in DCM eventually to obtain 1-(3-fluoroazetidin-1-yl)-4-((tetrahydro-2H-pyran-2-yl)oxy)but-2-yn-1-one 49 (1.2 g, 37% over 2 steps) as colorless oil. LCMS (ESI): m/z [M+H]⁺ calc'd for C₁₂H₁₇FNO₃ 242.12; found 242.17.

### Step 37

Synthesis of 1-(3-fluoroazetidin-1-yl)-4-hydroxybut-2-yn-1-one (50): To a stirred solution of 1-(3-fluoroazetidin-1-yl)-4-((tetrahydro-2H-pyran-2-yl)oxy)but-2-yn-1-one 49 (1.2 g, 4.97 mmol) in EtOH (25 mL) was added PTSA (90 mg, 0.1 eq, 0.497 mmol), and the reaction mixture was stirred at 60° C. for 1 h. After completion of the reaction (monitoring by TLC), the reaction mixture was evaporated under reduced pressure and diluted with EtOAc (100 mL). The organic layer was washed with saturated aq. solution of NaHCO₃ (3×50 mL) followed by brine (2×50 mL). The combined organic layer was dried over anhydrous Na₂SO₄, filtered, and concentrated under reduced pressure to get a crude, which was purified by Combi-flash column chromatography over silica gel using 30-50% EtOAc in n-heptane to obtain 1-(3-fluoroazetidin-1-yl)-4-hydroxybut-2-yn-1-one 50 (600 mg, 77%) as colorless liquid. ¹H NMR (400 MHz, CDCl₃): δ 5.30 (t, J=6 Hz, 1H), 4.30 (d, J=6.1 Hz, 2H), 4.10-3.92 (m, 5H).

### Step 38

Synthesis of 4-(3-fluoroazetidin-1-yl)-4-oxobut-2-ynal (51): To a stirred solution of 1-(3-fluoroazetidin-1-yl)-4-hydroxybut-2-yn-1-one 50 (500 mg, 3.18 mmol) in DCM (15 mL) at 0° C. was added DMP (2.0 g, 1.5 eq, 4.77 mmol) portion wise. The reaction mixture was allowed to warm to rt and stirred for 1 h. After completion of reaction (monitoring by TLC), the reaction mixture was diluted with DCM (100 mL), filtered through a Celite pad, and washed thoroughly with DCM. The combined filtrates were concentrated under reduced pressure to get the crude 4-(3-fluoroazetidin-1-yl)-4-oxobut-2-ynal 51 (300 mg, contaminated with DMP impurities) as a brown sticky liquid, which which was used in the next step without further purification.

### Step 39

Synthesis of (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-1-(4-(3-fluoroazetidin-1-yl)-4-oxobut-2-yn-1-yl)-N-methylpyrrolidine-3-carboxamide (Compound 118): To a stirred solution of (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide 32 (200 mg, 0.237 mmol) and 4-(3-fluoroazetidin-1-yl)-4-oxobut-2-ynal 51 (67 mg, 2 eq, 0.475 mmol) in methanol (5 mL) was added AcOH (catalytic, 10 μL) at 0° C. Subsequently, NaCNBH₃ (29 mg, 2 eq, 0.594 mmol) was added and the reaction mixture was stirred for 15 min. After completion of reaction (monitored by TLC and LCMS), the reaction mixture was diluted with water (10 mL) and extracted with 10% MeOH in DCM (2×50 mL). The combined organic layer was dried over anhydrous Na₂SO₄, filtered and concentrated under reduced pressure to get a crude which was purified by RP-HPLC to eventually afford (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-1-(4-(3-fluoroazetidin-1-yl)-4-oxobut-2-yn-1-yl)-N-methylpyrrolidine-3-carboxamide, Compound 118 (7 mg, 3% w.r.t Cp 32, 98.03% purity) as a white solid. ¹H NMR (400 MHz, DMSO-d6): δ 8.75 (dd, J=1.6, 4.4 Hz, 1H), 8.48 (d, J=4.8 Hz, 1H), 7.85-7.77 (m, 2H), 7.73 (d, J=8.4 Hz, 1H), 7.56 (d, J=8.4 Hz, 1H), 7.51 (dd, J=4.8, 7.6 Hz, 1H), 5.51-5.31 (m, 2H), 5.13-5.04 (m, 1H), 4.74 (d, J=10.8 Hz, 1H), 4.52-4.44 (m, 1H), 4.35-4.28 (m, 2H), 4.24-4.16 (m, 4H), 4.11 (q, J=5.2 Hz, 2H), 4.02-4.01 (m, 1H), 3.66-3.64 (m, 3H), 3.59-3.51 (m, 3H), 3.28-3.26 (m, 4H), 3.16 (d, J=5.2 Hz, 3H), 3.11-3.10 (m, 1H), 2.95-2.83 (m, 5H), 2.59-2.57 (m, 1H), 2.43-2.36 (m, 2H), 2.10-2.06 (m, 3H), 1.79-1.63 (m, 2H), 1.51-1.46 (m, 1H), 1.36 (d, J=6 Hz, 3H), 1.23 (s, 2H), 0.96-0.89 (m, 6H), 0.84-0.75 (m, 6H) [along with minor peaks, arising presumably due to rotameric components, intensity of which reduced in VT ¹H NMR]; LCMS (ESI): m/z [M+H]⁺ calc'd for C₅₂H₆₇FN₉O₇S 980.49; found 980.81.

### Additional Exemplary Compounds

The representative compounds prepared or can be prepared from readily available starting materials using the general methods and procedures described herein are depicted in Table 1A and Table 1B.

### Example B1a

### KRas G12C In Vitro Inhibitory Activity

The present example evaluates the ability of compounds provided herein to inhibit proliferation of KRAS mutant cells.

The inhibitory effect on cell proliferation was investigated in the human KRAS G12C mutated cell line MIA PaCa-2 (Yunis et al., 1977, Int. J. Cancer 19(1):128-135; ATCC catalog no. CRL-1420). Cells were maintained in RPMI-1640 medium (ThermoFisher catalog no. 61870036) supplemented with 10% of Heat Inactivated FBS (ThermoFisher catalog no. A31605) and 1% Pen-Strep (ThermoFisher catalog no. 10378016) and cultured at 37° C. in a humidified incubator with 5% CO₂. Cells were grown by adhering to culture flasks, and maintained at 70%-80% confluency.

ATP is present in all metabolically active cells and is considered as a marker for viable cells. The number of metabolically active live cells in culture was determined using the CellTiter-Glo kit (Promega catalog no. G7572), an ATP monitoring system based on the production of luminescence by the reaction of ATP with added UltraGlo® recombinant luciferase (Kawano et al., 2016, PLOS One, 8; 11(7):e0158888), according to the supplier's experimental recommendations. The cell proliferation assay was based on a 96 well plate format.

Test compounds were dissolved at 10 mM in DMSO (Sigma catalog no. D8418; purity >99.9%) and stored at −20° C. Eight concentrations of test compound were assessed in duplicate in an individual test occasion in parallel in the selected cell lines. MRTX-849 and AMG-510 were used as reference compounds and tested in duplicate at eight concentrations. 100% of proliferation is represented by the untreated cells (0.2% DMSO).

On the day of the experiment (TO), cells were detached with TrypLE (ThermoFisher catalog no. 12604054), quantified using the cell viability analyser NucleoCounter (Chemometec NC-200) and resuspended in fresh medium at a cell density of about 5000 cells per 200 μL medium. 200 μL of cell suspension were added to each well of a 96 well plate.

Two hours after seeding cells, compounds were added at desired concentrations using a D300e compound dispenser (Life Sciences Tecan D300) with shaking after each compound addition. Assay plates were incubated at 37° C. in a humidified incubator with 5% CO₂.

To measure 3 day inhibition of cell proliferation on day 3 (T3), assay plates were equilibrated to room temperature for approximately 15 min, and the Promega CellTiterGlo® reagent was added at 40 μL/well. Contents were mixed for 5 min on an orbital shaker to induce cell lysis, and then incubated at room temperature for an additional 10 min in the dark to stabilize the luminescent signal. Luminescence was read using a GloMax GM300 plate reader (Promega) using the luminescence for 96 well plate standard protocol.

To measure 7 day inhibition of cell proliferation on day 3 (T3), 150 μL of growth media was removed from each well of the assay plates and replaced with 150 uL fresh growth media, and fresh compounds added using a D300e compound dispenser (Life Sciences Tecan D300) at desired concentrations with shaking between each compound addition. Assay plates were incubated at 37° C. in a humidified incubator with 5% CO₂. On day 7 (T7) assay plates were equilibrated to room temperature for approximately 15 min, and the Promega CellTiterGlo® reagent was added at 40 μL/well. Contents were mixed for 5 min on an orbital shaker to induce cell lysis, and then incubated at room temperature for an additional 10 min in the dark to stabilize the luminescent signal. Luminescence was read using a GloMax GM300 plate reader (Promega) using the luminescence for 96 well plate standard protocol.

Data was expressed as % of inhibition compared to the 0.2% DMSO negative no drug control, and is calculated as follows: % inhibition=100−[(RLU sample)×100/(RLU average controls*)], where * indicates the average for 0.2% DMSO.

### Example B1b

### KRas G12D In Vitro Inhibitory Activity

The present example evaluates the ability of compounds provided herein to inhibit proliferation of KRAS mutant cells.

The proliferation inhibitory effect was investigated in the human KRAS G12D mutated cell line SUIT-2 (Iwamura et al., 1987, Jpn. J. Cancer Res. 78.54-62; JCRB catalog no. JCRB1094). Cells were maintained in RPMI-1640 medium (ThermoFisher catalog no. 61870036) supplemented with 10% of Heat Inactivated FBS (ThermoFisher catalog no. A31605) and 1% Pen-Strep (ThermoFisher catalog no. 10378016) and cultured at 37° C. in a humidified incubator with 5% CO₂. Cells were grown by adhering to culture flasks, and maintained at 70%-80% confluency.

ATP is present in all metabolically active cells and is considered as a marker for viable cells. The number of metabolically active live cells in culture was determined using the CellTiter-Glo kit (Promega catalog no. G7572), an ATP monitoring system based on the production of luminescence by the reaction of ATP with added UltraGlo® recombinant luciferase (Kawano et al., 2016, PLOS One, 8; 11(7):e0158888), according to the supplier's experimental recommendations. The cell proliferation assay was based on a 96 well plate format.

Test compounds were dissolved at 10 mM in DMSO (Sigma catalog no. D8418; purity >99.9%) and stored at −20° C. Eight concentrations of test compound were assessed in duplicate in an individual test occasion in parallel in the selected cell lines. MRTX-849 and AMG-510 were used as reference compounds and tested in duplicate at eight concentrations. 100% of proliferation is represented by the untreated cells (0.1% DMSO).

On the day of the experiment (TO), cells were detached with TrypLE (ThermoFisher catalog no. 12604054) quantified using the cell viability analyzer NucleoCounter (Chemometec NC-200) and resuspended in fresh medium at a cell density of about 1000 cells per 200 μL medium. 200 μL of cell suspension were added to each well of a 96 well plate.

Two 2 hours after seeding cells, compounds were added at desired concentrations using a D300e compound dispenser (Life Sciences Tecan D300) with shaking after each compound addition. Assay plates were incubated at 37° C. in a humidified incubator with 5% CO₂.

On day 3 (T3), 150 μL of growth media was removed from each well of the assay plates and replaced with 150 uL fresh growth media, and fresh compounds added using a D300e compound dispenser (Life Sciences Tecan D300) at desired concentrations with shaking between each compound addition. Assay plates were incubated at 37° C. in a humidified incubator with 5% CO₂. On day 7 (T7), assay plates were equilibrated to room temperature for approximately 15 min, and Promega CellTiterGlo® reagent was added at 40 μL/well. Contents were mixed for 5 min on an orbital shaker to induce cell lysis, and then incubated at room temperature for an additional 10 min in the dark to stabilize the luminescent signal. Luminescence was read using a GloMax GM300 plate reader (Promega) using the luminescence for 96 well plate standard protocol.

Data was expressed as % of inhibition compared to the 0.2% DMSO no drug control, and is calculated as follows: % inhibition=100-[(RLU sample)×100/(RLU average controls*)], where * indicates the average for 0.2% DMSO.

Results were analyzed by GraphPad (Prism) and IC₅₀ values were calculated by non-linear regression using four parameter-logistic equation. IC₅₀ (nM) values are reported in the data table below for 4 and/or 7-day incubations: **** <1, ***>1 to >10, **>10 to >100, and * >100

Compounds of the disclosure provided the following IC₅₀ values:

TABLE 1A
Representative compounds and their IC₅₀ values

| | | | MIA | MIA | |
|---|---|---|---|---|---|
| | | | PaCa-2 | PaCa-2 | SUIT-2 |
| | | | Day 7 | Day 3 | Day 7 |
| | Structure | MW | IC50 | IC50 | IC50 |
| 1 | 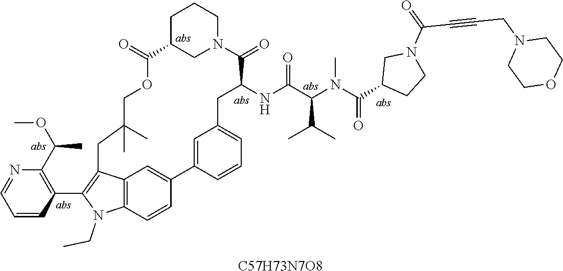 | 984.23 | ** | | * |
| 2 | 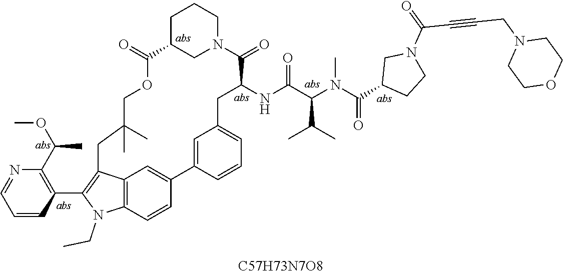 | 984.23 | * | * | — |
| 3 | 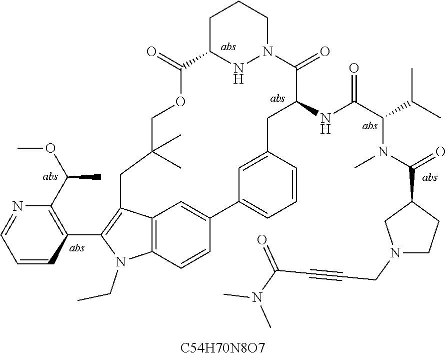 | 943.18 | *** | *** | * |
| 4 | 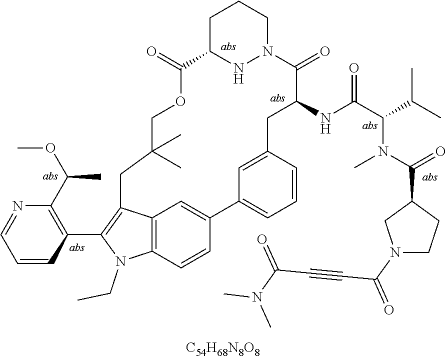 | 957.17 | * | | * |
| 5 | 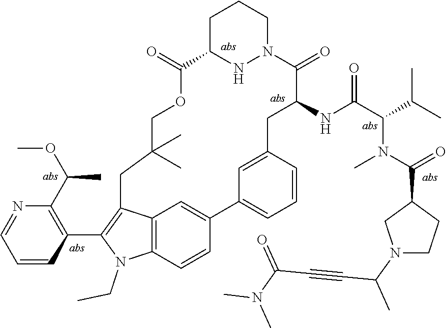 | 957.21 | ** | *** | * |

*PK45H cell line

TABLE 1B
Representative compounds and their IC₅₀ values

| | | | MIA | MIA |
|---|---|---|---|---|
| | | | PaCa-2 | PaCa-2 |
| Pat | | | Day 7 | Day 3 |
| ID | Structure | MW | IC50 | IC50 |
| 101 | 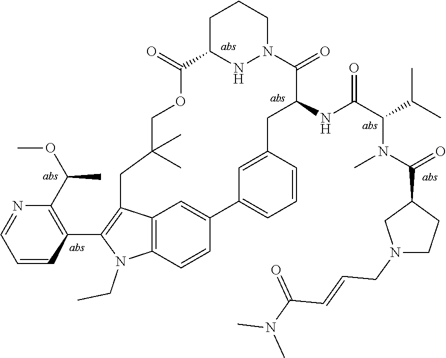 | 945.2 | ** | ** |
| 102 | 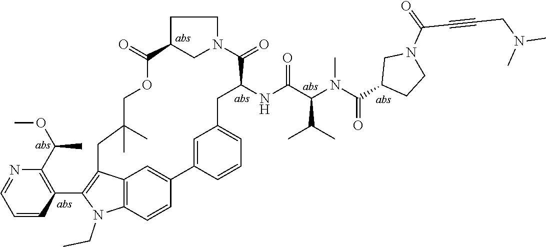 | 928.17 | | * |
| 103 | 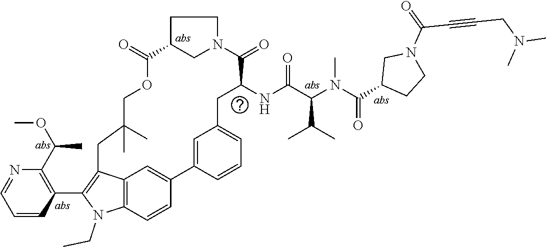 | 928.17 | | * |
| 104 | 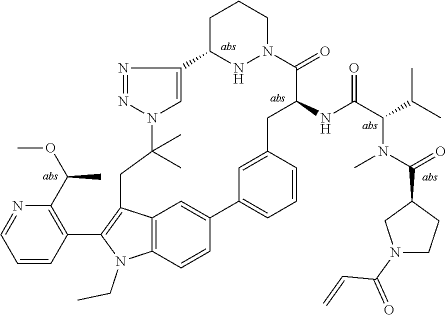 [SMILES: C=CC(=O)N1CC[C@H](C(=O)N(C)[C@H](C(=O)N[C@H]2Cc3cccc(c3)-c3ccc4c(c3)c(c(-c3cccnc3[C@H](C)OC)n4CC)CC(C)(C)n3cc(nn3)[C@@H]3CCCN(N3)C2=O)C(C)C)C1] | 897.12 | | * |
| 105 | 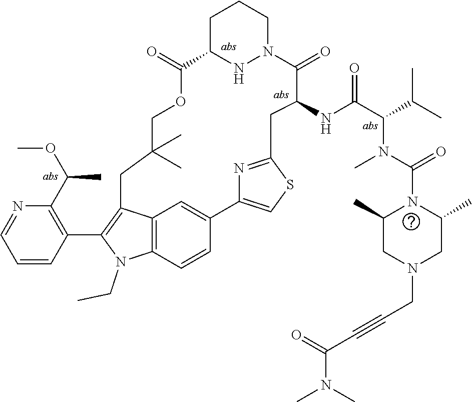 [SMILES: CCn1c(-c2cccnc2[C@H](C)OC)c2c3cc(ccc31)-c1csc(n1)C[C@H](NC(=O)[C@H](C(C)C)N(C)C(=O)N1[C@H](C)CN(CC#CC(=O)N(C)C)C[C@H]1C)C(=O)N1CCC[C@H](N1)C(=O)OCC(C)(C)C2] | 993.27 | ** | ** |
| 106 | 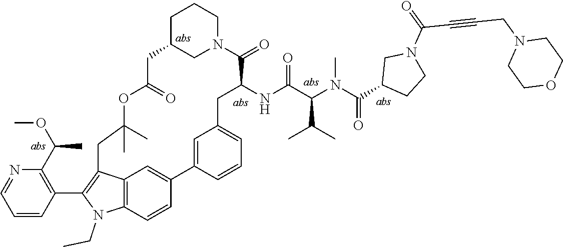 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#CCN3CCOCC3)C1)C(=O)N1CCC[C@@H](CC(=O)OC(C)(C)C2)C1] | 984.23 | | * |
| 107 | 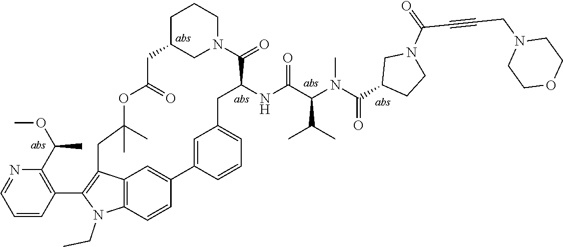 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#CCN3CCOCC3)C1)C(=O)N1CCC[C@@H](CC(=O)OC(C)(C)C2)C1] | 984.23 | * | |
| 108 | 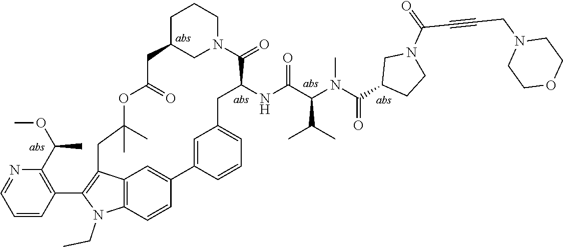 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#CCN3CCOCC3)C1)C(=O)N1CCC[C@H](CC(=O)OC(C)(C)C2)C1] | 984.23 | | * |
| 109 | 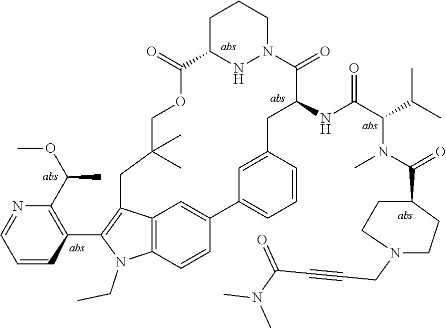 | 957.21 | | *** |
| 110 | 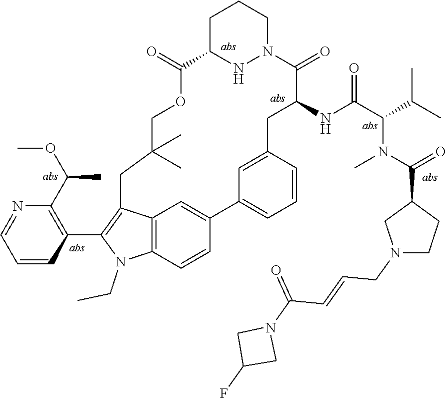 | 975.2 | | **** |
| 111 | 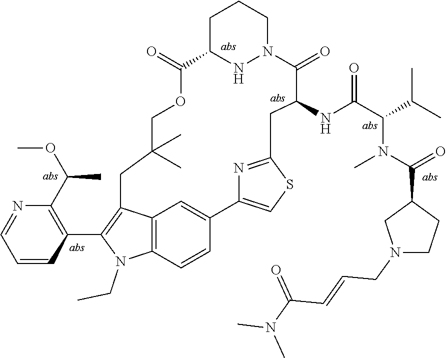 | 952.21 | | ** |
| 112 | 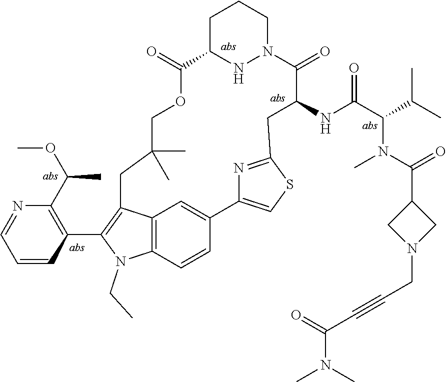 | 936.17 | | *** |
| 113 | 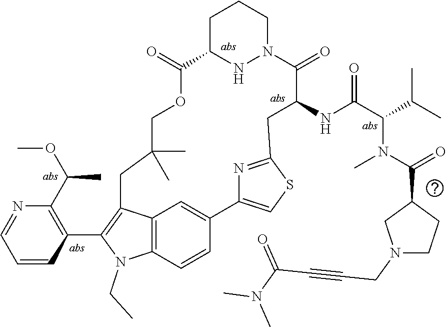 | 950.2 | | **** |
| 114 | 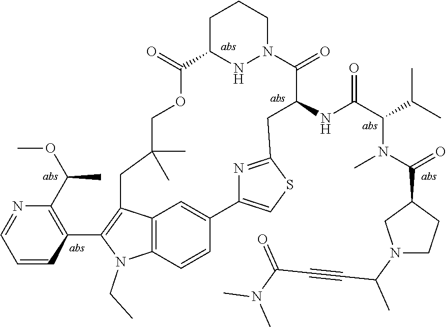 | 964.23 | | **** |
| 115 | 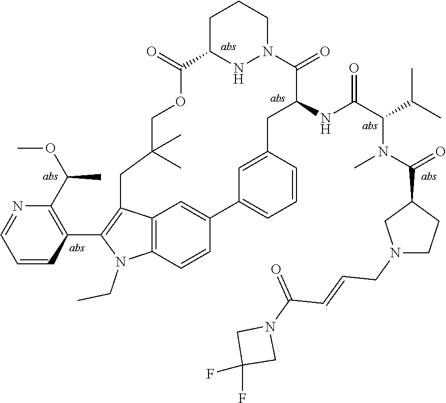 | 993.19 | | **** |
| 116 | 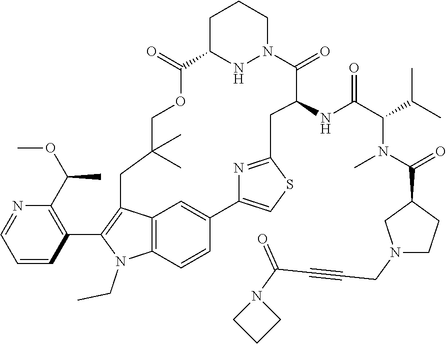 | 962.49 | *** | * |
| 117 | 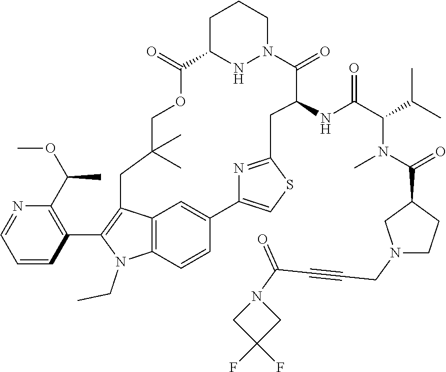 | 998.48 | *** | * |
| 118 | 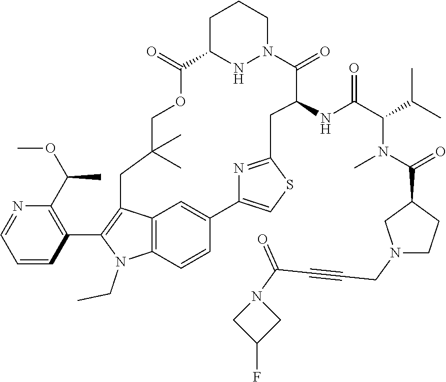 | 980.49 | *** | * |

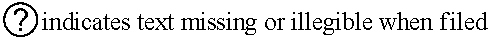

### Example B2: Pharmaceutical Compositions

The compositions described below are presented with a compound of Formula (B-I), (I)—(XXVIIId) for illustrative purposes.

### Example B2a: Parenteral Composition

To prepare a parenteral pharmaceutical composition suitable for administration by injection, 100 mg of a water-soluble salt of a compound of Formula (B-I), (I)—(XXVIIId) is dissolved in DMSO and then mixed with 10 mL of 0.9% sterile saline. The mixture is incorporated into a dosage unit form suitable for administration by injection.

### Example B2b: Oral Composition

To prepare a pharmaceutical composition for oral delivery, 100 mg of a compound of Formula (B-I), (I)—(XXVIIId) is mixed with 750 mg of starch. The mixture is incorporated into an oral dosage unit for, such as a hard gelatin capsule, which is suitable for oral administration.

### Example B2c: Sublingual (Hard Lozenge) Composition

To prepare a pharmaceutical composition for buccal delivery, such as a hard lozenge, mix 100 mg of a compound of Formula (B-I), (I)—(XXVIIId) with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example B2d: Inhalation Composition

To prepare a pharmaceutical composition for inhalation delivery, 20 mg of a compound of Formula (B-I), (I)—(XXVIIId) is mixed with 50 mg of anhydrous citric acid and 100 mL of 0.9% sodium chloride solution. The mixture is incorporated into an inhalation delivery unit, such as a nebulizer, which is suitable for inhalation administration.

### Example B2e: Rectal Gel Composition

To prepare a pharmaceutical composition for rectal delivery, 100 mg of a compound of Formula (B-I), (I)—(XXVIIId) is mixed with 2.5 g of methylcellulose (1500 mPa), 100 mg of methylparapen, 5 g of glycerin and 100 mL of purified water. The resulting gel mixture is then incorporated into rectal delivery units, such as syringes, which are suitable for rectal administration.

### Example B2f: Topical Gel Composition

To prepare a pharmaceutical topical gel composition, 100 mg of a compound of Formula (B-I), (I)—(XXVIIId) is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topic administration.

### Example B2g: Ophthalmic Solution Composition

To prepare a pharmaceutical ophthalmic solution composition, 100 mg of a compound of Formula (B-I), (I)—(XXVIIId) is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

At least some of the chemical names of compounds provided herein as given and set forth in this application, may have been generated on an automated basis by use of a commercially available chemical naming software program, and have not been independently verified In the instance where the indicated chemical name and the depicted structure differ, the depicted structure will control. In the chemical structures where a chiral center exists in a structure but no specific stereochemistry is shown for the chiral center, both enantiomers associated with the chiral structure are encompassed by the structure.

What is claimed is:

1. A compound according to formula (B-I): 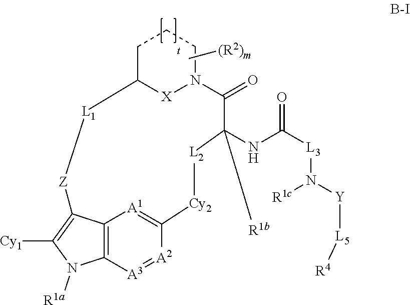
or a stereoisomer or a pharmaceutically acceptable salt thereof,
wherein:
each A¹, A², and A³ is independently —CR³=, or —N=; each R³ is independently H, halo, cyano, alkyl, haloalkyl, hydroxy, or alkoxy;
X is —CR^{1e}R^{1f}—, or —NR^{1g}—;
Z is absent or substituted or unsubstituted C₁-C₄ alkylene;
each Cy¹ and Cy² is independently substituted or unsubstituted aryl or heteroaryl;
Cy³ is substituted or unsubstituted cycloalkylene, or substituted or unsubstituted heterocycloalkyl;
L₁ is —C(O)—O—, —C(O)—N(R^{1d})—, or -Cy⁴-; Cy⁴ is substituted or unsubstituted heteroaryl; and —O— of —C(O)—O—, or —N(R^{1d})— of —C(O)—N(R^{1d})— is attached to Z; or —O— of —C(O)—O—, or —N(R^{1d})— of —C(O)—N(R^{1d})— is attached to the pyrrolo ring when Z is absent;
each L₂, and L₃, is independently substituted or unsubstituted C₁-C₄ alkylene;
Y is —C(O)—, —C(H)(CH₂F)—, —C(H)(CHF₂)—, or —C(H)(CF₃)—;
L₅ is substituted or unsubstituted C₁-C₆ alkylene, or Cy₃;
each R^{1a}, R^{1b}, R^{1c}, R^{1d}, R^{1e}, R^{1f}, and R^{1g} is independently H, or substituted or unsubstituted C₁-C₄ alkyl;
each R² is independently halo, cyano, alkyl, haloalkyl, or alkoxy; m is 0, 1, or 2;
R⁴ is any one of groups selected from i), and ii):
i) —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl;
ii) -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e},
L₄ is C(O), substituted or unsubstituted C₁-C₄ alkylene, or substituted or unsubstituted cycloalkylene;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, unsubstituted or substituted with one or more groups selected from substituted or unsubstituted amino, and substituted or unsubstituted heterocycloalkyl having 1-2 heteroatoms independently selected from nitrogen, oxygen, and sulfur;
R^{6d} is substituted or unsubstituted amino, substituted or unsubstituted heterocycloalkyl having 1-2 heteroatoms independently selected from nitrogen, oxygen, and sulfur, substituted or unsubstituted aryl, substituted or unsubstituted heteroaryl, or 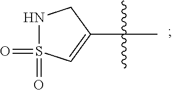 [SMILES: *C1=CS(=O)(=O)NC1]
R^{6e} is substituted or unsubstituted alkyl, substituted or unsubstituted aryl, substituted or unsubstituted heteroaryl, or 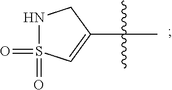 [SMILES: *C1=CS(=O)(=O)NC1]
each dotted bond is a single or a double bond; and
the subscript t is 0, 1, or 2;
provided that when
i) X is —NR^{1g}—, R^{1g} is H, each dotted bond is a single bond, and t is 1; then R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

2. The compound according claim 1, wherein L₅ is substituted or unsubstituted C₁-C₆ alkylene.

3. The compound according claim 1, wherein L₅ is substituted or unsubstituted Cy₃.

4. The compound according to any one of claims 1-3, wherein t is 0.

5. The compound according to any one of claims 1-3, wherein t is 1.

6. The compound according to any one of claims 1-3, wherein t is 2.

7. The compound according to any one of claims 1-3, wherein the compound is according to formula (I): 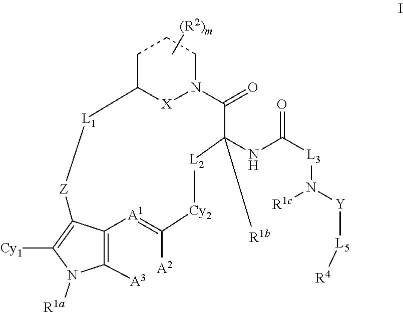
or a stereoisomer or a pharmaceutically acceptable salt thereof,
wherein:
each A¹, A², and A³ is independently —CR³=, or —N=; each R³ is independently H, halo, cyano, alkyl, haloalkyl, hydroxy, or alkoxy;
X is —CR^{1e}R^{1f}—, or —NR^{1g}—;
Z is absent or substituted or unsubstituted C₁-C₄ alkylene;
each Cy¹ and Cy² is independently substituted or unsubstituted aryl or heteroaryl;
Cy³ is substituted or unsubstituted heterocycloalkyl;
L¹ is —C(O)—O—, —C(O)—N(R^{1d})—, or -Cy⁴-; Cy⁴ is substituted or unsubstituted heteroaryl; and —O— of —C(O)—O—, or —N(R^{1d})— of —C(O)—N(R^{1d})— is attached to Z; or —O— of —C(O)—O—, or —N(R^{1d})— of —C(O)—N(R^{1d})— is attached to the pyrrolo ring when Z is absent;
each L², L³, and L⁴ is independently substituted or unsubstituted C₁-C₄ alkylene;
Y is —C(O)—, or —C(H)CH₂F—, or —C(H)CF₃—;
each R^{1a}, R^{1b}, R^{1c}, R^{1d}, R^{1e}, R^{1f}, and R^{1g} is independently H, or substituted or unsubstituted C₁-C₄ alkyl;
each R² is independently halo, cyano, alkyl, haloalkyl, or alkoxy; m is 0, 1, or 2;
R⁴ is any one of groups selected from i), and ii):
i) —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl;
ii) -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e},
L₄ is C(O), substituted or unsubstituted C₁-C₄ alkylene, or substituted or unsubstituted cycloalkyl;
each R^{6a} and R^{6b} is independently H, halo, CN, or C₁₋₆ alkyl; or R^{6a} and R^{6b} are joined together to form a bond; R^{6c} is H, halo, CN, or C₁₋₆ alkyl, unsubstituted or substituted with one or more groups selected from substituted or unsubstituted amino, and substituted or unsubstituted heterocycloalkyl having 1-2 heteroatoms independently selected from nitrogen, oxygen, and sulfur;
R^{6a} is substituted or unsubstituted amino, substituted or unsubstituted heterocycloalkyl having 1-2 heteroatoms independently selected from nitrogen, oxygen, and sulfur, substituted or unsubstituted aryl, substituted or unsubstituted heteroaryl, or 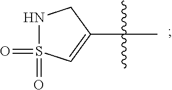 [SMILES: *C1=CS(=O)(=O)NC1]
R^{6e} is substituted or unsubstituted alkyl, substituted or unsubstituted aryl, substituted or unsubstituted heteroaryl, or 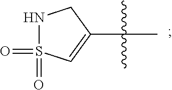 [SMILES: *C1=CS(=O)(=O)NC1]
and each dotted bond is a single or a double bond;
provided that when
i) X is —NR^{1g}—, R^{1g} is H, and each dotted bond is a single bond; then R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

8. The compound according any one of claims 1-7, wherein each A¹, A², and A³ is independently —CH═.

9. The compound according any one of claims 1-8, wherein L₁ is —C(O)—O—.

10. The compound according any one of claims 1-9, wherein L₂ is —CH₂—, —C(Me)H—, or —C(Me)₂-.

11. The compound according any one of claims 1-10, wherein Cy₂ is substituted or unsubstituted heteroaryl.

12. The compound according any one of claims 1-10, wherein Cy₂ is substituted or unsubstituted pyrrolyl, imidazolyl, thiazolyl, oxazolyl, or isoxazolyl.

13. The compound according any one of claims 1-10, wherein Cy₂ is substituted or unsubstituted thiazolyl.

14. The compound according any one of claims 1-10, wherein Cy₂ is substituted or unsubstituted pyridyl, or pyrimidinyl.

15. The compound according any one of claims 1-10, wherein Cy₂ is substituted or unsubstituted phenyl.

16. The compound according any one of claims 1-10, wherein Cy₂ is unsubstituted phenyl.

17. The compound according any one of claims 1-16, wherein Z is substituted or unsubstituted C₃-C₄ alkylene.

18. The compound according any one of claims 1-16, wherein Z is substituted or unsubstituted C₃ alkylene.

19. The compound according any one of claims 1-16, wherein Z is C₃ alkylene, substituted with Me, Et, i-Pr, hydroxy, or hydroxymethyl.

20. The compound according any one of claims 1-16, wherein Z is —CH₂—C(Me)₂-CH₂—.

21. The compound according any one of claims 1-20, wherein each dotted bond is a single bond.

22. The compound according any one of claims 1-20, wherein the compound is according to formula (IIa) or (IIb): 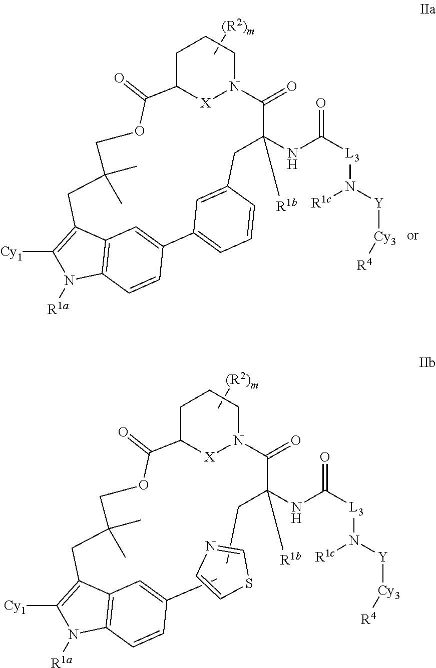
or a stereoisomer or a pharmaceutically acceptable salt thereof.

23. The compound according any one of claims 1-21, wherein L₃ is —CH₂—, —C(Me)H—, —C(Et)H—, —C(n-Pr)H—, —C(i-Pr)H—, or —C(Me)₂-.

24. The compound according any one of claims 1-21, wherein L₃ is —C(i-Pr)H—.

25. The compound according any one of claims 1-23, wherein Y is —C(O)—.

26. The compound according any one of claims 1-23, wherein the compound is according to formula (IIIa) or (IIIb): 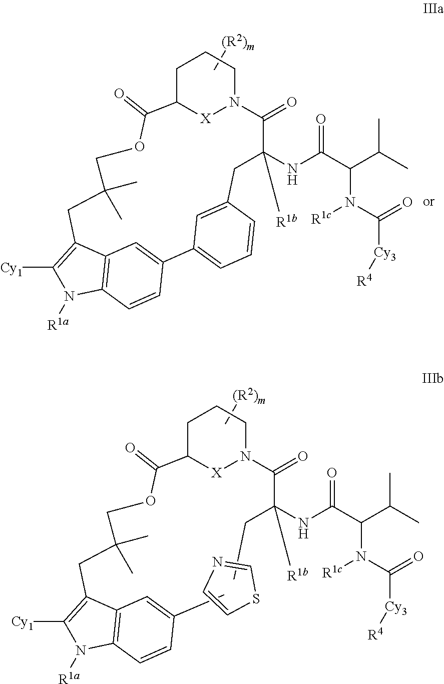
or a stereoisomer or a pharmaceutically acceptable salt thereof.

27. The compound according any one of claims 1-25, wherein R^{1b} is H, Me, or Et.

28. The compound according any one of claims 1-25, wherein R^{1b} is H, or Me.

29. The compound according any one of claims 1-25, wherein R^{1b} is H.

30. The compound according any one of claims 1-25, wherein R^{1c} is H, Me, or Et.

31. The compound according any one of claims 1-25, wherein R^{1c} is H, or Me.

32. The compound according any one of claims 1-25, wherein R^{1c} is Me.

33. The compound according any one of claims 1-31, wherein the compound is according to formula (IVa) or (IVb): 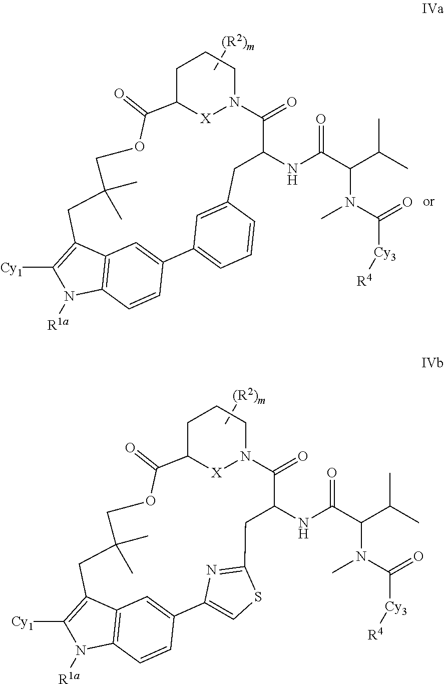
or a stereoisomer or a pharmaceutically acceptable salt thereof.

34. The compound according any one of claims 1-32, wherein R^{1a} is H, Me, Et, or i-Pr.

35. The compound according any one of claims 1-32, wherein R^{1a} is H, Me, or Et.

36. The compound according any one of claims 1-32, wherein R^{1a} is Et.

37. The compound according any one of claims 1-35, wherein m is 1, or 2.

38. The compound according any one of claims 1-35, wherein m is 1, or 2; and each R² is independently alkyl or haloalkyl.

39. The compound according any one of claims 1-35, wherein m is 1, or 2; and each R² is independently Me or CF₃.

40. The compound according any one of claims 1-35, wherein m is 0.

41. The compound according any one of claims 1-39, wherein the compound is according to formula (Va) or (Vb): 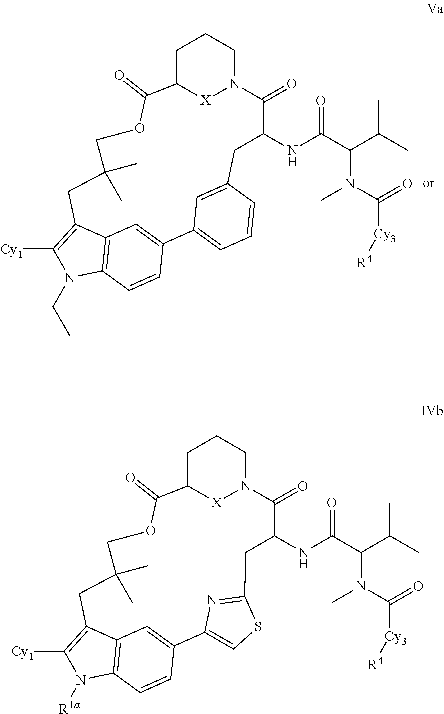
or a stereoisomer or a pharmaceutically acceptable salt thereof.

42. The compound according any one of claims 1-40, wherein Cy₁ is substituted or unsubstituted pyrrolyl, furanyl, imidazolyl, thiazolyl or thiadiazolyl.

43. The compound according any one of claims 1-40, wherein Cy₁ is substituted or unsubstituted phenyl, pyridyl, naphthalenyl, pyrimidinyl, or pyrazinyl.

44. The compound according any one of claims 1-40, wherein Cy₁ is substituted or unsubstituted phenyl, or pyridyl.

45. The compound according any one of claims 1-40, wherein Cy₁ is substituted or unsubstituted pyridyl.

46. The compound according any one of claims 1-40, wherein Cy₁ is pyridyl, substituted with Me, Et, i-Pr, n-Pr, t-Bu, CF₃, OMe, OCF₃, CHF₂, CH₂OMe, CH₂—CH₂OMe, or C(OMe)(Me)H.

47. The compound according any one of claims 1-45, wherein the compound is according to formula (VIa) or (VIb): 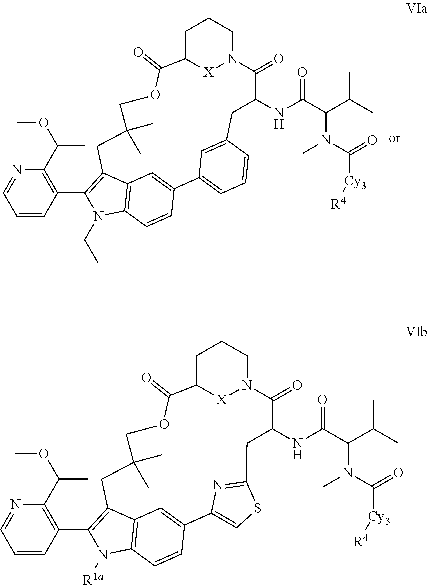
or a stereoisomer or a pharmaceutically acceptable salt thereof.

48. The compound according any one of claims 1-46, wherein X is NR^{1g}, and R^{1g} is H, Me, Et, or i-Pr.

49. The compound according any one of claims 1-46, wherein X is NR^{1g}, and R^{1g} is H.

50. The compound according to any one of claims 1-46, wherein X is —NR^{1g}—, R^{1g} is H, each dotted bond is a single bond, t is 1; R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

51. The compound according any one of claims 1-49, wherein the compound is according to formula (VIIa) or (VIIb): 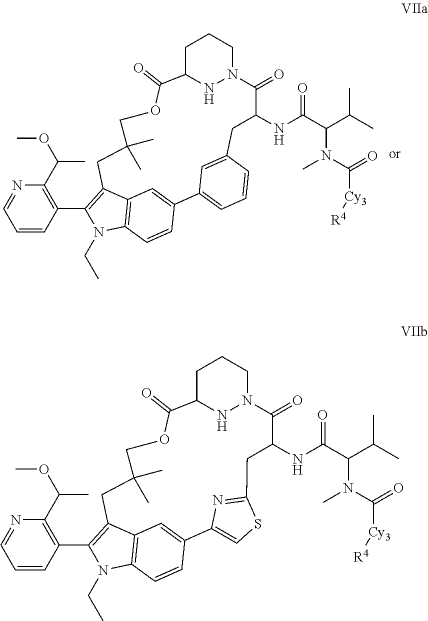
or a stereoisomer or a pharmaceutically acceptable salt thereof, and wherein R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

52. The compound according any one of claims 1-51, wherein X is CR^{1e}R^{1f}, and each R^{1e} and R^{1f} is independently H, Me, Et, or i-Pr.

53. The compound according any one of claims 1-51, wherein X is CR^{1e}R^{1f}, and each R^{1e} and R^{1f} is H.

54. The compound according any one of claims 1-51, wherein the compound is according to formula (VIIIa) or (VIIIb): 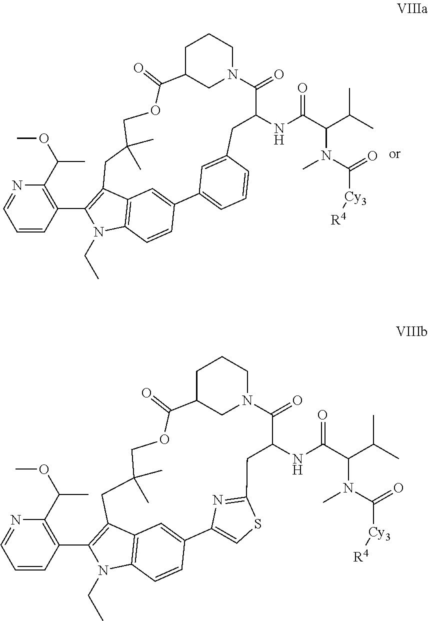
or a stereoisomer or a pharmaceutically acceptable salt thereof, and wherein R⁴ is —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl; -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}.

55. The compound according any one of claims 1-54, wherein Cy³ is substituted or unsubstituted cycloalkylene.

56. The compound according any one of claims 1-54, wherein Cy³ is substituted or unsubstituted cyclopropyl, cyclobutyl, cyclopentyl or cyclohexyl.

57. The compound according any one of claims 1-54, wherein Cy³ is substituted or unsubstituted N containing C₃-C₁₁ heterocycloalkyl.

58. The compound according any one of claims 1-54, wherein Cy³ is substituted or unsubstituted N containing C₃-C₁₁ heterocycloalkyl; and R⁴ is attached to the N.

59. The compound according any one of claims 1-54, wherein Cy³ is substituted or unsubstituted N containing C₃-C₁₁ heterocycloalkyl, and the heterocycloalkyl is a monocyclic, bicyclic, bridged, fused, or partially saturated heterocyclic ring.

60. The compound according any one of claims 1-54, wherein Cy³ is substituted or unsubstituted pyrrolidinyl, piperidinyl, piperazinyl, morpholinyl, aziridinyl, azetidinyl, azepinyl, diazepinyl,

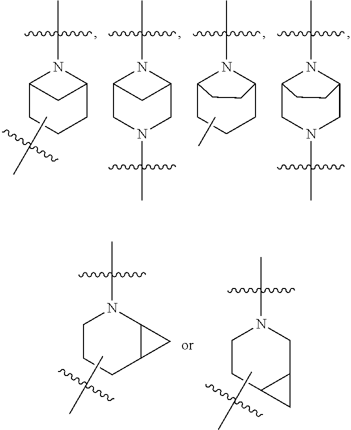

61. The compound according any one of claims 1-54, wherein Cy³ is substituted or unsubstituted pyrrolidinyl, piperidinyl, piperazinyl, or morpholinyl.

62. The compound according any one of claims 1-54, wherein Cy³ is substituted or unsubstituted pyrrolidinyl.

63. The compound according any one of claims 1-62, wherein the compound is according to formula (IXa), (IXb), (IXc) or (IXd): 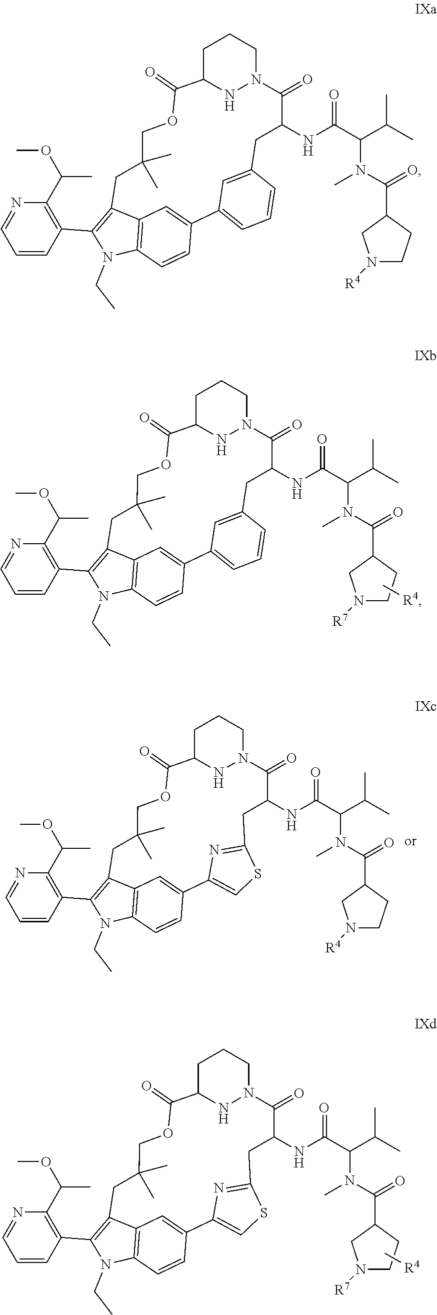
or a stereoisomer or a pharmaceutically acceptable salt thereof; and wherein R⁴ is -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—CR^{6e}; and R7 is H, or C1-C4 alkyl.

64. The compound according any one of claims 1-62, wherein the compound is according to formula (Xa), (Xb), (Xc), or (Xd): 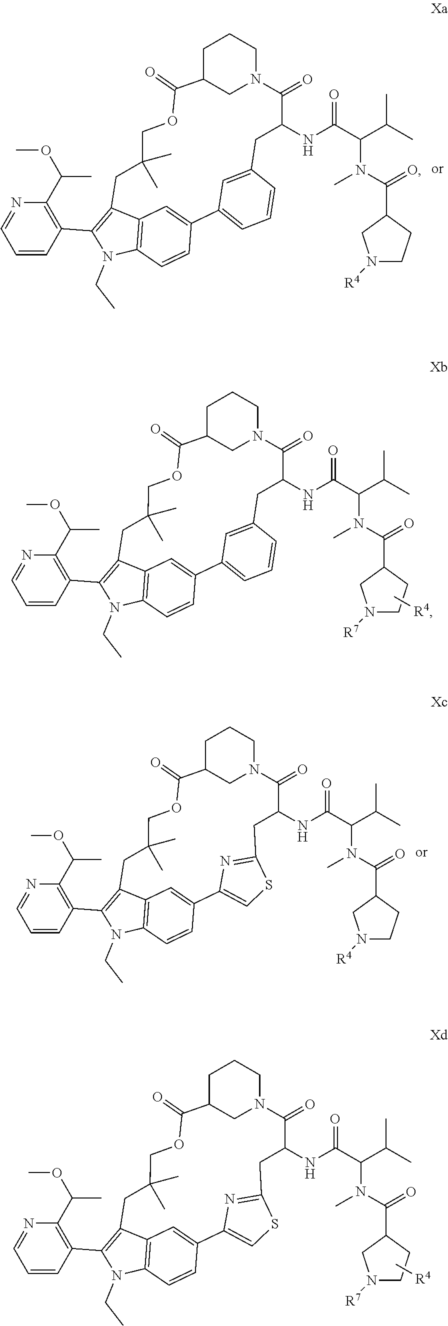
or a stereoisomer or a pharmaceutically acceptable salt thereof, and wherein R⁴ is —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl; -L₄-R^{6d}, -L₄-C(R^{6a})═C(R^{6b})—C(O)—R^{6d}, -L₄-C(R^{6a})═C(R^{6b})—S(O)—R^{6d}, -L₄-C(R^{6a})═C(R^{6b})—S(O)₂—R^{6d}; -L₄-C≡C—R^{6e}, or -L₄-C≡C—R^{6e}; and R7 is H, or C1-C4 alkyl.

65. The compound according to any one of claims 63-64, wherein R⁷ is H, Me, Et, or i-Pr.

66. The compound according to any one of claims 63-64, wherein R⁷ is Me.

67. The compound according to any one of claims 1-66, wherein R⁴ is -L₄-R^{6d}.

68. The compound according to any one of claims 1-66, wherein R⁴ is -L₄-C≡C—R^{6e}.

69. The compound according to any one of claims 1-66, wherein L₄ is —CH₂—, —CH₂—CH₂—, —CH₂—CH₂—CH₂—, —C(O)—, —C(Me)H, —CMe₂-, or

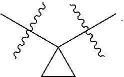 [SMILES: *C1(*)CC1]

70. The compound according to any one of claims 1-66, wherein L₄ is —CH₂—.

71. The compound according to any one of claims 1-66, wherein L₄ is —C(O)—.

72. The compound according any one of claims 1-71, wherein the compound is according to formula (XIa), (XIb), or (XIc): 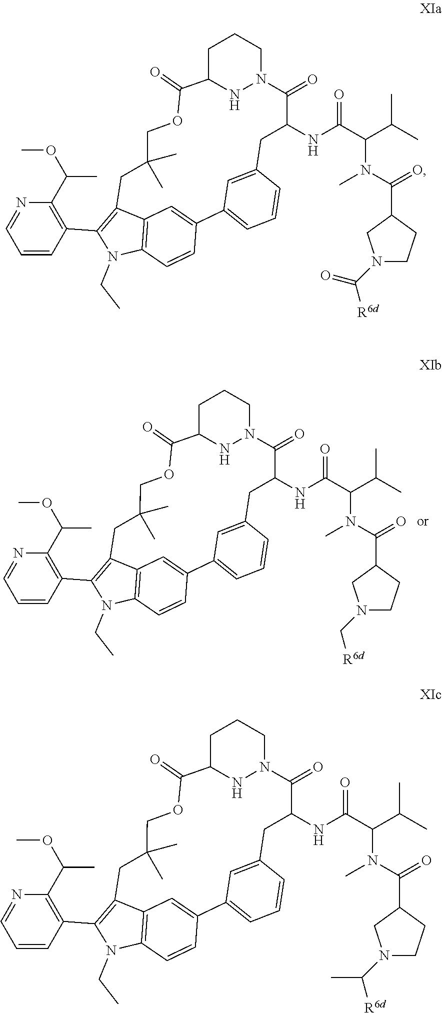
or a stereoisomer or a pharmaceutically acceptable salt thereof.

73. The compound according any one of claims 1-72, wherein the compound is according to formula (XIIa), (XIIb), or (XIIc): 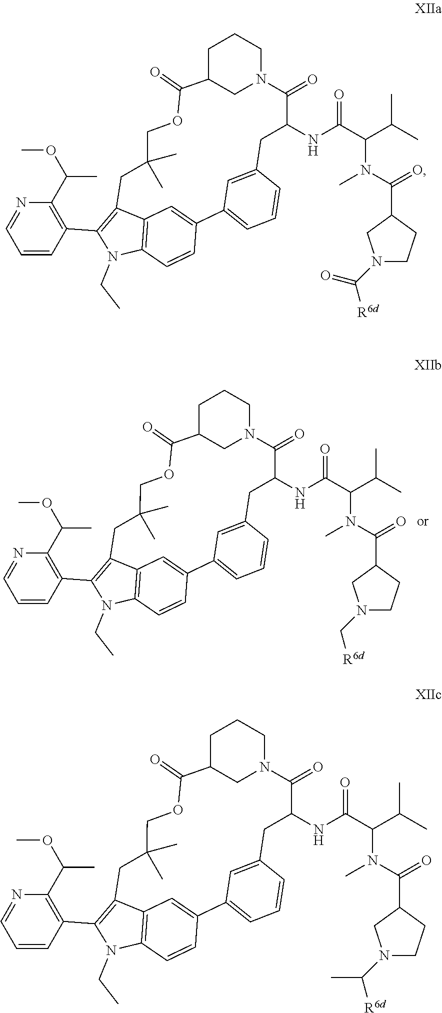
or a stereoisomer or a pharmaceutically acceptable salt thereof.

74. The compound according any one of claims 1-73, wherein R^{6d} is

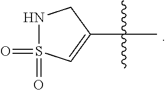 [SMILES: *C1=CS(=O)(=O)NC1]

75. The compound according any one of claims 1-73, wherein the compound is according to formula (XIIIa) or (XIIIb): 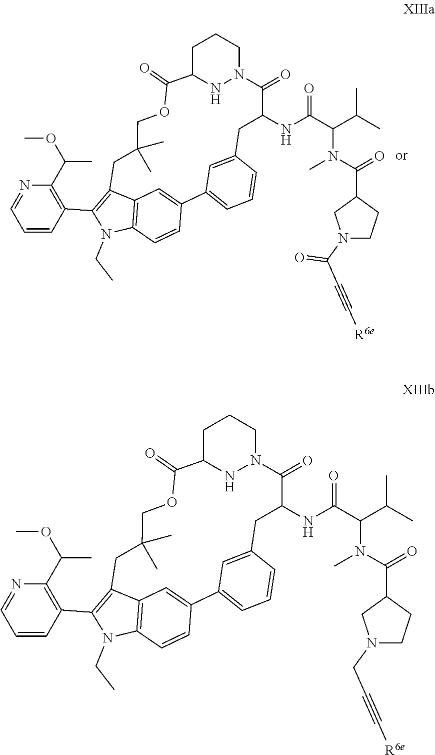
or a stereoisomer or a pharmaceutically acceptable salt thereof.

76. The compound according any one of claims 1-73, wherein the compound is according to formula (XIVa) or (XIVb): 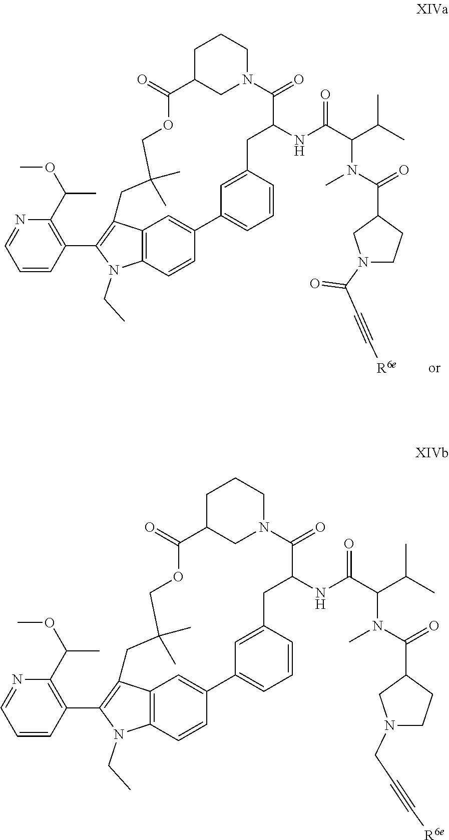
or a stereoisomer or a pharmaceutically acceptable salt thereof.

77. The compound according any one of claims 1-76, wherein R^{6e} is substituted or unsubstituted alkyl.

78. The compound according any one of claims 1-76, wherein R^{6e} is Me, Et.

79. The compound according any one of claims 1-76, wherein R^{6e} is substituted or unsubstituted aryl, substituted or unsubstituted heteroaryl.

80. The compound according any one of claims 1-76, wherein R^{6e} is substituted or unsubstituted heteroaryl.

81. The compound according any one of claims 1-76, wherein R^{6e} is substituted or unsubstituted benzoxazolyl, benzthiazolyl, benzimidazolyl, indazolyl, or indolyl.

82. The compound according any one of claims 1-76, wherein R^{6e} is substituted or unsubstituted benzoxazolyl.

83. The compound according to any one of claims 1-82, wherein R⁴ is -L₄-C(R^{6a})═C(R^{6b})—C(O)—R^{6d}, -L₄-C(R^{6a})═C(R^{6b})—S(O)—R^{6d}, or -L₄-C(R^{6a})═C(R^{6b})—S(O)₂—R^{6d}; and L₄ is —CH₂—, —CH₂—CH₂—, —CH₂—CH₂—CH₂—, —C(O)—, —C(Me)H, —CMe₂-, or

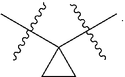 [SMILES: *C1(*)CC1]

84. The compound according to any one of claims 1-82, wherein R⁴ is —CH₂—C(R^{6a})═C(R^{6b})—C(O)—R^{6d}, —CH₂—C(R^{6a})═C(R^{6b})—S(O)—R^{6d}, or —CH₂—C(R^{6a})═C(R^{6b})—S(O)₂—R^{6d}.

85. The compound according to any one of claims 1-82, wherein R⁴ is —C(O)—C(R^{6a})═C(R^{b})—C(O)—R^{6d}, —C(O)—C(R^{6a})═C(R^{6b})—S(O)—R^{6d}, or —C(O)—C(R^{6a})═C(R^{6b})—S(O)₂—R^{6d}.

86. The compound according to any one of claims 1-85, wherein each of R^{6a}, and R^{6b} is H.

87. The compound according to any one of claims 1-85, wherein each of R^{6a} and R^{6b} is H or F.

88. The compound according to any one of claims 1-85, wherein one of R^{6a} and R^{6b} is CN.

89. The compound according to any one of claims 1-66, wherein R⁴ is —CH₂—CH═CH—C(O)—R^{6d}, —CH₂—CH═CH—S(O)—R^{6d}, or —CH₂—CH═CH—S(O)₂—R^{6d}.

90. The compound according to any one of claims 1-66, wherein R⁴ is —C(Me)H—CH═CH—C(O)—R^{6d}, —C(Me)H—CH═CH—S(O)—R^{6d}, or —C(Me)H—CH═CH—S(O)₂—R^{6d}.

91. The compound according to any one of claims 1-66, wherein R⁴ is —C(O)—CH═CH—C(O)—R^{6d}, —C(O)—CH═CH—S(O)—R^{6d}, or —C(O)—CH═CH—S(O)₂—R^{6d}.

92. The compound according any one of claims 1-91, wherein the compound is according to formula (XVa), (XVb), (XVc), or (XVd): 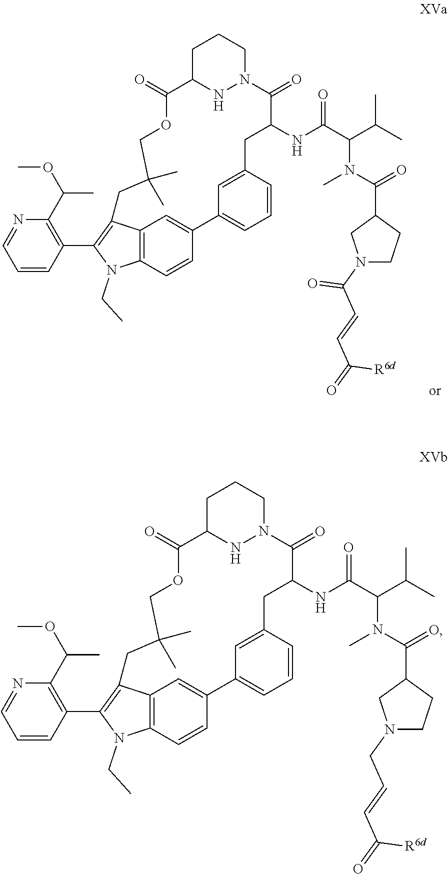 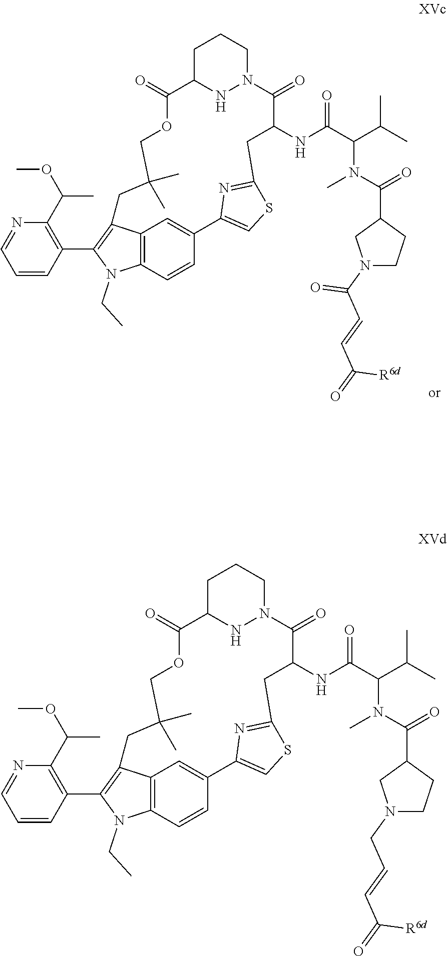
or a stereoisomer or a pharmaceutically acceptable salt thereof.

93. The compound according any one of claims 1-91, wherein the compound is according to formula (XVIa), (XVIb), (XVIc), or (XVId): 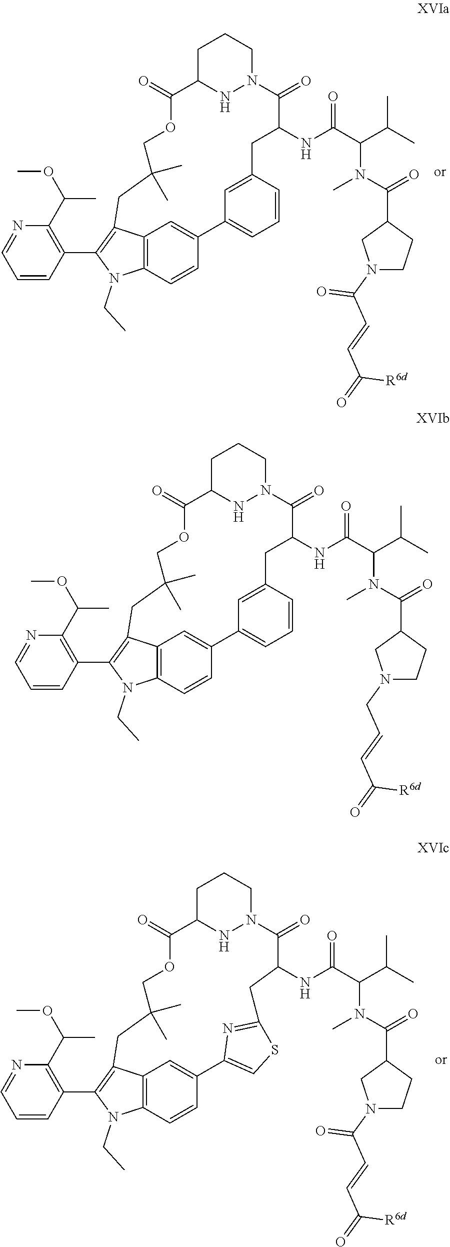 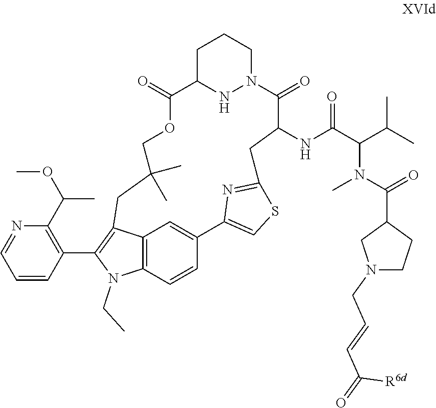
or a stereoisomer or a pharmaceutically acceptable salt thereof.

94. The compound according to any one of claims 1-93, wherein R^{6a} and R^{6b} form a bond.

95. The compound according to any one of claims 1-93, wherein R⁴ is —CH₂—C═C≡C(O)—R^{6d}, —CH₂—C≡C—S(O)—R^{6d}, or —CH₂—C≡C—S(O)₂—R^{6d}.

96. The compound according to any one of claims 1-93, wherein R⁴ is —C(Me)H—C═C≡C(O)—R^{6a}, —C(Me)H—C≡C—S(O)—R^{6d}, or —C(Me)H—C≡C—S(O)₂—R^{6d}.

97. The compound according to any one of claims 1-93, wherein R⁴ is —C(O)—C═C≡C(O)—R^{6d}, —C(O)—C≡C—S(O)—R^{6d}, or —C(O)—C≡C—S(O)₂—R^{6d}.

98. The compound according any one of claims 1-97, wherein the compound is according to formula (XVIIa), (XVIIb), (XVIIc), or (XVIId): 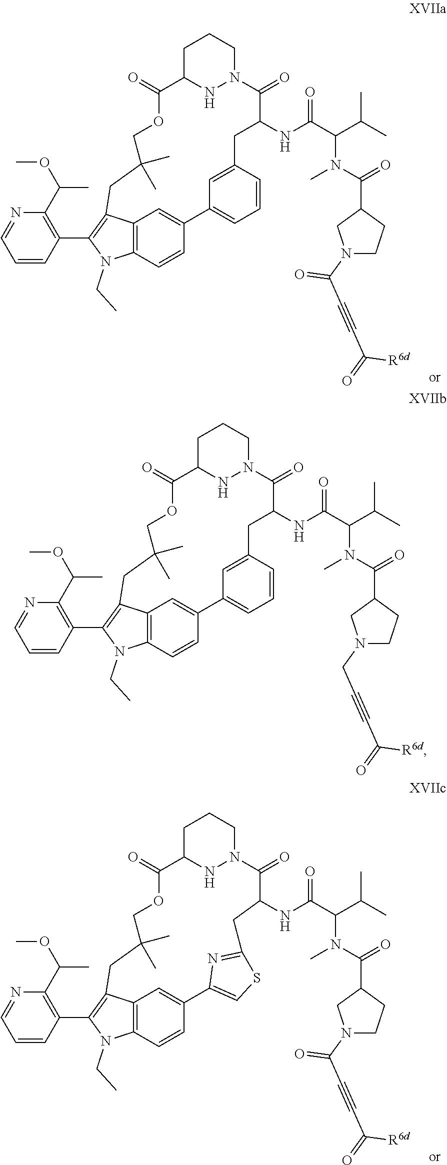 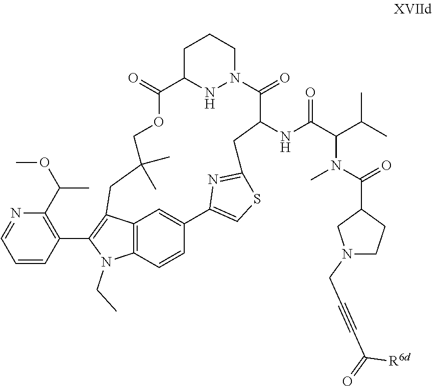
or a stereoisomer or a pharmaceutically acceptable salt thereof.

99. The compound according any one of claims 1-97, wherein the compound is according to formula (XVIIIa) or (XVIIIb): 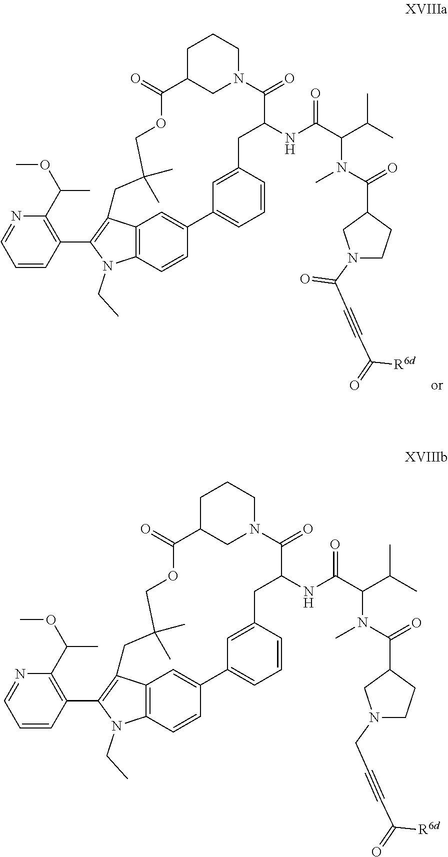
or a stereoisomer or a pharmaceutically acceptable salt thereof.

100. The compound according any one of claims 1-99, wherein R^{6d} is substituted or unsubstituted amino, substituted or unsubstituted heterocycloalkyl having 1-2 heteroatoms independently selected from nitrogen, oxygen, and sulfur, substituted or unsubstituted aryl, substituted or unsubstituted heteroaryl, or

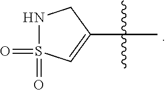 [SMILES: *C1=CS(=O)(=O)NC1]

101. The compound according any one of claims 1-99, wherein R^{6d} is

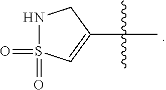 [SMILES: *C1=CS(=O)(=O)NC1]

102. The compound according any one of claims 1-99, wherein R^{6d} is substituted or unsubstituted amino.

103. The compound according any one of claims 1-99, wherein R^{6d} is dialkylamino.

104. The compound according any one of claims 1-99, wherein R^{6d} is dimethylamino.

105. The compound according any one of claims 1-99, wherein R^{6d} is substituted or unsubstituted heterocycloalkyl having 1-2 heteroatoms independently selected from nitrogen, oxygen, and sulfur.

106. The compound according any one of claims 1-99, wherein R^{6d} is substituted or unsubstituted pyrrolidinyl, piperidinyl, piperizinyl, or morpholinyl.

107. The compound according any one of claims 1-99, wherein R^{6d} is substituted or unsubstituted pyrrolidinyl.

108. The compound according any one of claims 1-99, wherein R^{6d} is substituted or unsubstituted morpholinyl.

109. The compound according any one of claims 1-99, wherein R^{6d} is substituted or unsubstituted piperidinyl.

110. The compound according any one of claims 1-99, wherein R^{6d} is substituted or unsubstituted aryl.

111. The compound according any one of claims 1-99, wherein R^{6d} is substituted or unsubstituted heteroaryl.

112. The compound according any one of claims 1-111, wherein the compound is according to formula (X): 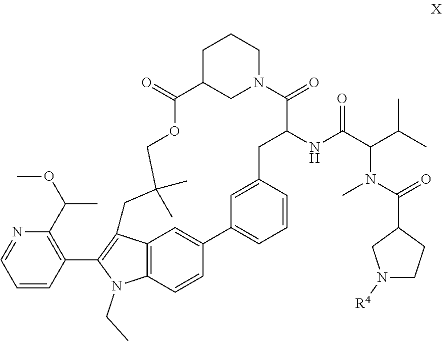
or a stereoisomer or a pharmaceutically acceptable salt thereof, and wherein R⁴ is —C(O)—C(R^{6a})═C(R^{6b})(R^{6c}), —S(O)—C(R^{6a})═C(R^{6b})(R^{6c}), —S(O)₂—C(R^{6a})═C(R^{6b})(R^{6c}), or S(O)₂—CH₂—Cl.

113. The compound according to any one of claims 1-112, wherein R⁴ is —C(O)—C(R^{6a})═C(R^{6b})(R^{6c}).

114. The compound according to any one of claims 1-112, wherein R⁴ is —S(O)₂—C(R^{6a})═C(R^{6b})(R^{6c}).

115. The compound according to any one of claims 1-112, wherein R⁴ is S(O)₂—CH₂—Cl.

116. The compound according any one of claims 1-115, wherein the compound is according to formula (XXIa) or (XXIb): 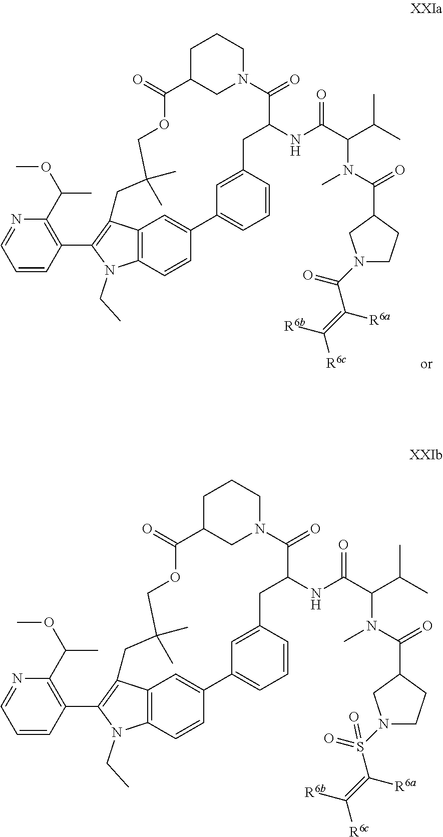
or a stereoisomer or a pharmaceutically acceptable salt thereof.

117. The compound according to any one of claims 1-116, wherein each of R^{6a}, and R^{6b} is H.

118. The compound according to any one of claims 1-116, wherein one of R^{6a}, and R^{6b} is F and the other is H.

119. The compound according to any one of claims 1-116, wherein one of R^{6a}, and R^{6b} is alkyl and the other is H.

120. The compound according to any one of claims 1-116, wherein one of R^{6a}, and R^{6b} is Me or Et, and the other is H.

121. The compound according to any one of claims 1-116, wherein one of R^{6a}, and R^{6b} is CN and the other is H.

122. The compound according any one of claims 1-116, wherein the compound is according to formula (XXIIa) or (XXIIb): 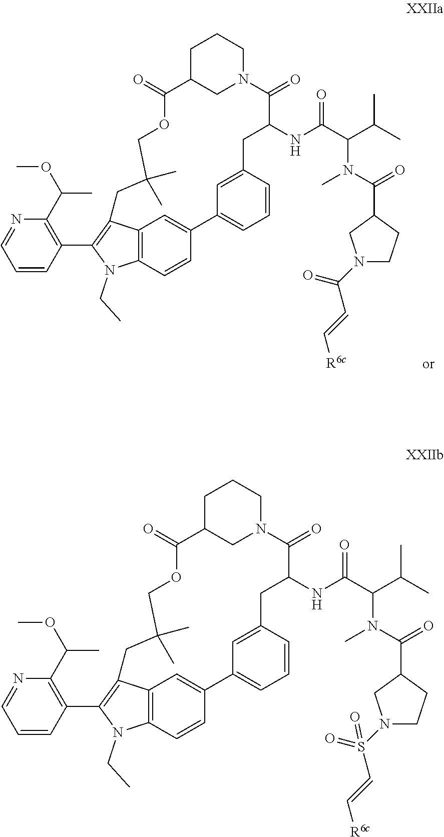
or a stereoisomer or a pharmaceutically acceptable salt thereof.

123. The compound according to any one of claims 1-122, wherein R^{6a} and R^{6b} form a bond.

124. The compound according any one of claims 1-116, wherein the compound is according to formula (XXIIIa) or (XXIIIb): 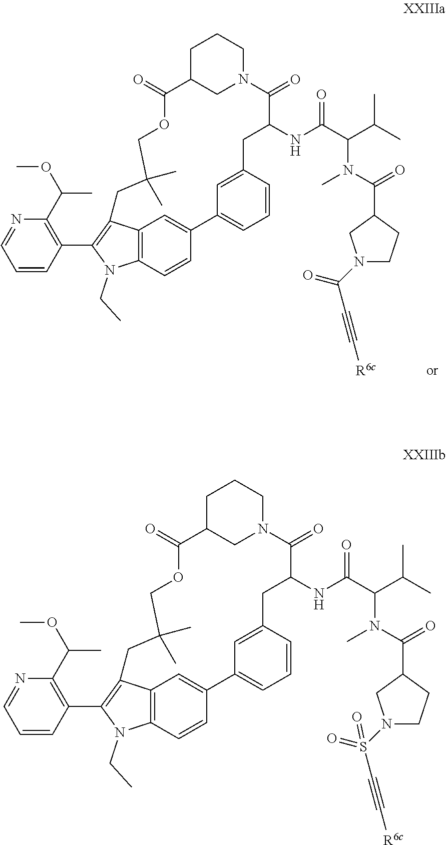
or a stereoisomer or a pharmaceutically acceptable salt thereof.

125. The compound according to any one of claims 1-124, wherein R^{6c} is H.

126. The compound according to any one of claims 1-124, wherein R^{6c} is substituted or unsubstituted alkyl.

127. The compound according to any one of claims 1-124, wherein R^{6c} is H, or substituted or unsubstituted alkyl.

128. The compound according to any one of claims 1-124, wherein R^{6c} is unsubstituted alkyl.

129. The compound according to any one of claims 1-124, wherein R^{6c} is Me, or Et.

130. The compound according to any one of claims 1-124, wherein R^{6c} is Me.

131. The compound according to any one of claims 1-124, wherein R^{6c} is substituted alkyl.

132. The compound according to any one of claims 1-124, wherein R^{6c} is alkyl substituted with amino, alkylamino or dialkylamino.

133. The compound according to any one of claims 1-124, wherein R^{6c} is alkyl substituted with dimethylamino.

134. The compound according to any one of claims 1-124, wherein R^{6c} is —CH₂NMe₂.

135. The compound according to any one of claims 1-124, wherein R^{6c} is alkyl substituted with hydroxy, CN, or substituted or unsubstituted alkoxy.

136. The compound according to any one of claims 1-124, wherein R^{6c} is alkyl substituted with OH, OMe, CN, or OCF3.

137. The compound according to any one of claims 1-124, wherein R^{6c} is alkyl substituted with cycloalkyl.

138. The compound according to any one of claims 1-124, wherein R^{6c} is alkyl substituted with cyclopropyl, cyclobutyl, cyclopentyl or cyclohexyl.

139. The compound according to any one of claims 1-124, wherein R^{6c} is alkyl substituted with heterocycloalkyl.

140. The compound according to any one of claims 1-124, wherein R^{6c} is —(CH₂)_{q}-heterocycloalkyl; and q is 1, 2, 3, or 4.

141. The compound according to any one of claims 1-124, wherein R^{6c} is —(CH₂)_{q}-heterocycloalkyl; and q is 1.

142. The compound according to any one of claims 1-124, wherein R^{6c} is —(CH₂)_{q}-heterocycloalkyl; and q is 2.

143. The compound according to any one of claims 1-124, wherein R^{6c} is —(CH₂)_{q}-heterocycloalkyl; and q is 3.

144. The compound according to any one of claims 1-124, wherein R^{6c} is —CH₂-azetidin-1-yl, —CH₂-pyrrolidin-1-yl, —CH₂-piperidin-1-yl, or —CH₂-morpholin-1-yl.

145. The compound according any one of claims 1-144, wherein the compound is according to formula (XXIVa) or (XXIVb): 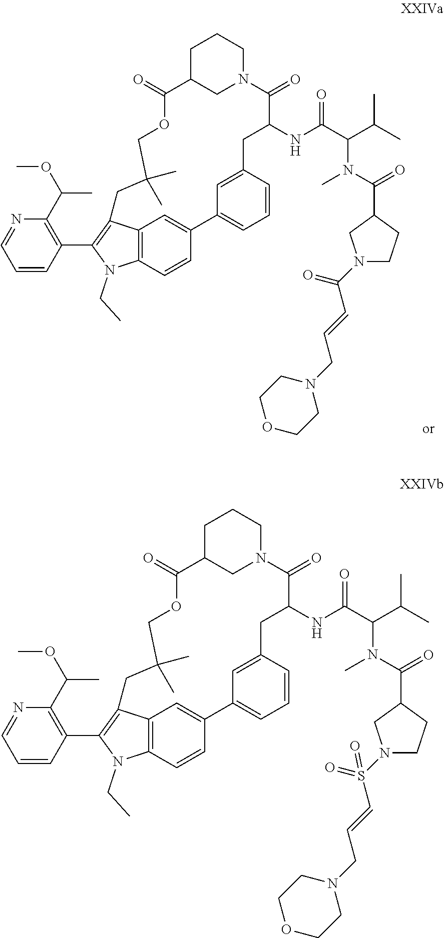 [SMILES: CCn1c(-c2cccnc2C(C)OC)c2c3cc(ccc31)-c1cccc(c1)CC(NC(=O)C(C(C)C)N(C)C(=O)C1CCN(C(=O)/C=C/CN3CCOCC3)C1)C(=O)N1CCCC(C1)C(=O)OCC(C)(C)C2 ; CCn1c(-c2cccnc2C(C)OC)c2c3cc(ccc31)-c1cccc(c1)CC(NC(=O)C(C(C)C)N(C)C(=O)C1CCN(S(=O)(=O)/C=C/CN3CCOCC3)C1)C(=O)N1CCCC(C1)C(=O)OCC(C)(C)C2]
or a stereoisomer or a pharmaceutically acceptable salt thereof.

146. The compound according any one of claims 1-144, wherein the compound is according to formula (XXVa) or (XXVb): 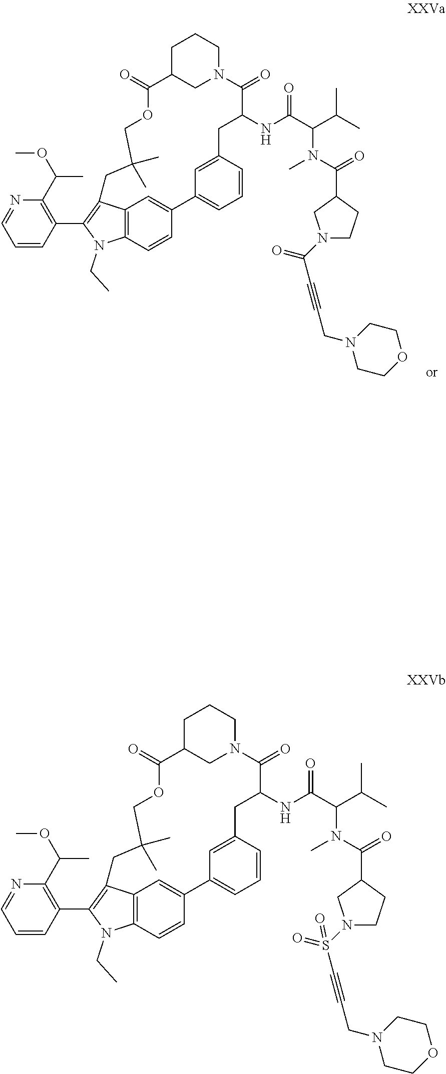 [SMILES: CCn1c(-c2cccnc2C(C)OC)c2c3cc(ccc31)-c1cccc(c1)CC(NC(=O)C(C(C)C)N(C)C(=O)C1CCN(C(=O)C#CCN3CCOCC3)C1)C(=O)N1CCCC(C1)C(=O)OCC(C)(C)C2 ; CCn1c(-c2cccnc2C(C)OC)c2c3cc(ccc31)-c1cccc(c1)CC(NC(=O)C(C(C)C)N(C)C(=O)C1CCN(S(=O)(=O)C#CCN3CCOCC3)C1)C(=O)N1CCCC(C1)C(=O)OCC(C)(C)C2]
or a stereoisomer or a pharmaceutically acceptable salt thereof.

147. The compound according claim 1, wherein the compound is according to formula (XXVIa), (XXVIb), (XXVIc), or (XXVId): 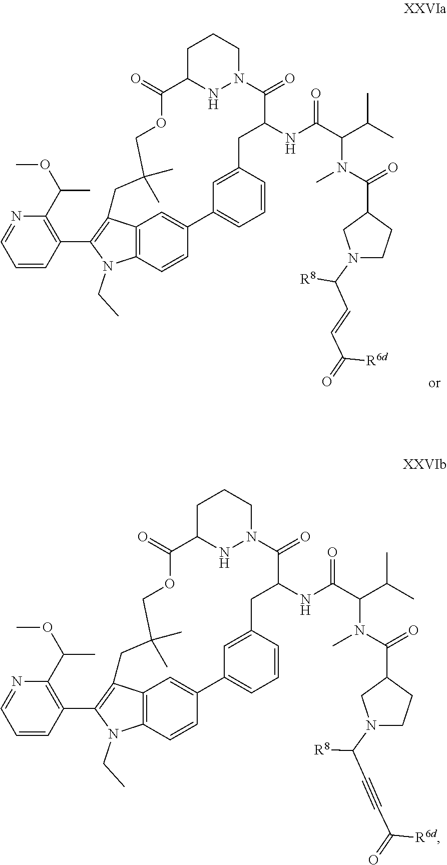 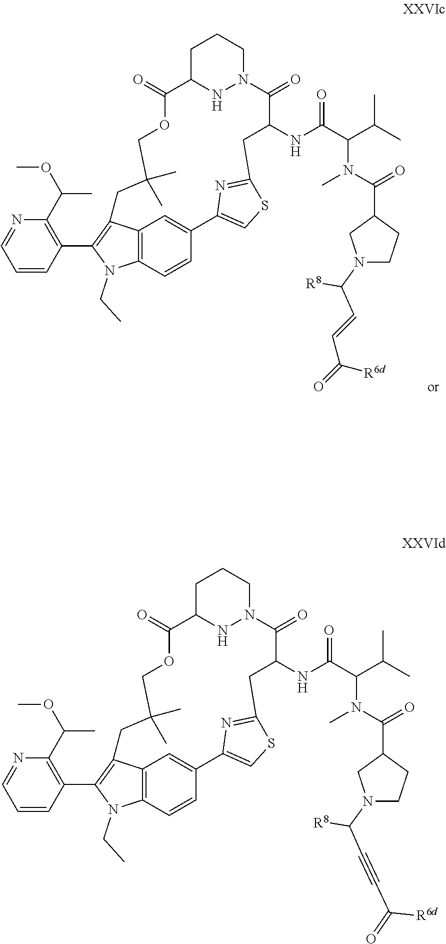
or a stereoisomer or a pharmaceutically acceptable salt thereof; wherein
R⁸ is H, or C₁-C₄ alkyl; and R^{6d} is alkylamino or dialkylamino, or a substituted or unsubstituted heterocycle.

148. The compound according claim 1, wherein the compound is according to formula (XXVIIa), (XXVIIb), (XXVIIc), or (XXVIId): 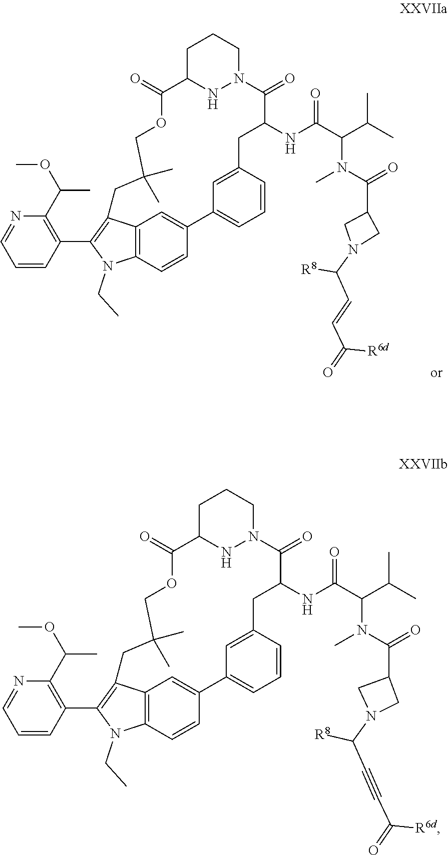 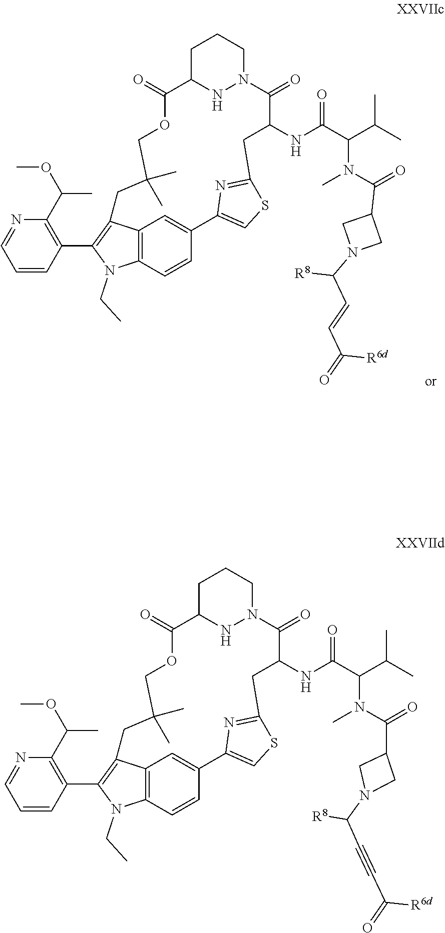
or a stereoisomer or a pharmaceutically acceptable salt thereof, wherein
R⁸ is H, or C₁-C₄ alkyl; and R^{6d} is alkylamino or dialkylamino, or a substituted or unsubstituted heterocycle.

149. The compound according claim 1, wherein the compound is according to formula (XXVIIIa), (XXVIIIb), (XXVIIIc), or (XXVIIId): 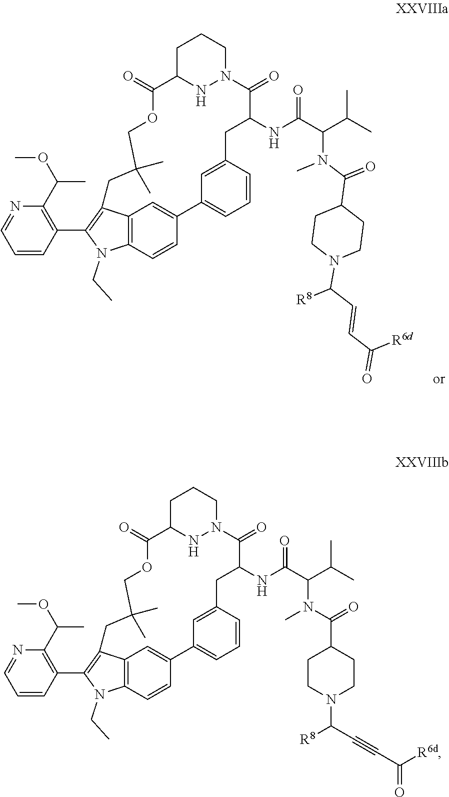 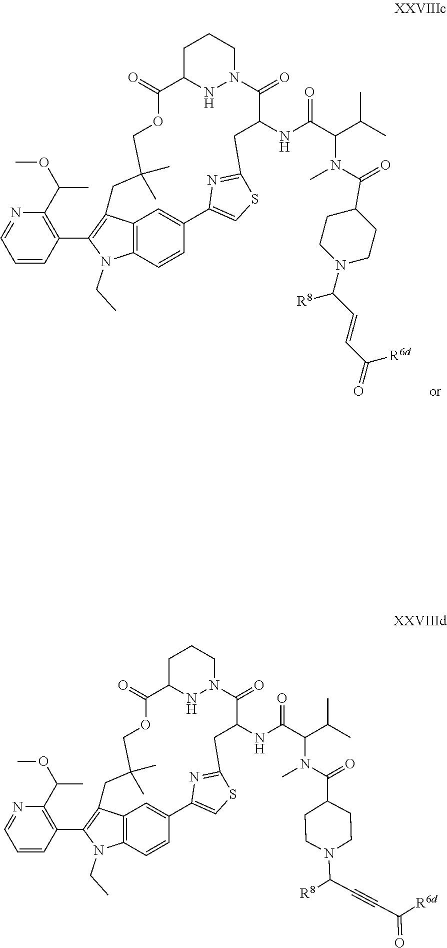
or a stereoisomer or a pharmaceutically acceptable salt thereof, wherein
R⁸ is H, or C₁-C₄ alkyl; and R^{6d} is alkylamino or dialkylamino, or a substituted or unsubstituted heterocycle.

150. The compound according to any one of claims 147-149, wherein R^{6d} is NHMe, or NMe₂.

151. The compound according to any one of claims 147-149, wherein R^{6d} is substituted or unsubstituted heterocycle.

152. The compound according to any one of claims 147-149, wherein R^{6d} is substituted or unsubstituted azetidinyl.

153. The compound according to any one of claims 147-149, wherein R^{6d} is azetidinyl, substituted with F or diF.

154. The compound according to any one of claims 147-149, wherein R^{6d} is 3-fluoroazetidinyl, or 3,3-difluoro azetidinyl.

155. The compound according to any one of claims 147-154, wherein R^{8d} is H.

156. The compound according to any one of claims 147-154, wherein R^{8d} is Me.

157. The compound according to claim 1, wherein the compound is selected from any one of compounds listed in Table 1A and Table 1B.

158. The compound according to claim 1, wherein the compound is any compound selected from Table 1A and Table 1B; or a pharmaceutically acceptable stereoisomer or salt thereof.

159. A pharmaceutical composition comprising a therapeutically effective amount of a compound of any one of claims 1-158; or a pharmaceutically acceptable salt, solvate, or prodrug thereof, and a pharmaceutically acceptable excipient.

160. The pharmaceutical composition of claim 159 that is formulated for a route of administration selected from oral administration, parenteral administration, buccal administration, nasal administration, topical administration, or rectal administration.

161. A method for treating an autoimmune disease or condition comprising administering to a patient in need thereof a therapeutically effective amount of the pharmaceutical composition of claim 159 or 161.

162. A method for treating a heteroimmune disease or condition comprising administering to a patient in need thereof the pharmaceutical composition of claim 159 or 160.

163. A method for treating a cancer comprising administering to a patient in need thereof a therapeutically effective amount of the pharmaceutical composition of claim 159 or 160.

164. The method of claim 163, wherein the cancer is a B-cell proliferative disorder.

165. The method of claim 164, wherein the B-cell proliferative disorder is diffuse large B cell lymphoma, follicular lymphoma, chronic lymphocytic leukemia, lymphoid leukemia, ALL, soft tissue tumor, Glioblastoma, pancreatic tumor or renal cell cancer.

166. The use of a compound, or a metabolite, a solvate, a pharmaceutically acceptable salt, or a prodrug thereof, according to any one of claims 1-158, or a pharmaceutical composition of either of claims 159 or 160, in the manufacture of a medicament.

167. A compound, or a metabolite, a solvate, a pharmaceutically acceptable salt, or a prodrug thereof, according to any one of claims 1-158, or a pharmaceutical composition of either of claims 159 or 160, for use as a medicament.

168. A compound, or a metabolite, a solvate, a pharmaceutically acceptable salt, or a prodrug thereof, according to any one of claims 1-158, or a pharmaceutical composition of either of claims 159 or 160, for use in the treatment, prevention or prophylaxis of autoimmune diseases, heteroimmune diseases, proliferative diseases, and inflammatory conditions.

169. A compound, or a metabolite, a solvate, a pharmaceutically acceptable salt, or a prodrug thereof, according to any one of claims 1-158, or a pharmaceutical composition of either of claims 159 or 160, for use in the treatment, prevention or prophylaxis of cancer, mastocytosis, and B-cell lymphoma.

170. The use of a compound, or a metabolite, a solvate, a pharmaceutically acceptable salt, or a prodrug thereof, according to any one of claims 1-158 in the preparation of a medicament for the treatment, prevention or prophylaxis of autoimmune diseases, heteroimmune diseases, proliferative diseases, and inflammatory conditions.

171. The use of a compound, or a metabolite, a solvate, a pharmaceutically acceptable salt, or a prodrug thereof, according to any one of claims 1-158 in the preparation of a medicament for the treatment, prevention or prophylaxis of cancer, mastocytosis, B-cell lymphoma, lupus, and osteoporosis/bone resorption.

172. The compound according to any one of claims 1-158, or the pharmaceutical composition of claim 159 or 160, or the method according to any one of claims 165-169, or the use according to any one of claims 160-161, wherein the compound is an inhibitor of KRas G12C.

173. The compound according to any one of claims 1-158, or the pharmaceutical composition of claim 159 or 160, or the method according to any one of claims 165-169, or the use according to any one of claims 170-171, wherein the compound is an inhibitor of KRas G12D.
